SEQUENCE LISTING (136 sequences)

SEQ ID NO: 1 - DNA, 35 bp, synthetic construct
  source 1..35: mol_type = other DNA
nnnnnnnnnnnnnnnnnwwwwwwaggagnnnnaaa

SEQ ID NO: 2 - DNA, 18 bp, synthetic construct
  source 1..18: mol_type = other DNA
aatatcatatagaagtca

SEQ ID NO: 3 - DNA, 18 bp, synthetic construct
  source 1..18: mol_type = other DNA
tagaagtcatcgaaatag

SEQ ID NO: 4 - DNA, 18 bp, synthetic construct
  source 1..18: mol_type = other DNA
agatcatcaaggaagtaa

SEQ ID NO: 5 - DNA, 18 bp, synthetic construct
  source 1..18: mol_type = other DNA
atcaaaacaaataaaaca

SEQ ID NO: 6 - DNA, 18 bp, synthetic construct
  source 1..18: mol_type = other DNA
ataagaacaacaacaaat

SEQ ID NO: 7 - DNA, 18 bp, synthetic construct
  source 1..18: mol_type = other DNA
aaaatatcatagcacaac

SEQ ID NO: 8 - DNA, 18 bp, synthetic construct
  source 1..18: mol_type = other DNA
agaaaatcaagaaggaca

SEQ ID NO: 9 - DNA, 18 bp, synthetic construct
  source 1..18: mol_type = other DNA
gagcaagcaagatatttg

SEQ ID NO: 10 - DNA, 18 bp, synthetic construct
  source 1..18: mol_type = other DNA
aagaaatcaaaagaataa

SEQ ID NO: 11 - DNA, 18 bp, synthetic construct
  source 1..18: mol_type = other DNA
aagaaatcaaacaactaa

SEQ ID NO: 12 - DNA, 18 bp, synthetic construct
  source 1..18: mol_type = other DNA
attacgttacaagaacac

SEQ ID NO: 13 - DNA, 18 bp, synthetic construct
  source 1..18: mol_type = other DNA
tagctactacccctatta

SEQ ID NO: 14 - DNA, 18 bp, synthetic construct
  source 1..18: mol_type = other DNA
agatcgttaaggaatagt

SEQ ID NO: 15 - DNA, 18 bp, synthetic construct
  source 1..18: mol_type = other DNA
aaagacttatacaagaag

SEQ ID NO: 16 - DNA, 18 bp, synthetic construct
  source 1..18: mol_type = other DNA
gtaaaaatacagaactct

SEQ ID NO: 17 - DNA, 18 bp, synthetic construct
  source 1..18: mol_type = other DNA
aaagaaccacagaaaaat

SEQ ID NO: 18 - DNA, 18 bp, synthetic construct
  source 1..18: mol_type = other DNA
aagtatttaccgtctaaa

SEQ ID NO: 19 - DNA, 18 bp, synthetic construct
  source 1..18: mol_type = other DNA
ctcctattaacggtttga

SEQ ID NO: 20 - DNA, 18 bp, synthetic construct
  source 1..18: mol_type = other DNA
tagaaaagaaaggatagg

SEQ ID NO: 21 - DNA, 18 bp, synthetic construct
  source 1..18: mol_type = other DNA
atataaccaaacagaccg

SEQ ID NO: 22 - DNA, 18 bp, synthetic construct
  source 1..18: mol_type = other DNA
atataaccaatttcaata

SEQ ID NO: 23 - DNA, 18 bp, synthetic construct
  source 1..18: mol_type = other DNA
aaagaattaaatataatc

SEQ ID NO: 24 - DNA, 18 bp, synthetic construct
  source 1..18: mol_type = other DNA
aatgcaccaaacacaaga

SEQ ID NO: 25 - DNA, 18 bp, synthetic construct
  source 1..18: mol_type = other DNA
acaagatcaactaagaac

SEQ ID NO: 26 - DNA, 18 bp, synthetic construct
  source 1..18: mol_type = other DNA
ggaaattcatacacaaca

SEQ ID NO: 27 - DNA, 18 bp, synthetic construct
  source 1..18: mol_type = other DNA
tacacaacaacagaacca

SEQ ID NO: 28 - DNA, 18 bp, synthetic construct
  source 1..18: mol_type = other DNA
gtctccccattgtgcagc

SEQ ID NO: 29 - DNA, 18 bp, synthetic construct
  source 1..18: mol_type = other DNA
gcagcgataaggaacatt

SEQ ID NO: 30 - DNA, 18 bp, synthetic construct
  source 1..18: mol_type = other DNA
tgcacaatatttcaagct

SEQ ID NO: 31 - DNA, 18 bp, synthetic construct
  source 1..18: mol_type = other DNA
aatatttcaagctatacc

SEQ ID NO: 32 - DNA, 18 bp, synthetic construct
  source 1..18: mol_type = other DNA
ttcaagctataccaagca

SEQ ID NO: 33 - DNA, 18 bp, synthetic construct
  source 1..18: mol_type = other DNA
aagcatacaatcaactat

SEQ ID NO: 34 - DNA, 18 bp, synthetic construct
  source 1..18: mol_type = other DNA
taagcaacattttataca

SEQ ID NO: 35 - DNA, 18 bp, synthetic construct
  source 1..18: mol_type = other DNA
aacattttatacattttt

SEQ ID NO: 36 - DNA, 18 bp, synthetic construct
  source 1..18: mol_type = other DNA
gtaagaacatcacacaaa

SEQ ID NO: 37 - DNA, 18 bp, synthetic construct
  source 1..18: mol_type = other DNA
agaacatcacacaaagat

SEQ ID NO: 38 - DNA, 18 bp, synthetic construct
  source 1..18: mol_type = other DNA
agaaaaacatctaacata

SEQ ID NO: 39 - DNA, 18 bp, synthetic construct
  source 1..18: mol_type = other DNA
atacggtcaacgaactat

SEQ ID NO: 40 - DNA, 18 bp, synthetic construct
  source 1..18: mol_type = other DNA
aaaacaccaagaacttag

SEQ ID NO: 41 - DNA, 18 bp, synthetic construct
  source 1..18: mol_type = other DNA
aaaaaaccaagcaactgc

SEQ ID NO: 42 - DNA, 18 bp, synthetic construct
  source 1..18: mol_type = other DNA
gggagaatattcgcaatt

SEQ ID NO: 43 - DNA, 18 bp, synthetic construct
  source 1..18: mol_type = other DNA
tttctttcataacaccaa

SEQ ID NO: 44 - DNA, 18 bp, synthetic construct
  source 1..18: mol_type = other DNA
ataacaccaagcaactaa

SEQ ID NO: 45 - DNA, 18 bp, synthetic construct
  source 1..18: mol_type = other DNA
aagaaagcatagcaatct

SEQ ID NO: 46 - DNA, 18 bp, synthetic construct
  source 1..18: mol_type = other DNA
aaattactatacttctat

SEQ ID NO: 47 - DNA, 18 bp, synthetic construct
  source 1..18: mol_type = other DNA
agaactataacacataga

SEQ ID NO: 48 - DNA, 18 bp, synthetic construct
  source 1..18: mol_type = other DNA
tatgtgttaaatttattg

SEQ ID NO: 49 - DNA, 18 bp, synthetic construct
  source 1..18: mol_type = other DNA
gcagaaacaacaacaaca

SEQ ID NO: 50 - DNA, 161 bp, synthetic construct
  source 1..161: mol_type = other DNA
gctaaaaagagctagtacccgcgcctagcatgtgacctccttgaaactgaaatttacacaaaacttaagagcaacgcattaactttataaaagagcactgttgggcgtgagtggaggcgccggtttttaatatcatatagaagtcatttttaactaactaa

SEQ ID NO: 51 - DNA, 161 bp, synthetic construct
  source 1..161: mol_type = other DNA
aaaaacattttttttttaccgcgccgggggcggtggctcaacggctagcatgtgacatttccctaaaaaatagtttcgttttttttataaaagcgtaggagtactcgatggtacagatgagcattttttagaagtcatcgaaatagtttttaactaactaa

SEQ ID NO: 52 - DNA, 161 bp, synthetic construct
  source 1..161: mol_type = other DNA
tttttctttttttttagaccgcgccactgaaatttgctcaacggctagcatgtgaaaaagtttttgctatttttgatttttcgtttataaaagaacgatctaccgactgtttcgcagagggcctttttagatcatcaaggaagtaatttttaactaactaa

SEQ ID NO: 53 - DNA, 161 bp, synthetic construct
  source 1..161: mol_type = other DNA
attttttttttcggcgccccgcgccgggggcggtgactgaaatttgctcaacggcttcttcttaacactttttgcaggaaaaaagtataaaagccgatagggtgggcgaaggggcgcaggtcctttttatcaaaacaaataaaacatttttaactaagtaa

SEQ ID NO: 54 - DNA, 161 bp, synthetic construct
  source 1..161: mol_type = other DNA
acttttttttttgtactcccgcgccacagaggggctagcatgtgagggggcggtgaaaaaagcaaaaaagaaaaagatttttttttataaaagggccttggtctgaaactcctgcgtctcgcgtttttataagaacaacaacaaattttttaactaagtaa

SEQ ID NO: 55 - DNA, 161 bp, synthetic construct
  source 1..161: mol_type = other DNA
cccgcttttttttttcgaccgcgccgctcaacggccctccttgaaactgaaatttagttaccttttttttttttaagctttttcctataaaagggtccctgggtttgcgtactttatccgtcatttttaaaatatcatagcacaactttttaactaagtaa

SEQ ID NO: 56 - DNA, 161 bp, synthetic construct
  source 1..161: mol_type = other DNA
ttaactttagcctaaataccgcgcctagcatgtgacctccttgaagggggcggtggttcagaatcacccgcgaatacgtagtaattataaaagcgcggtggctccattaaattgctccttccttttttagaaaatcaagaaggacatttttaagtaactaa

SEQ ID NO: 57 - DNA, 161 bp, synthetic construct
  source 1..161: mol_type = other DNA
gtttttccttttttttttccgcgccgggggcggtgacagaggggcgctcaacggccgcagaactatttttttagagtaactcgtttataaaagcaatacttgggtcgacttgttatacgcggatttttgagcaagcaagatatttgtttttaactaagtaa

SEQ ID NO: 58 - DNA, 161 bp, synthetic construct
  source 1..161: mol_type = other DNA
attttttttttttgtctcccgcgccacagaggggcgggggcggtgactgaaattttttttgacaagtcaagtcaggaaaaaaaaatataaaagggcgctgcgtaaggagtgctgccaggtggttttttaagaaatcaaaagaataatttttaagtaactaa

SEQ ID NO: 59 - DNA, 161 bp, synthetic construct
  source 1..161: mol_type = other DNA
ctcgctcttttttttttaccgcgccgctcaacggcactgaaattttagcatgtgataagttcgctaaaaagccatttttttctagtataaaagagcactgttgggcgtgagtggaggcgccggtttttaagaaatcaaacaactaatttttaagtaactaa

SEQ ID NO: 60 - DNA, 161 bp, synthetic construct
  source 1..161: mol_type = other DNA
aatttttttaaaaaaggcccgcgccgctcaacggccctccttgaagggggcggtgtttttgaaaaaaagaagcaaaaactatatttataaaagcgtaggagtactcgatggtacagatgagcatttttattacgttacaagaacactttttaactaactaa

SEQ ID NO: 61 - DNA, 161 bp, synthetic construct
  source 1..161: mol_type = other DNA
ttttttttttttccttccccgcgccactgaaatttgggggcggtggctcaacggcatttttgaggagaagtttttacaaaaaaactataaaagaacgatctaccgactgtttcgcagagggccttttttagctactacccctattatttttaagtaactaa

SEQ ID NO: 62 - DNA, 161 bp, synthetic construct
  source 1..161: mol_type = other DNA
atattcttttttttcgaaccgcgccactgaaatttcctccttgaagctcaacggccttttttaaaaataaactttttccaacatatataaaagccgatagggtgggcgaaggggcgcaggtcctttttagatcgttaaggaatagttttttaactaactaa

SEQ ID NO: 63 - DNA, 161 bp, synthetic construct
  source 1..161: mol_type = other DNA
gtctctatcttaatcgtaccgcgccacagaggggcgggggcggtgcctccttgaaaagttattagcgacgagtaaatcctcaacgtataaaagggccttggtctgaaactcctgcgtctcgcgtttttaaagacttatacaagaagtttttaactaagtaa

SEQ ID NO: 64 - DNA, 161 bp, synthetic construct
  source 1..161: mol_type = other DNA
gccccaacggccggactaccgcgccgggggcggtgacagaggggcactgaaatttggcccaaaaccatagggtataacccagaaatataaaagggtccctgggtttgcgtactttatccgtcatttttgtaaaaatacagaactcttttttaactaactaa

SEQ ID NO: 65 - DNA, 161 bp, synthetic construct
  source 1..161: mol_type = other DNA
tctaacgacggtcctacaccgcgccgggggcggtggctcaacggccctccttgaattaaccgtactcgtaggactcaagagtacatataaaagcgcggtggctccattaaattgctccttccttttttaaagaaccacagaaaaattttttaactaactaa

SEQ ID NO: 66 - DNA, 171 bp, synthetic construct
  source 1..171: mol_type = other DNA
attttttcatttttttaaccgcgccactgaaattttagcatgtgaacagaggggccctccttgaacgatttttcacaaagaaaaaaagttttttatataaaagcaatacttgggtcgacttgttatacgcggatttttaagtatttaccgtctaaatttttaactaactaa

SEQ ID NO: 67 - DNA, 171 bp, synthetic construct
  source 1..171: mol_type = other DNA
ggttttttttttttttacccgcgccacagaggggcgctcaacggctagcatgtgagggggcggtgaaaaagaaattaaaaaaaaaaaattccatatataaaagggcgctgcgtaaggagtgctgccaggtggttttttctcctattaacggtttgatttttaactaactaa

SEQ ID NO: 68 - DNA, 171 bp, synthetic construct
  source 1..171: mol_type = other DNA
cacttttttttttttttaccgcgccactgaaatttgctcaacggcacagaggggcgggggcggtgaatttccaattaatctttttattactcgtatataaaagagcactgttgggcgtgagtggaggcgccggttttttagaaaagaaaggataggtttttaactaagtaa

SEQ ID NO: 69 - skipped

SEQ ID NO: 70 - DNA, 171 bp, synthetic construct
  source 1..171: mol_type = other DNA
tttttttttatatatcgcccgcgcccctccttgaaactgaaatttacagaggggcgctcaacggcatcgaaaaaaaaacacaaagtcgtttttcttataaaagcgtaggagtactcgatggtacagatgagcatttttatataaccaaacagaccgtttttaactaagtaa

SEQ ID NO: 71 - DNA, 171 bp, synthetic construct
  source 1..171: mol_type = other DNA
tcgcataaggactattaaccgcgccgggggcggtggctcaacggcacagaggggctagcatgtgacaagattccttcgtaaaacttctttctcagtataaaagaacgatctaccgactgtttcgcagagggcctttttatataaccaatttcaatatttttaagtaagtaa

SEQ ID NO: 72 - DNA, 171 bp, synthetic construct
  source 1..171: mol_type = other DNA
ccgtttttttttttttgcccgcgccacagaggggcgctcaacggcgggggcggtgtagcatgtgattttttctaataaccaaactttttttttgatataaaagccgatagggtgggcgaaggggcgcaggtcctttttaaagaattaaatataatctttttaactaactaa

SEQ ID NO: 73 - DNA, 171 bp, synthetic construct
  source 1..171: mol_type = other DNA
gaatttttttttttttttccgcgccgctcaacggctagcatgtgaactgaaatttgggggcggtgggcaatccaagagtttttttatttttcttttataaaagggccttggtctgaaactcctgcgtctcgcgaaaaaaatgcaccaaacacaagatttttaactaactaa

SEQ ID NO: 74 - DNA, 171 bp, synthetic construct
  source 1..171: mol_type = other DNA
tttttttttttttttatcccgcgccgctcaacggcacagaggggcactgaaattttagcatgtgagcgttatttttttttaaacttctttttaaatataaaagggtccctgggtttgcgtactttatccgtcatttttacaagatcaactaagaactttttaactaactaa

SEQ ID NO: 75 - DNA, 171 bp, synthetic construct
  source 1..171: mol_type = other DNA
ttttttttttataaaagcccgcgcctagcatgtgagctcaacggcgggggcggtgactgaaatttaatttcggttaaaatttttcgtttcactattataaaagcgcggtggctccattaaattgctccttccttttttggaaattcatacacaacatttttaactaactaa

SEQ ID NO: 76 - DNA, 171 bp, synthetic construct
  source 1..171: mol_type = other DNA
gaaaaaattttttttttcccgcgccgctcaacggctagcatgtgaacagaggggcactgaaattttacgagttaaagtcgaagttttttaaaaaatataaaagcaatacttgggtcgacttgttatacgcggattttttacacaacaacagaaccatttttaagtaactaa

SEQ ID NO: 77 - DNA, 171 bp, synthetic construct
  source 1..171: mol_type = other DNA
tattcgttctacagtaacccgcgccactgaaatttgggggcggtgcctccttgaatagcatgtgacagaaagagatacgtagcatttcagactaatataaaagggcgctgcgtaaggagtgctgccaggtggttttttgtctccccattgtgcagctttttaactaagtaa

SEQ ID NO: 78 - DNA, 171 bp, synthetic construct
  source 1..171: mol_type = other DNA
cccagaatagtactccacccgcgccacagaggggcgctcaacggcactgaaatttgggggcggtgtagatcggtaagacgattcttcactacttatataaaagagcactgttgggcgtgagtggaggcgccggtttttgcagcgataaggaacatttttttaactaagtaa

SEQ ID NO: 79 - DNA, 171 bp, synthetic construct
  source 1..171: mol_type = other DNA
ttttttttttttttttcaccgcgccgctcaacggccctccttgaaacagaggggctagcatgtgagttttttttgacaaaaatcaagggttatactataaaagcgtaggagtactcgatggtacagatgagcattttttgcacaatatttcaagcttttttaactaagtaa

SEQ ID NO: 80 - DNA, 171 bp, synthetic construct
  source 1..171: mol_type = other DNA
gacgccaagtatcaggaaccgcgcctagcatgtgaactgaaatttgctcaacggcgggggcggtgttaggtcaaaacgctaactcattagaatactataaaagaacgatctaccgactgtttcgcagagggcctttttaatatttcaagctatacctttttaagtaagtaa

SEQ ID NO: 81 - DNA, 171 bp, synthetic construct
  source 1..171: mol_type = other DNA
cggtctactcgagttagaccgcgccacagaggggccctccttgaagctcaacggcactgaaatttgcatcttactctcttagggtccaaaccctatataaaagccgatagggtgggcgaaggggcgcaggtccaaaaattcaagctataccaagcatttttaactaactaa

SEQ ID NO: 82 - DNA, 171 bp, synthetic construct
  source 1..171: mol_type = other DNA
attttttttatctttttcccgcgcctagcatgtgacctccttgaaacagaggggcactgaaatttacttttttcttttttaggatcctttttttatataaaagggccttggtctgaaactcctgcgtctcgcgtttttaagcatacaatcaactattttttaagtaagtaa

SEQ ID NO: 83 - DNA, 181 bp, synthetic construct
  source 1..181: mol_type = other DNA
tcttttttttttttttgaccgcgccactgaaatttacagaggggctagcatgtgacctccttgaagctcaacggctttttgctccactaaaaacgcatttaaaaatataaaagggtccctgggtttgcgtactttatccgtcattttttaagcaacattttatacatttttaactaagtaa

SEQ ID NO: 84 - DNA, 181 bp, synthetic construct
  source 1..181: mol_type = other DNA
tatttttttttctactaaccgcgcctagcatgtgagctcaacggcactgaaatttcctccttgaagggggcggtgaaaaaaagcttaacttactcgctttttttttataaaagcgcggtggctccattaaattgctccttccttttttaacattttatacattttttttttaagtaagtaa

SEQ ID NO: 85 - DNA, 181 bp, synthetic construct
  source 1..181: mol_type = other DNA
gcacttttttttttttacccgcgcccctccttgaagggggcggtggctcaacggcactgaaatttacagaggggcaagaagttacgaaaaaaaatctttttttattataaaagcaatacttgggtcgacttgttatacgcggatttttgtaagaacatcacacaaatttttaactaagtaa

SEQ ID NO: 86 - DNA, 181 bp, synthetic construct
  source 1..181: mol_type = other DNA
tcgccacgtttaaatcgaccgcgccactgaaatttcctccttgaaacagaggggcgggggcggtggctcaacggcagcttcttagtttttcacgtatccactttatataaaagggcgctgcgtaaggagtgctgccaggtggttttttagaacatcacacaaagattttttaagtaagtaa

SEQ ID NO: 87 - DNA, 181 bp, synthetic construct
  source 1..181: mol_type = other DNA
tctccctaaacagccctaccgcgccacagaggggcactgaaatttgggggcggtgtagcatgtgagctcaacggcacgtacaggctagatttcaactaataaccatataaaagagcactgttgggcgtgagtggaggcgccggtttttagaaaaacatctaacatatttttaagtaagtaa

SEQ ID NO: 88 - DNA, 181 bp, synthetic construct
  source 1..181: mol_type = other DNA
catttttttttttttttaccgcgcccctccttgaaactgaaattttagcatgtgagggggcggtggctcaacggcaactcctttttaatcatcataaaaattttatataaaagcgtaggagtactcgatggtacagatgagcatttttatacggtcaacgaactattttttaagtaactaa

SEQ ID NO: 89 - DNA, 181 bp, synthetic construct
  source 1..181: mol_type = other DNA
ttcatttttttttttttcccgcgccgctcaacggccctccttgaatagcatgtgaacagaggggcgggggcggtgttttttttttcacgttttttacccttcaattataaaagaacgatctaccgactgtttcgcagagggcctttttaaaacaccaagaacttagtttttaactaactaa

SEQ ID NO: 90 - DNA, 181 bp, synthetic construct
  source 1..181: mol_type = other DNA
gcgtttttttttttttccccgcgcccctccttgaatagcatgtgagggggcggtgactgaaatttacagaggggctaatccaaaaaaattttttctttttttccgtataaaagccgatagggtgggcgaaggggcgcaggtcctttttaaaaaaccaagcaactgctttttaagtaactaa

SEQ ID NO: 91 - DNA, 181 bp, synthetic construct
  source 1..181: mol_type = other DNA
gtttttttttttttttcaccgcgccgctcaacggcgggggcggtgacagaggggctagcatgtgaactgaaatttgactttttttgttttttatttttatttcactataaaagggccttggtctgaaactcctgcgtctcgcgtttttgggagaatattcgcaatttttttaagtaagtaa

SEQ ID NO: 92 - DNA, 181 bp, synthetic construct
  source 1..181: mol_type = other DNA
ttctacttttttttttttccgcgccacagaggggccctccttgaagggggcggtgactgaaattttagcatgtgaaaaaattttttttaatcctcattttttaaatataaaagggtccctgggtttgcgtactttatccgtcattttttttctttcataacaccaatttttaactaagtaa

SEQ ID NO: 93 - DNA, 181 bp, synthetic construct
  source 1..181: mol_type = other DNA
gtttttttttttatttttccgcgcccctccttgaagggggcggtgtagcatgtgaactgaaatttacagaggggcaatttttgttttttcattaacgtttaacactataaaagcgcggtggctccattaaattgctccttccttttttataacaccaagcaactaatttttaactaagtaa

SEQ ID NO: 94 - DNA, 181 bp, synthetic construct
  source 1..181: mol_type = other DNA
aaaaatttttttttttcaccgcgccgctcaacggcacagaggggccctccttgaatagcatgtgaactgaaatttatctcatttttttttttttatttcgcgtaatataaaagcaatacttgggtcgacttgttatacgcggatttttaagaaagcatagcaatcttttttaactaactaa

SEQ ID NO: 95 - DNA, 181 bp, synthetic construct
  source 1..181: mol_type = other DNA
cttttttttttttttacaccgcgcctagcatgtgagggggcggtgcctccttgaaactgaaatttgctcaacggcccataatatttttttttttttttaatctcgtataaaagggcgctgcgtaaggagtgctgccaggtggttttttaaattactatacttctattttttaactaagtaa

SEQ ID NO: 96 - DNA, 181 bp, synthetic construct
  source 1..181: mol_type = other DNA
tagtactcagccacaagaccgcgccgggggcggtgactgaaatttcctccttgaatagcatgtgagctcaacggctttaccgaaggtcttagtagcagtactctttataaaagagcactgttgggcgtgagtggaggcgccggtttttagaactataacacatagatttttaactaagtaa

SEQ ID NO: 97 - DNA, 181 bp, synthetic construct
  source 1..181: mol_type = other DNA
ctcttctcgctctcgcgcccgcgcctagcatgtgagggggcggtgacagaggggccctccttgaaactgaaatttgcgaattaagtagggtcaagtcttaaggcatataaaagcgtaggagtactcgatggtacagatgagcattttttatgtgttaaatttattgtttttaactaactaa

SEQ ID NO: 98 - DNA, 181 bp, synthetic construct
  source 1..181: mol_type = other DNA
tccgtcaggtctaccgaaccgcgccgggggcggtgtagcatgtgaacagaggggcgctcaacggccctccttgaatacatcattaccgactacagagttatccactataaaagaacgatctaccgactgtttcgcagagggcctttttgcagaaacaacaacaacatttttaagtaactaa

SEQ ID NO: 99 - DNA, 61 bp, synthetic construct
  source 1..61: mol_type = other DNA
taccggtgataccagcatcgtcttgatgcccttggcagcaccctgctaaggaggcaacaag

SEQ ID NO: 100 - skipped

SEQ ID NO: 101 - DNA, 144 bp, synthetic construct
  source 1..144: mol_type = other DNA
aagtgataccagcatcgtcttgatgcccttggcagcacttcatttacatactcggtaaactgaagtgctgccattttttttggtaccggtgataccagcatcgtcttgatgcccttggcagcaccctgctaaggaggcaacaag

SEQ ID NO: 102 - DNA, 81 bp, synthetic construct
  source 1..81: mol_type = other DNA
gacgggactctcaccaggtaccggagataccagcatcgtcttgatgcccttggcagctccagctgctaaggaggtatcaag

SEQ ID NO: 103 - DNA, 117 bp, synthetic construct
  source 1..117: mol_type = other DNA
gacgggactctcaccaggtaccggagataccagcatcgtcttgatgcccttggcagctccagctgctaaggaggtatcaagatggaagacgccaaaaacataaagaaaggcccggcg

SEQ ID NO: 104 - DNA, 624 bp, Escherichia coli
  source 1..624: mol_type = genomic DNA
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaa

SEQ ID NO: 105 - DNA, 624 bp, synthetic construct
  source 1..624: mol_type = other DNA
atgtcaaggctggataaatcaaaagtaatcaatagcgcgctggaactgctgaacgaggtcggcatcgaaggtctgaccacccgcaagctggcgcaaaaactgggcgtcgaacaaccgacgctgtactggcacgtaaaaaataagcgtgcgctgctggacgcactggcaattgaaatgctggatcgtcaccacacccacttctgtccgctggagggtgaatcatggcaagatttccttcgcaacaacgcgaagtcatttcgctgcgcgctgctgagccaccgcgatggagcaaaagttcatctgggcacccgcccaacggagaaacaatatgaaacgctggaaaaccagcttgccttcctgtgccagcagggtttcagccttgagaacgcgctgtacgcgctgagcgccgtaggtcacttcaccctgggctgtgttctggaagaccaagaacatcaagtagcaaaagaagagcgagaaacccctacgaccgattcgatgccgccgctgctgcgtcaggcgattgaactgttcgatcaccagggcgcggaaccggcattcctgtttggtctggaacttattatatgcggcctagaaaaacaactgaagtgcgaaagcggtagctaa

SEQ ID NO: 106 - DNA, 2652 bp, Escherchia phage T7
  source 1..2652: mol_type = genomic DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 107 - DNA, 2652 bp, synthetic construct
  source 1..2652: mol_type = other DNA
atgaacacgattaacatcgctaagaacgacttctctgacatcgaactggctgctatcccgttcaacactctggctgaccattacggtgagcgtttggctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttgcaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttgcagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttggagtcggacttcgcgttcgcgtaa

SEQ ID NO: 108 - DNA, 23 bp, synthetic construct
  source 1..23: mol_type = other DNA
taatacgactcactatagggaga

SEQ ID NO: 109 - DNA, 23 bp, synthetic construct
  source 1..23: mol_type = other DNA
taatacgactcactaatactgaa

SEQ ID NO: 110 - DNA, 2712 bp, synthetic construct
  source 1..2712: mol_type = other DNA
tcagaattggttaattggttgtaacactggttggcagcacaatggtaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 111 - DNA, 2743 bp, synthetic construct
  source 1..2743: mol_type = other DNA
tcagaattggttaattggttgtaacactggtaccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 112 - DNA, 2826 bp, synthetic construct
  source 1..2826: mol_type = other DNA
tcagaattggttaattggttgtaacactggaagtgataccagcatcgtcttgatgcccttggcagcacttcatttacatactcggtaaactgaagtgctgccattttttttggtaccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 113 - DNA, 2849 bp, synthetic construct
  source 1..2849: mol_type = other DNA
tcagaattggttaattggttgtaacactggtaatacgactcactaatactgaaaagtgataccagcatcgtcttgatgcccttggcagcacttcatttacatactcggtaaactgaagtgctgccattttttttggtaccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 114 - DNA, 3575 bp, synthetic construct
  source 1..3575: mol_type = other DNA
tcagaattggttaattggttgtaacactggtctatcattgataggtataaattaatacgactcactaatactgaacctatcagtgatagatccaaacccaaaaacacaggagtttttagaatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagtgataccagcatcgtcttgatgcccttggcagcacttcatttacatactcggtaaactgaagtgctgccattttttttggtaccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaaagcttgatgggggatcccatggtacgcgtgctag

SEQ ID NO: 115 - DNA, 3512 bp, synthetic construct
  source 1..3512: mol_type = other DNA
tcagaattggttaattggttgtaacactggtctatcattgataggtataaattaatacgactcactaatactgaacctatcagtgatagatccaaacccaaaaacacaggagtttttagaatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaagcggatctttacagattctataccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaaagcttgatgggggatcccatggtacgcgtgctag

SEQ ID NO: 116 - DNA, 3512 bp, synthetic construct
  source 1..3512: mol_type = other DNA
tcagaattggttaattggttgtaacactggtctatcattgataggtataaattaatacgactcactatagggagacctatcagtgatagatccaaacccaaaaacacaggagtttttagaatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaagcggatctttacagattctataccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaaagcttgatgggggatcccatggtacgcgtgctag

SEQ ID NO: 117 - DNA, 3575 bp, synthetic construct
  source 1..3575: mol_type = other DNA
tcagaattggttaattggttgtaacactggtctatcattgataggtataaattaatacgactcactatagggagacctatcagtgatagatccaaacccaaaaacacaggagtttttagaatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagtgataccagcatcgtcttgatgcccttggcagcacttcatttacatactcggtaaactgaagtgctgccattttttttggtaccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaaagcttgatgggggatcccatggtacgcgtgctag

SEQ ID NO: 118 - DNA, 2766 bp, synthetic construct
  source 1..2766: mol_type = other DNA
tcagaattggttaattggttgtaacactggtaatacgactcactaatactgaataccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 119 - DNA, 2766 bp, synthetic construct
  source 1..2766: mol_type = other DNA
tcagaattggttaattggttgtaacactggtaatacgactcactatagggagataccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 120 - DNA, 3565 bp, synthetic construct
  source 1..3565: mol_type = other DNA
tcagaattggttaattggttgtaacactggtctatcattgataggtataaattaatacgactcactaatactgaacctatcagtgatagatccaaacccaaaaacacaggagtttttagaatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaagcggatctttacagattctagacgggactctcaccaggtaccggagataccagcatcgtcttgatgcccttggcagctccagctgctaaggaggtatcaagatggaagacgccaaaaacataaagaaaggcccggcgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaaagcttgatgggggatcccatggtacgcgtgctag

SEQ ID NO: 121 - DNA, 3544 bp, synthetic construct
  source 1..3544: mol_type = other DNA
tcagaattggttaattggttgtaacactggtctatcattgataggtataaattaatacgactcactaatactgaacctatcagtgatagatccaaaccctcgttaggggagcgtctaattttaggagatccaaaatgtcaaggctggataaatcaaaagtaatcaatagcgcgctggaactgctgaacgaggtcggcatcgaaggtctgaccacccgcaagctggcgcaaaaactgggcgtcgaacaaccgacgctgtactggcacgtaaaaaataagcgtgcgctgctggacgcactggcaattgaaatgctggatcgtcaccacacccacttctgtccgctggagggtgaatcatggcaagatttccttcgcaacaacgcgaagtcatttcgctgcgcgctgctgagccaccgcgatggagcaaaagttcatctgggcacccgcccaacggagaaacaatatgaaacgctggaaaaccagcttgccttcctgtgccagcagggtttcagccttgagaacgcgctgtacgcgctgagcgccgtaggtcacttcaccctgggctgtgttctggaagaccaagaacatcaagtagcaaaagaagagcgagaaacccctacgaccgattcgatgccgccgctgctgcgtcaggcgattgaactgttcgatcaccagggcgcggaaccggcattcctgtttggtctggaacttattatatgcggcctagaaaaacaactgaagtgcgaaagcggtagctaagcggatctttacagattctataccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttggctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttgcaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttgcagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttggagtcggacttcgcgttcgcgtaaaagcttgatatcgaattcctgcagccccggggatcccatggtacgcgtgctag

SEQ ID NO: 122 - DNA, 3526 bp, synthetic construct
  source 1..3526: mol_type = other DNA
tcagaattggttaattggttgtaacactggtctatcattgataggtataaattaatacgactcactatagggagacctatcagtgatagatccaaaccctcgttaggggagcgtctaattttaggagatccaaaatgtcaaggctggataaatcaaaagtaatcaatagcgcgctggaactgctgaacgaggtcggcatcgaaggtctgaccacccgcaagctggcgcaaaaactgggcgtcgaacaaccgacgctgtactggcacgtaaaaaataagcgtgcgctgctggacgcactggcaattgaaatgctggatcgtcaccacacccacttctgtccgctggagggtgaatcatggcaagatttccttcgcaacaacgcgaagtcatttcgctgcgcgctgctgagccaccgcgatggagcaaaagttcatctgggcacccgcccaacggagaaacaatatgaaacgctggaaaaccagcttgccttcctgtgccagcagggtttcagccttgagaacgcgctgtacgcgctgagcgccgtaggtcacttcaccctgggctgtgttctggaagaccaagaacatcaagtagcaaaagaagagcgagaaacccctacgaccgattcgatgccgccgctgctgcgtcaggcgattgaactgttcgatcaccagggcgcggaaccggcattcctgtttggtctggaacttattatatgcggcctagaaaaacaactgaagtgcgaaagcggtagctaagcggatctttacagattctataccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttggctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttgcaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttgcagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttggagtcggacttcgcgttcgcgtaaaagcttgatgggggatcccatggtacgcgtgctag

SEQ ID NO: 123 - DNA, 3461 bp, synthetic construct
  source 1..3461: mol_type = other DNA
tcagaattggttaattggttgtaacactggtctatcattgataggtataaattaatacgactcactaatactgaacctatcagtgatagatccaaacccaaaaacacaggagtttttagaatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaattggcagcacaatggtaaggaggcaacaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaaagcttgatgggggatcccatggtacgcgtgctag

SEQ ID NO: 124 - DNA, 3547 bp, synthetic construct
  source 1..3547: mol_type = other DNA
tcagaattggttaattggttgtaacactggtctatcattgataggtataaattaatacgactcactaatactgaacctatcagtgatagataccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttggctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttgcaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttgcagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttggagtcggacttcgcgttcgcgtaaaagcttgatatcgaattcctgcagccccggggatcccatggtacgcgtgctagtaatacgactcactaatactgaatccaaaccctcgttaggggagcgtctaattttaggagatccaaaatgtcaaggctggataaatcaaaagtaatcaatagcgcgctggaactgctgaacgaggtcggcatcgaaggtctgaccacccgcaagctggcgcaaaaactgggcgtcgaacaaccgacgctgtactggcacgtaaaaaataagcgtgcgctgctggacgcactggcaattgaaatgctggatcgtcaccacacccacttctgtccgctggagggtgaatcatggcaagatttccttcgcaacaacgcgaagtcatttcgctgcgcgctgctgagccaccgcgatggagcaaaagttcatctgggcacccgcccaacggagaaacaatatgaaacgctggaaaaccagcttgccttcctgtgccagcagggtttcagccttgagaacgcgctgtacgcgctgagcgccgtaggtcacttcaccctgggctgtgttctggaagaccaagaacatcaagtagcaaaagaagagcgagaaacccctacgaccgattcgatgccgccgctgctgcgtcaggcgattgaactgttcgatcaccagggcgcggaaccggcattcctgtttggtctggaacttattatatgcggcctagaaaaacaactgaagtgcgaaagcggtagctaa

SEQ ID NO: 125 - DNA, 7988 bp, synthetic construct
  source 1..7988: mol_type = other DNA
aagcttgatgggggatctaatacgactcactatagggagatttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctggtcttcgcgcccatcatgaggtggcgccgaaggcttgcccttcgttcgaccttaagcgttggtgggagaagaacgaactggtcacttctgaccgtggataagatcccatggtacgcgtgctagaggcatcaaataaaacgaaaggctcagtcgaaagactgggcctttcgttttatctgttgtttgtcggtgaacgctctcctgagtaggacaaatccgccgccctagacctaggggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagttgcagaaataaaaaggcctgcgattaccagcagtcctgttattagctcagtaaagctttatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctgagatcttgatcccctgcgccatcagatccttggcggcaagaaagccatccagtttactttgcagggcttcccaaccttaccagagggcgcgaaggcgaagcggcatgcatttacgttgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtacctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagagcgctggatggttgatacaggtatcgaagtacctgagcaaggggagttcgaccttaacgaaatcatggattctgaatacgtatttgcctaattgacggctagctcagtcctaggtacagtgctagcagctaaagctatataatttaattaggagaagtaaaatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatctaa

SEQ ID NO: 126 - DNA, 9380 bp, synthetic construct
  source 1..9380: mol_type = other DNA
gggtgccagggcgtgcccgtgggctccccgggcgcgtatagggataacagggtaatacgtcaaattctatcataattgtggtttcaaaatcggctccgtcgatactatgttatacgccaactttgaaaacaactttgaaaaggctgttttctgtatttaaggttttagaatgcaaggaacagtgaattggagttcgtcttgttataattagcttcttggggtatctttaaatactgtagaaaaagaaattgcatacctttgttcctcggttatatgtttgctcatctgcaagacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggcacctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccaaaaatttgtaattaagaaggagtgattacatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgtccagatgcgaagttaagtgcgcagaaagtaatatcatgcgtcaatcgtatgtgaatgctggtcgctatactgctctagcataaccccgcggggcctctttcggggatctcgcggggttttttgctgaaagaagcttcaaataaaacgaaaggctcagtcgaaagactgggcctttcgttatgttgttgtcgctgcggccgcactcgagcaccaccaccaccaccactgggatccggctgctaacaaagcccgaaaggaagctgagttggctgctgccaccgctgagcaataactagcataaccccttggggcctctaaacgggtcttgaggggttttttgctgaaggccatcatggcctaatacgactcactatagggagatcctgcaggccaatgtgatggacactgagacctccacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaggtctctagagccaccacagtggttaattaaggcctgtgaggccggaccaaaacgaaaaaaggcccccctttcgggaggcctcttttctggaatttggtaccgaggggtgccagggcgtgccccagggctccccgggcgcgtataatcgatttaaattagtagcccgcctaatgagcgggcttttttttaattcccctatttgtttatttttctaaatacattcaaatatgtatccgctcatgagacaataaccctgataaatgcttcaataatattgaaaaaggaagagtatgagcattcagcattttcgtgtggcgctgattccgttttttgcggcgttttgcctgccggtgtttgcgcatccggaaaccctggtgaaagtgaaagatgcggaagatcaactgggtgcgcgcgtgggctatattgaactggatctgaacagcggcaaaattctggaatcttttcgtccggaagaacgttttccgatgatgagcacctttaaagtgctgctgtgcggtgcggttctgagccgtgtggatgcgggccaggaacaactgggccgtcgtattcattatagccagaacgatctggtggaatatagcccggtgaccgaaaaacatctgaccgatggcatgaccgtgcgtgaactgtgcagcgcggcgattaccatgagcgataacaccgcggcgaacctgctgctgacgaccattggcggtccgaaagaactgaccgcgtttctgcataacatgggcgatcatgtgacccgtctggatcgttgggaaccggaactgaacgaagcgattccgaacgatgaacgtgataccaccatgccggcagcaatggcgaccaccctgcgtaaactgctgacgggtgagctgctgaccctggcaagccgccagcaactgattgattggatggaagcggataaagtggcgggtccgctgctgcgtagcgcgctgccggctggctggtttattgcggataaaagcggtgcgggcgaacgtggcagccgtggcattattgcggcgctgggcccggatggtaaaccgagccgtattgtggtgatttataccaccggcagccaggcgacgatggatgaacgtaaccgtcagattgcggaaattggcgcgagcctgattaaacattggtaaaccgatacaattaaaggctccttttggagcctttttttttggacgacccttgtccttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgtaggccggccggcgcgccgatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcctttaaggtttaacggttgtggacaacaagccagggatgtaacgcactgagaagcccttagagcctctcaaagcaattttgagtgacacaggaacacttaacggctgacatggggcgcgcccaggtcagtcacagtggagcctagcactcgctcagcgtgacggctcagagcagaattcacgagccagaaatagtaacttttgcctaaatcacaaattgcaaaatttaattgcttgcaaaaggtcacatgcttataatcaacttttttaaaaatttaaaatacttttttattttttatttttaaacataaatgaaataatttatttattgtttatgattaccgaaacataaaacctgctcaagaaaaagaaactgttttgtccttggaaaaaaagcactacctaggagcggccaaaatgccggcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaagccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccgaattccagtcaggctgctagcaccagagctacgtgaccgcaggactagctccagctgagcgacagcggcaagaaagccatccacgccgaaaaccccgcttcggcggggttttgccgcatcttgccgtaactgacaaattaaccgaaggtttcttccggccactgggatgcaatgaccttaccaacaacgctacaggattcgttgcacgggatcattgggtattgcggattcagcggctgcaggaaaacttggccagagtcgcggatcagcttcttaaaggtgaattcatcaccacccaggcgcgcaatgcaaaagtctcccggctcaaccgcctgctcaggatcaaccaggatcagcataccatccggaaatgacggcttgctgcccgtcggggccgtcatgctgttgccttctacttccagccaaaacgcgctatcgctcgcttttttggtagtcgatacccaacgctcagcgtcacctttggtgaaggtacgcagttccgggctaaacatacctgcttgaacgtggctgaagaccgggtattcgtattcagagcgcagagacggttgcatgctaaccgcttcatacatttcatagatttcacgagctatagaaggagagaactcctcgacggaaactttcagaatcttggtcagcagtgcggcgttgtacgcgttcagcgcattaataccgttaaacagagcgccaacgccgctctgacccatgcccatcttgtcggctacgctttcctgagacagacccagctcattctttttcttctcatagattgctttcagacgacgtgcgtcctctagctgctcttgtgtaagcggctttttcttcgttgacattttgatcctcctttatatggaggtactaaatcggaacgttaaatctatcaccgcaagggataaatatctaacaccgtgcgtgttgactattttacctctggcggtgataatggttgcatcaaatttgcgcgccacattattattcatacctttgtggaccgtattacaaagagaagtgttaaatcaaacaaaaaggaggattaatcatggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtaatctatagtgtcacctaaat

SEQ ID NO: 127 - DNA, 9936 bp, synthetic construct
  source 1..9936: mol_type = other DNA
taacaggttggatgataagtccccggtctgagcacccattagttcaacaaacgaaaattggataaagtgggatatttttaaaatatatatttatgttacagtaatattgacttttaaaaaaggattgattctaatgaagaaagcagacaagtaagcctatttaaatttgtgtctcaaaatctctgatgttacattgcacaagataaaaatatatcatcatgaacaataaaactgtctgcttacataaacagtaattacttttgttagatatatttttttgtgtaattttgtaatcgttatgcggcagtaaaaggatctattataaggaggcactcaccatgcaatacgaatggcgaaaagccgagctcatcggtcagcttctcaaccttggggttacccccggcggtgtgctgctggtccacagctccttccgtagcgtccggcccctcgaagatgggccacttggactgatcgaggccctgcgtgctgcgctgggtccgggagggacgctcgtcatgccctcgtggtcaggtctggacgacgagccgttcgatcctgccacgtcgcccgttacaccggaccttggagttgtctctgacacattctggcgcctgccaaatgtaaagcgcagcgcccatccatttgcctttgcggcagcggggccacaggcagagcagatcatctctgatccattgcccctgccacctcactcgcctgcaagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcgctttgccttggcggacaggtggctcaaggagaagagccttcagaaggaaggtccagtcggtcatgcctttgctcggttgatccgctcccgcgacattgtggcgacagccctgggtcaactgggccgagatccgttgatcttcctgcatccgccagaggcgggatgcgaagaatgcgatgccgctcgccagtcgattggctgagctcataatagggataatcagaattggttaattggttgtaacactggtctatcattgataggtataaattaatacgactcactaatactgaacctatcagtgatagataccggtgataccagcatcgtcttgatgcccttggcagcaccctgctaaggaggcaacaagatgaacacgattaacatcgctaagaacgacttctctgacatcgaactggctgctatcccgttcaacactctggctgaccattacggtgagcgtttggctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttgcaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttgcagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttggagtcggacttcgcgttcgcgtaaaagcttgatatcgaattcctgcagccccggggatcccatggtacgcgtgctagtaatacgactcactaatactgaatccaaaccctcgttaggggagcgtctaattttaggagatccaaaatgtcaaggctggataaatcaaaagtaatcaatagcgcgctggaactgctgaacgaggtcggcatcgaaggtctgaccacccgcaagctggcgcaaaaactgggcgtcgaacaaccgacgctgtactggcacgtaaaaaataagcgtgcgctgctggacgcactggcaattgaaatgctggatcgtcaccacacccacttctgtccgctggagggtgaatcatggcaagatttccttcgcaacaacgcgaagtcatttcgctgcgcgctgctgagccaccgcgatggagcaaaagttcatctgggcacccgcccaacggagaaacaatatgaaacgctggaaaaccagcttgccttcctgtgccagcagggtttcagccttgagaacgcgctgtacgcgctgagcgccgtaggtcacttcaccctgggctgtgttctggaagaccaagaacatcaagtagcaaaagaagagcgagaaacccctacgaccgattcgatgccgccgctgctgcgtcaggcgattgaactgttcgatcaccagggcgcggaaccggcattcctgtttggtctggaacttattatatgcggcctagaaaaacaactgaagtgcgaaagcggtagctaacgccgaaaaccccgcttcggcggggttttgccgcataacagggtaatccccaactggggtaacctgtgagttctctcagttggggaaaaaaaaaccccgcccctgacagggcggggtttttttttttcgccgcgttggctagtaatacgactcactatagggagatctcttaagtataaggaggaaaaaatatgagcaagggcgaagaactgtttacgggcgtggtgccgattctggtggaactggatggtgatgtcaatggtcacaaattcagcgtgcgcggcgaaggtgaaggcgatgcaaccaatggtaaactgacgctgaagtttatttgcaccacgggtaaactgccggttccgtggccgaccctggtcaccacgctgacgtatggtgttcagtgtttcagtcgttacccggatcacatgaaacgccacgactttttcaagtccgcgatgccggaaggttatgtccaagaacgtaccatctcatttaaagatgacggcacctacaaaacgcgcgccgaagtgaaattcgaaggtgatacgctggttaaccgtattgaactgaaaggcatcgattttaaggaagacggtaatattctgggccataaactggaatataacttcaattcgcacaacgtgtacatcaccgcagataagcagaagaacggtatcaaggctaacttcaagatccgccataatgtggaagatggcagcgttcaactggccgaccactatcagcaaaacaccccgattggtgatggcccggtcctgctgccggacaatcattacctgagcacgcagtctgtgctgagtaaagatccgaacgaaaagcgtgaccacatggtcctgctggaattcgtgaccgcggccggcatcacgcacggtatggacgaactgtataaaggctcagatatatcgggcggtatggtttttactctggaagattttgttggcgattggcgtcagaccgcgggttataatttggatcaagtcctggaacagggtggcgtaagctctctgttccagaacctgggtgtgagcgtgacgccgattcagcgcatcgttctgtccggcgagaacggtctgaaaattgatattcatgtgatcatcccgtacgaaggcctgagcggtgaccaaatgggtcaaatcgagaaaatctttaaagtcgtctacccagttgacgatcaccacttcaaggttatcttgcattacggtacgctggtgattgatggtgtgaccccgaatatgattgactatttcggccgtccgtatgaaggcattgccgtttttgacggtaaaaagatcaccgtcaccggtaccctgtggaatggcaataagattattgacgagcgtctgattaacccggacggcagcctgctgttccgcgtgaccatcaacggtgtcacgggttggcgtctgtgcgagcgcatcctggcataatctagaccccaactggggtaacctcagagttctctcagttggggtagaccggggacttatcatccaacctgttactgtctatagtgtcacctaaattaatcgatttaaattagtagcccgcctaatgagcgggcttttttttaattcccctatttgtttatttttctaaatacattcaaatatgtatccgctcatgagacaataaccctgataaatgcttcaataatattgaaaaaggaagagtatgagcattcagcattttcgtgtggcgctgattccgttttttgcggcgttttgcctgccggtgtttgcgcatccggaaaccctggtgaaagtgaaagatgcggaagatcaactgggtgcgcgcgtgggctatattgaactggatctgaacagcggcaaaattctggaatcttttcgtccggaagaacgttttccgatgatgagcacctttaaagtgctgctgtgcggtgcggttctgagccgtgtggatgcgggccaggaacaactgggccgtcgtattcattatagccagaacgatctggtggaatatagcccggtgaccgaaaaacatctgaccgatggcatgaccgtgcgtgaactgtgcagcgcggcgattaccatgagcgataacaccgcggcgaacctgctgctgacgaccattggcggtccgaaagaactgaccgcgtttctgcataacatgggcgatcatgtgacccgtctggatcgttgggaaccggaactgaacgaagcgattccgaacgatgaacgtgataccaccatgccggcagcaatggcgaccaccctgcgtaaactgctgacgggtgagctgctgaccctggcaagccgccagcaactgattgattggatggaagcggataaagtggcgggtccgctgctgcgtagcgcgctgccggctggctggtttattgcggataaaagcggtgcgggcgaacgtggcagccgtggcattattgcggcgctgggcccggatggtaaaccgagccgtattgtggtgatttataccaccggcagccaggcgacgatggatgaacgtaaccgtcagattgcggaaattggcgcgagcctgattaaacattggtaaaccgatacaattaaaggctccttttggagcctttttttttggacgacccttgtccttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccgtaggccggttactctacgtaattgccctccagcgcgatacagcgattgtaacgaccttccagtttcttaatgccgttctggtaatattctttcggcttcgcttcaaagtacgcttcggtttcagcgataacttcttcgttgcaaccaaattttttacccgccagcatacgcttcaggtcgctaaacagaaaaaaatccgacggagccagatccgggctgtacggcgggtgcggcagcagttcgaaacccagctcatgaatcttcgccatggtacgcagggacttatgacagggggcgttgtcctggtgaaacagtaccttcttctttttcatgtgcgggcgttttgccgcgatttcaactttcaggcgttccagcagcgccatatagtaatcggaatttatggtcttacccttttccaggtagtcaatgaagataataccatgtgcatcccaaaatacgctcgccatgactttgcccgcgctcttttgggttttgccgcgtttcgggctcggttcgccagttgccgtccattctgctgactgacggttagactccggggtatagtggtgcagccacgtctcatccatggttacgtagcggcggaagaactccggcgtgttgcgggtcagcagctgcaggcaacgcttagaatcgtcgacacgacgctgtttctgatcaaaggtcagttcgcgcggaacccatttcgcacacagtttacgcatgtcaagatattggtggatgatgtgaccaacgcgctctttgctaattttcagagcttccgcaatttcgatcagcttcattttgcggtcgttcagaatcattttgtgaattttttttatattttcgtcggtgacaacctccttcggacgaccggaacgctcaccatcttcggtcgacatttcaccacgcttaaacttggcgtaccagtcgataatggttgatttacccggggcgctgtccggaaattcgttatccagccacgtctttgcttcgacggtgttcttacccttcagaaagcagtattttataagtaccctaaattcttttttttccattatttaagcctcctttttaaattaagatattgtcactttgtaatacggtccacaaaggtatgaataataatgtggcgcgcaaatttgatgcaaccattatcaccgccagaggtaaaatagtcaacacgcacggtgttagatatttatcccttgcggtgatagatttaacgttccgatttagtacctccatataaaggaggatcaaaatgtcaacgaagaaaaagccgcttacacaagagcagctagaggacgcacgtcgtctgaaagcaatctatgagaagaaaaagaatgagctgggtctgtctcaggaaagcgtagccgacaagatgggcatgggtcagagcggcgttggcgctctgtttaacggtattaatgcgctgaacgcgtacaacgccgcactgctgaccaagattctgaaagtttccgtcgaggagttctctccttctatagctcgtgaaatctatgaaatgtatgaagcggttagcatgcaaccgtctctgcgctctgaatacgaatacccggtcttcagccacgttcaagcaggtatgtttagcccggaactgcgtaccttcaccaaaggtgacgctgagcgttgggtatcgactaccaaaaaagcgagcgatagcgcgttttggctggaagtagaaggcaacagcatgacggccccgacgggcagcaagccgtcatttccggatggtatgctgatcctggttgatcctgagcaggcggttgagccgggagacttttgcattgcgcgcctgggtggtgatgaattcacctttaagaagctgatccgcgactctggccaagttttcctgcagccgctgaatccgcaatacccaatgatcccgtgcaacgaatcctgtagcgttgttggtaaggtcattgcatcccagtggccggaagaaaccttcggttaatttgtcagttacggcaagatccggccggcgcgccgatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcctttaaggtttaacggttgtggacaacaagccagggatgtaacgcactgagaagcccttagagcctctcaaagcaattttgagtgacacaggaacacttaacggctgacatggggcgcgcccag

SEQ ID NO: 128 - DNA, 89 bp, synthetic construct
  source 1..89: mol_type = other DNA
agattgccttgaatatattgacaatactgataagataatatataatatatctttactaccaagacgataaatgcgtcggaaaagtttaa

SEQ ID NO: 129 - DNA, 97 bp, synthetic construct
  source 1..97: mol_type = other DNA
atttaaatttgtgtctcaaaatctctgatgttacattgcacaagataaaaatatatcatcatgaacaataaaactgtctgcttacataaacagtaat

SEQ ID NO: 130 - DNA, 151 bp, synthetic construct
  source 1..151: mol_type = other DNA
agcacccattagttcaacaaacgaaaattggataaagtgggatatttttaaaatatatatttatgttacagtaatattgacttttaaaaaaggattgattctaatgaagaaagcagacaagtaagcctcttaagtataaggaggaaaaaat

SEQ ID NO: 131 - DNA, 44 bp, synthetic construct
  source 1..44: mol_type = other DNA
gttcttgacagcggaaccaatgtttagctaaactagagtctcct

SEQ ID NO: 132 - DNA, 84 bp, synthetic construct
  source 1..84: mol_type = other DNA
gagctgttgacaattaatcatcggctcgtataatgtgtggaattgtgagcggataacaattcttaagtataaggaggaaaaaat

SEQ ID NO: 133 - DNA, 84 bp, synthetic construct
  source 1..84: mol_type = other DNA
acgttaaatctatcaccgcaagggataaatatctaacaccgtgcgtgttgactattttacctctggcggtgataatggttgcat

SEQ ID NO: 134 - DNA, 714 bp, synthetic construct
  source 1..714: mol_type = other DNA
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 135 - DNA, 749 bp, synthetic construct
  source 1..749: mol_type = other DNA
tccgatttagtacctccatataaaggaggatcaaaatgtcaacgaagaaaaagccgcttacacaagagcagctagaggacgcacgtcgtctgaaagcaatctatgagaagaaaaagaatgagctgggtctgtctcaggaaagcgtagccgacaagatgggcatgggtcagagcggcgttggcgctctgtttaacggtattaatgcgctgaacgcgtacaacgccgcactgctgaccaagattctgaaagtttccgtcgaggagttctctccttctatagctcgtgaaatctatgaaatgtatgaagcggttagcatgcaaccgtctctgcgctctgaatacgaatacccggtcttcagccacgttcaagcaggtatgtttagcccggaactgcgtaccttcaccaaaggtgacgctgagcgttgggtatcgactaccaaaaaagcgagcgatagcgcgttttggctggaagtagaaggcaacagcatgacggccccgacgggcagcaagccgtcatttccggatggtatgctgatcctggttgatcctgagcaggcggttgagccgggagacttttgcattgcgcgcctgggtggtgatgaattcacctttaagaagctgatccgcgactctggccaagttttcctgcagccgctgaatccgcaatacccaatgatcccgtgcaacgaatcctgtagcgttgttggtaaggtcattgcatcccagtggccggaagaaaccttcggttaa

SEQ ID NO: 136 - DNA, 3532 bp, synthetic construct
  source 1..3532: mol_type = other DNA
tcagaattggttaattggttgtaacactggtctatcattgataggtataaattaatacgactcactaatactgaacctatcagtgatagatccaaacccaaaaacacaggagtttttagaatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaagcggatctttacagattctagacgggactctcaccaggtaccggagataccagcatcgtcttgatgcccttggcagctccagctgctaaggaggtatcaagatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaaaagcttgatgggggatcccatggtacgcgtgctag

(19) United States
(12) Patent Application Publication
(10) Pub. No.: US 2025/0207124 A1
(43) Pub. Date: Jun. 26, 2025

(54) COMPOSITIONS AND METHODS FOR EXPRESSING SYNTHETIC GENETIC ELEMENTS ACROSS DIVERSE MICROORGANISMS

(71) Applicant: Yale University, New Haven, CT (US)

(72) Inventors: Farren Isaacs, Stamford, CT (US); Jason Crawford, Shelton, CT (US); Jaymin Patel, New Haven, CT (US); Joonseok Oh, New Haven, CT (US); Shenqi Wang, New Haven, CT (US)

(21) Appl. No.: 18/848,065

(22) PCT Filed: Mar. 17, 2023
(86) PCT No.: PCT/US2023/064640

§ 371 Date: Sep. 17, 2024

Related U.S. Application Data
(60) Provisional application No. 63/321,073, filed on Mar. 17, 2022.

(51) Int. Cl. C12N 15/10; C12N 15/63; C12N 15/67; G16B 15/30; G16B 20/50; G16B 35/20; G16B 50/30

(52) U.S. Cl. CPC C12N 15/1089; C12N 15/635; C12N 15/67; G16B 15/30; G16B 20/50; G16B 35/20; G16B 50/30

(57) ABSTRACT

Computational strategies and compositions and methods of use thereof and formed therefrom are provided. Included are hybrid transcriptional expression signals for both prokaryotes and eukaryotes, and compositions and methods of introducing and mobilizing SGEs into multiple kingdoms. The strategies are particularly advantageous for hierarchically redesigning multigene biological pathways for mobilization, expression, and characterization in versatile organisms. Orphan biosynthetic gene clusters (BGCs) can be computationally redesigned into synthetic genetic elements (SGEs) and functionalized for expression across diverse hosts.

### CROSS-REFERENCE TO RELATED APPLICATIONS

This application claims the benefit of and priority to U.S. Provisional Application No. 63/321,073 filed on Mar. 17, 2022, the contents of which is incorporated herein in its entirety.

### STATEMENT REGARDING FEDERALLY SPONSORED RESEARCH

This invention was made with government support under GM067543 and CA215553 awarded by National Institutes of Health and under 1923321 awarded by the National Science Foundation. The government has certain rights in the invention.

### REFERENCE TO THE SEQUENCE LISTING

The Sequence Listing submitted as a text file named “YU_8252_PCT_ST26.xml” created on Mar. 17, 2023, and having a size of 214,373 bytes is hereby incorporated by reference pursuant to 37 C.F.R. § 1.834(c)(1).

### FIELD OF THE INVENTION

The disclosed invention is generally in the field of recombinant expression systems and specifically in the area of multigene pathways.

### BACKGROUND OF THE INVENTION

High-throughput DNA sequencing has revealed the complete genome sequences of many organisms, establishing a fundamental understanding of genetic variation associated with phenotypic diversity. Phenotypic diversity endows organisms with rich biosynthetic and molecular capabilities (Tobias and Bode, 2019) and allows them to adapt to diverse environments (Agrawal, 2001; Rainey and Travisano, 1998). Establishing systematic causal relationships between genotypes and phenotypes can be facilitated by the development of synthetic biology technologies capable of probing and manipulating diverse biological systems at the genetic, metabolic, and regulatory levels (Lee and Kim, 2015). Harnessing this diversity has tremendous potential to solve global challenges, such as producing new drugs and programmable cells (Farkona et al., 2016; Leventhal et al., 2020) to alleviate human diseases (Isabella et al., 2018) and synthesizing new chemicals (Austin and Rosales, 2019) and materials (Xu et al., 2018) to ensure environmental sustainability.

A predominant mediator in the genotype-phenotype axis is the rich arsenal of structurally complex secondary metabolites that often mediate interspecies interactions in various ecological niches, such as the human microbiome (Donia and Fischbach, 2015; Shine and Crawford, 2021; Vizcaino et al., 2014). These specialized metabolites, or natural products (NPs), tend to harbor distinct scaffolds that underlie diverse biological activities (Davison and Brimble, 2019), and therefore, provide valuable molecular leads for agriculture, biotechnology, and medicine (Newman and Cragg, 2020; Shen, 2015). Advanced biosynthetic pathway prediction algorithms (Blin et al., 2019; Navarro-Muñoz et al., 2020; Skinnider et al., 2017) have revealed massive untapped microbial biosynthetic capacity for the production of new bioactive small molecules (Cimermancic et al., 2014). Integrated microbial genomes—atlas of biosynthetic gene clusters (IMG-ABC), the largest public database of biosynthetic gene clusters (BGCs) (Palaniappan et al., 2019), currently catalogs 411,011 predicted gene clusters, 96% of which are from bacteria sourced from only 60,445 genomes. Of these, only 1,285 BGCs have been experimentally verified. Despite this diversity, the tools needed to functionally interrogate and structurally characterize the growing body of “orphan” (i.e., structurally uncharacterized) pathways are limited (Covington et al., 2021).

Characterization of BGCs endogenously in their native hosts is impeded by numerous factors. A significant fraction of environmental strains are not readily cultured (Bodor et al., 2020). When cultivation is tractable, most BGCs are silenced under standard laboratory conditions (Ren et al., 2017; Scherlach and Hertweck, 2021). Although these silent BGCs can be activated through strain engineering (Sidda et al., 2014; Zhang et al., 2017), this strategy relies on the existence of genetic tools for each strain of interest. Additionally, advances in de novo genome assembly directly from metagenomic extracts permits culture-independent prediction of orphan BGCs (Sugimoto et al., 2019).

Accordingly, heterologous expression in model hosts is an important strategy for BGC characterization. This technique transplants BGCs into tractable model organisms (Li et al., 2015; Ross et al., 2015) by cloning them on episomal vectors (Hover et al., 2018). To overcome expression bottlenecks, pathways have been refactored transcriptionally (Yamanaka et al., 2014) and through complete operon redesign (Smanski et al., 2014). In addition to discovery, heterologous expression has facilitated new routes to access highly desired known natural products (Ajikumar et al., 2010; Galanie et al., 2015; Paddon et al., 2013). However, selection of heterologous host is unpredictable because BGCs can fail to function due to numerous factors, which include the lack of correct substrate inputs, improper protein folding, or divergent metabolic outputs (Casini et al., 2018; Craig et al., 2010). For example, even within the same genus, different isolates can significantly differ in both the expression and chemical outputs of identical gene clusters (Iqbal et al., 2016; Santos et al., 2013; Wang et al., 2019a). Given the intrinsic promiscuity of biosynthetic enzymes (Glasner et al., 2020), molecular outputs can be influenced by the broader metabolic context of the host. As an example, the genotoxin colibactin (Nougayrede et al., 2006; Xue et al., 2019) produced by E. coli requires a chaperone Hsp90E, for production to protect from clpQ-mediated proteolytic cleavage of biosynthetic proteins, highlighting the strain-dependent complexity of pathway productivity (Garcie et al., 2016). Similarly, in a “pressure test” of synthetic biological foundries tasked to heterologously produce various complex small molecules, production host choice was a prominent design consideration (Casini et al., 2018). This makes sense given that intracellular metabolism, gene regulation, protein folding, availability of input metabolites, and toxicity vary among organisms. These challenges encourage new approaches to readily access and domesticate phylogenetically diverse organisms for heterologous expression of BGCs (Brophy et al., 2018; Wang et al., 2019a).

Some progress has been made in the field of synthetic biology to facilitate the engineering of organisms through the development of biological parts, devices, and systems to assemble complex genetic circuits and expression platforms to achieve remarkable control of biological systems (Elowitz and Leibler, 2000; Khalil and Collins, 2010; Lopatkin and Collins, 2020). These include the development of logic gates (Nielsen et al., 2016), biosensors (Riglar et al., 2017), recoded genetic codes (Fredens et al., 2019; Lajoie et al., 2013; Ostrov et al., 2016), and synthetic metabolic networks (Choe et al., 2020). There is considerable interest in expanding the tractability of non-model organisms, motivated by the need to overcome the aforementioned challenges of studying complex biosynthetic pathways in non-native contexts.

Biological diversity intrinsically challenges the ability to port synthetic genetic programs from one chassis to another, especially across taxonomic domains. Due to tremendous phylogenetic differences in the maintenance, regulation, and expression of genetic elements, these efforts typically require specialized solutions and optimization for each host, and thus remain a defining challenge for the field. Several layers of regulation impede the functional mobility of genetic parts. Pathways for specialized metabolites are often controlled at the transcriptional level resulting in strain and environment-dependent expression (Seyedsayamdost, 2014). Similarly, translation bottlenecks can occur due to differences in codon usage and translation initiation signals (Lithwick and Margalit, 2003). Additionally, a major challenge is in the mobilization, delivery, and stable inheritance of genetic elements into diverse hosts. In this regard, several strategies have made progress. For example, plasmid libraries mobilized by RK2-mediated conjugation have transferred fluorescent reporters to phylogenetically diverse bacteria; however, the fluorescent signal was quickly lost from populations due to plasmid loss (Ronda et al., 2019). To augment stability, engineered integrative and conjugative elements (ICE) could be used to self-mobilize and chromosomally integrate heterologous cargo in a variety of environmental Bacilli strains (Brophy et al., 2018). In a similar vein, chassis-independent recombinase-assisted genome engineering (CRAGE) allowed the dissemination of genetic elements to Proteobacteria and Actinobacteria species (Wang et al., 2019a). However, these and other integrative strategies—e.g., phage-assisted integration, and site-specific integrases (Du et al., 2015). For example, engineered Cas-transposases (Chen and Wang, 2019) can potentially augment host choice by allowing strain-specific targeting of cargo.

Thus, it is an object of the invention to provide strategies, compositions, and methods for mobilizing synthetic genetic elements across diverse microorganisms.

### BRIEF SUMMARY OF THE INVENTION

Methods of recoding a nucleic acid coding sequence are provided. The methods can include comprising two, three, four, five, or all six of steps: (1) selecting the codons of the coding sequence, (2) implementing N-terminal codon bias; (3) creating a synthetic or hybrid 5′ regulatory element; (4) screening for internal ribosome binding sites (RBSs); (5) randomizing one or more codons upstream of internal RBSs, and (6) screening for internal terminators. Typically, the recoding improves expression of the nucleic acid coding sequence in one or more heterologous organisms of interest. The original nucleic acid coding sequence is typically a naturally occurring sequence and the recoded sequence is typically a synthetic sequence. The coding sequence can be any coding sequence. In some embodiments, the coding sequence encodes a polypeptide. In some embodiments, the polypeptide is part of a biosynthetic pathway that works in concert with other polypeptides encoded in a biosynthetic gene cluster.

In some embodiments, step (1) is based partially or completely on the preferred codon distribution in the heterologous organism(s). For example, codon usage can be selected based on that of highly expressed genes in the heterologous organism(s). Codon usage information can be derived from the genome sequence of a strain(s) of the heterologous organism or downloaded directly from a database(s). Step (1) can additionally or alternatively include depletion of canonically-inhibiting codons, optionally wherein the inhibiting codons are selected from TTA, AGG, CTA, CGA, CGG, CGA, TTG and/or GTG, or a combination thereof.

In some embodiments, step (2) includes recoding the nucleic acid sequence encoding the N-terminus of a polypeptide encoded by the nucleic acid coding sequence to reduce secondary and/or tertiary structure. Reducing secondary structure can include recoding a 5′ terminal stretch of 15-75 base pairs, or any subrange or specific integer therebetween, of the nucleic acid coding sequence. Step (2) can include using a hybrid codon distribution that biases toward privileged or preferred codons encoding the N-terminus that correlate with high expression levels in the heterologous organism(s). In some embodiments, the recoding of the nucleic acid sequence encoding the N-terminus of a polypeptide includes the codon adaptation index (CAI) approach and/or the tRNA adaptation index (TAI). Typically, the synthetic or hybrid regulatory element is designed for versatile regulation across diverse prokaryotes and eukaryotes, and may include creation of hybrid of eukaryotic and prokaryotic element(s) that can impact gene expression in one, two, three, or more microbial taxa, optionally wherein one or more of the taxa include the heterologous organism(s). In some embodiments, step (3) includes utilizing a thermodynamic translation initiation model optionally wherein the thermodynamic translation initiation model defines sequence and/or structural determinants of ribosomal entry, optionally bacterial ribosome entry, and allows predictions of translation initiation rates using a ribosomal binding site (RBS) calculator. Step (3) can include consideration of parameters that increase the range of host cells in which the nucleic acid coding sequence can be expressed, optionally highly expressed, optionally wherein the such parameters include incorporation of Shine-Dalgarno sequence requirements and/or start codon spacing preferences for the heterologous organism(s). In some embodiments, step (3) includes maintaining or recoding the nucleic acid sequence to enrich for poly AT sequence and/or a “AAA” sequence motif immediately upstream of the start codon. In some embodiments, step (3) includes maintaining, recoding, or adding to the nucleic acid sequence a synthetic 5′ untranslated region comprising N₁₇(A/U)₆AGGAGN₄AAA (SEQ ID NO:1), and optionally iteratively mutating/varying ‘N’ positions until a desired translation initiation strength is reached, optionally wherein the translation initiation strength is reached by prediction or empirically determined.

Step (4) can include recoding one or more alternative NTG start codon(s), one or more internal RBS(s), one or more terminator(s), or a combination thereof. Internal RBSs can be NTG sites throughout the CDS in all three coding frames. Step (4) can include recoding the sequence upstream of one or more RBS(s) to structurally reduce internal ribosomal entry. Step (4) can include predicting ribosome binding strength, calculating thermodynamic parameters, or a combination thereof.

In some embodiments, the method includes iteratively repeating steps (4) and (5) in two or more cycles. In some embodiments, initiation strength is predicted or determined empirically after each cycle, and wherein the cycles are terminated when a desired translation initiation strength is reached.

Any one or more steps, or aspects thereof, can be computer implement. In some embodiments, the entire method is computer implemented.

Recoded nucleic acid sequences prepared according to the disclosed methods are also provided.

Also provided are inducible expression circuits. In some embodiments, the expression circuits include seed elements or a seed promoter operably linked to an RNA polymerase promoter operable linked to the polymerase coding sequence, wherein the seed element drive initial transcription of the RNA polymerase, and subsequent transcription is auto-regulated through a positive and/or negative regulation of the RNA polymerase promoter. In some embodiments, the circuit includes one or more of a repressor/operator pair, CRISPRi and/or CRISPRa. In some embodiments, the promoter is pT7 and the RNA polymerase is T7/RNAP the promoter is pT3 and the RNA polymerase T3/RNAP, or the promoter is pSP6 and the RNA polymerase SP6 RNA polymerase.

In some embodiments, the circuit includes a tetO tet-on tetracycline-controlled transcriptional activator sequence, an anhydrotetracyline (aTc) responsive TetR repressor, Tet-off tetracycline-controlled transcriptional repressor, riboswitch (e.g., a theophylline-responsive translational riboswitch), or a combination thereof. In some embodiments, the circuit includes a vanO van-on Vanillin acid-controlled transcriptional activator sequence, an vanillin acid responsive VanR repressor, Van-off tetracycline-controlled transcriptional repressor, riboswitch (e.g., a theophylline-responsive translational riboswitch), or a combination thereof. Such a system can be regulated essentially exclusively by theophylline.

Synthetic genetic elements (SGEs) are also provided. The SGEs typically include a coding sequence (CDS) operably linked to a hybrid regulatory element suitable for expressing the coding sequence in organisms from two or more different kingdoms. In some embodiments, one of the kingdoms is Monera and another is Animalia, Plantae, Fungi, or Protista. Preferably, the hybrid regulatory element is suitable for expressing the CDS in prokaryotes and eukaryotes. The hybrid regulatory element can include one or more of a promoter, a 5′ UTR, and 3′ terminator. The regulatory element can include one or more upstream activity sequences (UASs), a core sequence, a TATA box, one or more spacer sequence, or a combination thereof. In some embodiments, the hybrid regulatory element(s) includes 1-10 UASs operably linked to the promoter. In some embodiments, the hybrid regulatory element includes one or more spacer sequence, optionally comprising poly-A or poly-T in an effective amount to deplete the probability of nucleosome occupancy at a TATA box (e.g., TATAAAG) and/or a transcriptional start site (TSS). In some embodiments, the promoter is a natural or synthetic eukaryotic promoter, optionally a natural or synthetic yeast promoter, or a variant thereof. In some embodiments, the hybrid regulatory element includes a transcription start site (TSS), optionally including the consensus motif [A(A_{rich})₅ NPy A (A/T)NN(A_{rich})₆]. In some embodiments, the hybrid regulatory element includes any one of SEQ ID NOS:50-98, or variant thereof with at least 70% sequence identity thereto.

The SGE can optionally further include one or more intervening terminators, optionally flanking the promotor sequence.

In some embodiments, the SGE includes two or more CDS, wherein each CDS is operatively linked its own hybrid regulatory element, wherein the hybrid regulatory element of each CDS is the same, different, or a combination thereof. Coding sequences are discussed above and elsewhere herein. Thus, in some embodiments, the two or more CDS together form part or all of a biosynthetic pathway. In some embodiments, the biosynthetic pathway is present as a gene cluster in an organism's genome.

In some embodiments, the regulatory element is characterized in having:
(i) no pair of UASs is used more than 5, 4, 3, 2, or, 1 time, optionally no more than 3 times, and optionally no triplet of UASs is used more than once;
(ii) promoters range from 100 bp to 250 bp inclusive, or any subrange thereof, or specific integer therefore, optionally 161 bp to 181 bp, in length;
(iii) no spacer or TSS sequence is used more than once;
(iv) no ‘NTG’ sequence is used in any spacer to avoid internal start codons; and/or
(v) predicted terminators and RBSs (e.g., as discussed above) in promoters are removed by randomly inserting or substituting mutating spacer sequences.

In some embodiments, a SGE includes a prokaryotic RBS, a bacterial promoter, a eukaryotic promoter for each CDS, and a eukaryotic terminator. The SGE can further include an inducible polymerase promoter expression circuit.

In some embodiments, the SGE is flanked by integration sequences, e.g., asymmetrical attB sites. Such SGE may be free from a prokaryotic RBS, a bacterial promoter, and inducible expression circuit, and or a eukaryotic terminator.

Also provided are vectors encoding or including SGE and optionally further encoding an integrase such as phiC31 integrase and/or a selectable marker.

Landing pads for SGEs are also provided. A landing pad typically includes a nucleic acid cassette having a nucleic acid sequence encoding an inducible expression control circuit, a promoter operably linked to a reporter gene, a selectable marker, and integration sites flanking the reporter gene. The landing pad can further include transposase terminal repeats flanking the cassette, followed by a sequence encoding the transposase, preferably which itself does not mobilize into the recipient genome. Preferably, the transposase is independent of host-specific factors and shows little bias in random integration such as Himar or Tn5. In some embodiments, the sequence encoding the selectable marker (e.g., an antibiotic selectable marker) is operably linked to a seed promoter.

Vectors encoding or including a landing pad are also provided.

Methods of introducing a landing pad into a host organism are also provided and can include introducing into the host cell a landing pad, for example, by transformation or transfection of a vector encoding the landing pad into a first host organism, expressing the transposase, and introduction of the landing pad into a second host organism by conjugation with the first host organism.

Methods of introducing a synthetic genetic element into a host cell are also provided and typically include conjugation of a host cell including an SGE vector to another cell with a landing pad integrated therein. Typically an integrase is expressed and facilitates integration of the SGE into the landing pad, optionally wherein the SGE replaces the landing pad's selectable marker.

Thus, host cells including the disclosed SGEs and landing pads are also provided. The SGEs and/or landing pads can be integrated into the host's genome, or extrachromosomal.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1F are schematics illustrating the disclosed computational and experimental strategy to hierarchically redesign multigene biological pathways for mobilization, expression, and characterization in versatile organisms. In FIGS. 1A and 1B, orphan biosynthetic gene clusters are sourced and each CDS is redesigned. In FIG. 1C, the redesign appends hybrid synthetic expression sequences functional in bacterial and yeast heterologous hosts. In FIG. 1D, the redesigned synthetic genetic elements (SGEs) are mobilized using integrative shuttle vectors into cross-kingdom hosts. In FIG. 1E, pathway-targeted metabolomics is used to identify pathway and gene-dependent metabolic signatures. In FIG. 1F, the metabolites are purified for structural and functional characterization.

FIGS. 2A and 2B are graphical representations illustrating the design process of the Synthetic Gene Elements (SGEs). FIG. 2A shows that the overall SGE design includes redesigning each CDS within a multigene pathway and appending with hybrid eukaryotic and prokaryotic regulatory elements, compiled back into a synthetic operon. FIG. 2B outlines the CDS redesign and optimization whereby codon selection is utilized to recreate N-terminal codon bias patterns seen in native genes, create synthetic 5′ hybrid UTRs, and screen to avoid internal start and termination signals. FIGS. 2C and 2D illustrate the codon usage distribution used in Example 1 for CDS redesign. In FIG. 2C, the codon distribution of highly expressed genes (HEGs) from E. coli is used to assign the probability that a given codon is used for each amino acid. The codons highlighted in red are universally excluded due to reported translational inhibitory activities (TTA, CTA, CGA, CGG, AGG) and to prevent alternative start codons (GTG, TTG). In FIG. 2D, to quantify N-terminal codon bias in each microbial strain used in Example 1, the MFE RNA folding energy (in kcal/mol) is measured across 200 randomly selected wild-type CDSs using a 30 bp sliding window. For each sliding window base pair position, these values are averaged across all tested CDSs; the nucleotide position noted is the center of the 30 bp sliding window. In FIG. 2E, a test set of E. coli genes is used to quantify N-terminal codon bias in native and recoded genes. The test set contains all CDSs that lack an upstream overlapping CDS within 35 bp. The MFE RNA folding energy (in kcal/mol) is measured across each CDS using a 30 bp sliding window. For each sliding window base pair position, these values are averaged across all tested CDSs; the nucleotide position noted is the center of the 30 bp sliding window. This analysis is performed for native wildtype E. coli gene sequences and for recoded genes with and without accounting for N-terminal codon bias. In FIG. 2F, the impacts of codon usage at the N-terminal 12 amino acids were evaluated by designing eGFP variants with unstructured 5′-RNA ends (folding energy of first 30 bp is <3.5 kcal/mol). Here, unstructured RNAs were created using highly used codons (HEG codons) in E. coli or using rarer codons that were found to be enriched in HEGs (Goodman et al., 2013) (FACSseq-enriched codons). FIG. 2F is a dot plot of results demonstrating that the use of HEG codons to create unstructured RNAs resulted in more highly expressed GFP variants than the use of FACSseq-enriched codons (n=12 GFP variants for each condition). In FIG. 2G, the remainder of the CDS was recoded with various base codon distributions. FIG. 2G is a dot plot showing no significant correlation between codon adaptation index (CAI) and gene expression, indicating that the bulk ORF is generally permissive regarding codon usage (n=12 GFP variants for each condition). FIG. 2H is a dot plot showing results demonstrating that recoding GFP genes to match E. coli's codon usage performed comparably in E. coli to those recoded to match B. subtilis codon usage, or were neutrally randomized, or were enriched in inhibitory codons to create poor CAI values (n=12 GFP variants for each condition). FIG. 2I is a schematic overview of the design principles for hybrid prokaryotic/eukaryotic 5′-UTRs to promote efficient translation initiation. These upstream elements also include sequences engineered to promote eukaryotic transcription through depletion of nucleosome occupancy around the TSS. In FIG. 2J, each CDS in the test set is recoded with and without actively screening for internal bacterial RBSs (with a translation initiation rate (TIR) cutoff of >100). For each method, the frequency of internal RBS occurrence is plotted as a frequency distribution. After screening, the number of internal TIRs falls from 3.8 to 0.6 per gene. Each CDS in the test set is recoded 100 times to quantify the prevalence of transcriptional terminators appearing during the recoding process, and the fraction of recoding attempts that resulted in transcriptional terminators is plotted.

FIGS. 3A-3C are schematics illustrating the development of a library of synthetic yeast promoters for cross-kingdom multigene pathway expression. FIG. 3A is an overview of synthetic operon architecture for cross-domain expression. In FIG. 3B, individual open reading frames are flanked with synthetic 5′-UTRs adapted for translation initiation, as well as yeast promoters and terminators. FIG. 3C demonstrates that synthetic yeast promoters include combinatorial arrays of upstream activating sequences (UASs), cores, TATA boxes, and TSSs. Spacer sequences are then further modified to deplete nucleosome occupancy at the TATA box and TSS.

FIGS. 4A-4D are graphical representations illustrating the systematic depletion of the probability of Nucleosome Occupancy. In FIG. 4A, using NuPoP to predict the probability of nucleosome occupancy, three commonly used native S. cerevisiae promoters—cyc1, adh1, and tef1—were evaluated to highlight depletion of occupancy at promoter regions. The annotated Transcription Start Site (TSS) is indicated by the dashed line; 400 bp of sequence flanking the TSS is used for analysis. In FIG. 4B, an initial test synthetic promoter is created. Nucleosome occupancy is predicted before and after algorithmic manipulation of sequence to deplete occupancy. FIG. 4C shows a nucleosome occupancy prediction, before and after algorithmic depletion, is shown for all 48 synthetic promoters. Depletion could not be achieved for YP17, YP37, and YP46. FIG. 4D is a bar graph showing the impact of UAS number and nucleosome depletion gauged in S. cerevisiae on an initial test promoter design driving the production mUkGFP. Promoter strength is benchmarked against the cyc1, adh1, and tef1 promoters.

FIG. 5A is a schematic of the pYP backbone in a S. cerevisiae-E. coli shuttle vector used to clone and characterize synthetic yeast promoters upstream of a GFP reporter gene. In FIG. 5B, an expanded set of 48 synthetic promoters, cloned upstream mUkGFP are tested via flow cytometry, and benchmarked against the cyc1 (C), adh1 (A), and tef1 (T) promoters. Promoters are developed with and without nucleosome depletion (red and grey, respectively) and with 3, 4, or 5 UASs (blue, green, and purple, respectively). FIG. 5C is a bar graph showing the results for given individual promoters; additional UASs can increase expression levels, as was demonstrated with YP2 and YP7, an effect was not observed with YP8. In FIG. 5D, mRNA levels are quantified by qRT-PCR for a subset of promoters (YP1, YP13, YP14, YP18, YP23, YP30, YP41, YP45) and plotted against GFP fluorescence to measure the linear correlation between protein and mRNA levels. In FIG. 5E, the same constructs are measured in E. coli BL21(DE3), where GFP is transcribed from a fixed T7 promoter. Variability in fluorescence is observed. Mean and standard deviation of fluorescence across all constructs is denoted with solid and dashed lines, respectively. Fluorescence values are linearly normalized so that cyc1=100. In FIG. 5F, reproducibility of promoter strength is gauged by comparing mUkGFP fluorescence, via flow cytometry, to a distinct eGFP that shares no detectible sequence homology. A linear correlation is calculated to report an r² correlation value. In FIG. 5G, the correlation between the strength of the S. cerevisiae promoter and the expression of this hybrid promoter in E. coli BL21(DE3) is evaluated by plotting fluorescence in S. cerevisiae against the fluorescence in E. coli for each promoter. A very weak linear correlation is seen (r²=0.18), indicating that attenuated expression in E. coli is not related to the strength of the yeast element. In FIG. 511, mRNA level of 8 representative pT7/yeast promoters hybrids (YP1, YP13, YP14, YP18, YP23, YP30, YP41, YP45) transcribing mUkGFP are evaluated by qRT-PCR in E. coli. Values are plotted against mUkGFP fluorescence driven from each promoter. In FIG. 5I, pT7/yeast promoter hybrids are used to transcribe two distinct fluorescent genes in E. coli, which share no nucleotide sequence similarity—eGFP and mUkGFP. Fluorescence values for each synthetic promoter were collected.

FIGS. 6A and 6B are schematics illustrating the development of a host factor independent T7 RNA polymerase expression circuit. FIG. 6A illustrates the final expression circuit design, featuring auto-inducing positive feedback from the RNAP, negative feedback from a TetR repressor, and expression titration via a theophylline translational riboswitch. FIG. 6B exemplifies the various circuit architectures that were developed during the design-build-test-learn process. In FIGS. 6C-6D, the pT7RNAP backbone is used to clone the variants of the T7 RNAP circuit. The pT7GFP plasmid enables a readout of the T7 RNAP circuit by encoding a pT7-transcribed eGFP reporter gene. FIG. 6E is a bar graph demonstrating a comparison of the RNAP circuit variants using a GFP reporter driven by a T7 promoter. Each design is quantified with and without induction. FIG. 6F is a bar graph demonstrating modulation of positive feedback strength by comparing a wt T7 promoter with an attenuated mutant (H9). Both promoters are used to drive an eGFP reporter in E. coli BL21(DE3) to benchmark differences in strength.

FIG. 7A is a schematic illustrating the development of a host factor independent T7 RNA polymerase expression circuit. pBroad is ultra-broad-host range vector capable of replicating in Gram-negative bacteria (RSF1010 origin) and Gram-positive bacteria (pAMβ1 origin) and mobilized via the conjugative RP6 oriT. This vector episomally carries the T7 RNAP, along with a pT7-GFP/nanoluc reporter. This reporter is flanked with phiC31 attP sites for site-specific insertion of BGCs. FIG. 7B is a bar graph demonstrating inducible expression in both Gram-negative E. coli and Gram-positive B. subtilis bacteria. Circuit variant T15 is inserted into a broad host-range shuttle vector containing RSF1010 and pAMBI origins of replication and a pT7-GFP reporter (pBroad). In all cases of positive theophylline induction, aTc concentration is fixed at 100 ng/mL.

FIGS. 8A-8C are schematics illustrating the construction of landing pads for SGE expression in diverse bacteria. In FIG. 8A, conjugative transposition was used to randomly introduce a landing pad into host bacterial genomes. This landing pad consists of the T7 RNAP circuit (variant T15), a pT7 GFP-nanoluc reporter to assay expression, and attP sites for site-specific integration of SGEs. “pX” refers to the “seeding” promoter driving the antibiotic resistance gene and the T7 RNAP circuits. “pX” is either host-range promoter kanR P1 from pIP433, or is absent, in which case “seeding” transcription is provided by basal transcription from the recipient genome locus. FIG. 8B exemplifies that upon establishment of a genomically-integrated landing pad, this site can be used to site-specifically integrate genetic cargo via a phiC31 integrase at the cognate attP sites. FIG. 8C illustrates the pLP vector, which carries a landing pad consisting of an antibiotic selectable marker, the T7 RNAP circuit, a pT7-GFP/nanoluc reporter, and phiC31 attP sites. This landing pad is integrated into recipient genomes through transposition (Tn5 or Himar). It is maintained on the R6K suicide origin of replication and conjugatively mobilized via the RP4 oriT.

FIG. 9 is a bar graph demonstrating a comparison between constitutive and inducible bacterial promoters used in Example 1 to seed the T7RNAP circuit and drive transposase. A series of bacterial promoters were cloned upstream of eGFP and transformed into E. coli Machi cells. Fluorescence is quantified by flow cytometry using a FACS aria. Constitutive promoters are highlighted in green. IPTG (1 mM)—inducible promoter pTac is highlighted in red. The temperature sensitive pR/CI857 system bacteriophage lambda is highlighted in blue; here, wildtype CI857 (light blue), and recoded CI857 with synthetic RBS (dark blue) are compared to quantify increase in activity due to induction at 37 C or 42 C. Sequences of exact promoter sequences used can be found in Table 1(i.e., SEQ ID NO:2-49).

FIG. 10A is a schematic illustrating the multifunction pInh plasmid. The multifunctional pInh plasmid silences mobile elements within conjugation donor strains to prevent toxicity and instability. The SP6 RNAP silences transposases by promoting an anti-sense transcript on pLP, the Tn5 inhibitor silences the Tn5 transposase through dominant-negative inhibition, and the T7 Lysozyme silences basal activity from the T7 RNAP circuit. FIG. 10B is an area graph illustrating the clonal expression of the transposed populations with and without T7 RNAP circuit induction. The landing pad was transconjugated into E. coli MG1655 and approximately 2000 clones were pooled and assayed by flow cytometry. The distribution of uninduced and theophylline+aTc induced fluorescence in the population was quantified to demonstrate the extent of clonal heterogeneity. From the population, four individual clones were randomly picked and similarly quantified with and without induction. Expression strength and variability are indicated by the mean and coefficient of variation (CV).

FIG. 11A is a schematic illustrating the pPath vector; the entry vector for the cloning of SGEs. SGEs are cloned at the multiple cloning site, replacing the sacB counter-selectable marker. In yeast, this vector can replicate as a centrometric plasmid. In bacteria, the phiC31 integrase integrates the SGE into landing pads at cognate attP sites. FIG. 11B is a schematic of the biosynthetic pathway for the purple pigment violacein was used to demonstrate function. This pathway was cloned with its native sequence under its native promoter element, under the orthogonal T7 promoter, and as a fully redesigned SGE. FIG. 11C is a bar graph illustrating quantification of the production of violacein through absorbance in its native host Chromobacerium violaceum and in landing pad-domesticated Pseudomonas putida. Production of violet pigment was quantified by absorbance at 585 nm while cell density was quantified by absorbance at 660 nm. P. putida strains were induced with 1 mM theophylline+100 ng/mL aTc.

FIGS. 12A-12E are graphical representation demonstrating the characterization of a new class of nucleotide metabolites from the human microbiome. FIG. 12A provides an overview of the refactored orphan BGC from the vaginal isolate Lactobacillus iners LEAF 2052A-d (BGC08). In addition to the presumed core biosynthetic genes, a proximal downstream gene and PPTase were included elsewhere in the genome. Gene functions were predicted using BLAST and InterPro searches. The biosynthetic pathway was cloned as its native sequence, with an orthogonal T7 promoter, and as a fully redesigned SGE. FIG. 12B is a pair of heat maps quantifying production of metabolites (2 and 4) in landing pad-domesticated P. putida with each construct. FIG. 12C are EIC traces demonstrating genotype to metabolite relationships of enzyme-dependent metabolites For FIG. 12C, single gene knockouts were performed on the enzymatic genes in E. coli as a host. FIG. 12D-12E illustrates the proposed biosynthetic route of the tyrocitabines based on the single gene knockout data and analytical chemistry NMR and LC-MS/MS studies.

FIGS. 13A-13H show results from in vitro biochemical analyses of tyrocitabine biosynthesis. FIG. 13A shows a biosynthetic route to 4 is supported via in vitro biochemical reactions using purified enzymes. TybC was reacted with L-tyrosine and various candidate ribose donors to produce 1 and 2. NTPs, mixed nucleotide triphosphates; NMN, nicotinamide mononucleotide; Rib5′P, ribose 5′-phosphate. FIG. 13B shows results from reactions of TybE with both isolated 1 and 2 in the presence of putative cofactors NADH and NADPH to produce 3 and phospho-3, respectively; phospho-3 was not detected in cell extracts. FIGS. 13C-13D are bar graphs of results from experiments of tyrocitabine production enhancement through substrate feeding and detection in native host. FIG. 13C shows tyrolose (2) production in an E. coli heterologous host was enhanced by feeding L-tyrosine, supporting tyrosine as a substrate for biosynthesis. FIG. 13D shows tyrocitabine-626 (4) production was enhanced by feeding synthetic tyrolose 2 in the medium, supporting 2 as a substrate for conversion into 4. FIG. 13E shows that in a tybC knockout background, production of 4 can be rescued by feeding synthetic 2 (chemical complementation), supporting 2 as an authentic intermediate and substrate for reactions downstream of TybC. FIG. 13F shows results from reactions of TybB with 2 or 3 in the presence or absence of ATP to test for the ATP-dependent production of 4. Purified 4, synthetic 1 and 2, and cellular extracts were used as standards. FIG. 13G shows that the production of acylated tyrocitabine-752 (8) was enhanced by feeding octanoic acid, supporting the fatty acid as an acyl donor. FIG. 13H is a bar graph showing production of tyrocitabine-626. TybB was reacted with 2 or 3 in the presence or absence of ATP to test for the ATP-dependent production of 4. Purified 4, synthetic 1 and 2, and cellular extracts were used as standards. FIG. 13H are bar graphs showing detection of tyrolose (2). The native host of the tyb pathway, Lactobacillus iners LEAF 2052a-D, grown anaerobically in NYCIII medium. Production of tyrolose (2) was observed.

FIGS. 14A-14D are graphs illustrating inhibition of in vitro transcription/translation by tyrocitabines. In FIG. 14A, inhibition of an E. coli in vitro translation reaction was performed using tyrocitabine-626 (Compound 3) and erythromycin. In order to quantify inhibition of in vitro protein translation, DNA encoding eGFP, as well as compound (or H₂O vehicle) was added at various concentrations, with endpoint fluorescence measured after 4 hours. In FIGS. 14B-14C, production of eGFP from nucleic acid template is quantified using the NEB Purexpress in vitro transcription/translation system. Fluorescent values are normalized to the untreated control. Assay activity is measured with the use of an eGFP DNA template and RNA template to distinguish inhibitory activity by tyrocitabine 626 (3) at the transcription level vs translational level within the in vitro assay. In FIG. 14D, inhibition of activity is evaluated for tyrolose (2), tyrocitabine 626 (3), and the 2-carbon acylated tyrocitabine 669 (4).

FIGS. 15A and 15B are graphical representations of the cross-kingdom production of the tyrocitabines. In FIG. 15A, the SGE of this pathway was introduced into various Gram-negative, Gram-positive, and eukaryotic hosts. E. coli, K. aerogenes, P. putida, and S. enterica were domesticated with integrated landing pads for T7RNAP production, which was modulated with an induction gradient of theophylline. In B. subtilis, this landing pad was present on the pBroad vector. Pathways were cloned on the conjugative pPath vector, which site-specifically integrates into the landing pad in bacteria, and can be dually maintained centromerically in S. cerevisiae. In all cases of positive theophylline induction, aTc concentration was fixed at 100 ng/mL. In S. cerevisiae, production was constitutive. LC/MS ion counts of the most abundant pathway-dependent metabolites is quantified (m/z 314, 624, 669, and 753). Additionally, endpoint OD₆₀₀ is measured for all theophylline-inducible strain to highlight the fitness impacts of pathway induction. In FIG. 15B, 24 representative putative homologs of the tyb pathway are shown to highlight differences in accessory genes present within the operons. Harboring strains are sorted by taxonomy (red nodes=Firmicutes, blue nodes=Actinobacteria, black nodes=other candidate phyla). TybB-like abortive tRNA synthetases, tybC-like nbosyltransferases, and tybE-like dehydrogenases are highlighted in red, blue, and green, respectively. Accessory proteins with predicted function (by IMG-DOE) are highlighted in purple and putative functions are listed. Accessory proteins with unknown function are highlighted in black. The exact strain ID for each species listed is found in (Table 2). FIG. 15C is a schematic of the Interpro-predicted domains of canonical TyrRS from Lactobacillus iners LEAF2052A d compared with TybB.

FIGS. 16A and 16B are schematics of construct design for expression systems regulated by orthogonal RNA polymerases.

FIGS. 17A and 17B are heat maps illustrating the functional characterization of four polymerases: T3, SP6, KP34 and K11.

FIG. 18A is a schematic of a vanillic acid-regulated circuit. FIG. 18B is a bar graph showing GFP induction in a vanillic acid-inducible circuit.

FIG. 19A is a bar graph showing luminescence of a nanoluc-expressing landing pad UTEX2973 strains at different integration sites. FIG. 19B is a bar graph of luminescence of segregated S elongatus strains bearing a landing pad under different induction conditions. FIGS. 19C and 19D is a bar graph showing SGE function in Cupriavidus necator. 20 h post induction, n=1.

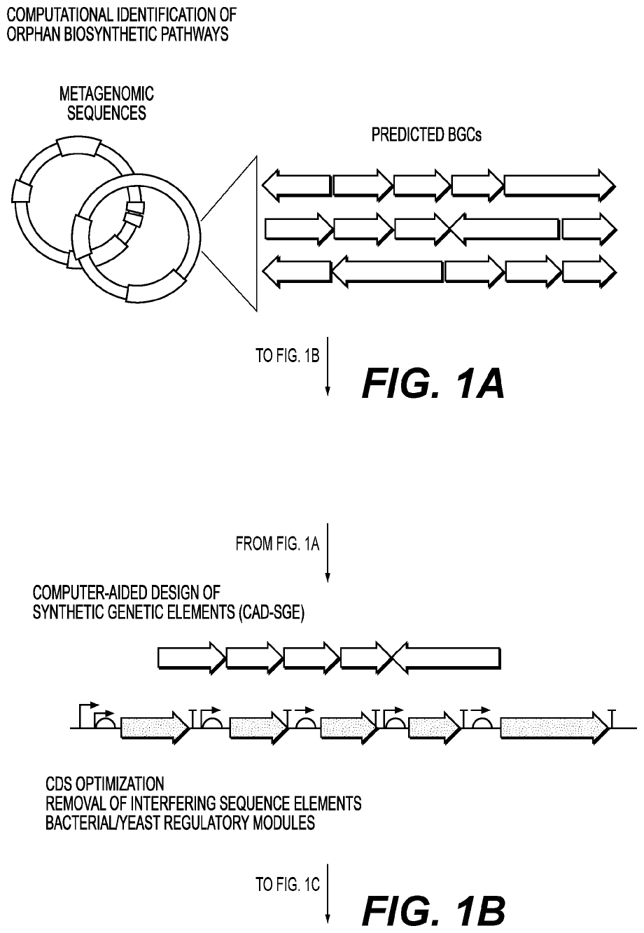

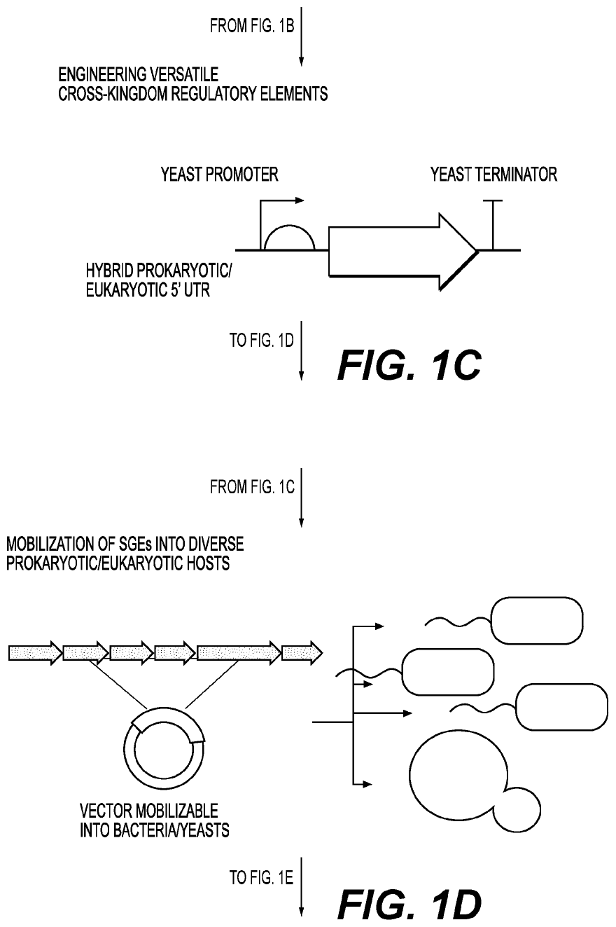

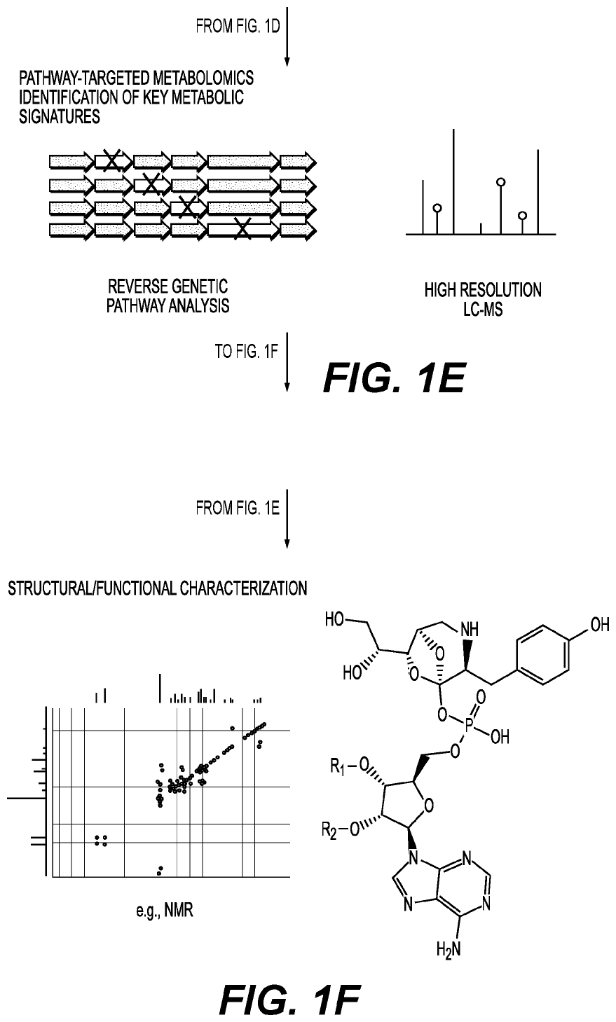

### DETAILED DESCRIPTION OF THE INVENTION

Disclosed herein are computational strategies and compositions and methods of use thereof for hierarchically redesigning multigene biological pathways for mobilization, expression, and characterization in versatile organisms. Using the disclosure, orphan biosynthetic gene clusters (BGCs) can be computationally redesigned into synthetic genetic elements (SGEs) and functionalized for expression across diverse hosts. This is facilitated by the development of hybrid transcriptional expression signals for both prokaryotes and eukaryotes provided herein. Compositions and methods of introducing and mobilizing SGEs into multiple kingdoms. For example, in exemplary embodiments, pathway-targeted metabolomics practiced on the mobilized SGEs can be used to identify key molecular features and characterize the structures and functions of output metabolites. This approach can productively animate orphan biosynthetic gene clusters and facilitated the discovery new routes of biosynthesis and/or identify and/or classify new compounds.

The computational strategies, compositions, and methods of use provided herein are modular, and can be used alone or in combinations, examples of which are exemplified in a non-limiting way throughout the disclosure and the experiments herein.

The compositions themselves are also modular and are expressly disclosed herein as discrete components alone and in combination with other disclosed components and/or other components available in the art.

Furthermore, many of the compositions include operably linked elements. Exemplary elements are provided, but such are also modular in nature, and alternative embodiments designed according to the disclosed strategies and guidelines having additional, alternative, or eliminated elements, including substitutable elements known in the art can be readily envisioned and also expressly provided herein.

Although the disclosed compositions are advantageous for expressing genes from biosynthetic pathways, the coding sequence can be any coding sequence alone or present in combination with any one or more other coding sequence. In some embodiments, the coding sequence(s) encodes a polypeptide. In some embodiments, the polypeptide is part of a biosynthetic pathway that works in concert with other polypeptides encoded in a biosynthetic gene cluster.

The disclosed methods and compositions can be understood more readily by reference to the following detailed description of particular embodiments and the Examples included therein and to the Figures and their previous and following description.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### I. Definitions

As used herein, the terms “polynucleotide” and “nucleic acid sequence” refers to a natural or synthetic molecule including two or more nucleotides linked by a phosphate group at the 3′ position of one nucleotide to the 5′ end of another nucleotide. The polynucleotide is not limited by length, and thus the polynucleotide can include deoxyribonucleic acid (DNA) or ribonucleic acid (RNA).

As used herein, the term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of gene to a transcriptional control element refers to the physical and functional relationship between the gene and promoter such that the transcription of the gene is initiated from the promoter by an RNA polymerase that specifically recognizes, binds to and transcribes the DNA.

As used herein, the terms “transformation” and “transfection” refer to the introduction of a polynucleotide, e.g., an expression vector, into a recipient cell including introduction of a polynucleotide to the chromosomal DNA of the cell.

As used herein, the term “transgenic organism” refers to any organism, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. Suitable transgenic organisms include, but are not limited to, bacteria, cyanobacteria, fungi, plants and animals. The nucleic acids described herein can be introduced into the host by methods known in the art, for example infection, transfection, transformation or transconjugation.

As used herein, the term “eukaryote” or “eukaryotic” refers to organisms or cells or tissues derived from these organisms belonging to the phylogenetic domain Eukarya such as animals (e.g., mammals, insects, reptiles, and birds), ciliates, plants (e.g., monocots, dicots, and algae), fungi, yeasts, flagellates, microsporidia, and protists.

As used herein, the term “prokaryote” or “prokaryotic” refers to organisms including, but not limited to, organisms of the Eubacteria phylogenetic domain, such as Escherichia coli, Thermus thermophilus, and Bacillus stearothermophilus, or organisms of the Archaea phylogenetic domain such as, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, and Aeuropyrum pernix.

As used herein, the term “construct” refers to a recombinant genetic molecule having one or more isolated polynucleotide sequences. Genetic constructs used for transgene expression in a host organism can include in the 5′-3′ direction, one or more of a promoter sequence; a sequence encoding a gene of interest; and a termination sequence. The construct may also include selectable marker gene(s) and other regulatory elements for expression.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product, for example a functional RNA that does not encode a protein or polypeptide (e.g., miRNA, tRNA, etc.). The term gene as used herein with reference to genomic DNA includes intervening, non-coding regions as well as regulatory regions and can include 5′ and 3′untranslated ends.

As used herein, the term “vector” refers to a polynucleotide capable of transporting into a cell another polynucleotide to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector.

As used herein, term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, enhancers, and terminators.

As used herein, the term “promoter” refers to a regulatory nucleic acid sequence, typically located upstream (5′) of a gene or protein coding sequence that, in conjunction with various elements, is responsible for regulating the expression of the gene or protein coding sequence. These include constitutive promoters, inducible promoters, tissue- and cell-specific promoters and developmentally-regulated promoters.

The term “endogenous” with regard to a nucleic acid refers to nucleic acids normally present in the host.

As used herein, the term “heterologous” refers to elements occurring where they are not normally found. For example, a promoter may be linked to a heterologous nucleic acid sequence, e.g., a sequence that is not normally found operably linked to the promoter. When used herein to describe a promoter element. heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompass “exogenous” and “non-native” elements.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E. and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

Unless otherwise indicated, the disclosure encompasses conventional techniques of molecular biology, microbiology, cell biology and recombinant DNA, which are within the skill of the art. Unless otherwise noted, technical terms are used according to conventional usage, and in the art, such as in the references cited herein, each of which is specifically incorporated by reference herein in its entirety.

### II. Methods of Making and Refining Synthetic Genetic Elements (SGEs) and SGE's Formed Thereby

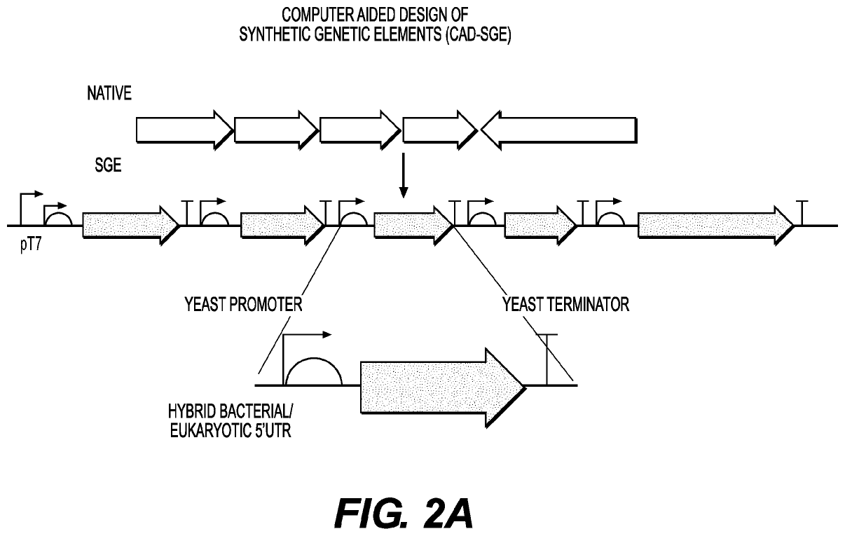

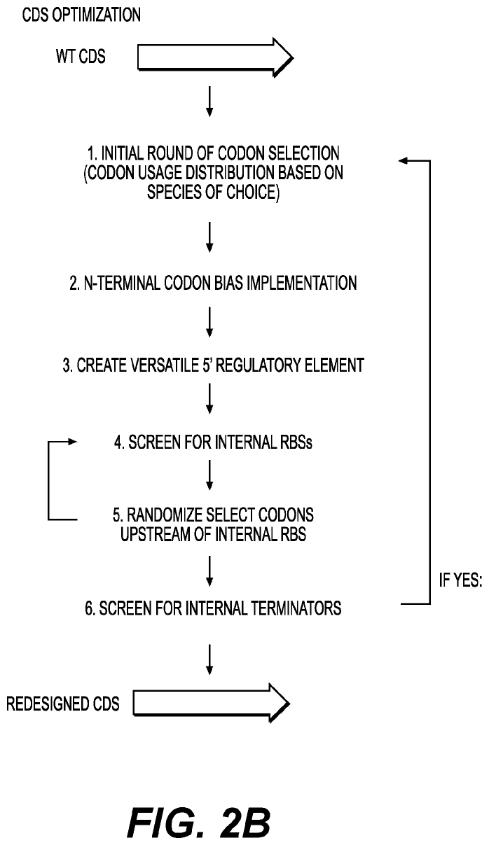

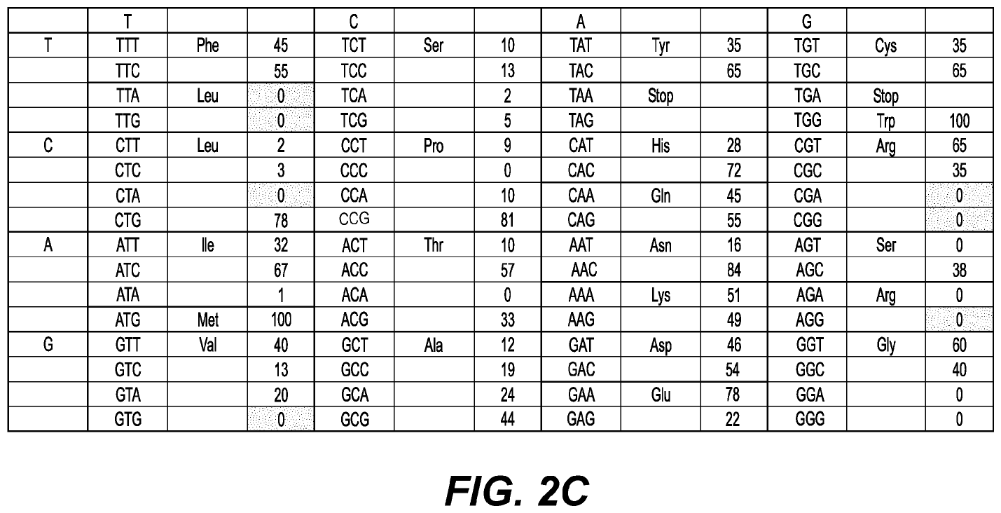

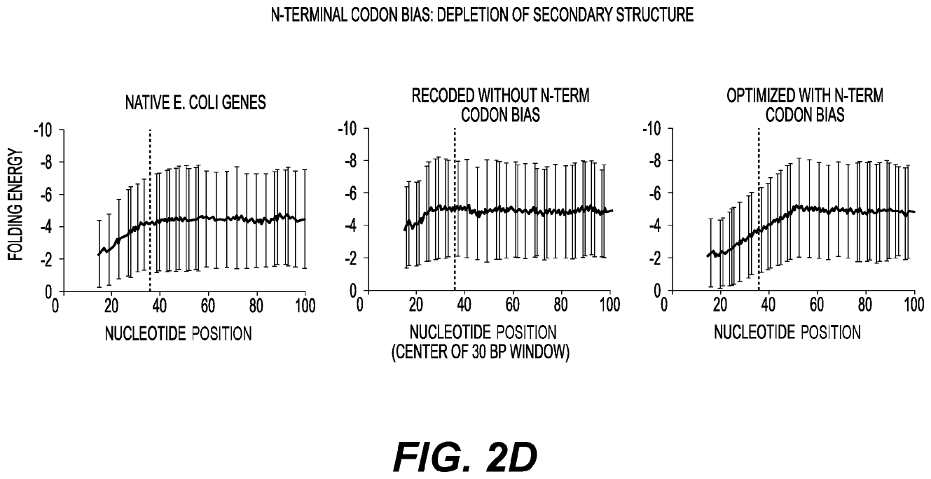

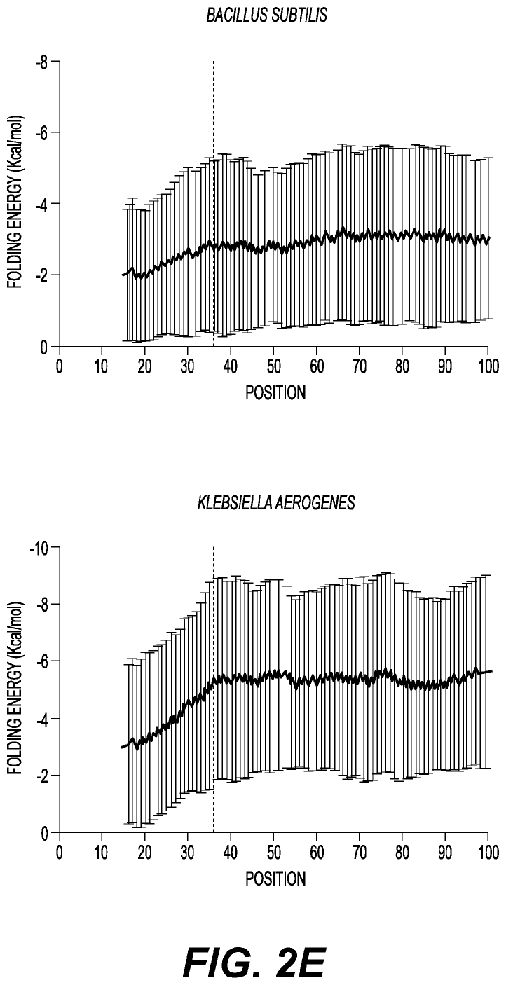

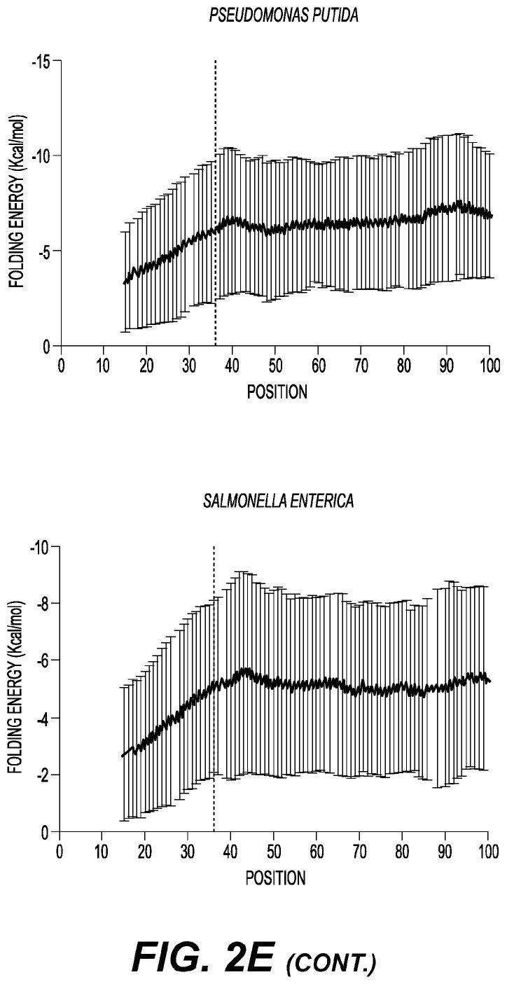

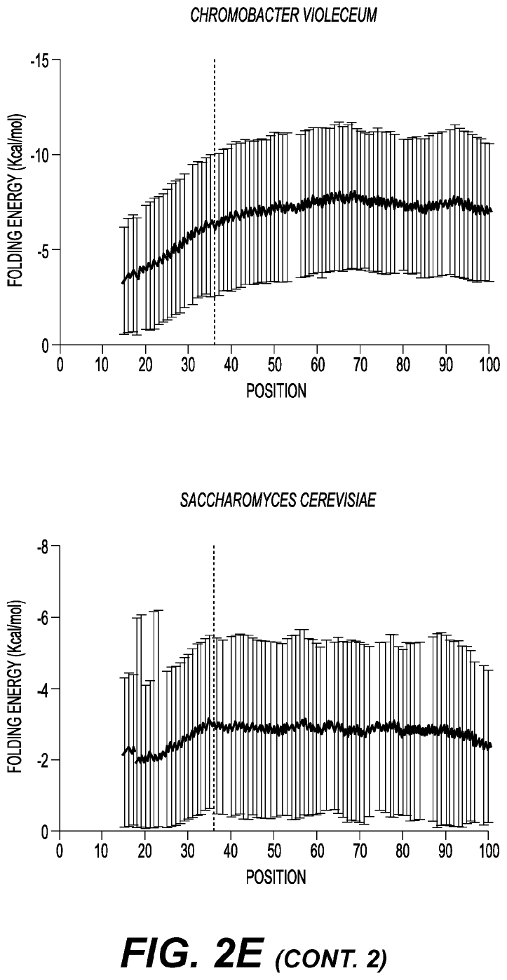

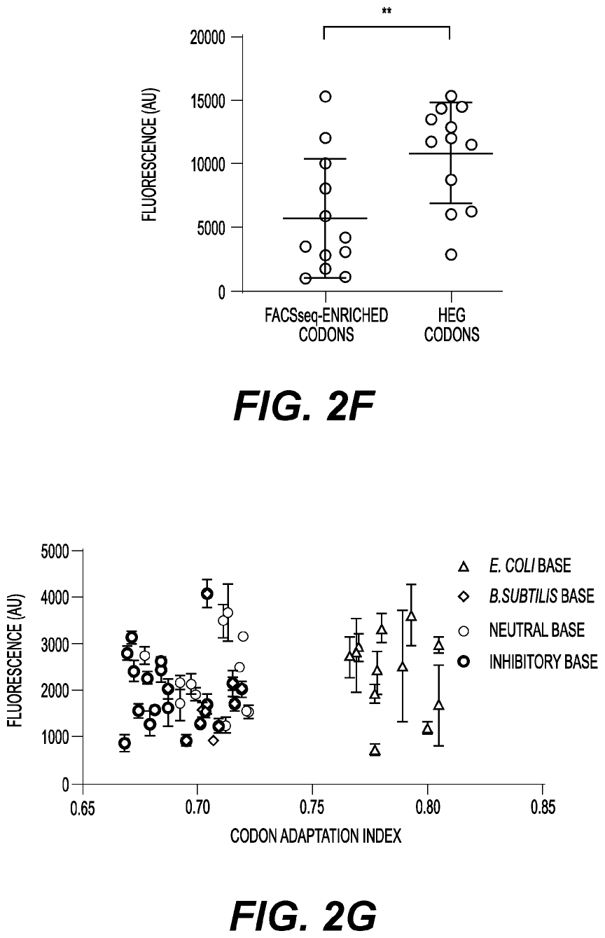

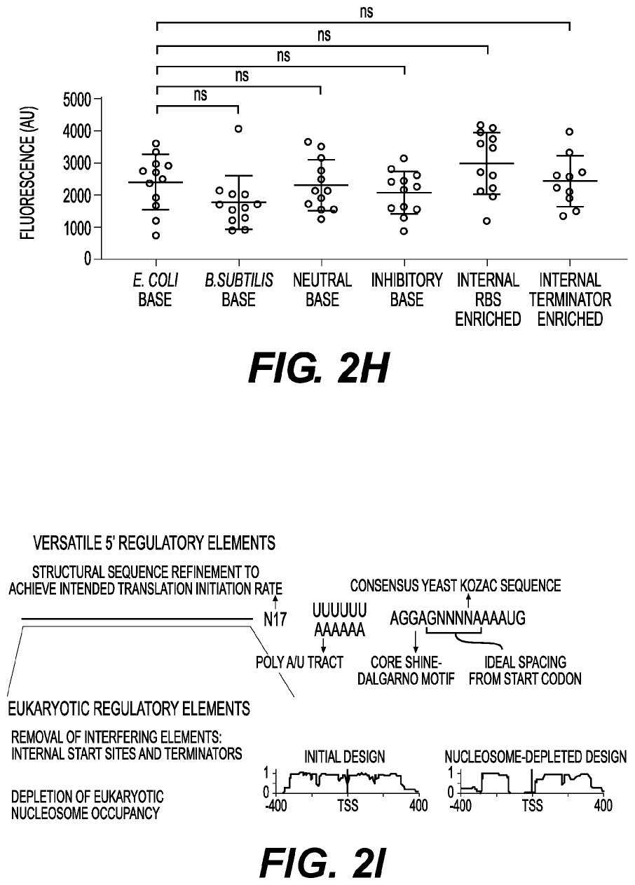

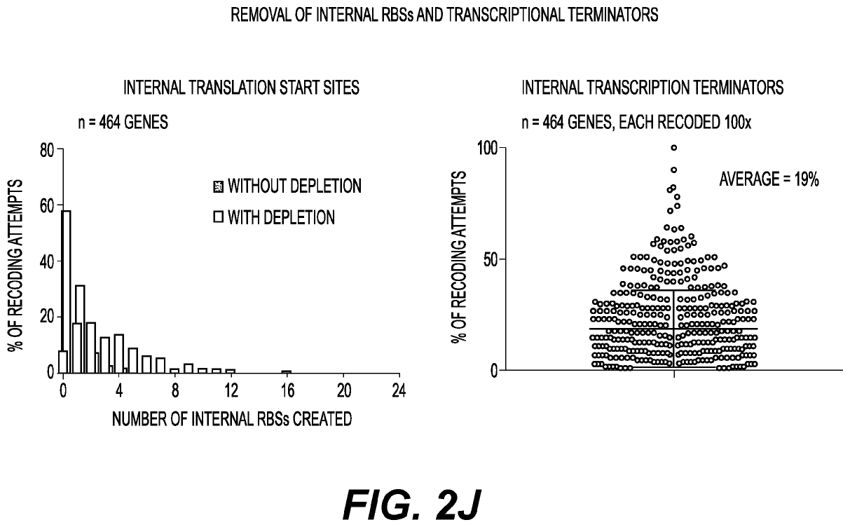

Biosynthetic gene clusters typically refers to genes and pathways that encode enzymes that play a role in biochemical reactions, especially metabolism. Expression from biosynthetic gene clusters (BGCs) and their associated metabolites involves sequential layers of control exerted at multiple levels: 1) transcription, through mRNA initiation, elongation, and stability; 2) translation, through ribosomal binding and codon usage; and 3) enzymatic activity, often mediated through posttranslational modification and the availability of input metabolites and metabolic flux (Temme et al., 2012). Through evolutionary divergence, regulation of these layers can be strain- and environment-specific. Thus, a major challenge in achieving host-range versatility is to decouple biosynthetic capacity from these regulatory layers. To solve this problem, a computer-aided design strategy was devised to redesign BGCs at the level of an individual coding sequence (CDS), transcription, and translation, establishing synthetic design principles to enable cross-kingdom host-range versatility. An overview of method steps and their impact on expression are illustrated in FIG. 2A-2J. The design strategy can include any one or more of the illustrated steps of FIGS. 2A-2J, and discussed in more detail below. Furthermore, although introduced as means for refining CDSs, one or more steps of the disclosed methodology can also be used to refine other components discussed herein including but not limited to inducible circuits, selectable markers and reporters, SGEs, vectors, etc.

The following sections provide methods for improving coding sequences and SGEs, as well as compositions and methods for introducing them into diverse host cells. Both the design strategies and methodologies, as well as the components and compositions form in accordance therewith, and/or containing the components and compositions are expressly provided. Any of the disclosed design strategies can be carried out on a computer, and thus in some embodiments, one or more or all of the design and/or refinement steps and/or simulations are carried out on a computer.

### A. Design and Refinement of Individual Coding Sequences (CDS)

The method can include redesigning one or more of the nucleic sequences. Although particularly advantageous for expressing multigene biosynthetic pathways, the disclosed strategies, compositions, and methods are not so limited, and the disclosed coding sequences can be any single gene alone or used in combination with other genes, which may or may not for part or all of a biosynthetic pathway or other gene cluster.

Referred to herein as the individual coding sequence (CDS), each of the coding sequences can be synonymously recoded to improve expression of the elements encoded therein in a heterologous organism. Although in some embodiments, the method employs a traditional codon optimization approach, these are not preferred. A constraint with traditional codon optimization approaches is that they are tailored for a target species. Additionally, the general utility of codon optimization for heterologous expression remains an unresolved subject, where large-scale screens fail to capture a general correlation between codon adaptation and expression levels (Kudla et al., 2009). Specifically, most strategies improve heterologous protein production by synonymously altering a gene's codon usage to match the more frequently used codons—i.e., the codon adaptation index (CAI) approach—or available tRNA pool of a single heterologous host—i.e., the tRNA adaptation index (TAI) approach (Mauro and Chappell, 2014). This classical paradigm is less preferred because the disclosed strategies aim to generate constructs for expression in diverse prokaryotic and eukaryotic taxa, each with greatly varying GC content, tRNA abundances, and codon usage patterns.

To address these constraints and facilitate versatile expression of SGEs, an alternative CDS-level improvement protocol was developed to capture more host-independent improvement parameters, and can include any one or more of the steps outlined in FIG. 2B. Thus, in some embodiments, redesigning a CDS includes one or more of (1) initial round of codon selection, which is optionally, but preferably based on the preferred codon distribution in the heterologous organism(s) of choice; (2)N-terminal codon bias implementation; (3) creating a versatile 5′ regulatory element; (4) screening for internal ribosome binding sites (RBSs); (5) randomizing select codons upstream of internal RBS, and optionally repeating (4) and (5) in cycles; and (6) screening for internal terminators, optionally wherein any one or more of (1)-(6) can be repeated in iterative cycles. In some embodiments, for each RBS identified in (4) from 1-100 or any specific number or subrange therebetween, optionally, 10-100, optionally 10-20, cycles of randomization (5) are performed until a solution is found. This maximum number of 20 cycles is sufficient for the vast majority of cases. Typically, a single cycle of step (6) is sufficient to find a solution that removes internal terminators.

### 1. Codon Selection

The methods can include codon selection, which is optionally, but preferably based on the preferred base and/or codon distribution in the heterologous organism(s) of choice. Individual CDSs can be converted from amino acid to nucleotide sequence. The baseline codon usage distribution can be based on that of highly expressed genes of a species of choice, and the amino acid sequence recoded accordingly. For the experiments discussed below, base selection was based on Escherichia coli (see, e.g., FIG. 2C), although the strategy allows for variable base selection base on the organism of choice. For example, codon usage information for different organisms can be computed directly from publicly available genome sequences for individual strains or downloaded directly from databases such as cbdb.info, the Dynamic Codon Biaser website.

Other factors that can optionally be included in base and/or codon selection and nucleic acid sequence recoding can include (a) depletion of canonically-inhibiting codons, including, but not limited to: (i) TTA, which is inefficiently decoded in a variety of Actinobacteria (Leskiw et al., 1991), (ii) AGG, CTA, and/or CGA, which are broadly depleted across highly diverse bacteria (Tian et al., 2017), (iii) CGG and/or CGA, which promote the formation of “inhibitory pairs” in S. cerevisiae (Ghoneim et al., 2019), or a combination thereof, and/or (b) depletion of TTG and/or GTG to disfavor alternative start codons.

### 2. N-terminal Codon Bias

Codon usage specifically encoding the N-terminus has been shown to significantly impact gene expression, largely attributed to 5′-RNA secondary structure among other factors (Angov, 2011). This feature is conserved in prokaryotic and eukaryotic phyla and serves as a useful parameter to promote host-range versatility. Codons that lower structure, thereby enhancing translational initiation at the start codon, promote stronger expression (Goodman et al., 2013). Thus, in some embodiments, the methods include recoding the N-terminus of the encoding nucleic acid sequence to lower second and/or tertiary structure.

In the experiments below, the impact of this step was investigated by analyzing the predicted 5′-mRNA structure of E. coli genes before and after recoding in silico. To avoid the confounding variable of translational coupling, analysis was limited to genes that did not overlap with upstream CDSs (n=464). Using Vienna RNA Suite (Lorenz et al., 2011), the minimum folding energy across each CDS was calculated using a 30 bp sliding window. The results show that the effect of depletion of secondary structure in native gene sequences, particularly in the 36 bp at the 5′-terminus, and illustrates its reproducibility across phyla.

If CDSs are recoded by the standard CAI approach (Mauro and Chappell, 2014), using the codon distribution of highly expressed E. coli genes, this 5′-thermodynamic property dissipates (FIG. 2E).

Thus, in some embodiments, reducing N-terminal bias includes depletion of secondary structure in native gene sequences and/or the recoded CDS following step (1) described above. In some embodiments, reducing N-terminal bias includes using a hybrid codon distribution that biases toward privileged or preferred N-terminal codons that correlate with high expression levels in the organism(s) of interest. In some embodiments, depletion of secondary structure is applied to 15-75 base pairs, or any subrange or specific integer therebetween, such as 30-40 bp or 36 bp, at the 5′ terminus of one or more CDSs. In some embodiments depletion of secondary structure includes recoding based on a CAI or TAI approach. Genes recoded with this approach computationally can recreate the depletion of 5′ structure seen in native genes (FIG. 2E). In some embodiments, CDSs that overlap with an upstream CDS are excluded from this step.

### 3. 5′ Regulatory Element(s)

In some embodiments, the methods include creating a synthetic 5′ regulatory element to facilitate versatile regulation across diverse prokaryotes and eukaryotes. In some embodiments, this step includes creation of a hybrid of eukaryotic and prokaryotic elements that are known to impact gene expression in one, two, three, or more microbial taxa, optionally wherein one or more of the taxa include the heterologous organism(s) in which the CDSs will be express. See, e.g., FIG. 2I.

In some embodiments, the step utilizes a thermodynamic translation initiation model which defines sequence and structural determinants of bacterial ribosome entry and allows predictions of translation initiation rates using the RBS calculator (Salis et al., 2009), which is specifically incorporated by reference herein in its entirety.

In some embodiments, this model is expanded with additional parameters to increase host range applicability. For example, Gram-positive bacteria are known to demonstrate a substantially stricter Shine-Dalgarno sequence requirement and start codon spacing preference when compared to Gram-negative bacteria (Vellanoweth and Rabinowitz, 1992), which is specifically incorporated by reference herein, consideration of which can be utilized in determining the final sequence. Preferably, upstream sequence is enriched in poly AT sequence, which mirrors UTRs in both bacterial phyla and eukaryotes (Cuperus et al., 2017). Preferably, a “AAA” sequence motif is maintained immediately upstream of the start codon to match the S. cerevisiae consensus Kozak sequence (Hamilton et al., 1987).

The experiments below report that integrating all of these design considerations results in a base UTR defined as N₁₇(A/U)₆AGGAGN₄AAA (SEQ ID NO:1) (FIG. 2I).

Thus, in some embodiments, this step includes or consists of beginning with a synthetic 5′ UTR of SEQ ID NO:1, and iteratively mutating/varying ‘N’ positions until a desired translation initiation strength is reached, which may be predicted or determined empirically. In this way, the translation initiation strength for each CDS can be specifically tailored.

### 4. Screening for Internal RBSs and Terminators

In some embodiments, the methods include screening for and optionally removing internal RBSs typically by recoding them. For example, the nucleotide sequences can be screened to remove or recode alternative NTG start codons, internal RBSs (e.g., NTG sites throughout the CDS in all three coding frames), and terminators.

Outputs of the initial CDS and 5′-UTR design methodology revealed sequences predicted to signal aberrant transcription termination and translation initiation, which are undesirable for heterologous expression. To evaluate this quantitatively, an E. coli gene test was set through our algorithm; each gene was recoded 100 times to derive a representative quantification of the outcome. Widespread emergence of internal prokaryotic translation start sites were predicted using the RBS thermodynamic parameters from the RBS calculator (Salis et al., 2009). An average of 3.8 internal RBSs appeared per gene recoding attempt (FIG. 2J). In native genes, aberrant internal translation initiation is largely disfavored, even in the presence of Shine Dalgarno motifs upstream of ATG codons, as demonstrated by ribosomal profiling experiments (Li et al., 2012). However, the mechanism and sequence features by which internal initiation is avoided is not understood (Saito et al., 2020). Additionally, deleterious rho-independent terminators spontaneously appeared during 19% of the recoding attempts, as identified using the predictive tool transtermHP (Kingsford et al., 2007) (FIG. 2J).

Accordingly, as an additional design principle, this issue can be circumvented by depleting NTG codons in all three forward coding frames. When an NTG codon cannot be avoided, the upstream sequence is then synonymously modified to structurally inhibit internal ribosome entry. These efforts significantly decrease the number of predicted internal translation initiation sites from 3.8 to 0.6 per gene (p<0.001 using a 2-tailed paired Z-test) (FIG. 2J) in the experiments below.

Additionally, the method can include scanning and removing the deleterious terminators as another design principle.

Prediction utilized for carrying out these steps can be carried out, for example, according the same or similar methods utilized in the experiments below, e.g., using tools described in (Salis et al., 2009), (Lorenz et al., 2011), and (Kingsford et al., 2007), each of which is specifically incorporated by reference in its entirety.

For example, in the experiments below, for ribosome binding site (RBS) strength predictions, thermodynamic parameters were calculated in accordance with previous studies (Salis et al., 2009). This calculation is summarized as:

$$Δ {G}_{tot} = Δ {G}_{mRNA : rRNA} + Δ {G}_{start} + Δ {G}_{spacing} - Δ {G}_{standby} - Δ {G}_{mRNA}$$

where β=0.45, and A=2500
ΔGₜₒₜ is the difference in Gibbs free energy between the initial state (folded mRNA transcript and the free 30S complex) and the final state (the assembled 30S pre-initiation complex bound on an mRNA transcript;
ΔG_{(mRNA:rRNA)} is the energy released when the last 9 nucleotides (nt) of the E. coli 16S rRNA ((3′-AUUCCUCCA-5′) hybridizes and co-folds to the mRNA sub-sequence;
ΔGₛₜₐᵣₜ is the energy released when the start codon hybridizes to the initiating tRNA anticodon loop (3′-UAC-5′);
ΔG_{spacing} is the free energy penalty caused by a non-optimal physical distance between the 16S rRNA binding site and the start codon;
ΔG_{standay} is the work required to unfold any secondary structures sequestering the standby site after the 30S complex assembly; and
ΔG_{mRNA} is the work required to unfold the mRNA sub-sequence when it folds to its most stable secondary structure, called the minimum free energy structure.

The Vienna RNA Suite was used to collect the Gibbs Free Energy values in accordance with previous studies (Lorenz et al., 2011). The following assumptions were made: (1) the relevant mRNA considered was +/−35 bp flanking the start codon, (2) the Ribosome unfolded the first 15 bp of the open reading frame, (3) the standby site was 4 bp upstream of the rRNA binding site, and (4) the relevant anti-Shine Dalgarno rRNA sequence considered was the terminal 9 bp of 16S rRNA (For E. coli, this sequence is “ACCUCCUUA”). The ΔGₛₜₐᵣₜ values used were: “AUG”:-1.194, “GUG”:-0.0748, “UUG”:-0.0435, “CUG”:-0.03406. To account for multiple mRNA:rRNA folding configuration possibilities, the RNAduplex program was used to duplex the rRNA to the region of the mRNA 3-13 bp upstream of the start codon. All possible duplexes+/−1.5 kcal/mol of the Minimum Free Energy (MFE) were considered. The ΔGₜₒₜ was calculated for each possible duplex. The duplex that minimized ΔGₜₒₜ was considered the equilibrium translation initiation configuration.

In the experiments below, the computational program TransTermHP (Kingsford et al., 2007) was used to predict rho-independent transcriptional terminators on both strands. Default parameters for stemloop and tail scoring were used. The Confidence threshold for calling a terminator was left as >76.

These various principles are illustrated in FIG. 2B steps 4, 5, and 6, which include screening for internal RBS (4), optionally randomizing select codons upstream of internal RBS (5), optionally iteratively repeating (4) and (5) in two or more cycles, and alternatively or further including screening for terminators and optionally recoding them (6) to until a desired translation initiation strength is reached, which may be predicted or determined empirically.

### B. Synthetic Genetic Elements

Synthetic genetic elements (SGE) including two or more CDSs and optionally, but preferably additional regulatory elements are also provided. The CDS may be the native sequences, but preferably are recoded according to one or more, preferably all, of the design methods described above or elsewhere herein. In some embodiments, CDS are also reorder and/or expression direction is a reversed so most of all coding sequences are expressed in the same direction (e.g., encoded by the same strand of double stranded DNA). See, e.g., FIG. 2A, which provides an overview of a recoded synthetic element including additional regulatory and expressing enhancing modifications including reversing the direction/orientation of a single gene encoded in the reverse direction relative to the four other genes of the multigene pathway that is the subject of the SGE.

### 1. Hybrid Prokaryotic-Eukaryotic Regulatory Element

Cross-kingdom transcription initiation can be enhanced by adding and/or modifying the expression control sequences; i.e., regulatory elements. For example, the disclosed SGEs typically include the necessary regulatory elements for expression in at least two different kingdoms, e.g., prokaryotes and eukaryotes. In prokaryotes, multiple genes (i.e., multiple CDS) can be concurrently transcribed as a polycistronic operon. However, each CDS needs a distinct promoter and terminator in eukaryotes. Given this requirement, the 5′ sequence of each CDS can be further extended to include regulatory elements to initiate eukaryotic (e.g., yeast, mammalian cell, etc.) transcription initiation and decrease nucleosome occupancy in eukaryotes. In the context of a multigene operon, this design therefore creates intergenic regions depleted in nucleosome occupancy, which is strongly correlated with both efficient transcription initiation and termination by polyA-capping in eukaryotes (Ichikawa et al., 2016; Morse et al., 2017) (FIG. 2I).

The sequences can be naturally occurring or synthetic. As discussed above and elsewhere herein, the coding sequence can be any coding sequence. In some embodiments, the coding sequence encoding a polypeptide including, but not limited to, those that form part of a biosynthetic pathways.

The sequence can be, or be derived from, any one or more of the organisms in which the SGE will be expressed. Suitable sequences are known in the art. For example, in the experiments below, a library of synthetic S. cerevisiae terminators (Curran et al., 2015; MacPherson and Saka, 2017; Wang et al., 2019b), each of which is specifically incorporated by reference herein in its entirety, was utilized. See also Curran, et al., Metab Eng., 19: 88-97 (2013), which is specifically incorporated by reference in its entirety. Such sequences can thus be used in the disclosed SGE.

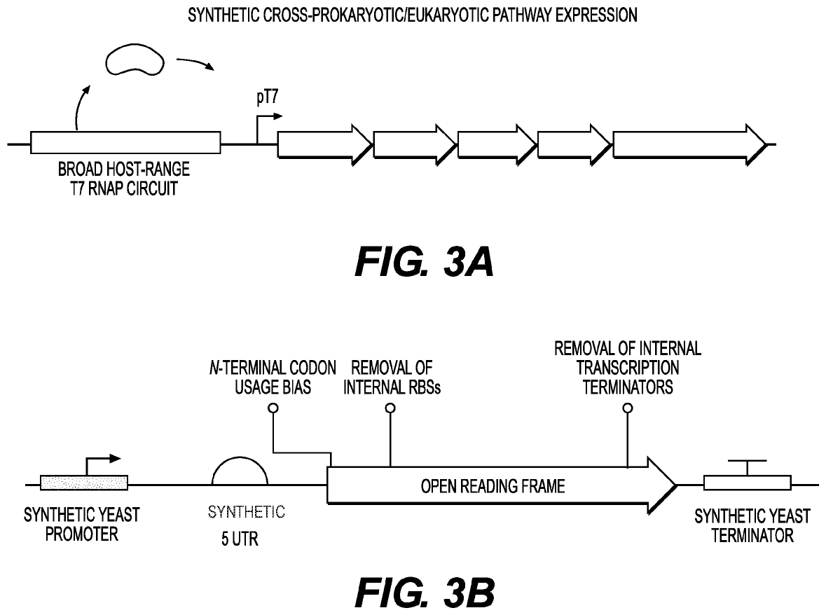

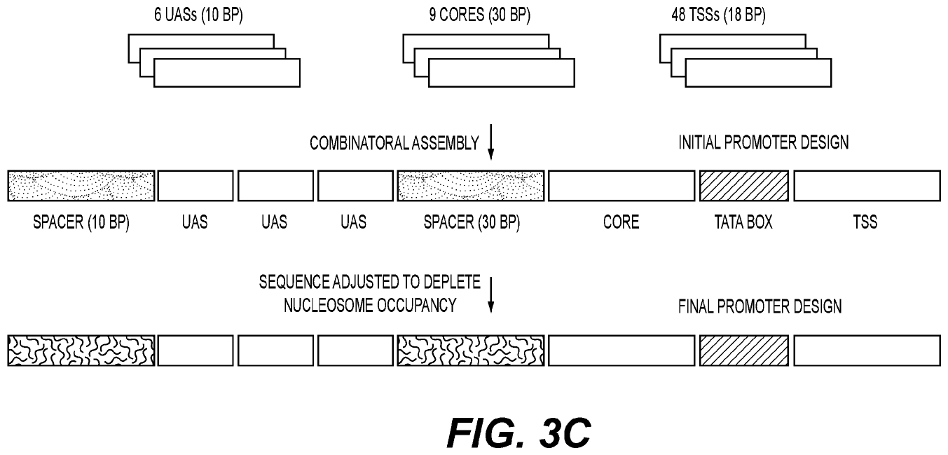

Sequences can also be created by the practitioner. For example, in the experiments below, to develop 5′ sequences designed to initiate transcription in both prokaryotes and eukaryotes, an expanded library of synthetic yeast promoters was developed that addressed three key requirements of cross-kingdom SGE design (FIG. 3A-3B).

In designing the regulatory sequences, one or more of several features can be considered. For example, elements are preferably efficient in one or more organisms of interest, without interfering, or at least not prohibiting expression in another organism of interest. In the experiments below, eukaryotic elements were selected and/or modified to limit or eliminate interference with bacterial expression at both the transcriptional and translational levels.

In some embodiments, sequence size is reduced or minimized to reduce synthesis costs, and to reduce the negative impact untranslated sequence has on bacterial mRNA stability (Cetnar and Salis, 2021).

In some embodiment, particularly for multigene operons, a large library with minimal sequence overlap is utilized to prevent deletions through homologous recombination.

Promoters meeting one or more of these constraints can be developed by any suitable means. For example, in the experiments below, a previously reported framework to achieve robust eukaryotic expression by arraying synthetic 10 bp upstream activity sequences (UASs) (6 distinct sequences), 30 bp core sequences (9 distinct sequences), a consensus TATA box (TATAAAG), and random spacers (FIG. 3C) (Redden and Alper, 2015), which is specifically incorporated by reference herein in its entirety, was utilized. 48 transcription start sites (TSSs) matching the known consensus motif [A(A_{rich})₅ NPy A (A/T)NN(A_{rich})₆]from the native S. cerevisiae genome (Zhang and Dietrich, 2005) were also mined. The sequences of these parts can be found in SEQ ID Nos: 2-49.

To terminate any translation initiation from inside the promoter sequence, promoters can be flanked with a three-frame stop codon, e.g., (TAANTAANTAA).

SGEs can include one or more UAS sequences associated with promoters. An upstream activating sequence or upstream activation sequence (UAS) is a cis-acting regulatory sequence. It is distinct from the promoter and increases the expression of a neighboring gene. In some embodiments, the promoter driving expression of one or more of CDSs of the SGE include 1-10 inclusive, or any subrange or specific integer thereof, UAS. Additionally or alternatively, the primary sequence of spacers can be interspaced with poly-A or poly-T (e.g., 5-mers) to deplete the probability of nucleosome occupancy at the TATA box (TATAAAG) and transcriptional start site (TSS).

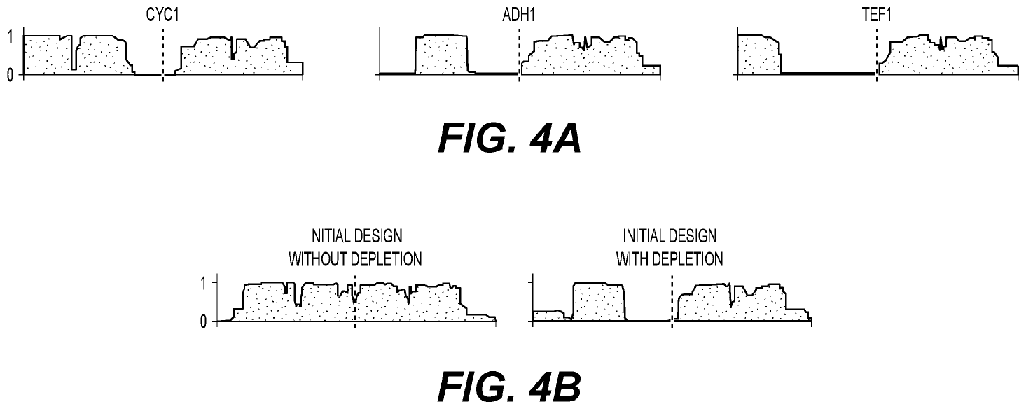

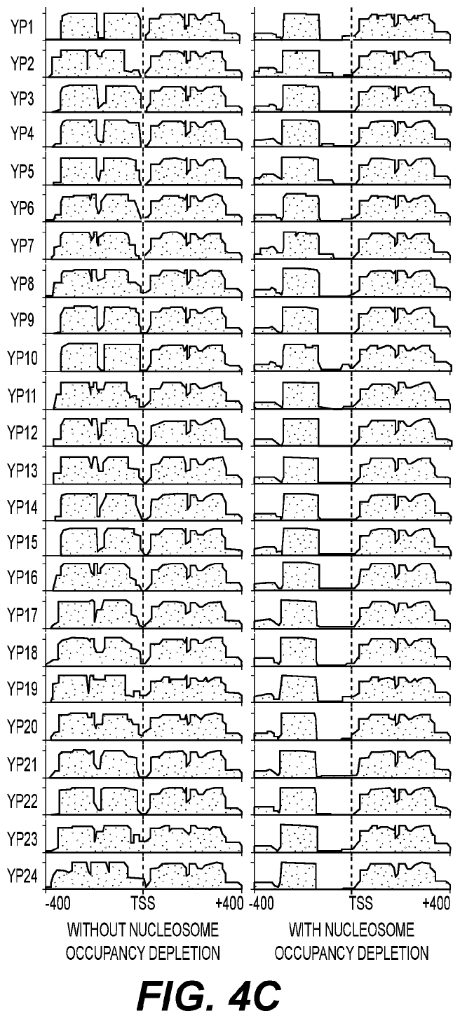

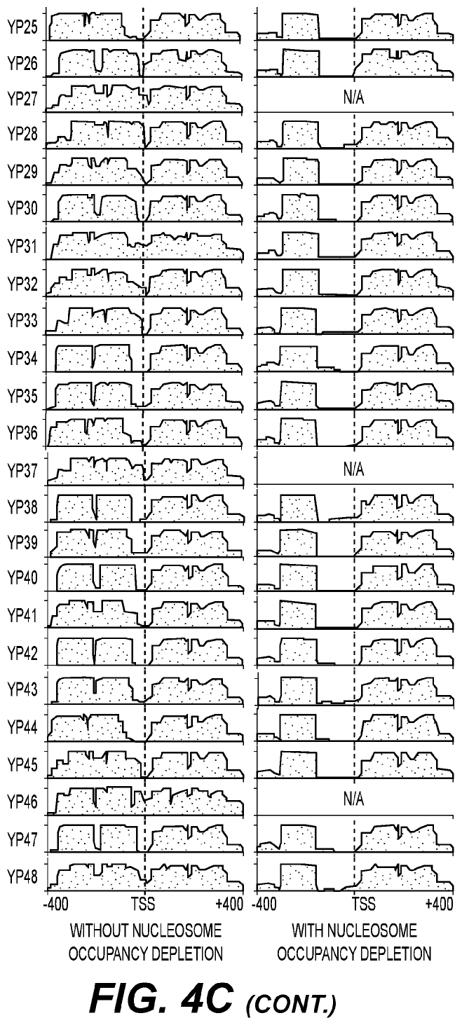

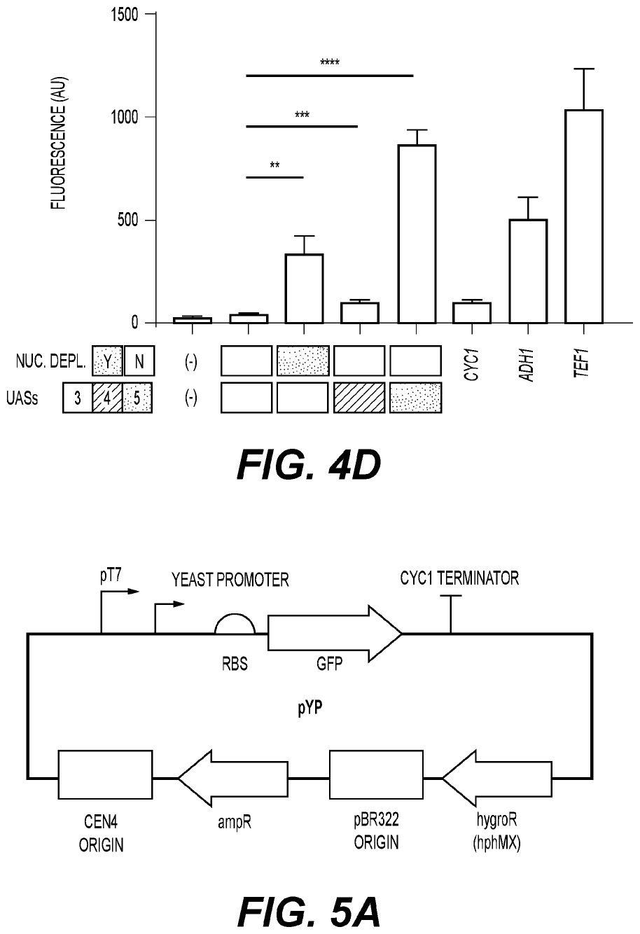

In the experiments below, the expression levels in S. cerevisiae, were investigated by exploring a range of 3-5 UASs per promoter and interspacing spacers with poly-A or poly-T 5-mers to deplete nucleosome occupancy at the TATA box and TSS (FIG. 4B).

Such sequence modifications can be carried out according to any suitable. For example, in the experiments below NuPop hidden Markov model was used for predicting nucleosome position (Xi et al., 2010), which is specifically incorporated by reference herein in its entirety. A test protein, e.g., a marker such green fluorescent protein, can be used to investigate the impact of these variables. In the examples below, increasing the number (3-5) of UASs increased expression levels 2.4-fold (p<0.001) and 21-fold (p<0.0001), respectively. With 5 UASs, expression was comparable to the strong tef1 promoter native to S. cerevisiae. Independently, nucleosome depletion could also increase expression levels 8.2-fold (p<0.01) (FIG. 4C). This indicates that these variable can be used to tune the expression levels in an organism of choice.

In some embodiments, one or more of additional sequence considerations are implemented in designing the SGE:
(i) no pair of UASs is used more than 5, 4, 3, 2, or, 1 preferably no more than 3 times, and optionally, but preferably, no triplet of UASs is used more than once per library to avoid repetitive sequences;
(ii) promoters range from 100 bp to 250 bp inclusive, or any subrange thereof, or specific integer therefore, for example 161 bp to 181 bp, in length; and/or
(iii) no spacer or TSS sequence is reused.

As a result of using these preferred design parameters, a maximum stretch of sequence similarity between any two promoters is 30 bp.

Additional design parameters that can be used alone or in combination with one or more of (i)-(iii) include:
(iv) no ‘NTG’ sequence is used in any spacer to avoid internal start codons; and/or
(v) promoters are further screened for predicted terminators and RBSs (e.g., as discussed above), which are removed by randomly mutating spacer sequences.

The SGE elements are typically operably linked to allow for expression of the one or CDSs in two or more organisms of interest, preferably organisms from two or more different kingdoms. For example, in a non-limiting example, the SGE includes a prokaryotic RBS, a bacterial promoter, one or more eukaryotic promoters, and a eukaryotic terminator. An exemplary illustration can be found in FIG. 3B. Any of the elements can be fixed or variable and screened for the most preferred combination(s) and/or to tune expression in one or more of the organisms of interest.

Additionally provided are 48 synthetic hybrid promoters created based on varying these parameters. Any of these synthetic promoters can be appended to the 5′ sequence of any CDSs, e.g., to activate BGCs in both E. coli and S. cerevisiae, or be utilized as a starting point for further recoding and optionally screening for desired expression results, e.g., as described herein (SEQ ID NO:59-98).

### C. Inducible RNA Polymerase Expression Circuit

An inducible T7 RNA polymerase expression circuit, and alternatives thereto are also provided both alone as a part of SGEs. As discussed in the experiments below, such a circuit can be utilized alongside hybrid eukaryotic-prokaryotic promoters to modulate transcription across diverse bacterial species, optionally but preferably in titratable manner.

Bacteriophage T7 RNA polymerase (T7RNAP) and cognate T7 promoter (pT7) system is a highly orthogonal, processive, and host-independent system (Tabor, 2001). Because transcription from pT7 is constrained by the cognate T7RNAP, a major challenge for using this system in the disclosed SGEs, is expressing the T7RNAP in a host versatile manner. The processivity of the T7RNAP can lead to fitness defects, which can be counterproductive to biosynthetic pathway functionality due to competition for cellular resources (Scott et al., 2010).

To provide a balance between robustness and titratability, the UBER system, which couples positive and negative feedback loops to modulate gene expression (Kushwaha and Salis, 2015), which is specifically incorporated by reference herein in its entirety, was expanded. In the original UBER framework, seeding transcription provided by (+)—strand transcription from upstream genes drives the initial production of T7RNAP. T7RNAP production is further auto-regulated through a positive feedback loop catalyzed by an upstream pT7. To prevent compounding RNAP amplification, a negative feedback loop proportionally produces an anhydrotetracyline (aTc) responsive TetR repressor to inhibit T7RNAP production. Prior work found that the translation initiation rate of the T7RNAP was the primary determinant controlling system output (Kushwaha and Salis, 2015). However, this original design was not demonstrated to have inducible activity, an important criterion for controlled expression of heterologous biosynthetic pathways that may variably exhibit cytotoxicity in diverse hosts. Thus, a theophylline-responsive translational riboswitch previously engineered to have broad host range can be utilized to impart tunable control generalizable to function across bacterial phyla (Espah Borujeni et al., 2016; Topp et al., 2010; Wachsmuth et al., 2013), each of which is specifically incorporated by reference herein in its entirety.

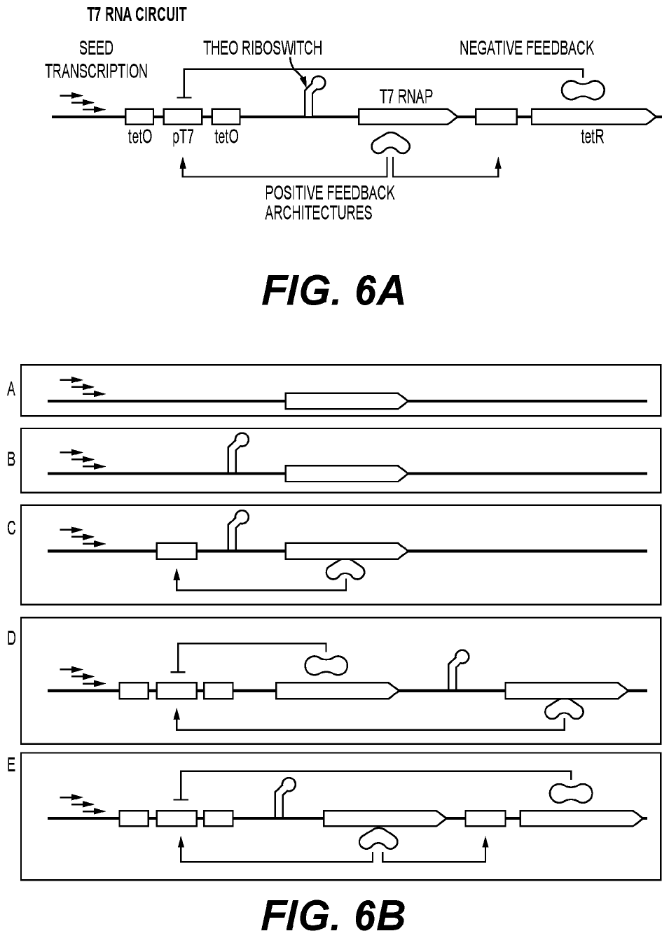

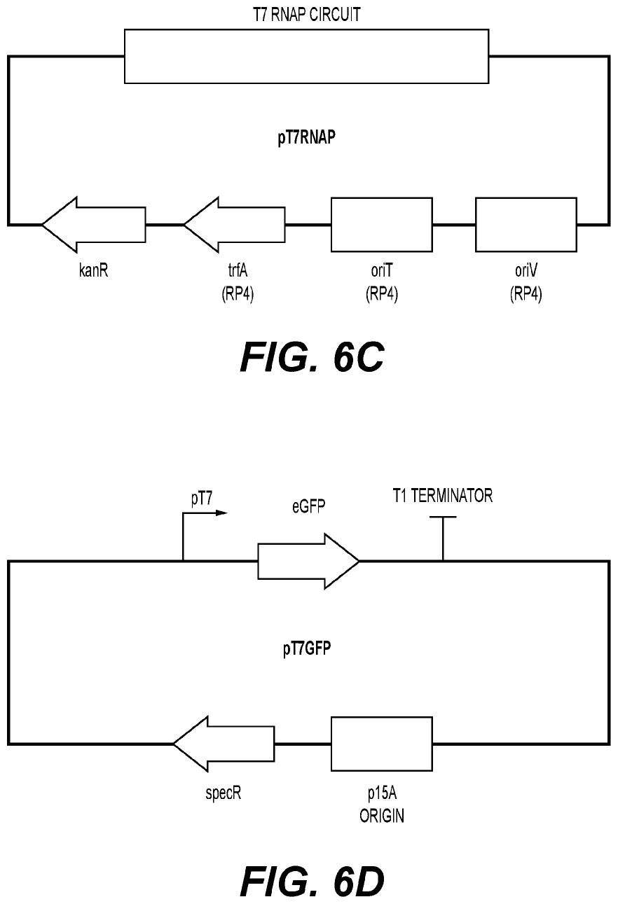

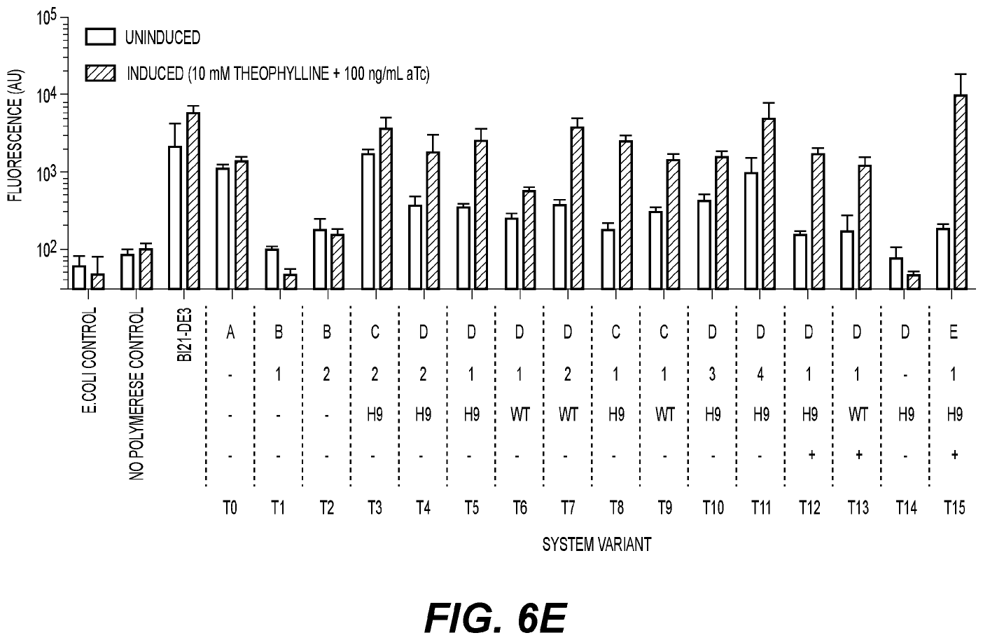

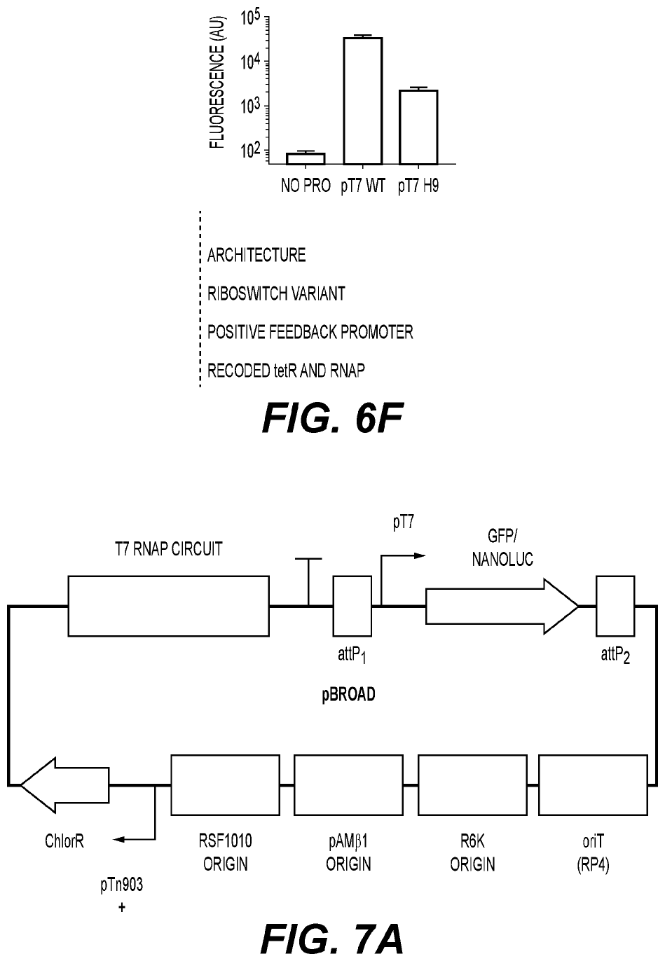

This additional module required rebalancing of the UBER framework. Five different variations of the expression circuit architecture were developed and tested (see, e.g., FIGS. 6A and 6B), and within these frameworks, a total of 16 variants (including variation of the architecture, riboswitch, positive feedback promoter, and recoding of tetR and RNAP) were tested to investigate how these variables influence the strength of positive-negative feedback, riboswitch variant, and general architecture (FIG. 6A, 6B). Although any of these architectures and variants may be used, the architecture of FIGS. 6A and 6B “e”, more particularly variant T15, was empirically determined to be the most preferred. The circuit includes a tetO tet-on tetracycline-controlled transcriptional activator sequence, a pT7 promoter driving expression of T7 RNAP through an intervening theophylline-responsive riboswitch, and a pT7 promoter driving expression of a tetR tetracycline repressor. Additionally or alternatively a Tet-off tetracycline-controlled transcriptional repressor sequence can added or substituted in the foregoing embodiment, or other embodiments disclosed herein. This architecture functions as an AND gate, relying on both theophylline and aTc for full induction, with theophylline acting as the stronger inducer.

The sequence differences between the various components used here can be found in SEQ ID NOS:99-124 and 136.

Other suitable elements and modules can be substituted to generate alternative circuits consistent with the same strategies. For example, in the T15 circuit, a theophylline riboswitch controls T7 RNAP expression levels to introduce titratable control. Alternatively, other ribsoswitches can also be used which respond to other ligands. Additionally or alternatively, CRISPRi or CRISPRa methods can be used to similarly titrate T7 RNAP expression levels within the circuit. In addition or alternative to the tetR discussed above, other negative feedback systems, such as other repressor protein/operator pairs, can be introduced. A particular alternative repressor is e.g., LacR. Other viral promoters beyond T7 can be used, and include, e.g., T3, SP6, KP34, K11, etc. In particular embodiments, the promoter is pT3 and the RNA polymerase T3/RNAP, or the promoter is pSP6 and the RNA polymerase SP6 RNA polymerase.

### III. Landing Pads for SGE Mobilization

Integration can increase genetic stability and biosynthetic pathway productivity (Tyo et al., 2009). Thus, compositions and methods for a SGE mobilization and chromosomal integration are also provided. SGE landing pads can be chromosomally integrated into the organisms of interest, and serve as target sites for facile and stable transfer of SGEs across diverse hosts. Thus provided are landing pad design strategies and structures, template landing pads, cells containing landing pads, methods of introducing new and substitute SGEs into cell-integrated landing pads, and cells including SGE-integrated landing pads.

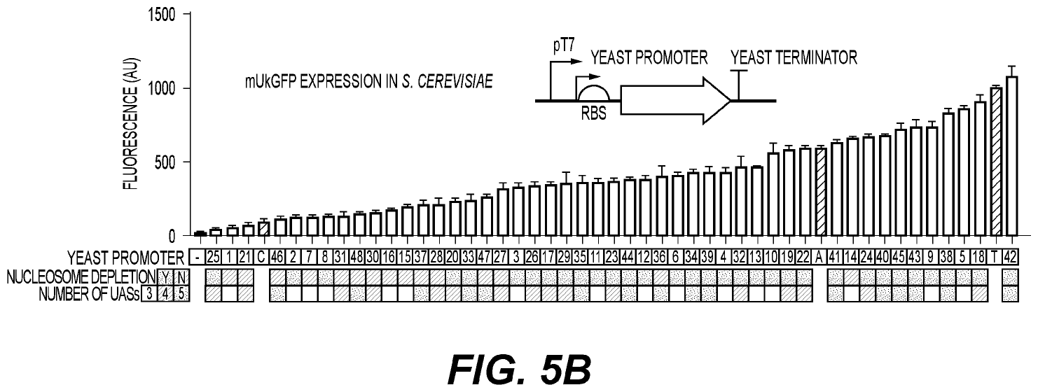

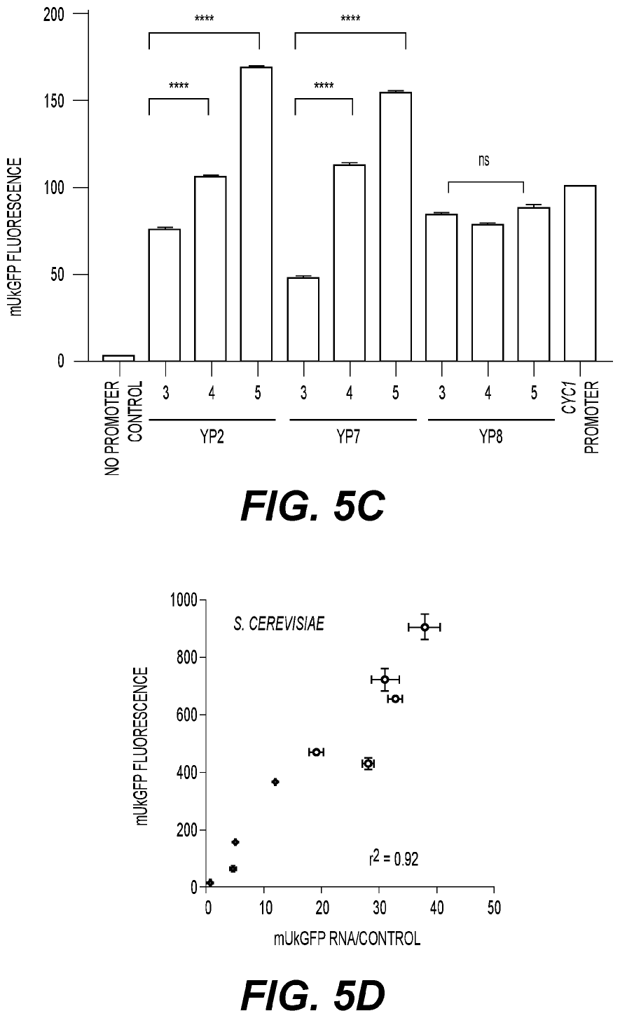

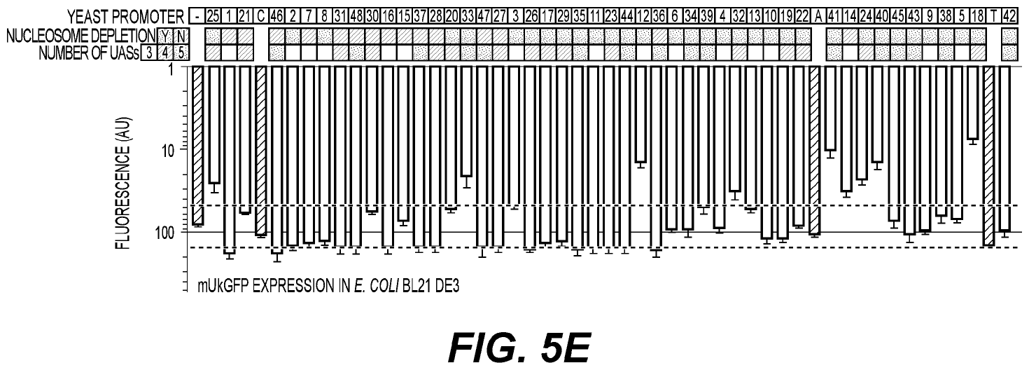

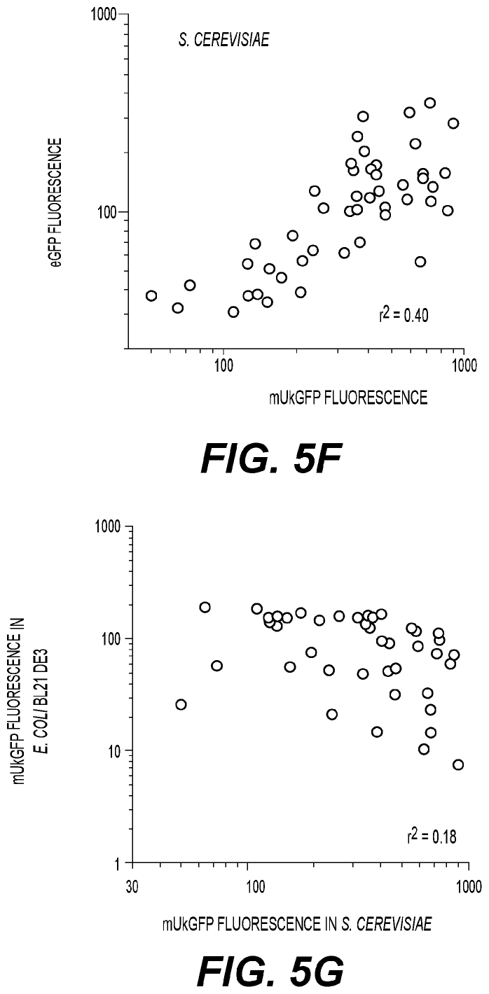

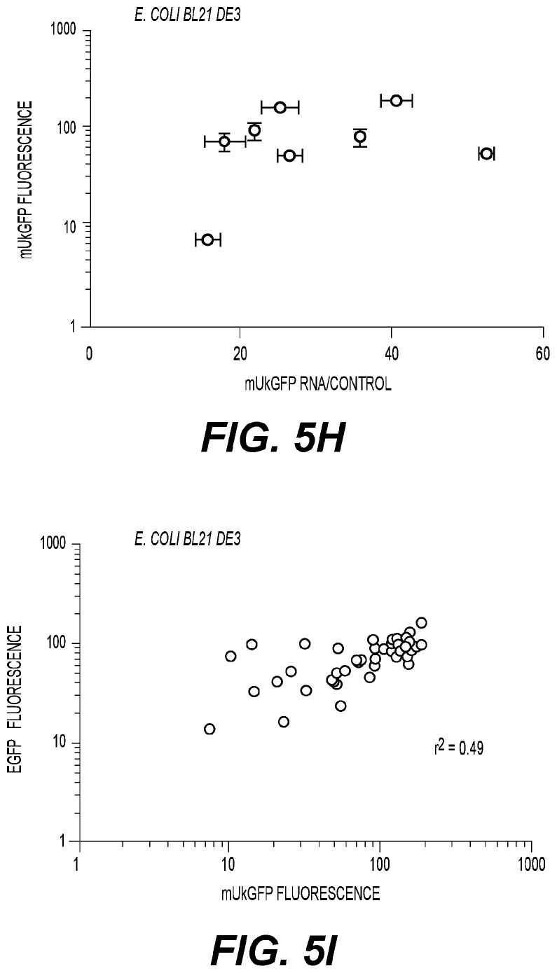

For example, the experiments below utilize a two-staged approach to integrate large SGEs into the genome. First, conjugative transposition is used to empirically identify safe landing sites that can stably express the T7 RNAP circuit (FIG. 5A). Second, site-specific integration is used to introduce SGEs into those safe landing sites (FIG. 5B).

### A. Land Pad Structure and Screening for Integration

A landing pad is a construct including SGE expression control sequences such as the T7RNAP circuit discussed above, that can serve as a location for versatile substitution of alternative SGEs within an organism of interest. This can be accomplished by first integrating the landing pad into the organism's genome. If an alternative SGE is later desired, it can be substituted for the initial SGE in a second step. The format of the landing strategy and illustration of its integration, and later SGE substitution are illustrated in FIGS. 5A and 5B, and described in more detail in a non-limiting example in the experiments below.

For example, a cassette can contain an expression control circuit such as a T7RNAP described above, (e.g., the titratable variant T15), a cognate promoter driving reporter gene (e.g., pT7-GFP-nanoluc luciferase fusion reporter in the experiments below), a selectable marker (e.g., an antibiotic selectable (e.g., apramycin resistance) marker in the experiments below) typically driven by a seen promoter (e.g., pX in the experiments below), and integration sites flanking the reporter gene (e.g., asymmetric phiC31 attP sites in the experiments below). This cassette can be further flanked by transposase terminal repeats, followed by the transposase gene, preferably which itself does not mobilize into the recipient genome. This transposase is preferably independent of host-specific factors and shows little bias in random integration. Examples of transposes include, but are not limited to the Himar and Tn5 transposases used in the experiments below. In preferred embodiments, the transposase is a Himar transposase requiring only a TA dinucleotide target (Lampe et al., 1999), which is specifically incorporated by reference herein in its entirety. Thus, isolated nucleic acids encoding any and all of these features alone and together are provided. As discussed in more detail below, the nucleic acid constructs can initially form part of extrachromosomal vectors, and be integrated into the chromosomes of cells.

Thus, nucleic acids encoding any and all of these features alone and together in the context of extrachromosomal vectors and cells including nucleic acids encoding any and all of these features alone and together in the context of an extrachromosomal vector and/or integrated into a chromosome of the cell are all expressly disclosed.

The cassette can be introduced into diverse cells, e.g., prokaryotic (e.g., bacterial) or eukaryotic cells, using any suitable means. A preferred means is a conjugation strategy in which a transposase is expressed and induces integration of the cassette into desired host cells.

In preferred embodiments, the transposase is transiently expressed and/or not integrated into the organisms of interest. A non-limiting strategy is as through a suicide vector, such as the R6K-based suicide plasmid was used for mobilization of the landing pad into diverse recipient bacteria via incP-mediated conjugation (Thomas and Smith, 1987) which is specifically incorporated by reference herein in its entirety, pLP (see, e.g., Figure S6E), as discussed in the experiments.

In some embodiments, transposases, promoters driving transposase expression, and other elements of the strategy are screened to fine tune the level of transposase expression, integration frequency and/or location, reduce mutation frequency (e.g., in the construct) and other elements of the system that may be different depending on the organism of interest and the size of the construct. For example, in some embodiments, the transposase is negatively regulated to reduce expression thereof and/or toxicity associated therewith. In the experiments below, hyperactive variants of both the Himar (Lampe et al., 1999) and Tn5 transposases (Martinez-Garcia et al., 2011) were tested, each of which of which are incorporated by reference herein in its entirety. Initially, these transposases were driven by a pTac promoter, which is highly active due to its consensus −10 and −35 promoter elements (de Boer et al., 1983), which is specifically incorporated by reference herein in its entirety. Factors include strong expression activity which may be counterbalanced by the exponentially decreasing efficiency associated with transposing large genetic constructs. Further, pTac transposase expression may be repressed in a LacR+E. coli conjugation donor strain, while derepressed in recipient strains.

However, use of the pTac promoter in this way may lead to mutations. Thus, in some embodiments, one or more of at least two different solutions can be utilized. In some embodiments, a trans-inhibiting construct can be utilized to fine tune transposase expression. In a non-limiting example in the experiments below, a trans-inhibiting plasmid, plnh, expressed a dominant-negative Tn5 inhibitor gene (de la Cruz et al., 1993) which is specifically incorporated by reference herein in its entirety, as well as a SP6 RNA Polymerase that produced an anti-sense silencing transcript of the transposase gene. This strategy can be used regardless of the transposase system that is selected. In some embodiments, this inhibitor plasmid is designed only to replicate in the conjugal donor strain. In the experiments below, presence of this plasmid in the conjugal donor strain facilitated cloning of landing pad constructs without mutation.

In a second strategy, a bacteriophage k pR promoter is used. This promoter can be repressed by a temperature sensitive CI857 gene (Valdez-Cruz et al., 2010), which is specifically incorporated by reference herein in its entirety. This promoter exhibited better repression in E. coli. As with other elements discussed herein, any of the landing pad elements can be subjected to recoding and/or any or all other steps of the CAD deign and refinement methodology discussed herein, to improve or otherwise modulate expression in the organism of interest. For example, recoding the CI857 gene and appending a strong synthetic RBS according the disclosed CAD methodology permitted stable construction and further reduced background by 25-fold (p<0.001) in the experiments below (FIG. 9). Taken together, these strategies successfully inhibited transposase activity in the conjugal donor, while allowing uninhibited transient expression in recipient microbes.

As introduced, above, these systems are modular and various selectable markers, seed promoters, inducible circuits, reporter genes, transposition and conjugation strategies, and host and target cells can be substituted for those used in the non-limiting examples provided, and utilized in the disclosed compositions and methods. However, these and other factors including, but not limited to, integration location and frequency, construct size, inducible circuit selection, promoter selection, reporter selection, strain selection, and other modular components of the system may impact the expression levels of the system, and may be different between organisms. Thus, in some embodiments, clones including various markers, inducible circuits, reporters, promotors, conjugation systems and attempts, integration locations and/or frequency and/or substitution of other modulator components of the system are screened, and cells of the organism(s) of interest having the desired expression characteristic are selected.

“Seed” promoter and transcription refers RNA transcription activity that initiates upstream of the RNA polymerase (e.g., T7 RNA Polymerase) and extends to produce an initial pool of mRNA (e.g., T7 RNA Polymerase mRNA). In some embodiments, this is a defined promoter placed upstream of the T7 RNA Polymerase or alternative polymerase including but not limited to those mentioned elsewhere herein. This promoter can be a native bacterial promoter or a synthetic bacterial promoter. Promoters can also be arrayed in tandem to increase the probability of expression in diverse microbes. In other embodiments, the polymerase sequence, e.g., T7 RNAP polymerase, is placed in a transcriptionally active region of a recipient microbial genome. Placement can be either though site-specific integration, or through random integration into the genome. In this embodiment, seeding transcription is provided by the host microbe.

For example, in the experiments below, an apramycin selectable landing pad was utilized, where seed transcription for the T7RNAP circuit was provided either by the active, broad host-range promoter P1 from pIP1433 (Trieu-Cuot et al., 1985) (FIG. 9) or by relying on background transcription at the host integration locus. Upon mobilizing this landing pad into E. coli MG1655 (transconjugation frequency=1.5×10⁻⁵ per recipient), flow cytometry was used to evaluate the transposed population with and without T7RNAP circuit induction (n˜2000 clones). The resulting population had broad fluorescence distributions evidenced by elevated coefficient of variation (CV) (FIG. 5C), indicating that there was substantial clonal heterogeneity in expression, attributable to the context-dependent effects of individual genomic locus integration sites. Heterogeneity may be present at several levels, including but not limited to, lower uninduced reporter or other target gene expression, tighter distributions, higher induction strength, and overall shape of the reporter or other target gene expression distribution. This approach allows the practitioner to leverage genetic context as a variable for tuning heterologous expression systems by selecting clones possessing the desired expression profile. By having the ability to survey multiple genetic loci, preferred (also referred to herein as “privileged”) clone(s) can be selected. The experiments below, which upon theophylline induction, the selected privileged clone, Clone 3, showed 20-fold stronger reporter (i.e., GFP) expression than the population average. This variability emerged in landing pads that both contained and lacked the pIP1433 seeding promoter, indicating that the presence of a strong promoter at the 5′ edge of the landing pad did not preclude heterogeneity caused by the integration locus.

The non-limiting examples below also show that these compositions and strategies can be effectively utilized in a diverse range of microbial organisms, wherein the conjugation-transposition system was tested and expression of the reporter construct was detected in Gammaproteobacterial clades—Klebsiella aerogenes, Salmonella enterica, Pseudomonas putida, Pseudomonas veronii, Cupriavidus necator, and cyanobacteria such as UTEX2973 and S. elongatus. However, the expression levels varied across different strains and even individual closes within a strain, illustrating the value in using a screen to select a clone in each organism of interest having the desired expression characteristics.

Sequence for pINH plasmid
(SEQ ID NO: 125)
AAGCTTGATGGGGGATCTAATACGACTCACTATAGGGAGAtttga
tagattaaaaaggaaaggaggaaagaaataatggctcgtgtacag
tttaaacaacgtgaatctactgacgcaatctttgttcactgctcg
gctaccaagccaagtcagaatgttggtgtccgtgagattcgccag
tggcacaaagagcagggttggctcgatgtgggataccactttatc
atcaagcgagacggtactgtggaggcaggacgagatgagatggct
gtaggctctcacgctaagggttacaaccacaactctatcggcgtc
tgccttgttggtggtatcgacgataaaggtaagttcgacgctaac
tttacgccagcccaaatgcaatcccttcgctcactgcttgtcaca
ctgctggctaagtacgaaggcgctggtcttcgcgcccatcatgag
gtggcgccgaaggcttgcccttcgttcgaccttaagcgttggtgg
gagaagaacgaactggtcacttctgaccgtggataaGATCCCATG
GTACGCGTGCTAGAGGCATCAAATAAAACGAAAGGCTCAGTCGAA
AGACTGGGCCTTTCGTTTTATCTGTTGTTTGTCGGTGAACGCTCT
CCTGAGTAGGACAAATCCGCCGCCCTAGAcctaggGGATATATTC
CGCTTCCTCGCTCACTGACTCGCTACGCTCGGTCGTTCGACTGCG
GCGAGCGGAAATGGCTTACGAACGGGGCGGAGATTTCCTGGAAGA
TGCCAGGAAGATACTTAACAGGGAAGTGAGAGGGCCGCGGCAAAG
CCGTTTTTCCATAGGCTCCGCCCCCCTGACAAGCATCACGAAATC
TGACGCTCAAATCAGTGGTGGCGAAACCCGACAGGACTATAAAGA
TACCAGGCGTTTCCCCCTGGCGGCTCCCTCGTGCGCTCTCCTGTT
CCTGCCTTTCGGTTTACCGGTGTCATTCCGCTGTTATGGCCGCGT
TTGTCTCATTCCACGCCTGACACTCAGTTCCGGGTAGGCAGTTCG
CTCCAAGCTGGACTGTATGCACGAACCCCCCGTTCAGTCCGACCG
CTGCGCCTTATCCGGTAACTATCGTCTTGAGTCCAACCCGGAAAG
ACATGCAAAAGCACCACTGGCAGCAGCCACTGGTAATTGATTTAG
AGGAGTTAGTCTTGAAGTCATGCGCCGGTTAAGGCTAAACTGAAA
GGACAAGTTTTGGTGACTGCGCTCCTCCAAGCCAGTTACCTCGGT
TCAAAGAGTTGGTAGCTCAGAGAACCTTCGAAAAACCGCCCTGCA
AGGCGGTTTTTTCGTTTTCAGAGCAAGAGATTACGCGCAGACCAA
AACGATCTCAAGAAGATCATCTTATTAATCAGATAAAATATTTCT
AGATTTCAGTGCAATTTATCTCTTCAAATGTAGCACCTGAAGTCA
GCCCCATACGATATAAGTTGTTactagttgcagaaataaaaaggc
ctgcgattaccagcagtcctgttattagctcagtaaagctTTATT
TGCCGACTACCTTGGTGATCTCGCCTTTCACGTAGTGGACAAATT
CTTCCAACTGATCTGCGCGCGAGGCCAAGCGATCTTCTTCTTGTC
CAAGATAAGCCTGTCTAGCTTCAAGTATGACGGGCTGATACTGGG
CCGGCAGGCGCTCCATTGCCCAGTCGGCAGCGACATCCTTCGGCG
CGATTTTGCCGGTTACTGCGCTGTACCAAATGCGGGACAACGTAA
GCACTACATTTCGCTCATCGCCAGCCCAGTCGGGCGGCGAGTTCC
ATAGCGTTAAGGTTTCATTTAGCGCCTCAAATAGATCCTGTTCAG
GAACCGGATCAAAGAGTTCCTCCGCCGCTGGACCTACCAAGGCAA
CGCTATGTTCTCTTGCTTTTGTCAGCAAGATAGCCAGATCAATGT
CGATCGTGGCTGGCTCGAAGATACCTGCAAGAATGTCATTGCGCT
GCCATTCTCCAAATTGCAGTTCGCGCTTAGCTGGATAACGCCACG
GAATGATGTCGTCGTGCACAACAATGGTGACTTCTACAGCGCGGA
GAATCTCGCTCTCTCCAGGGGAAGCCGAAGTTTCCAAAAGGTCGT
TGATCAAAGCTCGCCGCGTTGTTTCATCAAGCCTTACGGTCACCG
TAACCAGCAAATCAATATCACTGTGTGGCTTCAGGCCGCCATCCA
CTGCGGAGCCGTACAAATGTACGGCCAGCAACGTCGGTTCGAGAT
GGCGCTCGATGACGCCAACTACCTCTGATAGTTGAGTCGATACTT
CGGCGATCACCGCTTCCCTCATGATGTTTAACTTTGTTTTAGGGC
GACTGCCCTGCTGCGTAACATCGTTGCTGCTCCATAACATCAAAC
ATCGACCCACGGCGTAACGCGCTTGCTGCTTGGATGCCCGAGGCA
TAGACTGTACCCCAAAAAAACAGTCATAACAAGCCATGAAAACCG
CCACTGCGCCGTTACCACCGCTGCGTTCGGTCAAGGTTCTGGACC
AGTTGCGTGAGCGCATACGCTACTTGCATTACAGCTTACGAACCG
AACAGGCTTATGTCCACTGGGTTCGTGCCTTCATCCGTTTCCACG
GTGTGCGTCACCCGGCAACCTTGGGCAGCAGCGAAGTCGAGGCAT
TTCTGTCCTGGCTGagatcttgatcccctgcgccatcagatcctt
ggggcaagaaagccatccagtttactttgcagggcttcccaacct
taccagagggcgcGAAGGCGAAGCGGCATGCATTTACGTTGACAC
CATCGAATGGCGCAAAACCTTTCGCGGTATGGCATGATAGCGCCC
GGAAGAGAGTCAATTCAGGGTGGTGAATgtgAAACCAGTAACGTT
ATACGATGTCGCAGAGTATGCCGGTGTCTCTTATCAGACCGTTTC
CCGCGTGGTGAACCAGGCCAGCCACGTTTCTGCGAAAACGCGGGA
AAAAGTGGAAGCGGCGATGGCGGAGCTGAATTACATTCCCAACCG
CGTGGCACAACAACTGGCGGGCAAACAGTCGTTGCTGATTGGCGT
TGCCACCTCCAGTCTGGCCCTGCACGCGCCGTCGCAAATTGTCGC
GGCGATTAAATCTCGCGCCGATCAACTGGGTGCCAGCGTGGTGGT
GTCGATGGTAGAACGAAGCGGCGTCGAAGCCTGTAAAGCGGCGGT
GCACAATCTTCTCGCGCAACGCGTCAGTGGGCTGATCATTAACTA
TCCGCTGGATGACCAGGATGCCATTGCTGTGGAAGCTGCCTGCAC
TAATGTTCCGGCGTTATTTCTTGATGTCTCTGACCAGACACCCAT
CAACAGTATTATTTTCTCCCATGAAGACGGTACGCGACTGGGCGT
GGAGCATCTGGTCGCATTGGGTCACCAGCAAATCGCGCTGTTAGC
GGGCCCATTAAGTTCTGTCTCGGCGCGTCTGCGTCTGGCTGGCTG
GCATAAATATCTCACTCGCAATCAAATTCAGCCGATAGCGGAACG
GGAAGGCGACTGGAGTGCCATGTCCGGTTTTCAACAAACCATGCA
AATGCTGAATGAGGGCATCGTTCCCACTGCGATGCTGGTTGCCAA
CGATCAGATGGCGCTGGGCGCAATGCGCGCCATTACCGAGTCCGG
GCTGCGCGTTGGTGCGGATATCTCGGTAGTGGGATACGACGATAC
CGAAGACAGCTCATGTTATATCCCGCCGTTAACCACCATCAAACA
GGATTTTCGCCTGCTGGGGCAAACCAGCGTGGACCGCTTGCTGCA
ACTCTCTCAGGGCCAGGCGGTGAAGGGCAATCAGCTGTTGCCCGT
CTCACTGGTGAAAAGAAAAACCACCCTGGCGCCCAATACGCAAAC
CGCCTCTCCCCGCGCGTTGGCCGATTCATTAATGCAGCTGGCACG
ACAGGTTTCCCGACTGGAAAGCGGGCAGtgaGCGCAACGCAATTA
ATGTGAGTTAGCTCACTCATTAGGCACCCCAGGCTTTACACTTTA
TGCTTCCGGCTCGTATGTTGTGTGGaATTGTGAGCGGATAACAAT
TTCACACAGGAAACAGCTatgtttaatggtggcattcgtcgcttc
gaagcagatcaacaacgccagattgcagcaggtagcgagagcgac
acagcatggaaccgccgcctgttgtcagaacttattgcacctatg
gctgaaggcattcaggcttataaagaagagtacgaaggtaagaaa
ggtcgtgcacctcgcgcattggctttcttacaatgtgtagaaaat
gaagttgcagcatacatcactatgaaagttgttatggatatgctg
aatacggatgctacccttcaggctattgcaatgagtgtagcagaa
cgcattgaagaccaagtgcgcttttctaagctagaaggtcacgcc
gctaaatactttgagaaggttaagaagtcactcaaggctagccgt
actaagtcatatcgtcacgctcataacgtagctgtagttgctgaa
aaatcagttgcagaaaaggacgcggactttgaccgttgggaggcg
tggccaaaagaaactcaattgcagattggtactaccttgcttgaa
atcttagaaggtagcgttttctataatggtgaacctgtatttatg
cgtgctatgcgcacttatggcggaaagactatttactacttacaa
acttctgaaagtgtaggccagtggattagcgcattcaaagagcac
gtagcgcaattaagcccagcttatgccccttgcgtaatccctcct
cgtccttggagaactccatttaatggagggttccatactgagaag
gtagctagccgtatccgtcttgtaaaaggtaaccgtgagcatgta
cgcaagttgactcaaaagcaaatgccaaaggtttataaggctatc
aacgcattacaaaatacacaatggcaaatcaacaaggatgtatta
gcagttattgaagaagtaatccgcttagaccttggttatggtgta
ccttccttcaagccactgattgacaaggagaacaagccagctaac
ccggtacctgttgaattccaacacctgcgcggtcgtgaactgaaa
gagatgctatcacctgagcagtggcaacaattcattaactggaaa
ggcgaatgcgcgcgcctatataccgcagaaactaagcgcggttca
aagtccgccgccgttgttcgcatggtaggacaggcccgtaaatat
agcgcctttgaatccatttacttcgtgtacgcaatggatagccgc
agccgtgtctatgtgcaatctagcacgctctctccgcagtctaac
gacttaggtaaggcattactccgctttaccgagggacgccctgtg
aatggcgtagaagcgcttaaatggttctgcatcaatggtgctaac
ctttggggatgggacaagaaaacttttgatgtgcgcgtgtctaac
gtattagatgaggaattccaagatatgtgtcgagacatcgccgca
gaccctctcacattcacccaatgggctaaagctgatgcaccttat
gaattcctcgcttggtgctttgagtatgctcaataccttgatttg
gtggatgaaggaagggccgacgaattccgcactcacctaccagta
catcaggacgggtcttgttcaggcattcagcactatagtgctatg
cttcgcgacgaagtaggggccaaagctgttaacctgaaaccctcc
gatgcaccgcaggatatctatggggcggtggcgcaagtggttatc
aagaagaatgcgctatatatggatgcggacgatgcaaccacgttt
acttctggtagcgtcacgctgtccggtacagaactgcgagcaatg
gctagcgcatgggatagtattggtattacccgtagcttaaccaaa
aagcccgtgatgaccttgccatatggttctactcgcttaacttgc
cgtgaatctgtgattgattacatcgtagacttagaggaaaaagag
gcgcagaaggcagtagcagaagggcggacggcaaacaaggtacat
ccttttgaagacgatcgtcaagattacttgactccgggcgcagct
tacaactacatgacggcactaatctggccttctatttctgaagta
gttaaggcaccgatagtagctatgaagatgatacgccagcttgca
cgctttgcagcgaaacgtaatgaaggcctgatgtacaccctgcct
actggcttcatcttagaacagaagatcatggcaaccgagatgcta
cgcgtgcgtacctgtctgatgggtgatatcaagatgtcccttcag
gttgaaacggatatcgtagatgaagccgctatgatgggagcagca
gcacctaatttcgtacacggtcatgacgcaagtcaccttatcctt
accgtatgtgaattggtagacaagggcgtaactagtatcgctgta
atccacgactcttttggtactcatgcagacaacaccctcactctt
agagtggcacttaaagggcagatggttgcaatgtatattgatggt
aatgcgcttcagaaactactggaggagcatgaagagcgctggatg
gttgatacaggtatcgaagtacctgagcaaggggagttcgacctt
aacgaaatcatggattctgaatacgtatttgcctaattgacggct
agctcagtcctaggtacagtgctagcAGCTAAAGCTATATAATTT
AATTAGGAGAAGTAAAATGCAGGAAGGCGCGTATCGTTTTATTCG
TAATCCGAACGTGAGCGCGGAAGCGATTCGTAAAGCGGGTGCCAT
GCAGACCGTGAAACTGGCCCAGGAATTTCCGGAACTGCTGGCAAT
TGAAGATACCACCTCTCTGAGCTATCGTCATCAGGTGGCGGAAGA
ACTGGGCAAACTGGGTAGCATTCAGGATAAAAGCCGTGGTTGGTG
GGTGCATAGCGTGCTGCTGCTGGAAGCGACCACCTTTCGTACCGT
GGGCCTGCTGCATCAAGAATGGTGGATGCGTCCGGATGATCCGGC
GGATGCGGATGAAAAAGAAAGCGGCAAATGGCTGGCCGCTGCTGC
AACTTCGCGTCTGAGAATGGGCAGCATGATGAGCAACGTGATTGC
GGTGTGCGATCGTGAAGCGGATATTCATGCGTATCTGCAAGATAA
ACTGGCCCATAACGAACGTTTTGTGGTGCGTAGCAAACATCCGCG
TAAAGATGTGGAAAGCGGCCTGTATCTGTATGATCACCTGAAAAA
CCAGCCGGAACTGGGCGGCTATCAGATTAGCATTCCGCAGAAAGG
CGTGGTGGATAAACGTGGCAAACGTAAAAACCGTCCGGCGCGTAA
AGCGAGCCTGAGCCTGCGTAGCGGCCGTATTACCCTGAAACAGGG
CAACATTACCCTGAACGCGGTGCTGGCCGAAGAAATTAATCCGCC
GAAAGGCGAAACCCCGCTGAAATGGCTGCTGCTGACCAGCGAGCC
GGTGGAAAGTCTGGCCCAAGCGCTGCGTGTGATTGATATTTATAC
CCATCGTTGGCGCATTGAAGAATTTCACAAAGCGTGGAAAACGGG
TGCGGGTGCGGAACGTCAGCGTATGGAAGAACCGGATAACCTGGA
ACGTATGGTGAGCATTCTGAGCTTTGTGGCGGTGCGTCTGCTGCA
ACTGCGTGAATCTTTTACTCCGCCGCAAGCACTGCGTGCGCAGGG
CCTGCTGAAAGAAGCGGAACACGTTGAAAGCCAGAGCGCGGAAAC
CGTGCTGACCCCGGATGAATGCCAACTGCTGGGCTATCTGGATAA
AGGCAAACGCAAACGCAAAGAAAAAGCGGGCAGCCTGCAATGGGC
GTATATGGCGATTGCGCGTCTGGGCGGCTTTATGGATAGCAAACG
TACCGGCATTGCGAGCTGGGGTGCGCTGTGGGAAGGTTGGGAAGC
GCTGCAAAGCAAACTGGATGGCTTTCTGGCCGCGAAAGACCTGAT
GGCGCAGGGCATTAAAATCTAA

B. Substitution of SGE within Landing Pads

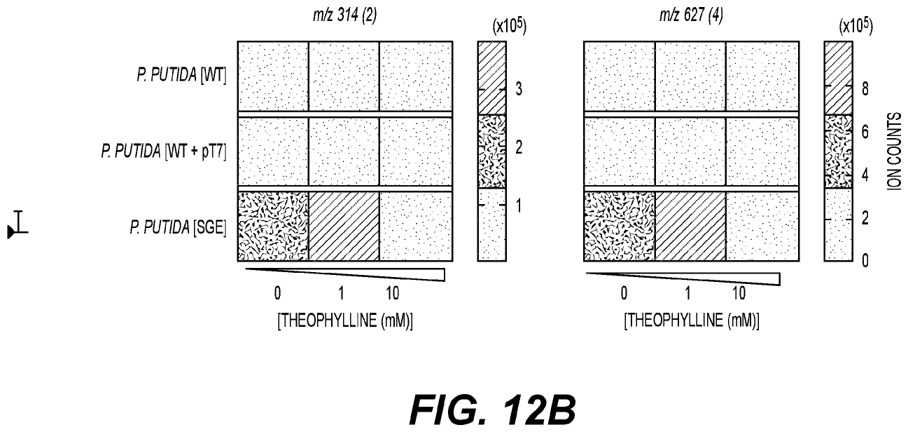

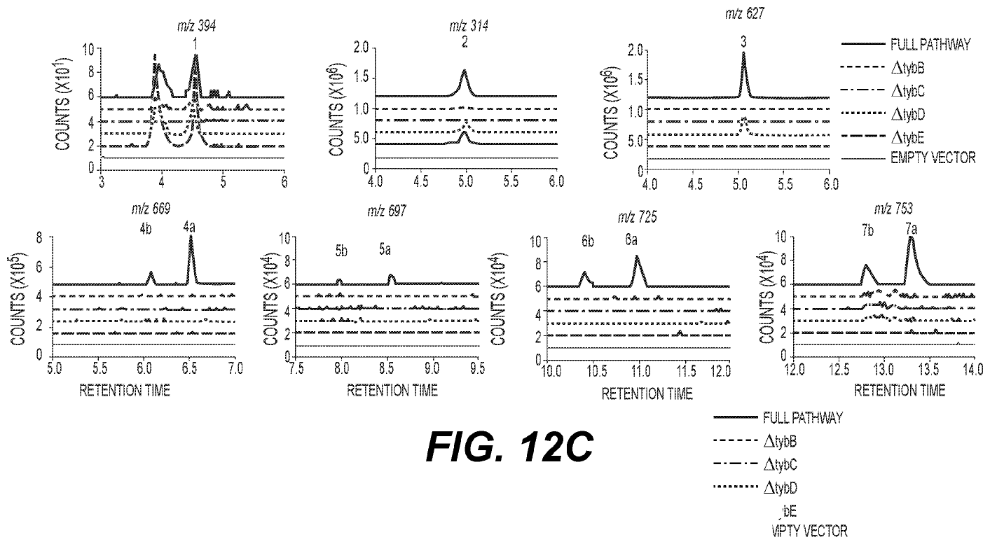

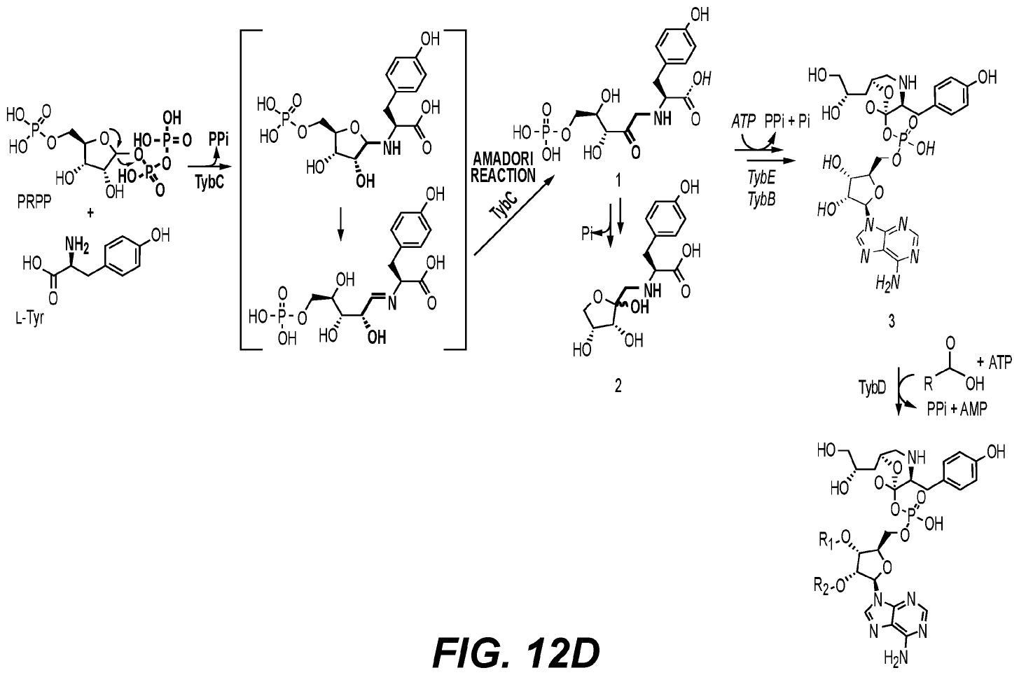

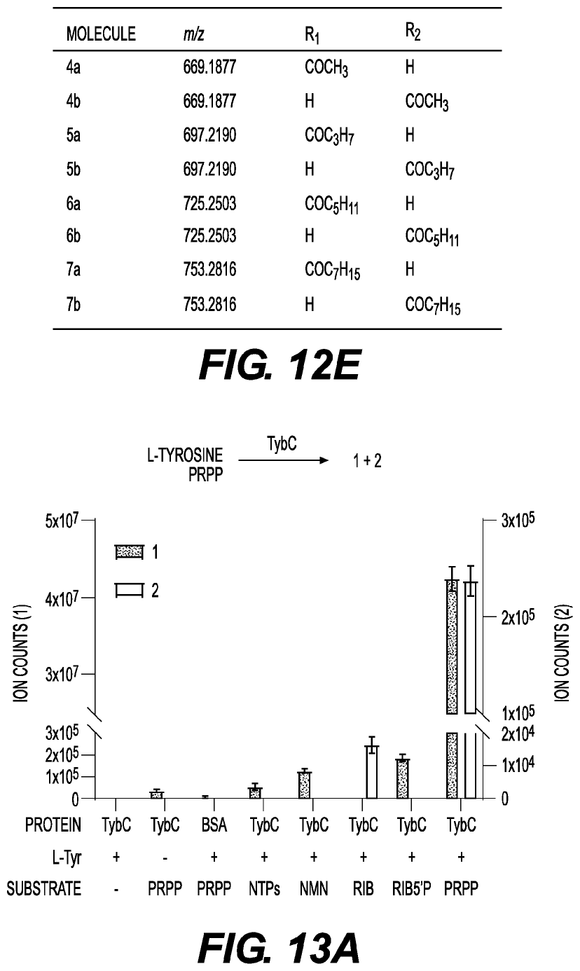

Once a landing pad is integrated into a target organism, also referred to herein a domesticated organism, the existing SGE can be readily introduce (e.g., substituted). For example, in some embodiments, the reporter gene and/or other SGE (e.g., series of CDSs), is replaced with a new SGE, by any suitable means, such by conjugation and site specific integration as illustrated in FIG. 5B. In the experiments below, SGEs were cloned into an R6K-based suicide vector, pPath (FIG. 12A), containing the phiC31 integrase and aminoglycoside resistance element functional in both prokaryotes (kanamycin) and S. cerevisiae (G418). SGE pathways were flanked with asymmetrical attB sites, such that when conjugated into recipient hosts, the site-specific integrase stably integrates the new SGE cargo into the landing pad, displacing the existing pathway or reporter (e.g., in the experiments below, the GFP-luciferase reporter).

Sequence for the Ppath plasmid
(SEQ ID NO: 126)
gggtgccagggcgtgcccGTgggctccccgggcgcgtaTAGGGAT
AACAGGGTAATacgtcaaattctatcataattgtggtttcaaaat
cggctccgtcgatactatgttatacgccaactttgaaaacaactt
tgaaaaggctgttttctgtatttaaggttttagaatgcaaggaac
agtgaattggagttcgtcttgttataattagcttcttggggtatc
tttaaatactgtagaaaaAGAAATTGCATACCTTTGTTCCTCGGT
TATATGTTTGCTCATCTGCAAgacatggaggcccagaataccctc
cttgacagtcttgacgtgcgcagctcaggggcatgatgtgactgt
cgcccgtacatttagcccatacatccccatgtataatcatttgca
tccatacattttgatggccgcacggcgcgaagcaaaaattacggc
acctcgctgcagacctgcgagcagggaaacgctcccctcacagac
gcgttgaattgtccccacgccgcgcccctgtagagaaatataaaa
ggttaggatttgccactgaggttcttctttcatatacttcctttt
aaaatcttgctaggatacagttctcacatcacatccgaacataaa
caaccaaaaatttgtaattaagaaggagtgattacATGGGTAAGG
AAAAGACTCACGTTTCGAGGCCGCGATTAAATTCCAACATGGATG
CTGATTTATATGGGTATAAATGGGCTCGCGATAATGTCGGGCAAT
CAGGTGCGACAATCTATCGATTGTATGGGAAGCCCGATGCGCCAG
AGTTGTTTCTGAAACATGGCAAAGGTAGCGTTGCCAATGATGTTA
CAGATGAGATGGTCAGACTAAACTGGCTGACGGAATTTATGCCTC
TTCCGACCATCAAGCATTTTATCCGTACTCCTGATGATGCATGGT
TACTCACCACTGCGATCCCCGGCAAAACAGCATTCCAGGTATTAG
AAGAATATCCTGATTCAGGTGAAAATATTGTTGATGCGCTGGCAG
TGTTCCTGCGCCGGTTGCATTCGATTCCTGTTTGTAATTGTCCTT
TTAACAGCGATCGCGTATTTCGTCTCGCTCAGGCGCAATCACGAA
TGAATAACGGTTTGGTTGATGCGAGTGATTTTGATGACGAGCGTA
ATGGCTGGCCTGTTGAACAAGTCTGGAAAGAAATGCATAAGCTTT
TGCCATTCTCACCGGATTCAGTCGTCACTCATGGTGATTTCTCAC
TTGATAACCTTATTTTTGACGAGGGGAAATTAATAGGTTGTATTG
ATGTTGGACGAGTCGGAATCGCAGACCGATACCAGGATCTTGCCA
TCCTATGGAACTGCCTCGGTGAGTTTTCTCCTTCATTACAGAAAC
GGCTTTTTCAAAAATATGGTATTGATAATCCTGATATGAATAAAT
TGCAGTTTCATTTGATGCTCGATGAGTTTTTCTAAtcagtactga
caataaaaagattcttgttttcaagaacttgtcatttgtatagtt
tttttatattgtagttgttctattttaatcaaatgttagcgtgat
ttatattttttttcgcctcgacatcatctgtccagatgcgaagtt
aagtgcgcagaaagtaatatcatgcgtcaatcgtatgtgaatgct
ggtcgctatactgctCTAGCATAACCCCGCGGGGCCTCTTTCGGG
GATCTCGCGGGGTTTTTTGCTGAAAGAAGCTTCAAATAAAACGAA
AGGCTCAGTCGAAAGACTGGGCCTTTCGTTATGTTGTTGTCGCTG
CGGCCGCACTCGAGCACCACCACCACCACCACTGGGATCCGGCTG
CTAACAAAGCCCGAAAGGAAGCTGAGTTGGCTGCTGCCACCGCTG
AGCAATAACTAGCATAACCCCTTGGGGCCTCTAAACGGGTCTTGA
GGGGTTTTTTGCTGAAGGCCatcatGGCCTAATACGACTCACTAT
AGGGAGAtcctgcaggccaatgtgatggACACtGAGACCTCCACA
TATACCTGCCGTTCACTATTATTTAGTGAAATGAGATATTATGAT
ATTTTCTGAATTGTGATTAAAAAGGCAACTTTATGCCCATGCAAC
AGAAACTATAAAAAATACAGAGAATGAAAAGAAACAGATAGATTT
TTTAGTTCTTTAGGCCCGTAGTCTGCAAATCCTTTTATGATTTTC
TATCAAACAAAAGAGGAAAATAGACCAGTTGCAATCCAAACGAGA
GTCTAATAGAATGAGGTCGAAAAGTAAATCGCGCGGGTTTGTTAC
TGATAAAGCAGGCAAGACCTAAAATGTGTAAAGGGCAAAGTGTAT
ACTTTGGCGTCACCCCTTACATATTTTAGGTCTTTTTTTATTGTG
CGTAACTAACTTGCCATCTTCAAACAGGAGGGCTGGAAGAAGCAG
ACCGCTAACACAGTACATAAAAAAGGAGACATGAACGATGAACAT
CAAAAAGTTTGCAAAACAAGCAACAGTATTAACCTTTACTACCGC
ACTGCTGGCAGGAGGCGCAACTCAAGCGTTTGCGAAAGAAACGAA
CCAAAAGCCATATAAGGAAACATACGGCATTTCCCATATTACACG
CCATGATATGCTGCAAATCCCTGAACAGCAAAAAAATGAAAAATA
TCAAGTTCCTGAATTCGATTCGTCCACAATTAAAAATATCTCTTC
TGCAAAAGGCCTGGACGTTTGGGACAGCTGGCCATTACAAAACGC
TGACGGCACTGTCGCAAACTATCACGGCTACCACATCGTCTTTGC
ATTAGCCGGAGATCCTAAAAATGCGGATGACACATCGATTTACAT
GTTCTATCAAAAAGTCGGCGAAACTTCTATTGACAGCTGGAAAAA
CGCTGGCCGCGTCTTTAAAGACAGCGACAAATTCGATGCAAATGA
TTCTATCCTAAAAGACCAAACACAAGAATGGTCAGGTTCAGCCAC
ATTTACATCTGACGGAAAAATCCGTTTATTCTACACTGATTTCTC
CGGTAAACATTACGGCAAACAAACACTGACAACTGCACAAGTTAA
CGTATCAGCATCAGACAGCTCTTTGAACATCAACGGTGTAGAGGA
TTATAAATCAATCTTTGACGGTGACGGAAAAACGTATCAAAATGT
ACAGCAGTTCATCGATGAAGGCAACTACAGCTCAGGCGACAACCA
TACGCTGAGAGATCCTCACTACGTAGAAGATAAAGGCCACAAATA
CTTAGTATTTGAAGCAAACACTGGAACTGAAGATGGCTACCAAGG
CGAAGAATCTTTATTTAACAAAGCATACTATGGCAAAAGCACATC
ATTCTTCCGTCAAGAAAGTCAAAAACTTCTGCAAAGCGATAAAAA
ACGCACGGCTGAGTTAGCAAACGGCGCTCTCGGTATGATTGAGCT
AAACGATGATTACACACTGAAAAAAGTGATGAAACCGCTGATTGC
ATCTAACACAGTAACAGATGAAATTGAACGCGCGAACGTCTTTAA
AATGAACGGCAAATGGTACCTGTTCACTGACTCCCGCGGATCAAA
AATGACGATTGACGGCATTACGTCTAACGATATTTACATGCTTGG
TTATGTTTCTAATTCTTTAACTGGCCCATACAAGCCGCTGAACAA
AACTGGCCTTGTGTTAAAAATGGATCTTGATCCTAACGATGTAAC
CTTTACTTACTCACACTTCGCTGTACCTCAAGCGAAAGGAAACAA
TGTCGTGATTACAAGCTATATGACAAACAGAGGATTCTACGCAGA
CAAACAATCAACGTTTGCGCCAAGCTTCCTGCTGAACATCAAAGG
CAAGAAAACATCTGTTGTCAAAGACAGCATCCTTGAACAAGGACA
ATTAACAGTTAACAAATAAGGTCTCtAGAGccaccacagtggtta
attaaGGCCtgtgaGGCCGGACCAAAACGAAAAAAGGCCCCCCTT
TCGGGAGGCCTCTTTTCTGGAATTTGGTACCGAGgggtgccaggg
cgtgcccCAgggctccccgggcgcgtataatcgatttaaattagt
agcccgcctaatgagcgggcttttttttaattcccctatttgttt
atttttctaaatacattcaaatatgtatccgctcatgagacaata
accctgataaatgcttcaataatattgaaaaaggaagagtatgag
cattcagcattttcgtgtggcgctgattccgttttttgcggcgtt
ttgcctgccggtgtttgcgcatccggaaaccctggtgaaagtgaa
agatgcggaagatcaactgggtgcgcgcgtgggctatattgaact
ggatctgaacagcggcaaaattctggaatcttttcgtccggaaga
acgttttccgatgatgagcacctttaaagtgctgctgtgcggtgc
ggttctgagccgtgtggatgcgggccaggaacaactgggccgtcg
tattcattatagccagaacgatctggtggaatatagcccggtgac
cgaaaaacatctgaccgatggcatgaccgtgcgtgaactgtgcag
cgcggcgattaccatgagcgataacaccgcggcgaacctgctgct
gacgaccattggcggtccgaaagaactgaccgcgtttctgcataa
catgggcgatcatgtgacccgtctggatcgttgggaaccggaact
gaacgaagcgattccgaacgatgaacgtgataccaccatgccggc
agcaatggcgaccaccctgcgtaaactgctgacgggtgagctgct
gaccctggcaagccgccagcaactgattgattggatggaagcgga
taaagtggcgggtccgctgctgcgtagcgcgctgccggctggctg
gtttattgcggataaaagcggtgcgggcgaacgtggcagccgtgg
cattattgcggcgctgggcccggatggtaaaccgagccgtattgt
ggtgatttataccaccggcagccaggcgacgatggatgaacgtaa
ccgtcagattgcggaaattggcgcgagcctgattaaacattggta
aaccgatacaattaaaggctccttttggagcctttttttttggac
gacccttgtccttttccgctgcataaccctgcttcggggtcatta
tagcgattttttcggtatatccatcctttttcgcacgatatacag
gattttgccaaagggttcgtgtagactttccttggtgtatccaac
ggcgtcagccgggcaggataggtgaagtaggcccacccgcgagcg
ggtgttccttcttcactgtcccttattcgcacctggcggtgctca
acgggaatcctgctctgcgaggctggccgtaggccggccggcgcg
ccgatctgaagatcagcagttcaacctgttgatagtacgtactaa
gctctcatgtttcacgtactaagctctcatgtttaacgtactaag
ctctcatgtttaacgaactaaaccctcatggctaacgtactaagc
tctcatggctaacgtactaagctctcatgtttcacgtactaagct
ctcatgtttgaacaataaaattaatataaatcagcaacttaaata
gcctctaaggttttaagttttataagaaaaaaaagaatatataag
gcttttaaagcctttaaggtttaacggttgtggacaacaagccag
ggatgtaacgcactgagaagcccttagagcctctcaaagcaattt
tgagtgacacaggaacacttaacggctgacatggggcgcgcccag
gtcagtcacagtggagcctagcactcgctcagcgtgacggctcag
agcagaattcacgagccagaaatagtaacttttgcctaaatcaca
aattgcaaaatttaattgcttgcaaaaggtcacatgcttataatc
aacttttttaaaaatttaaaatacttttttattttttatttttaa
acataaatgaaataatttatttattgtttatgattaccgaaacat
aaaacctgctcaagaaaaagaaactgttttgtccttggaaaaaaa
gcactacctaggagcggccaaaatgccggcttacattttatgtta
gctggtggactgacgccagaaaatgttggtgatgcgcttagatta
aatggcgttattggtgttgatgtaagcggaggtgtggagacaaat
ggtgtaaaagactctaacaaaatagcaaatttcgtcaaaaatgct
aagaaataggttattactgagtagtatttatttaagtattgtttg
tgcacttgcctgcaagccttttgaaaagcaagcataaaagatcta
aacataaaatctgtaaaataacaagatgtaaagataatgctaaat
catttggctttttgattgattgtacaggaaaatatacatcgcagg
gggttgacttttaccatttcaccgcaatggaatcaaacttgttga
agagaatgttcacaggcgcatacgctacaatgacccgattcttgc
tagccgaattccagtcaggctgctagcaccagagctacgtgaccg
caggactagctccagctgagcgacaGcggcaagaaagccatccac
gccgaaaaccccgcttcggggggttttgccgcATCTTGCCGTAAC
TGACAAATTAACCGAAGGTTTCTTCCGGCCACTGGGATGCAATGA
CCTTACCAACAACGCTACAGGATTCGTTGCACGGGATCATTGGGT
ATTGCGGATTCAGCGGCTGCAGGAAAACTTGGCCAGAGTCGCGGA
TCAGCTTCTTAAAGGTGAATTCATCACCACCCAGGCGCGCAATGC
AAAAGTCTCCCGGCTCAACCGCCTGCTCAGGATCAACCAGGATCA
GCATACCATCCGGAAATGACGGCTTGCTGCCCGTCGGGGCCGTCA
TGCTGTTGCCTTCTACTTCCAGCCAAAACGCGCTATCGCTCGCTT
TTTTGGTAGTCGATACCCAACGCTCAGCGTCACCTTTGGTGAAGG
TACGCAGTTcCGGGCTAAACATACCTGCTTGAACGTGGCTGAAGA
CCGGGTATTCGTATTCAGAGCGCAGAGACGGTTGCATGCTAACCG
CTTCATACATTTCATAGATTTCACGAGCTATAGAAGGAGAGAACT
CCTCGACGGAAACTTTCAGAATCTTGGTCAGCAGTGCGGCGTTGT
ACGCGTTCAGCGCATTAATACCGTTAAACAGAGCGCCAACGCCGC
TCTGACCCATGCCCATCTTGTCGGCTACGCTTTCCTGAGACAGAC
CCAGCTCATTCTTTTTCTTCTCATAGATTGCTTTCAGACGACGTG
CGTCCTCTAGCTGCTCTTGTGTAAGCGGCTTTTTCTTCGTTGACA
TTTTGATCCTCCTTTATATGGAGGTACTAAATCGGAACGTTAAAT
CTATCACCGCAAGGGATAAATATCTAACACCGTGCGTGTTGACTA
TTTTACCTCTGGCGGTGATAATGGTTGCATcaaatttgcgcgcca
cattattattcatacctttgtggaccgtattacaaagAGAAGTGT
TAAATCAAACAAAAAGGAGGATTAATCATGGACACGTACGCGGGT
GCTTACGACCGTCAGTCGCGCGAGCGCGAGAATTCGAGCGCAGCA
AGCCCAGCGACACAGCGTAGCGCCAACGAAGACAAGGCGGCCGAC
CTTCAGCGCGAAGTCGAGCGCGACGGGGGCCGGTTCAGGTTCGTC
GGGCATTTCAGCGAAGCGCCGGGCACGTCGGCGTTCGGGACGGCG
GAGCGCCCGGAGTTCGAACGCATCCTGAACGAATGCCGCGCCGGG
CGGCTCAACATGATCATTGTCTATGACGTGTCGCGCTTCTCGCGC
CTGAAGGTCATGGACGCGATTCCGATTGTCTCGGAATTGCTCGCC
CTGGGCGTGACGATTGTTTCCACTCAGGAAGGCGTCTTCCGGCAG
GGAAACGTCATGGACCTGATTCACCTGATTATGCGGCTCGACGCG
TCGCACAAAGAATCTTCGCTGAAGTCGGCGAAGATTCTCGACACG
AAGAACCTTCAGCGCGAATTGGGCGGGTACGTCGGCGGGAAGGCG
CCTTACGGCTTCGAGCTTGTTTCGGAGACGAAGGAGATCACGCGC
AACGGCCGAATGGTCAATGTCGTCATCAACAAGCTTGCGCACTCG
ACCACTCCCCTTACCGGACCCTTCGAGTTCGAGCCCGACGTAATC
CGGTGGTGGTGGCGTGAGATCAAGACGCACAAACACCTTCCCTTC
AAGCCGGGCAGTCAAGCCGCCATTCACCCGGGCAGCATCACGGGG
CTTTGTAAGCGCATGGACGCTGACGCCGTGCCGACCCGGGGCGAG
ACGATTGGGAAGAAGACCGCTTCAAGCGCCTGGGACCCGGCAACC
GTTATGCGAATCCTTCGGGACCCGCGTATTGCGGGCTTCGCCGCT
GAGGTGATCTACAAGAAGAAGCCGGACGGCACGCCGACCACGAAG
ATTGAGGGTTACCGCATTCAGCGCGACCCGATCACGCTCCGGCCG
GTCGAGCTTGATTGCGGACCGATCATCGAGCCCGCTGAGTGGTAT
GAGCTTCAGGCGTGGTTGGACGGCAGGGGGCGCGGCAAGGGGCTT
TCCCGGGGGCAAGCCATTCTGTCCGCCATGGACAAGCTGTACTGC
GAGTGTGGCGCCGTCATGACTTCGAAGCGCGGGGAAGAATCGATC
AAGGACTCTTACCGCTGCCGTCGCCGGAAGGTGGTCGACCCGTCC
GCACCTGGGCAGCACGAAGGCACGTGCAACGTCAGCATGGCGGCA
CTCGACAAGTTCGTTGCGGAACGCATCTTCAACAAGATCAGGCAC
GCCGAAGGCGACGAAGAGACGTTGGCGCTTCTGTGGGAAGCCGCC
CGACGCTTCGGCAAGCTCACTGAGGCGCCTGAGAAGAGCGGCGAA
CGGGCGAACCTTGTTGCGGAGCGCGCCGACGCCCTGAACGCCCTT
GAAGAGCTGTACGAAGACCGCGCGGCAGGCGCGTACGACGGACCC
GTTGGCAGGAAGCACTTCCGGAAGCAACAGGCAGCGCTGACGCTC
CGGCAGCAAGGGGCGGAAGAGCGGCTTGCCGAACTTGAAGCCGCC
GAAGCCCCGAAGCTTCCCCTTGACCAATGGTTCCCCGAAGACGCC
GACGCTGACCCGACCGGCCCTAAGTCGTGGTGGGGGCGCGCGTCA
GTAGACGACAAGCGCGTGTTCGTCGGGCTCTTCGTAGACAAGATC
GTTGTCACGAAGTCGACTACGGGCAGGGGGCAGGGAACGCCCATC
GAGAAGCGCGCTTCGATCACGTGGGCGAAGCCGCCGACCGACGAC
GACGAAGACGACGCCCAGGACGGCACGGAAGACGTAGCGGCGTAA
tctatagtgtcacctaaat

### IV. Isolated Nucleic Acids, Vectors, and Cells

As discussed extensively herein, the disclosed compositions and methods are designed to facilitate cross kingdom expression of diverse biosynthetic pathways including in rare and unusual organisms. Nucleic acids, vectors, and cells containing and/or embodying the disclosed elements and strategies are provided.

Exemplary host cells mentioned below, in the experiments, and elsewhere herein can be used, but should not be construed as limiting. Furthermore, as discussed extensively herein, the coding and expression control sequences and expression, conjugation, and integration strategies can utilize the one or more elements specifically disclosed herein, but are also modular in nature and thus may also be modified or unmodified elements of conventional expression, conjugation, and integration compositions and strategies. Thus, although non-limiting, specific exemplary hosts and new and conventional expression, conjugation, and integration compositions and strategies are provided herein and in the experiments below, and can be used.

### A. Isolated Nucleic Acid Molecules

### 1. Compositions

Isolated nucleic acids encoding part or all of any of the disclosed constructs, including, but not limited to individual CDSs, combinations of CDSs, expression control and other regulatory sequences, inducible circuits, integration and conjugation sequences, each individually and in all possible combinations are expressly disclosed. As used herein, “isolated nucleic acid” refers to a nucleic acid that is separated from other nucleic acid molecules that are present in a genome, including nucleic acids that normally flank one or both sides of the nucleic acid in the genome. The term “isolated” as used herein with respect to nucleic acids also includes the combination with any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule or an RNA molecule, provided one of the nucleic acid sequences normally found immediately flanking that DNA molecule in a naturally-occurring genome is removed or absent. Thus, an isolated nucleic acid includes, without limitation, a DNA molecule or RNA molecule that exists as a separate molecule independent of other sequences (e.g., a chemically synthesized nucleic acid, or a cDNA, or RNA, or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule or RNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

The disclosed nucleic acids may be optimized for expression in the expression host of choice as disclosed herein or alternatively or additional as is otherwise known in the art. For example as disclosed herein and elsewhere codons may be substituted with alternative codons encoding the same e.g., amino acid to account for differences in codon usage between the organism from which the nucleic acid sequence is derived and the expression host. In this manner, the nucleic acids may be synthesized using expression host-preferred codons.

Nucleic acids can be in sense or antisense orientation, or can be complementary to a reference sequence. Nucleic acids can be DNA, RNA, nucleic acid analogs, or combinations thereof. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

### 2. Methods for Producing Isolated Nucleic Acid Molecules

Isolated nucleic acid molecules can be produced by standard techniques, including, without limitation, common molecular cloning and chemical nucleic acid synthesis techniques. For example, polymerase chain reaction (PCR) techniques can be used to obtain an isolated nucleic acids. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995.

When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Nucleic acids can be mutated using standard techniques, including oligonucleotide-directed mutagenesis and/or site-directed mutagenesis through PCR.

### B. Expression Control Elements and Vectors

Vectors including the isolated nucleic acids are also provided. Nucleic acids, such as those described above, can be inserted into vectors for expression in cells. The vector can be a replicon, such as a plasmid, phage, virus or cosmid, into which another DNA segment may be inserted so as to bring about the replication of the inserted segment. The vectors can be integrative plasmids such as suicide vectors that are unable to replicate in the destination host and therefore must either integrate or disappear. Vectors can be expression vectors. An “expression vector” is a vector that includes one or more expression control sequences, and an “expression control sequence” is a DNA sequence that controls and regulates the transcription and/or translation of another DNA sequence.

The isolated nucleic acids including those in vectors and heterologously integrated in organism of interest can be operably linked to one or more expression control sequences. Operably linked means the disclosed sequences are incorporated into a genetic construct so that expression control sequences effectively control expression of a sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). In some embodiment, the expression control sequence(s) is one or more of those specifically mentioned herein including in the experimental examples. In some embodiments, the expression control sequence(s) additionally or alternatively are different expression control sequence(s) selected by the practitioner, preferably based on the desired result.

A promoter is a DNA regulatory region capable of initiating transcription of a gene of interest. Some promoters are “constitutive,” and direct transcription in the absence of regulatory influences. Some promoters are “tissue specific,” and initiate transcription exclusively or selectively in one or a few tissue types. Some promoters are “inducible,” and achieve gene transcription under the influence of an inducer. Induction can occur, e.g., as the result of a physiologic response, a response to outside signals, or as the result of artificial manipulation. Some promoters respond to the presence of tetracycline; “rtTA” is a reverse tetracycline controlled transactivator. Such promoters are well known to those of skill in the art.

To bring a coding sequence under the control of a promoter, it is advantageous to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein or other (e.g., RNA) element encoded by the coding sequence.

In some embodiments, one or more of the promoter is repressed by expression of a repressor. The repressor can, for example, be an agent encoded by gene introduced into the organism. The repressor can be driven by a promoter that can be constitutive, inducible, synthetic etc. Most typically, the promoter for the repressor is constitutively active so that the target gene is constitutively repressed unless the supplemental agent is present to block the repressor. Such systems are well known in the art. Two preferred examples are pLtetO and pLlacO. In the pLtetO system, TetR can be (e.g., constitutively) expressed by the organism, pLtetO, which drives expression of the target gene, is repressed by Tet Repressor Protein (TetR) unless a supplemental agent, anhydrotetracycline (ATc), is added to the culture conditions to block TetR repression. In the pLlacO system, lac Repressor (LacI) can be (e.g., constitutively) expressed by the organism. pLlacO, which drives expression of the target gene, is repressed by LacI unless a supplemental agent, isopropyl β-D-1-thiogalactopyranoside (IPTG), is added to the culture conditions to block LacI repression. These systems are others are discussed in, for example, Lutz and Bujard, Nucleic Acids Research, 25(6):1203-1210 (1997), and U.S. Pat. Nos. 4,495,280, 4,868,111, 5,362,646, 5,464,758, 5,589,362, 5,650.298, 5,654,168, 5,789,156, 5,814,618, 5,888,981, 5,922,927, 6,004,941, 6,087,166, 6,136,954, 6,242,667, 6,252,136, 6,271,341, 6,271,348, and 6,783,756.

Inducible promoters that are inactive unless activated by a supplemental agent are also known in the art and can be employed. For example, pAra is induced only in the presence of arabinose, and pRha which is induced only in the presence of rhamnose. These promoters and others can be used addition, combination, or alternative to pLlacO and pLtet to control expression of the crRNA-linked target gene and taRNA.

For example, in some embodiments, the expression circuit includes van-on Vanillin acid-controlled transcriptional activator sequence, an vanillin acid responsive VanR repressor, Van-off tetracycline-controlled transcriptional repressor, riboswitch (e.g., a theophylline-responsive translational riboswitch), or a combination thereof. Such a circuit can be controlled essentially by theophylline.

Although specific exemplary promoters are provided, the provided strategies are modular and can be used with any native or synthetic promoter as determined by the designer. For example, availability of inducible promoters for eukaryotic systems (e.g., Gal in yeast and Dox in mammalian systems) supports the application of strategies across a diverse range of microorganisms and cell types.

The vectors can be introduced into cells and/or microorganisms by standard methods including electroporation (From et al., Proc. Natl. Acad. Sci. USA 82, 5824 (1985), infection by viral vectors, high velocity ballistic penetration by small particles with the nucleic acid either within the matrix of small beads or particles, or on the surface (Klein et al., Nature 327, 70-73 (1987)). Methods of expressing recombinant proteins in various recombinant expression systems including bacteria, yeast, insect, and mammalian cells are known in the art, see for example Current Protocols in Protein Science (Print ISSN: 1934-3655 Online ISSN: 1934-3663, Last updated January 2012).

Plasmids can be high copy number or low copy number plasmids. In some embodiments, a low copy number plasmid generates between about 1 and about 20 copies per cell (e.g., approximately 5-8 copies per cell). In some embodiments, a high copy number plasmid generates at least about 100, 500, 1,000 or more copies per cell (e.g., approximately 100 to about 1,000 copies per cell).

Kits are commercially available for the purification of plasmids from bacteria, (see, e.g., GFX™ Micro Plasmid Prep Kit from GE Healthcare; Strataprep® Plasmid Miniprep Kit and StrataPrep® EF Plasmid Midiprep Kit from Stratagene; GenElute™ HP Plasmid Midiprep and Maxiprep Kits from Sigma-Aldrich, and, Qiagen plasmid prep kits and QIAfilter™ kits from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems.

Any of the constructs, including vectors, can include one or more phenotypic selectable marker genes. A phenotypic selectable marker gene is, for example, a gene encoding a protein that confers antibiotic resistance, supplies an autotrophic requirement, etc.

Following introduction of a by electroporation, lipofection, calcium phosphate, or calcium chloride co-precipitation, DEAE dextran, or other suitable transfection method, stable cell lines can be selected (e.g., by metabolic selection, or antibiotic resistance to G418, kanamycin, or hygromycin or by metabolic selection using the Glutamine Synthetase-NSO system). The transfected cells can be cultured such that the construct interest is expressed.

Methods of engineering a microorganism or cell line to incorporate a nucleic acid sequence into its genome are known in the art. Any of the disclosed nucleic acids can be incorporated and expressed from one or more genomic copies. For example, cloning vectors expressing a transposase and containing a nucleic acid sequence of interest between inverted repeats transposable by the transposase can be used to clone the stably insert the gene of interest into a bacterial genome (Barry, Gene, 71:75-84 (1980)). Stably insertion can be obtained using elements derived from transposons including, but not limited to Tn7 (Drahos, et al., Bio/Tech. 4:439-444 (1986)), Tn9 (Joseph-Liauzun, et al., Gene, 85:83-89 (1989)), Tn10 (Way, et al., Gene, 32:369-379 (1984)), and Tn5 (Berg, In Mobile DNA. (Berg, et al., Ed.), pp. 185-210 and 879-926. Washington, D.C. (1989)). Additional methods for inserting heterologous nucleic acid sequences in E. coli and other gram-negative bacteria include use of specialized lambda phage cloning vectors that can exist stably in the lysogenic state (Silhavy, et al., Experiments with gene fusions, Cold Spring Harbor Laboratory, Cold Spring Harbor, NY (1984)), homologous recombination (Raibaud, et al., Gene, 29:231-241 (1984)), and transposition (Grinter, et al., Gene, 21:133-143 (1983), and Herrero, et al., J. Bacteriology, 172(11):6557-6567 (1990)).

Methods of engineering other microorganisms or cell lines to incorporate a nucleic acid sequence into its genome are also known in the art. Nucleic acids that are delivered to cells which are to be integrated into the host cell genome can contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome. Techniques for integration of genetic material into a host genome are also known and include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods needed to promote homologous recombination are known to those of skill in the art.

Integrative plasmids can be used to incorporate nucleic acid sequences into host genomes. See for example, Taxis and Knop, Bio/Tech., 40(1):73-78 (2006), and Hoslot and Gaillardin, Molecular Biology and Genetic Engineering of Yeasts. CRC Press, Inc. Boca Raton, FL (1992). Methods of incorporating nucleic acid sequence into the genomes of mammalian lines are also well known in the art using, for example, engineered retroviruses such lentiviruses.

### C. Host Cells

Host cells, also referred to herein as organism(s) of interest, target organism, and which may be donor or recipient organisms transformed or transfected with the disclosed nucleic acids including, but not limited to, constructs and vectors which may be extrachromosomal or genomically integrated are also provided.

For example, prokaryotes useful as host cells include, but are not limited to, gram negative or gram positive organisms such as E. coli or Bacilli, cyanobacteria, and including, but not limited to, the specific organisms subject to the disclosed experiments or otherwise mentioned elsewhere herein (e.g., Klebsiella aerogenes, Salmonella enterica, Pseudomonas putida, Pseudomonas veronii, Cupriavidus necator, and cyanobacteria such as UTEX2973 and S. elongatus). Examples of useful expression vectors for prokarvotic host cells include those derived from commercially available plasmids such as the cloning vector pBR322 (ATCC 37017). pBR322 contains genes for ampicillin and tetracycline resistance and thus provides simple means for identifying transformed cells. To construct an expression vector using pBR322, an appropriate promoter and a DNA sequence are inserted into the pBR322 vector. Other commercially available vectors include, for example, T7 expression vectors from Invitrogen, pET vectors from Novagen and pALTERC vectors and PinPoint® R vectors from Promega Corporation.

Yeasts useful as host cells include, but are not limited to, those from the genus Saccharomyces, Pichia, K. Actinomycetes and Kluyveromyces. Yeast vectors will often contain an origin of replication sequence, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes (Holland et al., Biochem. 17:4900, (1978)) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Fleer et al., Gene, 107:285-195 (1991), in Li, et al., Lett Appl Microbiol. 40(5):347-52 (2005), Jansen, et al., Gene 344:43-51 (2005) and Daly and Hearn, J Mol. Recognit. 18(2):119-38 (2005). A yeast promoter is, for example, the ADH1 promoter (Ruohonen, et al., J Biotechnol. 1995 May 1; 39(3):193-203), or a constitutively active version thereof (e.g., the first 700 bp). Some embodiments include a terminator, such as the rpl41b terminator resulted in the highest GFP expression out of over 5300 yeast promoters tested (Yamaishi, et al., ACS Synth. Biol., 2013, 2 (6), pp 337-347). Other suitable promoters, terminators, and vectors for yeast and yeast transformation protocols are well known in the art.

In some embodiments, the host cells are non-yeast eukaryotic cells. For example, mammalian and insect host cell culture systems well known in the art can also be employed. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are easily obtained from a viral genome as a fragment which may also contain a viral origin of replication. Exemplary expression vectors for use in mammalian host cells are well known in the art. For example, eukaryotic expression vectors pCR3.1 (Invitrogen Life Technologies) and p91023(B) (see Wong et al. (1985) Science 228:810-815) are suitable for expression of recombinant proteins in, for example, Chinese hamster ovary (CHO) cells, COS-1 cells, human embryonic kidney 293 cells, NIH3T3 cells, BHK21 cells, MDCK cells, and human vascular endothelial cells (HUVEC). Additional suitable expression systems include the GS Gene Expression System™ available through Lonza Group Ltd.

### V. Applications

Disclosed herein are foundational technologies developed to decouple specialized metabolite BGCs from native layers of regulation, redesigning them into synthetic genetic elements with versatile cross-kingdom functionality. This technology utilized the integrated development of new computational and experimental methods. These included computer aided design of CDSs, the development of synthetic regulatory elements to promote transcription and translation in both prokaryotes and eukaryotes, and new mobilization methods to permit transfer into diverse species. Together, these advances facilitated the redesign of biosynthetic pathways and their expression in diverse microbes for the discovery of nucleotide metabolites from the human microbiome.

The disclosed strategies, compositions, and methods of use thus can be used to solves several problems of broad significance to biotechnology and drug discovery, spanning the fields of synthetic biology, molecular biology, microbiome engineering, natural product discovery, and host-microbe interaction communities. Historically, the ability to transform multigene pathways into diverse microbes was limited by constraints in mobilization and expression. These limitations usually require species-specific solutions for both functional expression and mobilization of genetic material into recipient strains. Solving this problem as disclosed herein facilitates the creation of new microbes to be domesticated for many, diverse commercial applications.

Exemplary uses of the disclosed strategies, compositions, and methods include:
Converting genomic sequence information from whole-genome or metagenome sequencing datasets into DNA constructs that can be introduced and expressed in diverse host microorganisms.
Design of genetic elements capable of functioning in diverse organisms.
Development of single regulatory elements (or promoters) capable of transcribing genes and multi-gene pathways in Gram-negative, Gram-positive and eukaryotic organisms.
The transfer of DNA fragments, including large (>10 kb) constructs, into diverse organisms, including non-model species.
Re-design, synthesis, mobilization, expression, and/or characterization of biosynthetic gene clusters (BGCs) including, but not limited to, the vast and untapped secondary metabolism of diverse microorganisms and plants including those that have been constrained due to technological limitations.
Re-design of the coding sequence (CDS), synthesis, mobilization, and/or expression in diverse microorganisms and communities, including the microbiomes of animals (e.g., human gut microbiome), plants, and environmental niches, including those that have been constrained due to technological limitations.
A comprehensive strategy that addresses a combination of some or all of the solutions described above into an integrated, high-throughput solution

These strategies, materials, and methods complement and advance current heterologous expression approaches, and can be used in combination therewith. These approaches include constructing combinatorial libraries of multigene pathways that incorporate different operon architectures, and transcription/translation signals that can survey differential expression levels (Ajikumar et al., 2010; Chan et al., 2005; Smanski et al., 2014). Additionally, biosynthetic pathways have been heterologously characterized by screening their metabolic activity in different model hosts (Craig et al., 2010; Wang et al., 2019a). Unifying the utility of both genetic refactoring and multi-host expression, the method appends pathways with synthetic, titratable transcriptional and translational signals specifically designed to be portable to diverse microbes.

Using the pigment violacein as a test case, the benefits of the approach were demonstrated in the experiments below, showing that redesign relieved transcriptional repression, further boosted activity post-transcriptionally though CDS optimization, and permitted transfer into diverse hosts. The fully redesigned pathway outperformed wildtype sequences in a heterologous context and produced more pigment than the native Chromobacterium producer. By porting the pathway into various heterologous hosts, differential expression across strains were empirically observed and strong pigment producers were identied. Pigment levels were quickly optomized by titrating expression with theophylline.

To further augment pathway engineering and optimization, the redesigned SGEs are amenable to rapid metabolic flux optimizations using computational guided flux balance analysis methods (Orth et al., 2010) or multiplex genome editing technologies (Anzalone et al., 2020; Wannier et al., 2021). Since SGE-based transcription and translation signals are modular and designed from the bottom-up, predictable tuning of gene expression is achievable in diverse hosts. More specifically, the 5′-UTRs can be predictably tuned at the thermodynamic level by introducing point mutations to modulate translation initiation.

Also, demonstrated is that the strength of the yeast promoters can also be predictably tuned simply by adding or removing 10-mer UTRs. Opportunities for future technological development include expanding the range of site-specific integrases that are used to augment the number of landing pads within a strain and testing the mobilization and expression of SGEs in more diverse hosts. A unique advantage of this approach is that strains domesticated with a landing pad can be used “off the shelf” for future heterologous expression of BGCs, other pathways, or any genetic element of interest.

Applying this procedure to deorphanize a human microbiome derived BGC to discover the bioactive nucleotide metabolites, tyrocitabines, demonstrated that the approach overcame two key challenges, reproducing outcomes observed with the violacein test case. First, without redesign into an SGE, metabolites could not be detected beyond the early intermediate 2 in P. putida, which would have prevented the complete elucidation and characterization of the L. iners BGC. Second, the ability to mobilize the de-orphaned pathway across multiple hosts simultaneously allowed for quick identification of productive heterologous hosts. For example, it was observed that the activity of the largest enzyme (the NRPS TybD) in the pathway was a bottleneck in several strains, causing the pathway to stall at tryrocitabine (3). Notably, production stalled in B. subtilis though it is phylogenetically closer to the native source of this gene cluster, L. iners. This finding—that a more phylogenetically distant heterologous host outperformed a less distant host—has precedence as previous studies have also observed similar results (Wang et al., 2019a). Ultimately, the findings highlight the benefit of being able to survey many distant hosts simultaneously. This multi-host strategy overcomes unpredictable limitations associated with heterologous expression, which include proper expression, folding, and localization of enzymes, availability of input substrates, and toxicity of metabolic intermediates.

Computational SEA analysis indicates that the tyrocitabines are nucleotide antimetabolites that could target proteins that use nucleotide substrates, such as the translational apparatus. It was validated that tyrocitabine, but not the acyl-tyrocitabines, inhibited the translational step using the PURExpress protein synthesis system (Tuckey et al., 2014). While these molecular studies now facilitate the biological study of these specific metabolites at the host-microbe interface in the context of vaginal homeostasis and disease, they also facilitate the identification of related uncharacterized pathways across a broad phylogenetic distribution. Indeed, observing the genomic context around the resulting BLAST hits, it is believed that the tyrocitabines represent the founding members of a much larger, yet previously elusive, class of specialized microbial nucleotide metabolites in the environment, including members of the human microbiome. Specifically, numerous instances of misannotated class Ic tRNA synthetases were found that not only lack the RNA binding domains, but also co-localize with anthranilate phosphoribosyltransferase-like enzymes. Als found were pathways that contain two tandem, yet sequence distinct class Ic tRNA synthetases, homologous to TrpRS and TyrRS, similarly lacking their RNA binding domains. This indicates the core tyrocitabine scaffold is likely highly diversified in nature, as the accessory proteins diverge substantially in the BGCs. This structural diversity could have profound implications on the cell type specificity, localization, and biological targets of the resulting functionalized molecules. Overall, these dedicated abortive tRNA synthetase reactions add a new dimension to specialized nucleotide metabolism, prompting further structural and biological characterization. In this study, the genome mining strategy used TybB as the search seed, which intrinsically biased the results toward the discovery of other Class Ic tRNA synthetase homologs. More broadly, this highlights a largely unexplored genome mining strategy—scrutinizing (misannotated) genes which are classically considered “central metabolism” and filtering for those with missing/added domains and unusual genome context. Such an approach could uncover the continual evolution and repurposing of otherwise ancestral genes for acquiring new functions and biochemistries.

Disclosed herein is a synthetic biology technology employed to elucidate orphan biosynthetic gene clusters. Given that only ˜10′ of ˜105 gene clusters currently predicted on DOE's IMG database have empirical elucidation, this approach is scalable toward the discovery of these uncharacterized BGCs. Beyond this application, the versatility of the disclosed redesign principles hold broad usefulness in rapidly domesticating diverse microbes for multiple applications. Fungal (Clevenger et al., 2017) and plant (Birchler, 2015) genomes are particularly rich in specialized metabolite biosynthetic potential; however, the portability of these biosynthetic genes into heterologous hosts can pose challenge. By rapidly surveying diverse hosts, privileged strains can be rapidly revealed to resolve heterologous bottlenecks. For example, this technology can be used in metabolic engineering applications that aim to maximize titers of high-value molecules in heterologous hosts (Paddon et al., 2013). Moreover, it has been demonstrated that cross-kingdom co-cultures of microbes can be leveraged to overcome challenges in heterologously producing difficult molecules, highlighting the usefulness in disseminating genetic cargo across taxonomic domains (Wu et al., 2021; Zhou et al., 2015). Finally, it is believed that the cross-species mobilization and expression of SGEs could enhance the engineering of living therapeutics (Zhou et al., 2020), which require transfer of genetic cargo into diverse environmental microbiome strains (Inda et al., 2019). Through the development of a technology for the design, mobilization, and expression of genetic elements, it is believed that this technology can aid in the domestication of non-model organisms and communities for diverse applications in medicine, environmental sustainability, and biotechnology.

The disclosed invention can be further understood by reference to the following numbered paragraphs:

1. A method of recoding a nucleic acid coding sequence including two, three, four, five, or all six of steps:
(1) selecting the codons of the coding sequence,
(2) implementing N-terminal codon bias;
(3) creating a synthetic or hybrid 5′ regulatory element;
(4) screening for internal ribosome binding sites (RBSs);
(5) randomizing one or more codons upstream of internal RBSs, and
(6) screening for internal terminators,
optionally, wherein the recoding improves expression of the nucleic acid coding sequence in one or more heterologous organisms of interest.

2. The method of paragraph 1, wherein the nucleic acid coding sequence is a naturally occurring sequence.

3. The method of paragraphs 1 or 2 including step (1), wherein codon selection is based partially or completely on the preferred codon distribution in the heterologous organism(s).

4. The method of paragraph 3, wherein codon usage is selected based on that of highly expressed genes in the heterologous organism(s).

5. The method of any one of paragraphs 1-4 including step (1), wherein codon selection is based on codon usage information derived from the genome sequence of a strain(s) of the heterologous organism or downloaded directly from a database(s).

6. The method of any one of paragraphs 3-5 including step (1), wherein step (1) includes depletion of canonically-inhibiting codons, optionally wherein the inhibiting codons are selected from TTA, AGG, CTA, CGA, CGG, CGA, TTG and/or GTG, or a combination thereof.

7. The method of any one of paragraphs 1-6 including step (2), wherein step (2) includes recoding the nucleic acid sequence encoding the N-terminus of a polypeptide encoded by the nucleic acid coding sequence to reduce secondary and/or tertiary structure.

8. The method of paragraph 7, wherein reducing secondary structure includes recoding a 5′ terminal stretch of 15-75 base pairs, or any subrange or specific integer therebetween, of the nucleic acid coding sequence.

9. The method of paragraphs 7 or 8 including step (2), wherein step (2) includes using a hybrid codon distribution that biases toward privileged or preferred codons encoding the N-terminus that correlate with high expression levels in the heterologous organism(s).

10. The method of any one of paragraph 7-9, wherein the recoding of the nucleic acid sequence encoding the N-terminus of a polypeptide includes the codon adaptation index (CAI) approach and/or the tRNA adaptation index (TAI).

11. The method of any one of paragraphs 1-10 including step (3) wherein the synthetic or hybrid regulatory element is designed for versatile regulation across diverse prokaryotes and eukaryotes.

12. The method of any one of paragraphs 1-11 including step (3), wherein step (3) includes creation of a hybrid of eukaryotic and prokaryotic element(s) that can impact gene expression in one, two, three, or more microbial taxa, optionally wherein one or more of the taxa include the heterologous organism(s).

13. The method of any one of paragraphs 1-11 including step (3), wherein step (3) includes utilizing a thermodynamic translation initiation model optionally wherein the thermodynamic translation initiation model defines sequence and/or structural determinants of ribosomal entry, optionally bacterial ribosome entry, and allows predictions of translation initiation rates using a ribosomal binding site (RBS) calculator.

14. The method of any one of paragraphs 1-13 including step (3), wherein step (3) includes consideration of parameters that increase the range of host cells in which the nucleic acid coding sequence can be expressed, optionally highly expressed, optionally wherein the such parameters include incorporation of Shine-Dalgamo sequence requirements and/or start codon spacing preferences for the heterologous organism(s).

15. The method of any one of paragraphs 1-14 including step (3), wherein step (3) includes maintaining or recoding the nucleic acid sequence to enrich for poly AT sequence and/or a “AAA” sequence motif immediately upstream of the start codon.

16. The method of any one of paragraphs 1-15 including step (3), wherein step (3) includes maintaining, recoding, or adding to the nucleic acid sequence a synthetic 5′ untranslated region including N₁₇(A/U)₆AGGAGN₄AAA (SEQ ID NO:1), and optionally iteratively mutating/varying ‘N’ positions until a desired translation initiation strength is reached, optionally wherein the translation initiation strength is reached by prediction or empirically determined.

17. The method of any one of paragraphs 1-16 including step (4), wherein step (4) includes recoding one or more alternative NTG start codon (s), one or more internal RBS (s), one or more terminator(s), or a combination thereof.

18. The method of paragraph 17, wherein internal RBSs are NTG sites throughout the CDS in all three coding frames.

19. The method of any one of paragraphs 1-18 including step (4), wherein step (4) includes recoding the sequence upstream of one or more RBS(s) to structurally reduce internal ribosomal entry.

20. The method of any one of paragraphs 1-19 including step (4), wherein step (4) includes predicting ribosome bind strength, calculating thermodynamic parameters, or a combination thereof.

21. The method of any one of paragraphs 1-20 including step (5).

22. The method of any one of paragraphs 1-21 including step (6), optionally wherein step (6) includes identifying and optionally recoding rho-independent transcriptional terminators.

23. The method of any one of paragraphs 1-22 including iteratively repeating steps (4) and (5) in two or more cycles.

24. The method of paragraph 23, wherein translation initiation strength is predicted or determined empirically after each cycle, and wherein the cycles are terminated when a desired translation initiation strength is reached.

25. The method of any one of paragraphs 1-24 including steps (1), (2), and (3).

26. The method of paragraph 25 including step (4).

27. The method of paragraphs 25 or 26 including step (5).

28. The method of any one of paragraphs 25-27 including step (6).

29. The method of any one of paragraphs 1-28, wherein one or more steps are computer implemented.

30. A recoded nucleic acid sequence prepared according to the method of any one of paragraphs 1-29.

31. An inducible polymerase promoter expression circuit including seed elements or a seed promoter operably linked to an RNA polymerase promoter operable linked to the polymerase coding sequence, wherein the seed element drive initial transcription of the RNA polymerase, and subsequent transcription is auto-regulated through a positive and/or negative regulation of the RNA polymerase promoter.

32. The expression circuit of paragraph 31, including one or more of repressor/operator pair, CRISPRi and/or CRISPRa.

33. The expression circuit of paragraphs 31 or 32, wherein the promoter is pT7 and the RNA polymerase is T7/RNAP, the promoter is pT3 and the RNA polymerase T3/RNAP, or the promoter is pSP6 and the RNA polymerase SP6 RNA polymerase.

34. The expression circuit of any one of paragraphs 31-33, including tetO tet-on tetracycline-controlled transcriptional activator sequence, an anhydrotetracyline (aTc) responsive TetR repressor, Tet-off tetracycline-controlled transcriptional repressor, riboswitch (e.g., a theophylline-responsive translational riboswitch), or a combination thereof,
or vanO van-on Vanillin acid-controlled transcriptional activator sequence, an vanillin acid responsive VanR repressor, Van-off tetracycline-controlled transcriptional repressor, riboswitch (e.g., a theophylline-responsive translational riboswitch), or a combination thereof.

35. The expression circuit according to any one of paragraphs 31-34 including the architecture of FIG. 4A or any of a, b, c, d, or e of FIG. 4B.

36. The expression circuit of paragraph 35 including a tetO tet-on tetracycline-controlled transcriptional activator sequence, a pT7 promoter driving expression of T7 RNAP through an intervening theophylline-responsive riboswitch, and a pT7 promoter driving expression of a tetR tetracycline repressor.

37. A synthetic genetic element including a coding sequence (CDS) operably linked to a hybrid regulatory element suitable for expressing the coding sequence in organisms from two or more different kingdoms.

38. The synthetic genetic element of paragraph 37, wherein one of the kingdoms is Monera.

39. The synthetic genetic element of paragraphs 37 and 38, wherein one of the kingdoms is Animalia, Plantae, Fungi, or Protista.

40. The synthetic genetic element of any one of paragraphs 37-39, wherein the hybrid regulatory element is suitable for expressing the CDS in prokaryotes and eukaryotes.

41. The synthetic genetic element of any one of paragraphs 37-40, wherein the hybrid regulatory element includes one or more of a promoter, a 5′ UTR, and 3′ terminator.

42. The synthetic genetic element of any one of paragraphs 37-41, including one or more upstream activity sequences (UASs), a core sequence, a TATA box, one or more spacer sequence, or a combination thereof.

43. The synthetic genetic element of paragraph 42 wherein, the hybrid regulatory element includes 1-10 UASs operably linked to the promoter.

44. The synthetic genetic element of any one of paragraphs 37-43, wherein the hybrid regulatory element(s) includes one or more spacer sequence, optionally including poly-A or poly-T in an effective amount to deplete the probability of nucleosome occupancy at a TATA box (e.g., TATAAAG) and/or a transcriptional start site (TSS).

45. The synthetic genetic element of any one of paragraphs 37-44, including a TATA box.

46. The synthetic genetic element of any one of paragraphs 41-44 wherein the promoter is a natural or synthetic eukaryotic promoter, optionally a natural or synthetic yeast promoter, or a variant thereof.

47. The synthetic genetic element of any one of paragraphs 37-46, wherein the hybrid regulatory element includes a transcription start site (TSS), optionally including the consensus motif [A(A_{rich})₅ NPy A (A/T)NN(A_{rich})₆].

48. The synthetic genetic element of any one of paragraphs 37-47, wherein the hybrid regulatory element includes any one of SEQ ID NOS:50-98, or variant thereof with at least 70% sequence identity thereto.

49. The synthetic genetic element of any one of paragraphs 37-48, optionally further including one or more intervening terminators, optionally flanking the promotor sequence.

50. The synthetic genetic element of any one of paragraphs 37-49, including two or more CDS, wherein each CDS is operatively linked its own hybrid regulatory element, wherein the hybrid regulatory element of each CDS are the same, different, or a combination thereof.

51. The synthetic genetic element of paragraph 50, wherein the two or more CDS together form part or all of a biosynthetic pathway.

52. The synthetic genetic element of paragraph 51, wherein the biosynthetic pathway is present as a gene cluster in an organism's genome.

53. The synthetic genetic element of any one of paragraphs 39-52, wherein
(i) no pair of UASs is used more than 5, 4, 3, 2, or, 1 time, optionally no more than 3 times, and optionally no triplet of UASs is used more than once;
(ii) promoters range from 100 bp to 250 bp inclusive, or any subrange thereof, or specific integer therefore, optionally 161 bp to 181 bp, in length; and/or
(iii) no spacer or TSS sequence is used more than once.

54. The synthetic genetic element of any one of paragraphs 37-53, wherein
(iv) no ‘NTG’ sequence is used in any spacer to avoid internal start codons; and/or
(v) predicted terminators and RBSs in promoters are removed by randomly inserting or substituting mutating spacer sequences.

55. The synthetic genetic element of any one of paragraphs 37-54, wherein one of more of CDS and optionally the hybrid regulatory sequence operably linked thereto are prepared according to the method of any one of paragraphs 1-30.

56. The synthetic genetic element of any one of paragraphs 37-55 including the recoded CDS of paragraph 30.

57. The synthetic genetic element of any one of paragraphs 37-56 including a prokaryotic RBS, a bacterial promoter, a eukaryotic promoter for each CDS, and a eukaryotic terminator.

58. The synthetic genetic element of any one of paragraphs 37-57 further including an inducible polymerase promoter expression circuit.

59. The synthetic genetic element of any one of paragraphs 37-58 further including an inducible polymerase promoter expression circuit of any one of paragraphs 31-36.

60. The synthetic genetic element of any one of paragraphs 37-59 including the architecture of one or more of FIG. 3A, 3B, or 3C.

61. A landing pad for a synthetic genetic element including a nucleic acid cassette including a nucleic acid sequence encoding an inducible expression control circuit, a promoter operably linked to a reporter gene, a selectable marker, and integration sites flanking the reporter gene.

62. The landing pad of paragraph 61, further including transposase terminal repeats flanking the cassette, followed by a sequence encoding the transposase, preferably which itself does not mobilize into the recipient genome.

63. The landing pad of paragraph 62, wherein the transposase is independent of host-specific factors and shows little bias in random integration, optionally wherein the transposase is Himar or Tn5.

64. The landing pad of paragraphs 61 and 62, wherein sequence encoding the selectable marker is operably linked to a seed promoter.

65. The landing pad of any one of paragraphs 61-64, wherein the selectable marker is antibiotic selectable.

66. The landing pad of any one of paragraphs 61-65 wherein the inducible expression control circuit is of any one of paragraphs 31-36.

67. The landing pad of any one of paragraphs 61-66 including the architecture of FIG. 5A.

68. A method of introducing a landing pad into a host organism including introducing into the host cell with the landing pad of any one of paragraphs 61-67.

69. The method of paragraph 68, wherein introduction includes transformation or transfection of a vector encoding the landing pad into a first host organism.

70. The method of paragraphs 68 and 69 including expressing the transposase.

71. The method of any one of paragraphs 68-70, further including introduction of the landing pad into a second host organism by conjugation with the first host organism.

72. The method of any one of paragraphs 68-71 including step 1 of FIG. 5A.

73. A host cell including the landing pad of any one of paragraphs 61-67 integrated into its genome.

74. The host cell of paragraph 73 prepared according to the method of any one of paragraphs 67-72.

75. The synthetic genetic element of any one of paragraphs 37-56 flanked by integration sequences.

76. The synthetic genetic element of paragraphs 75 wherein the integration sequences are asymmetrical attB sites.

77. The synthetic genetic element of paragraphs 75 or 76 including the architecture of cassette of FIG. 5B.

78. A vector, optionally a suicide vector, including encoding or including the synthetic genetic element of any one of paragraphs 75-77.

79. The vector of paragraph 78 further including a sequence encoding an integrase optionally phiC31 integrase.

80. The vector of paragraphs 78 and 79 including a sequence encoding a selectable marker.

81. A host cell including the vector of any one of paragraphs 78-80.

82. A method of introducing a synthetic genetic element into a host cell including conjugation of host cell of paragraph 81 with the host cell of paragraphs 73 or 74.

83. The method of paragraph 82, wherein the integrase is expressed is facilitates integration of the synthetic genetic element into the landing pad.

84. The method of paragraph 83, wherein the synthetic genetic element replaces the landing pad's selectable marker.

85. A host cell prepared according to the method of any one of paragraphs 82-84.

86. A host cell including the synthetic genetic element of any one of paragraphs 37-60.

87. Any one of sequences disclosed herein including, but not limited to, SEQ ID NOS: 1-136, or a variant thereof with at least 70% sequence identity thereto.

88. A hybrid yeast promoter including the sequence of any one of SEQ ID NOS:50-98, or a variant thereof with at least 70% sequence identity thereto.

89. A transcriptional start site including the sequence of any one of SEQ ID NOS:2-49.

90. A composition or method as disclosed herein in the text and/or the figures.

91. A use or application using the any of compositions or methods of any of paragraphs 1-90.

### EXAMPLES

It is understood that the disclosed method and compositions are not limited to the particular methodology, protocols, and reagents described as these can vary. It is also to be understood that the terminology used herein is for the purpose of describing particular embodiments only and is not intended to limit the scope of the present invention which will be limited only by the appended claims.

### Example 1: Generation and Characterization of Synthetic Genetic Elements (SGEs) for Cross-Kingdom Expression

### Materials and Methods

### Media

Cultures of E. coli and B. subtilis were maintained in Luria Broth (10 g/L Tryptone, 5 g/L NaCl, 5 g/L Yeast Extract) at 37° C. Cultures of K. aerogenes, P. putida, P. veronii, and S. enterica were maintained in Luria Broth at 30° C. S. cerevisiae cultures were maintained in YPD medium (10 g/L Yeast Extract, 20 g/L Peptone, 20 g/L Dextrose) at 30° C. When antibiotic selection was required, Kanamycin was used at 10 μg/mL in B. subtilis and 35 μg/mL in other strains, Chloramphenicol was used at 5 μg/mL in B. subtilis and 12.5 μg/mL in other strains, Apramycin was used at 10 ug/mL in B. subtilis and 50 μg/mL in other strains, Hygromycin B was used at 200 μg/mL in S. cerevisiae, G418 was used at 200 μg/mL in S. cerevisiae, and Spectinomycin was used at 95 μg/mL in E. coli. For inductions, theophylline stock solution was prepared at 50 mM in water, anhydrotetracycline (aTc) was prepared as 100 μg/mL in 100% Ethanol.

Single Gene Knockouts from Biosynthetic Pathways

Single gene knockouts within the BGC08 pathway were generated using E. coli EcNR1, which contains lambda red recombineering machinery integrated at the bioAB locus (Wang et al., 2009). To support the plasmid backbone, the R6K pir gene was inserted at a noncoding chromosomal locus (coordinate: 1,415,470) via recombineering. To avoid adding additional antibiotic resistance burden, the outer membrane protein, tolC dual selectable marker was used to perform all manipulations. As per previous studies, this tolC marker was selected for with 0.005% SDS, and against with Colicin E1 (DeVito, 2008). The native to/C locus was deleted and reintroduced to replace the open reading frame of individual genes in BGC08. Generally, for gene insertions, cassettes were amplified by PCR (Kapa HiFi Polymerase) using primers that appended 50 bp homology arms to the target. Cells were grown in Luria Broth at 34° C. until they reached an optical density (OD) of 0.6, then heat shocked in a 42° C. shaking water bath for 15 minutes. Cells were immediately placed on ice and 1 mL aliquots were washed 2 times with ice-cold double de-ionized water (ddH₂O) and resuspended in 50 μL ddH₂O+100 ng DNA template, before transferring to a 1 mm electrocuvette. Cells were pulsed at 1800V, 25 uF, 200Q (Bio-rad GenePulser) and recovered in 3 mL Luria Broth for 3 hours before plating on selective media. For deleting tolC, a similar procedure was used, but with the template being a 5′-phosphorothioated 90mer oligonucleotide containing 45 bp homology arms to the deletion loci.

### Plasmid Construction

All plasmids were constructed via Gibson Assembly (NEB). Native biosynthetic pathways for violacein and BGC08/tyrocytabine were PCR amplified from the gDNA of C. violeceum ATCC 12472 and L. iners LEAF2052A-d, respectively. Redesigned biosynthetic pathways were sourced as overlapping synthetic DNA fragments <3.2 kb in size. Both were cloned into the pPath integrating shuttle vector (linearized with the restriction enzyme sfiI) via Gibson Assembly and transformed into E. coli TransforMax™ EC100D™ pir+cells (Lucigen) for maintenance. Selection was performed with 5% sucrose to select against the parental plasmid.

### Transformation Conditions

For E. coli, electroporation was used to transform plasmid constructs. Briefly, 1 mL mid-log cell culture was washed 2 times in 10% ice-cold glycerol, concentrated to 50 μL, and loaded into a 1 mm electrocuvette and pulsed at 1800V, 25 uF, 2000 (Bio-rad GenePulser). For B. subtilis, natural transformation was used. Briefly, a single colony was picked into 1 mL Transformation Media (900 uL ddH2O, 100 uL 10×MMC, 3 mM MgSO4). The culture was grown at 37 C for 4 hours. To each 200 μL aliquot of culture, 100 ng DNA was added and grown further for 2 hours before plating on selective LB media. 10×MMC stock solution consisted of (10.7 g K2HPO4, 5.2 g KH2PO4, 20 g Glucose, 0.88 g Sodium Citrate, 2.2 g Potassium Glutamate, 1 ml 100× Ferric Ammonium Citrate (2.2% stock), and 1 g Casein Hydrolysate raised to 100 mL final volume with ddH2O). For S. cerevisiae, the Frozen-EZ Yeast Transformation II Kit (Zymo) was used. For other bacterial strains used in this study, Landing Pads and Biosynthetic Pathways were introduced via conjugation.

### Conjugation

The donor strain used for conjugation was E. coli BW19851 (Yale Coli Stock Center), and contains the incP RP4 conjugative machinery and chromosomally-integrated R6K pir replication gene. Lambda red recombineering via the pORTMAGE protocol (Nyerges et al., 2016) was used to knock out the Aspartate-semialdehyde dehydrogenase (asd) gene with apramycin resistance, producing a Diaminopimelic acid (DAP) auxotroph for post-conjugation counterselection. This strain was also transformed in the pInh to minimize the expression of Transposase and Integrase activity. Interspecies conjugations were performed by mixing 1 mL late log donor and recipient strains, washing away selective antibiotics with PBS, concentrating the mixture 10 fold, and spotting onto solid Luria Broth +30 μg/mL DAP, overlayed with a 0.45 μM nitrocellulose filter (Millipore). Conjugations proceeded for 6 hours, after which the filter paper was removed, bacteria were resuspended in Luria Broth media, and plated on selective DAP-free media.

### Prediction of Prokaryotic Transcriptional Terminators

The computational program TransTermHP (Kingsford et al., 2007) was used to predict rho-independent transcriptional terminators on both strands. Default parameters for stemloop and tail scoring were used. The Confidence threshold for calling a terminator was left as >76.

### Calculation of Prokaryotic Ribosome Binding Site Thermodynamic Predictions

For ribosome binding site (RBS) strength predictions, thermodynamic parameters were calculated in accordance with previous studies (Salis et al., 2009). This calculation is summarized as:

$$Δ {G}_{tot} = Δ {G}_{mRNA : rRNA} + Δ {G}_{start} + Δ {G}_{spacing} - Δ {G}_{standby} - Δ {G}_{mRNA}$$

where β=0.45, and A=2500
ΔGₜₒₜ is the difference in Gibbs free energy between the initial state (folded mRNA transcript and the free 30S complex) and the final state (the assembled 30S pre-initiation complex bound on an mRNA transcript;
ΔG_{(mRNA:rRNA)} is the energy released when the last 9 nucleotides (nt) of the E. coli 16S rRNA ((3′-AUUCCUCCA-5′) hybridizes and co-folds to the mRNA sub-sequence;
ΔGₛₜₐᵣₜ is the energy released when the start codon hybridizes to the initiating tRNA anticodon loop (3′-UAC-5′);
ΔG_{starting} is the free energy penalty caused by a non-optimal physical distance between the 16S rRNA binding site and the start codon;
ΔG_{standby} is the work required to unfold any secondary structures sequestering the standby site after the 30S complex assembly; and
ΔG_{mRNA} is the work required to unfold the mRNA sub-sequence when it folds to its most stable secondary structure, called the minimum free energy structure.

The Vienna RNA Suite was used to collect the Gibbs Free Energy values in accordance with previous studies (Lorenz et al., 2011). The following assumptions were made: (1) the relevant mRNA considered was +/−35 bp flanking the start codon, (2) the Ribosome unfolded the first 15 bp of the open reading frame, (3) the standby site was 4 bp upstream of the rRNA binding site, and (4) the relevant anti-Shine Dalgarno rRNA sequence considered was the terminal 9 bp of 16S rRNA (For E. coli, this sequence is “ACCUCCUUA”). The ΔGₛₜₐᵣₜ values used were: “AUG”:-1.194, “GUG”:-0.0748, “UUG”:-0.0435, “CUG”:-0.03406. To account for multiple mRNA:rRNA folding configuration possibilities, the RNAduplex program was used to duplex the rRNA to the region of the mRNA 3-13 bp upstream of the start codon. All possible duplexes+/−1.5 kcal/mol of the Minimum Free Energy (MFE) were considered. The ΔGₜₒₜ was calculated for each possible duplex. The duplex that minimized ΔGₜₒₜ was considered the equilibrium translation initiation configuration.

### Construction of Yeast Promoter Library

Yeast promoters were constructed from individual modular components. “Core” and “UAS” sequences were sourced from previous literature (Redden and Alper, 2015). Spacer sequences were constructed by creating random 30mers (that lacked NTG sequences to prevent internal start codons) and surveying for a lack of transcription factor binding sites derived from the YeastTract database (Monteiro et al., 2020). Transcription factor binding sites were pulled from native S. cerevisiae transcripts; binning was done for sites that had been empirically validated with 5; SAGE experiments and contained the canonical yeast transcription start site motif (Zhang and Dietrich, 2005). Yeast promoters were combinatorically assembled, ensuring that no permutation of three UASs was repeated in the library to minimize sequence similarity. Each promoter was scanned with the RBS predictor to highlight potential start sites, which were iteratively removed by altering spacer sequences. To deplete nucleosome occupancy, NuPoP was used to predict nucleosome occupancy. Each promoter was specifically assayed for the probability of nucleosome occupancy at the TATA box and Transcription Start Site. 5mer poly A or poly T sequences were added to spacers until nucleosome occupancy fell below 20% probability at both sites. Promoters were additionally scanned for rho-independent transcription termination using TransTermHP.

### Flow Cytometry Analysis

Fluorescent measurements (FIGS. 6E, 6F, 5B, 9) were performed with the BD FACS Aria. For experiments where higher throughput was needed (FIGS. 5A-5I), the Stratedigm 8 with a 96 well plate loader was used. For these analyses, cell cultures were grown, induced when necessary at OD 0.6, and cultured for a further 12 hours. Cells were diluted 1:10 in PBS and loader onto the instrument. Quantification was done with FlowJo v10. Briefly, the cell population was gated with the FSC and SSC channels, before quantifying fluorescent intensity in the FITC channel. Across all data sets, to assign a uniform Fluorescent Intensity value (as arbitrary units), the raw fluorescent mean value was normalized, via linear scalar, such that the intensity of the cyc1 promoter sample in each data set was “100”.

### Plate Reader Analysis

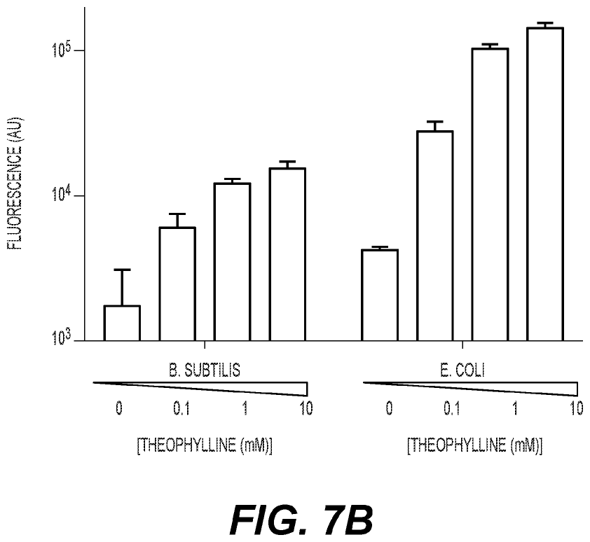

Fluorescent measurements for evaluating the performance of the T7 RNAP circuit (FIGS. 7B, 5C) were performed on a BioTek Synergy H1 plate reader. Here, cell cultures were diluted 1:50 into fresh LB+antibiotics. At OD 0.6, cultures were distributed across a 96 well plate (150 μL per well). As needed, inducers were added and the plate was cultured in a shaking incubator for an additional 12 hours. Cells were pelleted and resuspended in PBS and transferred to a black well clear bottom 96 well plate. OD₆₀₀ and GFP Fluorescence (488 nm ex, 525 nm em) were measured. To quantify cell density—normalized fluorescence, GFP values were divided by OD₆₀₀ values. These values were further background subtracted using the average GFP/OD₆₀₀ values of wildtype cells.

### Quantification of Violacein Production

Violacein pigment was quantified as “Violacein Units” (Blosser and Gray, 2000). Pigment producing cells were cultured in LB at 30° C. until mid-log optical density. Upon adding relevant inducers, culture was continued at 20° C. for 48 hours. 200 μL of the final culture was diluted in 800 uL PBS to measure OD₆₆₀ nm to quantify cell density. Another 200 μL of the culture was mixed with 200 μL 10% SDS for 5 minutes with vortexing. 900 μL Butanol was added and vortexed for 5 seconds to extract pigment. Samples were pelleted in 1.5 mL tubes at 13000 rpm for 5 minutes to pellet debris. The top organic layer was collected and Absorbance585 nm was measured to quantify violacein content. Violacein units are calculated as:

$$Violacein Units = {A}_{585 nm} / {OD}_{660 nm} \times 1000$$

### Analytical Chemistry Instrumentation Parameters

Ultraviolet/visible (UV/Vis) spectra were recorded on an Agilent 1260 Infinity system equipped with a photo diode array (PDA) detector (Agilent Technologies, CA, USA). The full nuclear magnetic resonance (NMR) spectroscopy data sets were recorded at 25° C. on an Agilent 600 MHz NMR spectrometer (DD2) equipped with an inverse cold probe (3 mm), employing standard NMR pulse libraries, including 1D ³¹P (202 MHz) and ¹H-³¹P decoupling experiments. Flash column chromatography was performed on LiChroprep RP₁₈ (40-63 mm, Merck, NJ, USA). High pressure liquid chromatography-mass spectrometry (HPLC-MS) analysis was conducted on an Agilent 1260 Infinity system using a Phenomenex Luna Cis(2) (100 Å) 5 μm (4.6×150 mm) (Phenomenex, CA, USA) column or a Hypercarb column (ThermoFisher Scientific Scientific, Waltham, MA, USA, 5 μm, 4.6×100 mm) using a PDA detector coupled with a single quadrupole electrospray ionization mass spectrometry instrument (ESI-MS, Agilent 6120). Purification of metabolites addressed in the study was performed using an Agilent Prepstar HPLC system using an Agilent Polaris Cis-A 5 μm (21×250 mm) column, a Phenomenex Luna Cis(2) (100 Å) 10 μm (10×250 mm) column, or a Hypercarb column (ThermoFisher Scientific; 5 m, 10.0×250 mm) column. High-resolution ESI-MS (HR-ESI-MS) data were recorded on an Agilent iFunnel 6550 quadrupole time-of-flight (QTOF) MS instrument fitted with an electrospray ionization (ESI) source linked to an Agilent 1290 Infinity HPLC system with the columns. XAD-7 HP resins for metabolite extraction were obtained from ThermoFisher Scientific.

### Metabolomics-Based Discovery of Pathway-Dependent Metabolites

Metabolomics was performed to investigate gene and pathway-dependent metabolites to promote discovery and characterization. For this characterization, redesigned BGC08 was transformed into E. coli BL21 DE3 (this strain was transformed with a plasmid-bound copy of the R6K pir gene to maintain the pPath vector carrying the pathway). A 5 mL Luria Bertani (LB) liquid culture with 50 μg/mL of spectinomycin and 50 μg/mL carbenicillin was prepared as starter cultures by inoculation of single colonies containing either the full pathway, single gene knockouts, or its empty vector, pPath. Upon overnight growth under aerobic conditions (37° C. and 250 rpm), each seed culture (50 μL) was used to inoculate 5×5 mL fresh M9 cultures (M9 medium supplemented with 5% casamino acids, 0.2% D-glucose, 1 mM MgSO4, 0.1 mM CaCl₂)) and incubated (37° C. and 250 rpm) until the OD₆₀₀ reached 0.8 absorbance units. Cultures were induced with IPTG induction (0.1 mM) on ice and then grown for an additional 48 hours (20° C. and 250 rpm). An M9 medium control was also treated under identical conditions. Cultures were then centrifuged at 14,000×g (r.t.) for 30 minutes, XAD-7 HP resins (20 μg/L) were added to each clarified supernatant, and the resin-supernatant mixtures were incubated for 2 hours at 37° C. and 250 rpm. The filtered resins were then extracted with MeOH (10 mL each), and the extracts were filtered and evaporated under reduced pressure to generate representative crude materials for medium controls, empty vector controls, and full pathway samples. These samples were subjected to QTOF-MS analysis followed by comparative metabolomics using Mass Profiler Professional (Agilent Technologies) and methods previously described (Vizcaino et al., 2014). The metabolomics analysis revealed pathway-dependent molecular features, and a large-scale cultivation was implemented to gamer a feasible amount of those metabolites by high-resolution mass-directed isolation for further studies (i.e., NMR-based structural elucidation, absolute configuration analysis, and bioactivity investigation). A starter culture of the full pathway prepared as described above was used to inoculate 1×24 L of the supplemented M9 medium, and cultivation was proceeded with identical conditions as used for the metabolomics studies. The culture was centrifuged at 14,000×g (r.t.) for 30 minutes, and the clarified supernatants were incubated with XAD-7 HP resins for 2 hours (37° C. and 180 rpm). The pooled filtered resins were extracted with MeOH (24 L in total), and the methanolic extract was filtered and evaporated under reduced pressure with a stream of nitrogen gas to produce the crude material. The crude extract (˜200 g) was subjected to a gravity column packed with LiChroprep RP₁₈ (500 g; 5×20 cm) with a step-gradient elution (0→100% MeOH in water, 10% MeOH increment, 500 mL each) to generate 11 fractions (Fraction 1-Fraction 11). Among these fractions, Fractions6 and 7 were found to contain target entities based upon single quad LC-MS analysis. These two fractions were combined (Fraction 6-7) and further purified employing prep RP HPLC equipped with an Agilent Polaris C₁₈-A column (5→50% MeCN in water with 0.01% TFA for 60 minutes, 8 mL/min, 1 minute collection interval). The LC-MS traces of these HPLC fractions showed that Fractions 6-7 and 15-25 possessed the targeted metabolites based on their masses and retention times. Repetitive semi-prep HPLC experiments (Phenomenex Luna Cis(2); 5→10% MeCN in water with 0.01% TFA) led to the individual purification of the targeted entities. Feeding studies to corroborate the biosynthetic pathway from 2 to 3 were performed with the addition of 1 mM of 2 to the pathway with tybC knocked out. 100 mM IPTG was used for induction. A similar protocol was employed to identify a fatty acid to comprise m/z 753; 1 mM of octanoate was added to the full pathway cultivation together with IPTG induction.

### Quantification of Tyrocitabine Production Across Strains

For gram-negative bacteria, pathway expressing strains were cultured in 5 mL M9 minimal media supplemented with 0.4% Glucose+0.2% casamino acids at 30° C. Upon reaching OD 0.6, inducers were added, and cultures grown further for 48 hours at 20° C. For gram-positive bacteria, pathway expressing strains were cultured in 5 mL LB at 30° C. Upon reaching OD 0.6, inducers were added, and cultures grown further for 48 hours at 30° C. For yeast, cultures were grown in 5 mL Complete Synthetic Media (CSM) with 2% glucose for 48 hours at 30° C. Complete cultures were then dried via vacuum using a GeneVac system at full vacuum and no added heat. Once dried, metabolites were extracted with 500 μL Methanol. Brief heating at 60° C. and sonication were applied until the extraction produced a homogenous slurry. The slurry was centrifuged at 15000 g for 10 minutes to pellet debris and clarified supernatant was loaded for LC/MS (Agilent QTOF 6550) analysis (1 μL injection volume). Resulting data was analyzed with Agilent Quantitative Analysis software—EIC integrations were performed with 20 ppm error, using exact m/z masses calculated by ChemDraw Pro.

### In Vitro Transcription/Translation Reaction

The PureExpress kit (NEB) was used in accordance with manufacture's protocol to assay for the in-vitro production of GFP. For each sample a 25 uL reaction was performed containing 100 ng DNA or 500 ng RNA template encoding GFP (transcription in this kit is via T7 RNA Polymerase), plus indicated amounts of purified compound dissolved in H2O. Reactions were loaded into a white 384 well plate and production of fluorescent GFP protein was monitored with a Synergy Ht Plate Reader (Bio-tek). Fluorescence reached an endpoint at 4 hours. To produce DNA template, a PCR product of the pT7-GFP gene was amplified (Kapa HiFi Polymerase) and purified by gel electrophoresis followed by gel purification (Qiagen). To produce RNA template, the DNA PCR product was transcribed with the HiScribe T7 High Yield RNA Synthesis kit (NEB), treated with DNasel, and purified by the Monarch RNA Purification Kit (NEB). RNA was quantified by Qubit.

### Isolation of Total RNA for RT-qPCR Experiments

To collect RNA from S. cerevisiae, 3 mL cultures were grown overnight at 30° C. in YPD media+hygromycin for selection. Cultures were back diluted 1:50 into fresh media and grown until OD 1.0. 1 mL of this culture was processed using the RNeasy Plus Kit (Qiagen), using the manufacturer's zymolase protocol for lysis. To collect RNA from E. coli, 3 mL cultures were grown overnight at 37° C. in LB+50 μg/mL carbenicillin for selection. Cultures were back diluted into fresh media and grown until OD 0.6. 0.5 mL of this culture was processed using the RNeasy Plus Kit (Qiagen) using the manufacturer's lysozyme protocol for lysis. In all cases, in-column DNase treatment and the gDNA removal column were used to eliminate gDNA. Total RNA was quantified by nanodrop. Approximately 100 ng RNA was used in each 20 μL qPCR reaction using the Luna one-step universal RT-qPCR kit (NEB) run on a CFX Connect RT system (Bio-Rad). The cycling conditions were: (1) 55° C. for 10 minutes (2) 95° C. for 1 minute (3) 95° C. for 10 seconds (4) 60° C. for 30 seconds (5) Measure SYBR (6) Go to step 3, 40× (7) Melt Curve analysis 60° C. to 95° C.

### Prediction of Tyb Pathway Homologs

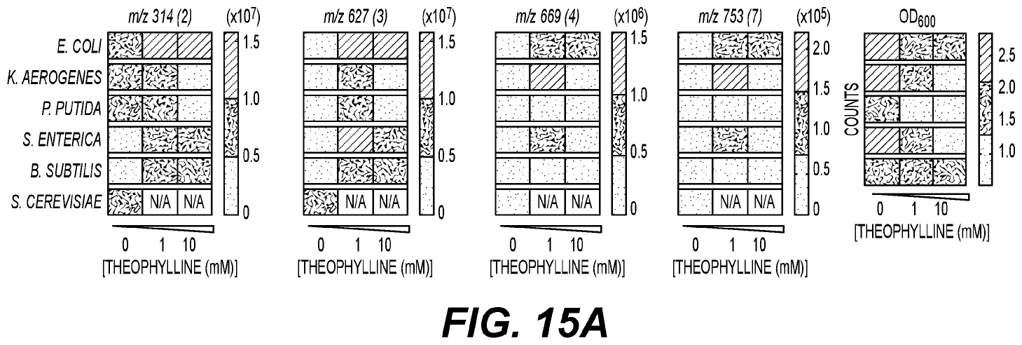

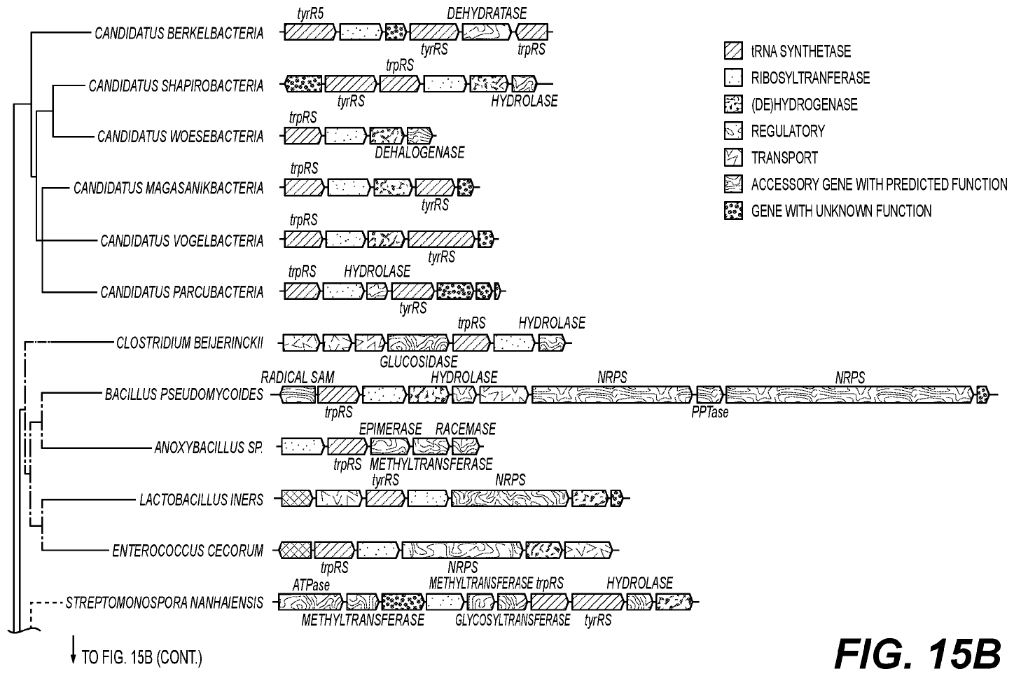

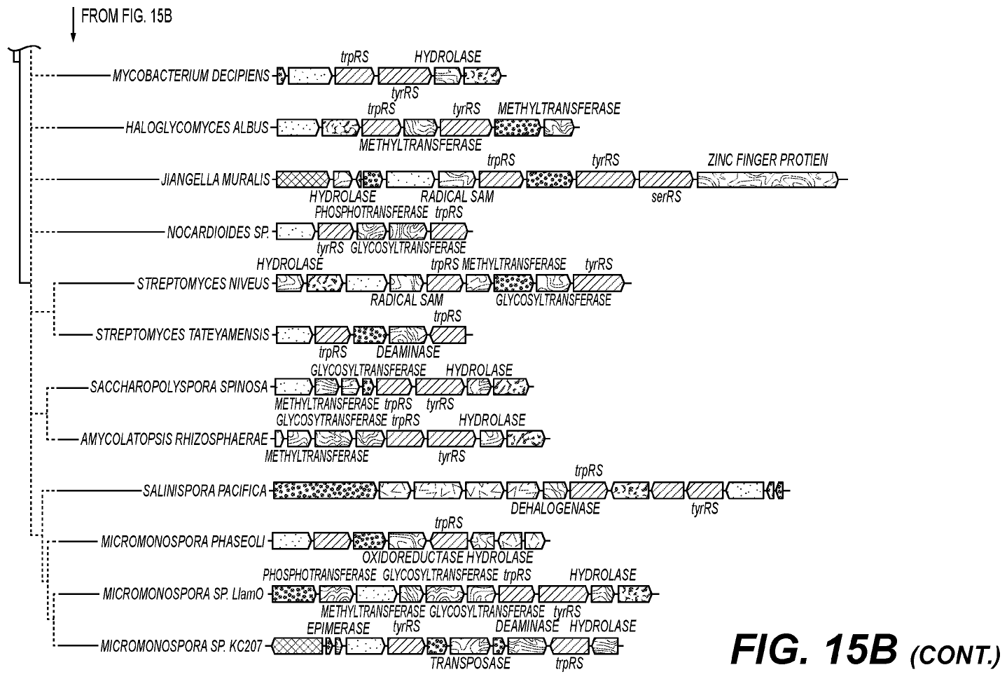

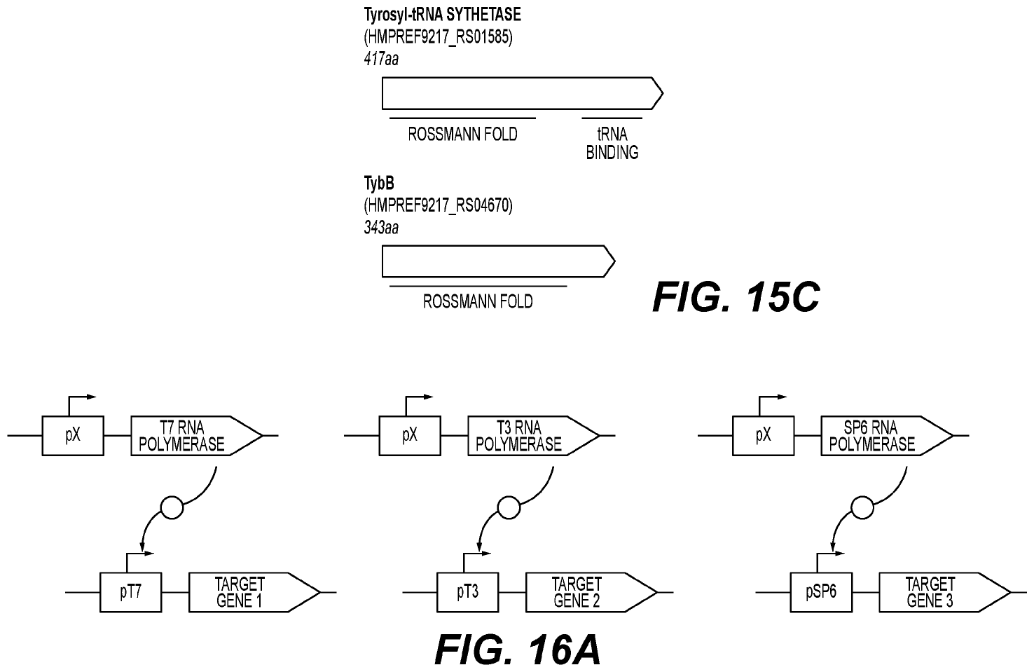

The amino acid sequence for the tybB tRNA synthetase gene was used to blast “All genomes” on the DOE Integrated Microbial Genomes & Microbes (IMG) database with an E-value cutoff of 1^{c-5}. Of the resulting 113 homologs found, each was manually curated to verify that the synthetase lacked a RNA binding domain, as predicted by the InterPro server, and that it was co-localized in an operon containing at least one additional biosynthetic enzyme, resulting in 92 hits. 24 general operon architectures were observed, which are shown in (FIG. 15B). The Phylogenetic tree was constructed with phyloT, populated with NCBI taxonomy data, and visualized with iTOL. Operon schematics are constructed with the python package DNAplotlib.

### Quantification and Statistical Analysis

### Statistical Notes

For all statistical analysis and curve fitting, the software Graphpad Prism was used. For determining r² correlation values (FIGS. 5D, 5F, 5G), ordinary least squares (OLS) linear regression was performed. For determining statistical significance, a 2-tailed t-test was performed. For statistical significance, the cutoff is * p<0.05. For all flow cytometry and plate reader data, individual conditions were collected as biological replicates, and for each replicate at least 50000 cells were quantified. Where error bars are shown, mean and standard deviation are plotted. Similarly, for all LC/MS quantification of metabolite production, and quantification of violacein production, 5 mL cultures were grown in biological triplicate and extracted, with significance quantified with a 2 tailed t-test.

For quantifying significance in the difference between the distributions in (FIG. 2J), a 2-tailed paired Z-test was performed using mean, variance, and sample size of the frequency distributions calculated by GraphPad Prism. The significance level tested against the null hypothesis was p<0.001.

Measurement of Relative Gene Expression with RT-qPCR

For the calculation of mRNA gene expression of the reporter mUkGFP used to evaluate the Yeast Promoters (FIG. 5D), the λACq method was used (Livak and Schmittgen, 2001). For these experiments, an equivalent plasmid backbone which lacked a defined yeast promoter upstream of GFP was used as the control. Values are plotted as a fold change over this no-promoter control. As internal reference genes for normalization, for S. cerevisiae UBC6 was used, and for E. coli cysG was used. Samples were collected as biological triplicates.

### Measurement of IC50 Values

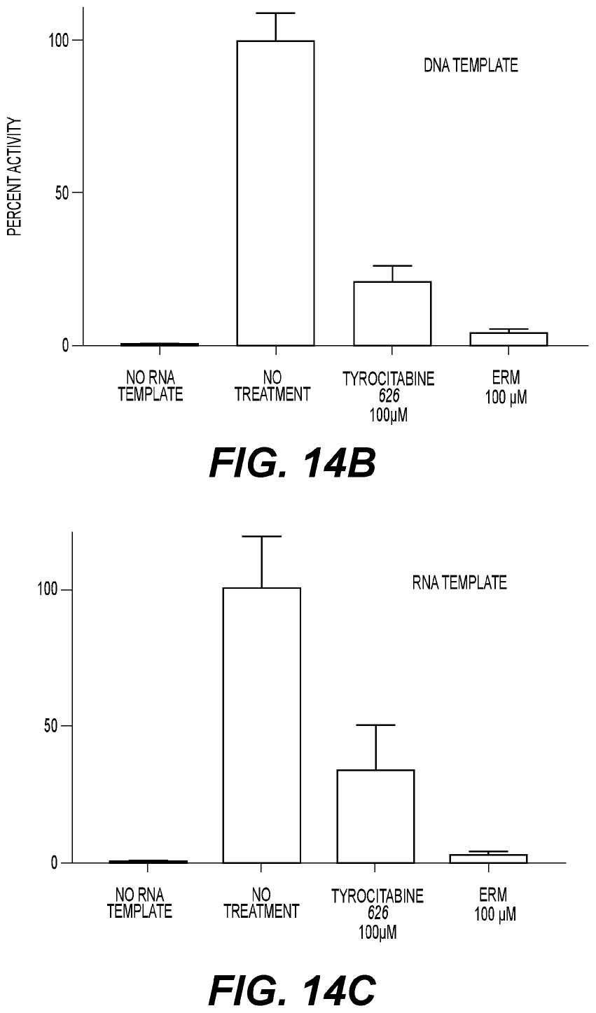

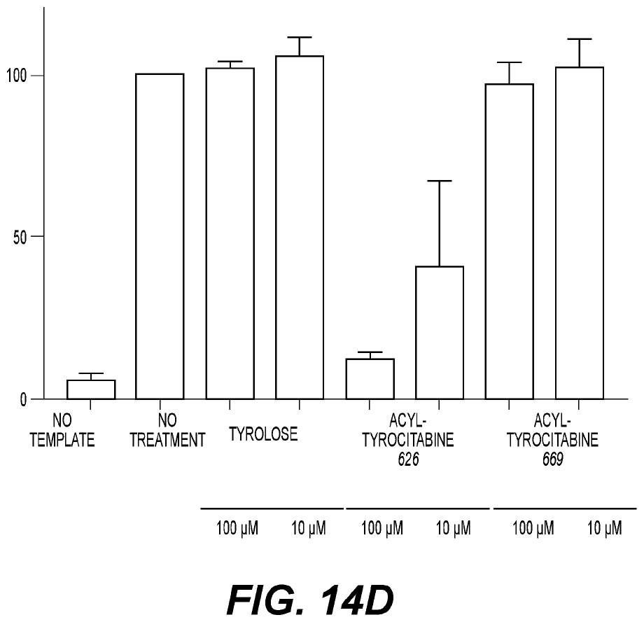

To quantify IC50 values for translation inhibition activity (FIG. 14A), raw fluorescence values were linearly converted to percent activity. The fluorescence of the no template control was used to background subtract the data set and activity was proportionally scaled relative to the no inhibitor control. Graphpad Prism was used to plot a hyperbolic non-linear regression.

### Results

### Computer-Aided Design of Synthetic Genetic Elements (SGEs)

Expression from biosynthetic gene clusters (BGCs) and their associated metabolites involves sequential layers of control exerted at multiple levels: 1) transcription, through mRNA initiation, elongation, and stability; 2) translation, through ribosomal binding and codon usage; and 3) enzymatic activity, often mediated through posttranslational modification and the availability of input metabolites and metabolic flux (Temme et al., 2012). Through evolutionary divergence, regulation of these layers is strain- and environment-specific. Thus, a major challenge in achieving host-range versatility is to decouple biosynthetic capacity from these regulatory layers. To address this challenge, a computer-aided design strategy was developed to redesign BGCs at the level of an individual coding sequence (CDS), transcription, and translation, establishing synthetic design principles to enable cross-kingdom host-range versatility (FIG. 2A).

A computer-aided design strategy was developed to redesign Biosynthetic Gene Clusters (BGCs) at the level of an individual coding sequence (CDS), transcription, and translation, establishing synthetic design principles to enable cross-kingdom host-range versatility (FIG. 2A)

To initiate the stepwise process, the redesign principles were assessed and redesigned at the individual CDS level (FIG. 2B). The constraint with traditional codon optimization approaches is that they are tailored for a target species. Also, the general utility of codon optimization for heterologous expression remains an unresolved subject, where large-scale screens fail to capture a general correlation between codon adaptation and expression levels (Kudla et al., 2009). Specifically, most strategies improve heterologous protein production by synonymously altering a gene's codon usage to match the more frequently used codons i.e., the codon adaptation index (CAI) approach or available tRNA pool of a single heterologous host i.e., the tRNA adaptation index (TAI) approach (Mauro and Chappell, 2014). For the current study, this classical paradigm is problematic to implement since designing constructs for diverse prokaryotic and eukaryotic taxa, each with greatly varying GC content, tRNA abundances, and codon usage patterns are simultaneously designed.

To address these constraints and enable versatile expression of synthetic genetic elements (SGEs), an alternative CDS-level optimization protocol was developed to capture more host-independent optimization parameters, accounting for six main factors (FIG. 2B) described as follows.

(1) The individual CDSs are converted from amino acid to nucleotide sequence; here, the baseline codon usage distribution is based on that of highly expressed genes of a species of choice (FIG. 2C). The base selection for the experiments in this study was Escherichia coli (E. coli), although the strategy allows for variable base selection. The base codon distribution is depleted of canonically-inhibiting codons, including: (1) TTA, which is inefficiently decoded in a variety of Actinobacteria (Leskiw et al., 1991), (2) AGG, CTA, and CGA, which are broadly depleted across highly diverse bacteria (Tian et al., 2017), and (3) CGG and CGA, which promote the formation of “inhibitory pairs” in S. cerevisiae (Ghoneim et al., 2019). The codons TTG and GTG are also depleted to disfavor alternative start codons.

(2) Codon usage specifically encoding the N-terminus has been shown to significantly impact gene expression, largely attributed to 5′-RNA secondary structure among other factors (Angov, 2011). This feature is conserved in prokaryotic and eukaryotic phyla and serves as a useful parameter to promote host-range versatility. Codons that lower structure, thereby enhancing translational initiation at the start codon, promote stronger expression (Goodman et al., 2013). To demonstrate this effect, the predicted 5′-mRNA structure of E. coli genes were analyzed before and after recoding in silico. To avoid the confounding variable of translational coupling, analysis was limited to genes that did not overlap with upstream CDSs. Using the Vienna RNA Suite (Lorenz et al., 2011), the minimum folding energy was calculated across each CDS using a 30 bp sliding window. This data highlights the depletion of secondary structure in native gene sequences, particularly in the 36 bp at the 5′-terminus, consistent with previous studies (FIG. 2E) (Goodman et al., 2013). Similar analyses of other microbial strains used in this study reveal that this depletion of structure is reproducible across phyla (FIG. 2D). For comparison, CDSs were re-coded by the standard CAI approach (Mauro and Chappell, 2014), using the codon distribution of highly expressed E. coli genes, which resulted in dissipation of the 5′-thermodynamic property (FIG. 2E). Annotated datasets from a previous study (Goodman et al., 2013) empirically determined codon usage patterns at the 5′-end of the CDS that promote high levels of gene expression. This data set was re-analyzed using the disclosed alternative CDS-level disclosed optimization protocol to rescue what is seen in native gene sequences. Specifically, for the first 36 nucleotides, the disclosed algorithm used a hybrid codon distribution that biases toward “privileged” N-terminal codons correlating with high expression levels. Genes re-coded with this approach computationally recreated the depletion of 5′ structure seen in native genes (FIG. 2E).

The impact of these genetic design parameters on 108 recoded GFP variants were investigated. It was found that the most significant impact on GFP expression came from codon usage at the 12 N-terminal amino acids (FIGS. 2F-2H). An enrichment of less adapted codons among the first 30-50 codons was reported in prior studies (Tuller et al., 2010). Through subsequent FACS-seq experiments, it was found that secondary structure was the predominant determinant, while codon usage was a tolerable covariate (Goodman et al., 2013). In these experiments, it was found that even when selecting for low (<3.5 kcal/mol) 5′-mRNA secondary structure, codon usage remained impactful (FIG. 2F), a finding distinct from the prior study. At the N-terminus, using codons enriched in E. coli highly expressed genes resulted in higher fluorescence than codons enriched in the FACS-seq assay. Additionally, for the remainder of the open reading frame, no significant correlation between CAI and protein expression was observed. This indicates that aggregate codon adaptation was not strongly predictive of expression strength. Indeed, GFP genes recoded to match E. coli 's codon usage performed comparably in E. coli to those recoded to match B. subtilis codon usage, were neutrally randomized, or were enriched in inhibitory codons to create poor CAI values. This lack of correlation indicates codon usage is not strictly a barrier for broad host-range expression.

(3) Expanding outwards from the CDS, synthetic 5′-UTR sequences were designed to enable versatile regulation across diverse prokaryotes and eukaryotes. With a focus on host range versatility, hybrid eukaryotic and prokaryotic elements that are known to impact gene expression in various microbial taxa were incorporated into the model (FIG. 2I). The implementation of the model stems from a previously described thermodynamic translation initiation model, which defines sequence and structural determinants of bacterial ribosome entry and allows predictions of translation initiation rates using the RBS calculator (Salis et al., 2009). This model was expanded with additional parameters to maximize broad host range applicability. For example, assumptions and parameters incorporated into the model include: (1) Gram-positive bacteria are known to demonstrate a substantially stricter Shine-Dalgarno sequence requirement and start codon spacing preference when compared to Gram-negative bacteria (Vellanoweth and Rabinowitz, 1992); (2) the upstream sequence is enriched in poly AT sequence, which mirrors UTRs in both bacterial phyla and eukaryotes (Cuperus et al., 2017); (3) the “AAA” sequence motif is maintained immediately upstream of the start codon to match the S. cerevisiae consensus Kozak sequence (Hamilton et al., 1987); and (4) sequences are strictly screened to remove alternative NTG start codons. Integrating all these design considerations results in a base UTR defined as N17(A/U)6AGGAGN4AAA (SEQ ID NO:1) (FIG. 2I). The variable ‘N’ positions are then iteratively mutated until desired predicted translation initiation strengths are reached, tailored specifically for each CDS.

(4) Outputs of the initial CDS and 5′-UTR design methodology revealed sequences predicted to signal aberrant transcription termination and translation initiation, which are undesirable for heterologous expression. To evaluate this quantitatively, the above-mentioned E. coli gene test was analyzed using the alternative CDS-level algorithm; each gene was recoded 100 times to derive a representative quantification of the outcome. The results revealed widespread emergence of internal prokaryotic translation start sites, predicted using the RBS thermodynamic parameters from the RBS calculator (Salis et al., 2009). An average of 3.8 internal RBSs appeared per gene recoding attempt (FIG. 2J). In native genes, aberrant internal translation initiation is largely disfavored, even in the presence of Shine Dalgarno motifs upstream of ATG codons, as demonstrated by ribosomal profiling experiments (Li et al., 2012). However, the mechanism and sequence features by which internal initiation is avoided is not understood (Saito et al., 2020).

(5) The data also revealed that deleterious rho-independent terminators spontaneously appear during 19% of the recoding attempts, as identified using the predictive tool transTermHP (Kingsford et al., 2007) (FIG. 2J). The fifth design principle circumvented this issue by algorithmically depleting NTG codons in all three forward coding frames. When an NTG codon cannot be avoided, the upstream sequence is then synonymously modified to structurally inhibit internal ribosome entry. These efforts significantly decrease the number of predicted internal translation initiation sites from 3.8 to 0.6 per gene (p<0.001 using a 2-tailed paired Z-test) (FIG. 2J).

(6) As the sixth design principle, the disclosed algorithm importantly scans and removes the deleterious terminators, bringing the computed value to 0%.

Establishing Eukaryotic Transcription with Synthetic Promoters Optimized for Cross-Kingdom Expression

Another step in the approach to designing multigene SGEs is focused on transcription initiation by designing a hybrid prokaryotic-eukaryotic regulatory element. In prokaryotes, multiple genes can be concurrently transcribed as a polycistronic operon. In eukaryotes, every CDS requires a distinct promoter and terminator. Given this requirement, the 5′ sequence of each CDS was further extended to include regulatory elements to initiate yeast transcription initiation and decrease nucleosome occupancy in eukaryotes. In the context of a multigene operon, this design therefore creates intergenic regions depleted in nucleosome occupancy, which is strongly correlated with both efficient transcription initiation and termination by polyA-capping in eukaryotes (Ichikawa et al., 2016: Morse et al., 2017) (FIG. 2I). For this study, datasets from previously described libraries of synthetic S. cerevisiae terminators (Curran et al., 2015; MacPherson and Saka, 2017; Wang et al., 2019b) were used for initial cross prokaryotic/eukaryotic pathway expression and combinatorial assembly.

To develop 5′ sequences designed to initiate transcription in both prokaryotes and eukaryotes, an expanded library of synthetic yeast promoters were constructed that addressed three key requirements of cross-kingdom SGE design (FIG. 3A-B) (1) these eukaryotic elements, in addition to being efficient in S. cerevisiae, could not interfere with bacterial expression at both the transcriptional and translational levels; (2) sequence size was minimized to reduce synthesis costs, and to minimize the negative impact untranslated sequence has on bacterial mRNA stability as reported by previous studies (Cetnar and Salis, 2021); and (3) for multigene operons, a large library with minimal sequence overlap was required to prevent deletions through homologous recombination. To develop promoters meeting these unique constraints, a previously reported framework (Redden and Alper, 2015) was adapted to achieve robust eukaryotic expression by arraying synthetic 10 bp upstream activity sequences (UASs) (6 distinct sequences), 30 bp core sequences (9 distinct sequences), a consensus TATA box (TATAAAG), and random spacers (FIG. 3C). For this study, 48 transcription start sites (TSSs) matching the known consensus motif [A(A rich)5 NPy A (A/T)NN(A rich)6]from the native S. cerevisiae genome (Zhang and Dietrich, 2005) were also mined. The sequences of these parts can be found as follows:

TABLE 1
Transcription Start Sites

| SEQ ID NO: | Transcription Start Site |
|---|---|
| 2 | AATATCATaTAGAAGTCA |
| 3 | TAGAAGTCaTCGAAATAG |
| 4 | AGATCATCaAGGAAGTAA |
| 5 | ATCAAAACaAATAAAACA |
| 6 | ATAAGAACaACAACAAAT |
| 7 | AAAATATCaTAGCACAAC |
| 8 | AGAAAATCaAGAAGGACA |
| 9 | GAGCAAGCaAGATATTTG |
| 10 | AAGAAATCaAAAGAATAA |
| 11 | AAGAAATCaAACAACTAA |
| 12 | ATTACGTTaCAAGAACAC |
| 13 | TAGCTACTaCCCCTATTA |
| 14 | AGATCGTTaAGGAATAGT |
| 15 | AAAGACTTaTACAAGAAG |
| 16 | GTAAAAATaCAGAACTCT |
| 17 | AAAGAACCaCAGAAAAAT |
| 18 | AAGTATTTaCCGTCTAAA |
| 19 | CTCCTATTaACGGTTTGA |
| 20 | TAGAAAAGaAAGGATAGG |
| 21 | ATATAACCaAACAGACCG |
| 22 | ATATAACCaATTTCAATA |
| 23 | AAAGAATTaAATATAATC |
| 24 | AATGCACCaAACACAAGA |
| 25 | ACAAGATCaACTAAGAAC |
| 26 | GGAAATTCaTACACAACA |
| 27 | TACACAACaACAGAACCA |
| 28 | GTCTCCCCaTTGTGCAGC |
| 29 | GCAGCGATaAGGAACATT |
| 30 | TGCACAATaTTTCAAGCT |
| 31 | AATATTTCaAGCTATACC |
| 32 | TTCAAGCTaTACCAAGCA |
| 33 | AAGCATACaATCAACTAT |
| 34 | TAAGCAACaTTTTATACA |
| 35 | AACATTTTaTACATTTTT |
| 36 | GTAAGAACaTCACACAAA |
| 37 | AGAACATCaCACAAAGAT |
| 38 | AGAAAAACaTCTAACATA |
| 39 | ATACGGTCaACGAACTAT |
| 40 | AAAACACCaAGAACTTAG |
| 41 | AAAAAACCaAGCAACTGC |
| 42 | GGGAGAATaTTCGCAATT |
| 43 | TTTCTTTCaTAACACCAA |
| 44 | ATAACACCaAGCAACTAA |
| 45 | AAGAAAGCaTAGCAATCT |
| 46 | AAATTACTaTACTTCTAT |
| 47 | AGAACTATaACACATAGA |
| 48 | TATGTGTTaAATTTATTG |
| 49 | GCAGAAACaACAACAACA |

Finally, promoters were flanked with a three-frame stop codon (TAANTAANTAA) to terminate any translation initiation from inside the promoter sequence.

To explore the expression levels in S. cerevisiae, two key variables were considered using an initial test promoter sequence. First, a range of 3-5 UASs per promoter was investigated. As observed in previous studies (Ichikawa et al., 2016), depletion of nucleosome occupancy is characteristic of strong eukaryotic promoters (FIG. 4A). Thus, second, the primary sequence of the spacers was interspaced with poly-A or poly-T 5-mers to deplete the probability of nucleosome occupancy at the TATA box (TATAAAG) and TSS to <20% (FIG. 4B). In accordance with previous studies (Xi et al., 2010), the NuPop hidden Markov model was used for predicting nucleosome position. The impact of these variables was measured using a previously described green fluorescent protein optimized for yeast expression (mUKGFP) (Kaishima et al., 2016). Increasing the number of UASs to 3-5 resulted in increased expression levels 2.4-fold (p<0.001) and 21-fold (p<0.0001), respectively (FIG. 4D). The presence of 5 UASs increased expression comparable to the strong tef1 promoter native to S. cerevisiae (FIG. 4D). Independently, nucleosome depletion could also increase expression levels 8.2-fold (p<0.01) (FIG. 4D).

In view of these preliminary data, the promoter library was expanded by constructing and characterizing 48 synthetic hybrid promoters (Table 2). To reinforce compatibility with the overall SGE design principles, three sequence considerations were implemented:

(1) No pair of UASs was used more than thrice, and no triplet of UASs was used more than once per library to avoid repetitive sequences. Promoters ranged from 161 bp to 181 bp in length. Also, no spacer or TSS sequence was reused. As a result, the maximum stretch of sequence similarity between any two promoters was 30 bp.
(2) No ‘NTG’ sequence is used in any spacer to avoid internal start codons.
(3) Promoters were further screened for predicted terminators and RBSs, which were removed by randomly mutating spacer sequences.

TABLE 2
Hybrid Yeast Promoter Sequences

GCTAAAAAGAGCTAGTACccgcgccTAGCATGTGACCTCCTTGAA
ACTGAAATTTacacaaaacttaagagcaacgcattaacttTATAA
AAGagcactgttgggcgtgagtggaggcgccggTTTTTAATATCA
TaTAGAAGTCAtttttaactaactaa
(SEQ ID NO: 50)
aaaaaCAttttttttTTAccgcgccGGGGGCGGTGGCTCAACGGC
TAGCATGTGAcatttccctaaaaaatagtttcgtttttttTATAA
AAGcgtaggagtactcgatggtacagatgagcaTTTTTTAGAAGT
CaTCGAAATAGtttttaactaactaa
(SEQ ID NO: 51)
tttttCTTtttttttAGAccgcgccACTGAAATTTGCTCAACGGC
TAGCATGTGAaaaagtttttgctatttttgatttttcgttTATAA
AAGaacgatctaccgactgtttcgcagagggccTTTTTAGATCAT
CaAGGAAGTAAtttttaactaactaa
(SEQ ID NO: 52)
ATtttttttttCGGCGCCccgcgccGGGGGCGGTGACTGAAATTT
GCTCAACGGCttcttcttaacactttttgcaggaaaaaagTATAA
AAGccgatagggtgggcgaaggggcgcaggtcCTTTTTATCAAAA
CaAATAAAACAtttttaactaagtaa
(SEQ ID NO: 53)
ACttttttttttGTACTCccgcgccACAGAGGGGCTAGCATGTGA
GGGGGCGGTGaaaaaagcaaaaaagaaaaagattttttttTATAA
AAGggccttggtctgaaactcctgcgtctcgcgTTTTTATAAGAA
CaACAACAAATtttttaactaagtaa
(SEQ ID NO: 54)
CCCGCttttttttttCGAccgcgccGCTCAACGGCCCTCCTTGAA
ACTGAAATTTagttaccttttttttttttaagctttttccTATAA
AAGggtccctgggtttgcgtactttatccgtcaTTTTTAAAATAT
CaTAGCACAACtttttaactaagtaa
(SEQ ID NO: 55)
TTAACTTTAGCCTAAATAccgcgccTAGCATGTGACCTCCTTGAA
GGGGGCGGTGgttcagaatcacccgcgaatacgtagtaatTATAA
AAGcgcggtggctccattaaattgctccttcctTTTTTAGAAAAT
CaAGAAGGACAtttttaagtaactaa
(SEQ ID NO: 56)
GtttttCCttttttttttccgcgccGGGGGCGGTGACAGAGGGGC
GCTCAACGGCcgcagaactatttttttagagtaactcgttTATAA
AAGcaatacttgggtcgacttgttatacgcggaTTTTTGAGCAAG
CaAGATATTTGtttttaactaagtaa
(SEQ ID NO: 57)
ATTTtttttttttGTCTCccgcgccACAGAGGGGGGGGGGGGTGA
CTGAAATTTtttttgacaagtcaagtcaggaaaaaaaaaTATAAA
AGggcgctgcgtaaggagtgctgccaggtggtTTTTTAAGAAATC
aAAAGAATAAtttttaagtaactaa
(SEQ ID NO: 58)
CTCGCTCttttttttttAccgcgccGCTCAACGGCACTGAAATTT
TAGCATGTGAtaagttcgctaaaaagccatttttttctagTATAA
AAGagcactgttgggcgtgagtggaggcgccggTTTTTAAGAAAT
CaAACAACTAAtttttaagtaactaa
(SEQ ID NO: 59)
AATTTTTTTaaaaaAGGCccgcgccGCTCAACGGCCCTCCTTGAA
GGGGGCGGTGtttttgaaaaaaagaagcaaaaactatattTATAA
AAGcgtaggagtactcgatggtacagatgagcaTTTTTATTACGT
TaCAAGAACACtttttaactaactaa
(SEQ ID NO: 60)
tttttTtttttTCCTTCCccgcgccACTGAAATTTGGGGGCGGTG
GCTCAACGGCatttttgaggagaagtttttacaaaaaaacTATAA
AAGaacgatctaccgactgtttcgcagagggcCTTTTTTAGCTAC
TaCCCCTATTAtttttaagtaactaa
(SEQ ID NO: 61)
ATATTCttttttttCGAAccgcgccACTGAAATTTCCTCCTTGAA
GCTCAACGGCcttttttaaaaataaactttttccaacataTATAA
AAGccgataggggggcgaaggggcgcaggtccTTTTTAGATCGTT
aAGGAATAGTtttttaactaactaa
(SEQ ID NO: 62)
GTCTCTATCTTAATCGTAccgcgccACAGAGGGGGGGGGGGGTGC
CTCCTTGAAaagttattagcgacgagtaaatcctcaacgTATAAA
AGggccttggtctgaaactcctgcgtctcgcgTTTTTAAAGACTT
aTACAAGAAGtttttaactaagtaa
(SEQ ID NO: 63)
GCCCCAACGGCCGGACTAccgcgccGGGGGCGGTGACAGAGGGGC
ACTGAAATTTggcccaaaaccatagggtataacccagaaaTATAA
AAGggtccctgggtttgcgtactttatccgtcaTTTTTGTAAAAA
TaCAGAACTCTtttttaactaactaa
(SEQ ID NO: 64)
TCTAACGACGGTCCTACAccgcgccGGGGGCGGTGGCTCAACGGC
CCTCCTTGAAttaaccgtactcgtaggactcaagagtacaTATAA
AAGcgcggtggctccattaaattgctccttcctTTTTTAAAGAAC
CaCAGAAAAATtttttaactaactaa
(SEQ ID NO: 65)
ATtttttCAtttttTTAAccgcgccACTGAAATTTTAGCATGTGA
ACAGAGGGGCCCTCCTTGAAcgatttttcacaaagaaaaaaagtt
ttttaTATAAAAGcaatacttgggtcgacttgttatacgcggaTT
TTTAAGTATTTaCCGTCTAAAtttttaactaactaa
(SEQ ID NO: 66)
GGttttttttttttttACccgcgccACAGAGGGGCGCTCAACGGC
TAGCATGTGAGGGGGCGGTGaaaaagaaattaaaaaaaaaaaatt
ccataTATAAAAGggcgctgcgtaaggagtgctgccaggtggtTT
TTTCTCCTATTaACGGTTTGAtttttaactaactaa
(SEQ ID NO: 67)
CACtttttttttttttTAccgcgccACTGAAATTTGCTCAACGGC
ACAGAGGGGGGGGGCGGTGaatttccaattaatctttttattact
cgtaTATAAAAGagcactgttgggcgtgagtggaggcgccggTTT
TTTAGAAAAGaAAGGATAGGtttttaactaagtaa
(SEQ ID NO: 68)
tttttttttATATATCGCccgcgccCCTCCTTGAAACTGAAATTT
ACAGAGGGGCGCTCAACGGCatcgaaaaaaaaacacaaagtcgtt
tttctTATAAAAGcgtaggagtactcgatggtacagatgagcaTT
TTTATATAACCaAACAGACCGtttttaactaagtaa
(SEQ ID NO: 70)
TCGCATAAGGACTATTAAccgcgccGGGGGCGGTGGCTCAACGGC
ACAGAGGGGCTAGCATGTGAcaagattccttcgtaaaacttcttt
ctcagTATAAAAGaacgatctaccgactgtttcgcagagggccTT
TTTATATAACCaATTTCAATAtttttaagtaagtaa
(SEQ ID NO: 71)
CCGtttttTTTtttttGCccgcgccACAGAGGGGCGCTCAACGGC
GGGGGCGGTGTAGCATGTGAttttttctaataaccaaactttttt
tttgaTATAAAAGccgatagggtgggcgaaggggcgcaggtccTT
TTTAAAGAATTaAATATAATCtttttaactaactaa
(SEQ ID NO: 72)
GAAtttttttttttttttccgcgccGCTCAACGGCTAGCATGTGA
ACTGAAATTTGGGGGCGGTGggcaatccaagagtttttttatttt
tctttTATAAAAGggccttggtctgaaactcctgcgtctcgcgaa
aaaAATGCACCaAACACAAGAtttttaactaactaa
(SEQ ID NO: 73)
tttttttttttttttATCccgcgccGCTCAACGGCACAGAGGGGC
ACTGAAATTTTAGCATGTGAgcgttatttttttttaaacttcttt
ttaaaTATAAAAGggtccctgggtttgcgtactttatccgtcaTT
TTTACAAGATCaACTAAGAACtttttaactaactaa
(SEQ ID NO: 74)
ttttttttttATAAAAGCccgcgccTAGCATGTGAGCTCAACGGC
GGGGGCGGTGACTGAAATTTaatttcggttaaaatttttcgtttc
actatTATAAAAGcgcggtggctccattaaattgctccttcctTT
TTTGGAAATTCaTACACAACAtttttaactaactaa
(SEQ ID NO: 75)
GAaaaaatttttTTTTTCccgcgccGCTCAACGGCTAGCATGTGA
ACAGAGGGGCACTGAAATTTtacgagttaaagtcgaagtttttta
aaaaaTATAAAAGcaatacttgggtcgacttgttatacgcggaTT
TTTTACACAACaACAGAACCAtttttaagtaactaa
(SEQ ID NO: 76)
TATTCGTTCTACAGTAACccgcgccACTGAAATTTGGGGGCGGTG
CCTCCTTGAATAGCATGTGAcagaaagagatacgtagcatttcag
actaaTATAAAAGggcgctgcgtaaggagtgctgccaggtggtTT
TTTGTCTCCCCaTTGTGCAGCtttttaactaagtaa
(SEQ ID NO: 77)
CCCAGAATAGTACTCCACccgcgccACAGAGGGGCGCTCAACGGC
ACTGAAATTTGGGGGCGGTGtagatcggtaagacgattcttcact
acttaTATAAAAGagcactgttgggcgtgagtggaggcgccggTT
TTTGCAGCGATaAGGAACATTtttttaactaagtaa
(SEQ ID NO: 78)
TtttttTttttttttTCAccgcgccGCTCAACGGCCCTCCTTGAA
ACAGAGGGGCTAGCATGTGAgttttttttgacaaaaatcaagggt
tatacTATAAAAGcgtaggagtactcgatggtacagatgagcaTT
TTTTGCACAATaTTTCAAGCTtttttaactaagtaa
(SEQ ID NO: 79)
GACGCCAAGTATCAGGAAccgcgccTAGCATGTGAACTGAAATTT
GCTCAACGGCGGGGGCGGTGttaggtcaaaacgctaactcattag
aatacTATAAAAGaacgatctaccgactgtttcgcagagggccTT
TTTAATATTTCaAGCTATACCtttttaagtaagtaa
(SEQ ID NO: 80)
CGGTCTACTCGAGTTAGAccgcgccACAGAGGGGCCCTCCTTGAA
GCTCAACGGCACTGAAATTTgcatcttactctcttagggtccaaa
ccctaTATAAAAGccgataggggggcgaaggggcgcaggtccAAA
AATTCAAGCTaTACCAAGCAtttttaactaactaa
(SEQ ID NO: 81)
ATTTTTTTTATCtttttCccgcgccTAGCATGTGACCTCCTTGAA
ACAGAGGGGCACTGAAATTTacttttttcttttttaggatccttt
ttttaTATAAAAGggccttggtctgaaactcctgcgtctcgcgTT
TTTAAGCATACaATCAACTATtttttaagtaagtaa
(SEQ ID NO: 82)
TCtttttTTTTtttttGAccgcgccACTGAAATTTACAGAGGGGC
TAGCATGTGACCTCCTTGAAGCTCAACGGCtttttgctccactaa
aaacgcatttaaaaaTATAAAAGggtccctgggtttgcgtacttt
atccgtcaTTTTTTAAGCAACaTTTTATACAtttttaactaagta
a
(SEQ ID NO: 83)
TATTtttttttCTACTAAccgcgccTAGCATGTGAGCTCAACGGC
ACTGAAATTTCCTCCTTGAAGGGGGCGGTGaaaaaaagcttaact
tactcgcttttttttTATAAAAGcgcggtggctccattaaattgc
tccttcctTTTTTAACATTTTaTACATTTTTtttttaagtaagta
a
(SEQ ID NO: 84)
GCACttttttttttttACccgcgccCCTCCTTGAAGGGGGCGGTG
GCTCAACGGCACTGAAATTTACAGAGGGGCaagaagttacgaaaa
aaaatctttttttatTATAAAAGcaatacttgggtcgacttgtta
tacgcggaTTTTTGTAAGAACaTCACACAAAtttttaactaagta
a
(SEQ ID NO: 85)
TCGCCACGTTTAAATCGAccgcgccACTGAAATTTCCTCCTTGAA
ACAGAGGGGCGGGGGCGGTGGCTCAACGGCagcttcttagttttt
cacgtatccactttaTATAAAAGggcgctgcgtaaggagtgctgc
caggtggtTTTTTAGAACATCaCACAAAGATtttttaagtaagta
a
(SEQ ID NO: 86)
TCTCCCTAAACAGCCCTAccgcgccACAGAGGGGCACTGAAATTT
GGGGGCGGTGTAGCATGTGAGCTCAACGGCacgtacaggctagat
ttcaactaataaccaTATAAAAGagcactgttgggcgtgagtgga
ggcgccggTTTTTAGAAAAACaTCTAACATAtttttaagtaagta
a
(SEQ ID NO: 87)
CAttttttttttttttTAccgcgccCCTCCTTGAAACTGAAATTT
TAGCATGTGAGGGGGCGGTGGCTCAACGGCaactcctttttaatc
atcataaaaattttaTATAAAAGcgtaggagtactcgatggtaca
gatgagcaTTTTTATACGGTCaACGAACTATtttttaagtaacta
a
(SEQ ID NO: 88)
TTCAttttttttttttTCccgcgccGCTCAACGGCCCTCCTTGAA
TAGCATGTGAACAGAGGGGCGGGGGCGGTGttttttttttcacgt
tttttacccttcaatTATAAAAGaacgatctaccgactgtttcgc
agagggccTTTTTAAAACACCaAGAACTTAGtttttaactaacta
a
(SEQ ID NO: 89)
GCGttttttttttttTCCccgcgccCCTCCTTGAATAGCATGTGA
GGGGGGGGTGACTGAAATTTACAGAGGGGCtaatccaaaaaaatt
ttttctttttttccgTATAAAAGccgatagggtgggcgaaggggc
gcaggtccTTTTTAAAAAACCaAGCAACTGCtttttaagtaacta
a
(SEQ ID NO: 90)
GTttttttttttttttCAccgcgccGCTCAACGGCGGGGGCGGTG
ACAGAGGGGCTAGCATGTGAACTGAAATTTgactttttttgtttt
ttatttttatttcacTATAAAAGggccttggtctgaaactcctgc
gtctcgcgTTTTTGGGAGAATaTTCGCAATTtttttaagtaagta
a
(SEQ ID NO: 91)
TTCTACTTTTttttttttccgcgccACAGAGGGGCCCTCCTTGAA
GGGGGCGGTGACTGAAATTTTAGCATGTGAaaaaattttttttaa
tcctcattttttaaaTATAAAAGggtccctgggtttgcgtacttt
atccgtcaTTTTTTTTCTTTCaTAACACCAAtttttaactaagta
a
(SEQ ID NO: 92)
GtttttttttttAtttttccgcgccCCTCCTTGAAGGGGGCGGTG
TAGCATGTGAACTGAAATTTACAGAGGGGCaatttttgttttttc
attaacgtttaacacTATAAAAGcgcggtggctccattaaattgc
tccttcctTTTTTATAACACCaAGCAACTAAtttttaactaagta
a
(SEQ ID NO: 93)
aaaaaTtttttttttTCAccgcgccGCTCAACGGCACAGAGGGGC
CCTCCTTGAATAGCATGTGAACTGAAATTTatctcattttttttt
ttttatttcgcgtaaTATAAAAGcaatacttgggtcgacttgtta
tacgcggaTTTTTAAGAAAGCaTAGCAATCTtttttaactaacta
a
(SEQ ID NO: 94)
CttttttttttttttACAccgcgccTAGCATGTGAGGGGGCGGTG
CCTCCTTGAAACTGAAATTTGCTCAACGGCccataatattttttt
ttttttttaatctcgTATAAAAGggcgctgcgtaaggagtgctgc
caggtggtTTTTTAAATTACTaTACTTCTATtttttaactaagta
a
(SEQ ID NO: 95)
TAGTACTCAGCCACAAGAccgcgccGGGGGCGGTGACTGAAATTT
CCTCCTTGAATAGCATGTGAGCTCAACGGCtttaccgaaggtctt
agtagcagtactcttTATAAAAGagcactgttgggcgtgagtgga
ggcgccggTTTTTAGAACTATaACACATAGAtttttaactaagta
a
(SEQ ID NO: 96)
CTCTTCTCGCTCTCGCGCccgcgccTAGCATGTGAGGGGGCGGTG
ACAGAGGGGCCCTCCTTGAAACTGAAATTTgcgaattaagtaggg
tcaagtcttaaggcaTATAAAAGcgtaggagtactcgatggtaca
gatgagcaTTTTTTATGTGTTaAATTTATTGtttttaactaacta
a
(SEQ ID NO: 97)
TCCGTCAGGTCTACCGAAccgcgccGGGGGCGGTGTAGCATGTGA
ACAGAGGGGCGCTCAACGGCCCTCCTTGAAtacatcattaccgac
tacagagttatccacTATAAAAGaacgatctaccgactgtttcgc
agagggccTTTTTGCAGAAACaACAACAACAtttttaagtaacta
a
(SEQ ID NO: 98)

To functionally test this promoter library in the different bacterial and yeast hosts, a single genetic element was constructed including mUkGFP, a fixed bacterial RBS, a fixed bacterial T7 promoter, a variable yeast promoter, and a fixed yeast terminator. This single genetic element was cloned onto a centromeric yeast-E. coli shuttle vector pYP (FIG. 5A). All 48 synthetic promoters spanned a 22-fold range of activity levels, with many reaching or exceeding the strength of the widely used strong tef1 and adh1 promoters (FIG. 5A). The stronger promoters were those that incorporated nucleosome depletion; for instance, 10 out of 11 promoters exceeding the strength of the robust adh1 promoter (Xiong et al., 2018) were nucleosome depleted. At the library level, promoters with 5 UASs did not necessarily exhibit higher expression than those with 3 or 4 UASs. Instead, it was observed that, for any given promoter, UASs can be reliably used to tune expression upward. To demonstrate, the number of UASs was increased from 3 to 5 in three weak promoters (YP2, YP7 and YP8). In two of the three promoters tested, this resulted in a significant dose-dependent increase in expression (p<0.0001) (FIG. 5C), providing a basis for rational pathway engineering through promoter tuning. Also, 6 out of 11 highly active promoters contained 5 UAS elements. GFP fluorescence in S. cerevisiae was strongly correlated with RNA levels (r²=0.92), confirming that differences in promoter strength were determined at the transcriptional level (FIG. 5D).

The hypothesis was fluorescence level would be steady when these constructs were shuttled into E. coli BL21(DE3), given that the bacterial transcription/translation signals were constant. Although most synthetic promoters showed strong expression in E. coli, a small subset of promoters exhibited attenuated expression (FIG. 5E). The degree of attenuation in E. coli was not meaningfully correlated with the expression strength in S. cerevisiae (FIG. 5G). The initial hypothesis was that these eukaryotic promoters may act at the transcriptional level in bacteria to impact RNA stability. qRT-PCR tests were performed and found that only some of the variability could be explained at the mRNA level, as RNA levels differed by less than 3-fold (FIG. 5H). These results indicate contributing effects at the translational level. To verify that these promoters functioned across multiple gene contexts, the mUkGFP reporter was swapped with an eGFP reporter. BLAST alignment reveals no significant similarities in nucleotide sequence between these two genes. Promoters were correlated in strength across the two reporters (r²=0.42) (FIG. 5F, 5I), indicating that expression level trends were largely independent of the downstream gene sequence. Overall, these data indicate attenuation is a combination of transcriptional and translational effects. Taken together, this new library of synthetic promoters can be appended to the 5′ sequence of the redesigned CDSs to activate BGCs in both E. coli and S. cerevisiae.

Expanding Bacterial Expression with an Inducible T7RNA Polymerase Expression Circuit

Given its orthogonality, processivity, and host-independence as in previous studies (Tabor, 2001), the bacteriophage T7 RNA polymerase (T7 RNAP) and cognate T7 promoter (pT7) system were selected to enable the hybrid eukaryotic-prokaryotic promoters to modulate transcription across diverse bacterial species. The major challenge was expressing the T7 RNAP in a host versatile manner because transcription from pT7 is constrained by the cognate T7 RNAP. The disclosed approach also sought to balance robust expression with titratable expression. As a result of the processivity of the T7 RNAP, overexpressed genes can accumulate to 30% of the total cellular protein and sequester 50% of translation capacity according to previous studies (Segall-Shapiro et al., 2014). This can result in fitness defects and be counterproductive to biosynthetic pathway functionality due to competition for cellular resources as previously reported (Scott et al., 2010). The Universal Bacterial Expression Resource (UBER) system was expanded to provide balance between robustness and titratability by coupling positive and negative feedback loops to modulate gene expression, and introduce an RNA riboswitch to modulate the levels of RNAP production. In the original UBER framework, seeding transcription provided by (+)—strand transcription from upstream genes drives the initial production of T7 RNAP (Kushwaha and Salis, 2015). T7 RNAP production is further auto-regulated through a positive feedback loop catalyzed by an upstream pT7. To prevent compounding RNAP amplification, a negative feedback loop proportionally produces an anhydrotetracyline (aTc) responsive TetR repressor to inhibit T7 RNAP production. Previous studies found that the translation initiation rate of the T7 RNAP was the primary determinant controlling system output (Kushwaha and Salis, 2015). However, a limitation of this design is a lack of inducible activity, an important criterion for controlled expression of heterologous biosynthetic pathways that may variably exhibit cytotoxicity in diverse hosts.

In accordance with previous studies (Espah Borujeni et al., 2016; Topp et al., 2010; Wachsmuth et al., 2013), it was hypothesized that a theophylline-responsive translational riboswitch could impart tunable control generalizable to function across bacterial phyla. The addition of this module required rebalancing the UBER framework. To achieve this, 16 variants of the UBER circuit necessary for optimized system performance were re-constructed by altering the strength of positive-negative feedback, riboswitch variant, and general architecture (FIGS. 12A and 6B). These variants were initially tested in E. coli Mach1 cells on a low copy miniRK2 vector pT7 RNAP (FIG. 6C). The circuit was oriented downstream of the vector's kanR gene to provide seeding transcription from its promoter. The output from the circuit was measured with a pT7 transcribed eGFP expressed on a second plasmid pT7GFP (FIG. 6D). These variants allowed the evaluation of the newly constructed circuit in a systematic and stepwise manner. Although T7 RNAP transcribed by seeding transcription alone (variant TO) was active, tests revealed highly attenuated signals from two theophylline riboswitch variants (variants T1 and T2) (FIG. 6E). The sequence differences between the various components are listed as SEQ ID Nos: 99-109. The sequences for the complete circuits are listed as SEQ ID Nos: 110-124. This attenuation was rescued by adding positive feedback (variant T3); albeit, at the cost of high uninduced background expression (FIG. 6E). Adding negative feedback (variants T4 and T5) substantially reduced background without compromising the induced expression level, motivating the characterization of additional variants (FIG. 6E). Positive feedback strength was adjusted by comparing the wildtype pT7WT with an attenuated mutant pT7H9 from previous studies (Jones et al., 2015) (FIG. 6F). Positive feedback strength of the pT7H9 mutant did not significantly affect reporter activity (FIG. 6F). Additionally, tetR and T7 RNAP genes were codon optimized, also without significant impact on expression levels (variants T12 and T13) (FIG. 6E). A significant increase in dynamic range was observed when the T7 RNAP and tetR genes were split from a bicistronic operon to a two-monocistronic architecture (variant T15) (FIG. 6E). This change removed the tetR gene from direct negative feedback, explaining the stronger background repression in the uninduced state (FIG. 6E). To benchmark this circuit, the pT7-eGFP plasmid was transformed into the commonly used E coli BL21(DE3), where T7 RNAP is produced by an IPTG-induced placUV5 promoter. Compared to BL21(DE3), variant T15 maintained equally induced expression, and thus was pursued in subsequent experimentation. To this end, a theophylline and aTC induction matrix was performed with circuit variant T15 E. coli Mach1 cells to demonstrate AND gate logic. Final OD₆₀₀ of the cultures were measured to highlight negative fitness impacts of over-induction. Plate reader analysis demonstrated that variant T15 functions as an AND gate, requiring both theophylline and aTc for full induction, with theophylline acting as the stronger inducer (Table 3). Plate reader analysis also indicated higher levels of GFP induction impair growth, consistent with previous reports on the fitness impacts of high-level gene expression. (Table 4) This new programmable circuit highlights the importance of titrating expression and the regulatory design principles required to achieve precise control of gene expression.

### Riboswitch Variant Sequences

SEQ ID NO: 99:
TACCGGTGATACCAGCATCGTCTTGATGCCCTTGGCAGCACCCTG
CTAAGGAGGCAACAAG
SEQ ID NO: 101:
aagtgataccagcatcgtcttgatgcccttggcagcacttcattt
acatactcggtaaactgaagtgctgccattttttttGGTACCGGT
GATACCAGCATCGTCTTGATGCCCTTGGCAGCACCCTGCTAAGGA
GGCAACAAG
SEQ ID NO: 102:
GACGGGACTCTCACCAGGTACCGGAGATACCAGCATCGTCTTGAT
GCCCTTGGCAGCTCCAGCTGCTAAGGAGGTATCAAG
SEQ ID NO: 103:
GACGGGACTCTCACCAGGTACCGGAGATACCAGCATCGTCTTGAT
GCCCTTGGCAGCTCCAGCTGCTAAGGAGGTATCAAGATGGAAGAC
GCCAAAAACATAAAGAAAGGCCCGGCG

tetR Variant Sequences:

Wild Type SEQ ID NO: 104:
ATGTCTAGATTAGATAAAAGTAAAGTGATTAACAGCGCATTAGAG
CTGCTTAATGAGGTCGGAATCGAAGGTTTAACAACCCGTAAACTC
GCCCAGAAGCTAGGTGTAGAGCAGCCTACATTGTATTGGCATGTA
AAAAATAAGCGGGCTTTGCTCGACGCCTTAGCCATTGAGATGTTA
GATAGGCACCATACTCACTTTTGCCCTTTAGAAGGGGAAAGCTGG
CAAGATTTTTTACGTAATAACGCTAAAAGTTTTAGATGTGCTTTA
CTAAGTCATCGCGATGGAGCAAAAGTACATTTAGGTACACGGCCT
ACAGAAAAACAGTATGAAACTCTCGAAAATCAATTAGCCTTTTTA
TGCCAACAAGGTTTTTCACTAGAGAATGCATTATATGCACTCAGC
GCTGTGGGGCATTTTACTTTAGGTTGCGTATTGGAAGATCAAGAG
CATCAAGTCGCTAAAGAAGAAAGGGAAACACCTACTACTGATAGT
ATGCCGCCATTATTACGACAAGCTATCGAATTATTTGATCACCAA
GGTGCAGAGCCAGCCTTCTTATTCGGCCTTGAATTGATCATATGC
GGATTAGAAAAACAACTTAAATGTGAAAGTGGGTCTTAA
Recoded SEQ ID NO: 105:
ATGTCAAGGCTGGATAAATCAAAAGTAATCAATAGCGCGCTGGAA
CTGCTGAACGAGGTCGGCATCGAAGGTCTGACCACCCGCAAGCTG
GCGCAAAAACTGGGCGTCGAACAACCGACGCTGTACTGGCACGTA
AAAAATAAGCGTGCGCTGCTGGACGCACTGGCAATTGAAATGCTG
GATCGTCACCACACCCACTTCTGTCCGCTGGAGGGTGAATCATGG
CAAGATTTCCTTCGCAACAACGCGAAGTCATTTCGCTGCGCGCTG
CTGAGCCACCGCGATGGAGCAAAAGTTCATCTGGGCACCCGCCCA
ACGGAGAAACAATATGAAACGCTGGAAAACCAGCTTGCCTTCCTG
TGCCAGCAGGGTTTCAGCCTTGAGAACGCGCTGTACGCGCTGAGC
GCCGTAGGTCACTTCACCCTGGGCTGTGTTCTGGAAGACCAAGAA
CATCAAGTAGCAAAAGAAGAGCGAGAAACCCCTACGACCGATTCG
ATGCCGCCGCTGCTGCGTCAGGCGATTGAACTGTTCGATCACCAG
GGCGCGGAACCGGCATTCCTGTTTGGTCTGGAACTTATTATATGC
GGCCTAGAAAAACAACTGAAGTGCGAAAGCGGTAGCTAA

### T7 RNAP Variant Sequences:

Wild Type SEQ ID NO: 106:
ATGAACACGATTAACATCGCTAAGAACGACTTCTCTGACATCGAA
CTGGCTGCTATCCCGTTCAACACTCTGGCTGACCATTACGGTGAG
CGTTTAGCTCGCGAACAGTTGGCCCTTGAGCATGAGTCTTACGAG
ATGGGTGAAGCACGCTTCCGCAAGATGTTTGAGCGTCAACTTAAA
GCTGGTGAGGTTGCGGATAACGCTGCCGCCAAGCCTCTCATCACT
ACCCTACTCCCTAAGATGATTGCACGCATCAACGACTGGTTTGAG
GAAGTGAAAGCTAAGCGCGGCAAGCGCCCGACAGCCTTCCAGTTC
CTGCAAGAAATCAAGCCGGAAGCCGTAGCGTACATCACCATTAAG
ACCACTCTGGCTTGCCTAACCAGTGCTGACAATACAACCGTTCAG
GCTGTAGCAAGCGCAATCGGTCGGGCCATTGAGGACGAGGCTCGC
TTCGGTCGTATCCGTGACCTTGAAGCTAAGCACTTCAAGAAAAAC
GTTGAGGAACAACTCAACAAGCGCGTAGGGCACGTCTACAAGAAA
GCATTTATGCAAGTTGTCGAGGCTGACATGCTCTCTAAGGGTCTA
CTCGGTGGCGAGGCGTGGTCTTCGTGGCATAAGGAAGACTCTATT
CATGTAGGAGTACGCTGCATCGAGATGCTCATTGAGTCAACCGGA
ATGGTTAGCTTACACCGCCAAAATGCTGGCGTAGTAGGTCAAGAC
TCTGAGACTATCGAACTCGCACCTGAATACGCTGAGGCTATCGCA
ACCCGTGCAGGTGCGCTGGCTGGCATCTCTCCGATGTTCCAACCT
TGCGTAGTTCCTCCTAAGCCGTGGACTGGCATTACTGGTGGTGGC
TATTGGGCTAACGGTCGTCGTCCTCTGGCGCTGGTGCGTACTCAC
AGTAAGAAAGCACTGATGCGCTACGAAGACGTTTACATGCCTGAG
GTGTACAAAGCGATTAACATTGCGCAAAACACCGCATGGAAAATC
AACAAGAAAGTCCTAGCGGTCGCCAACGTAATCACCAAGTGGAAG
CATTGTCCGGTCGAGGACATCCCTGCGATTGAGCGTGAAGAACTC
CCGATGAAACCGGAAGACATCGACATGAATCCTGAGGCTCTCACC
GCGTGGAAACGTGCTGCCGCTGCTGTGTACCGCAAGGACAAGGCT
CGCAAGTCTCGCCGTATCAGCCTTGAGTTCATGCTTGAGCAAGCC
AATAAGTTTGCTAACCATAAGGCCATCTGGTTCCCTTACAACATG
GACTGGCGCGGTCGTGTTTACGCTGTGTCAATGTTCAACCCGCAA
GGTAACGATATGACCAAAGGACTGCTTACGCTGGCGAAAGGTAAA
CCAATCGGTAAGGAAGGTTACTACTGGCTGAAAATCCACGGTGCA
AACTGTGCGGGTGTCGATAAGGTTCCGTTCCCTGAGCGCATCAAG
TTCATTGAGGAAAACCACGAGAACATCATGGCTTGCGCTAAGTCT
CCACTGGAGAACACTTGGTGGGCTGAGCAAGATTCTCCGTTCTGC
TTCCTTGCGTTCTGCTTTGAGTACGCTGGGGTACAGCACCACGGC
CTGAGCTATAACTGCTCCCTTCCGCTGGCGTTTGACGGGTCTTGC
TCTGGCATCCAGCACTTCTCCGCGATGCTCCGAGATGAGGTAGGT
GGTCGCGCGGTTAACTTGCTTCCTAGTGAAACCGTTCAGGACATC
TACGGGATTGTTGCTAAGAAAGTCAACGAGATTCTACAAGCAGAC
GCAATCAATGGGACCGATAACGAAGTAGTTACCGTGACCGATGAG
AACACTGGTGAAATCTCTGAGAAAGTCAAGCTGGGCACTAAGGCA
CTGGCTGGTCAATGGCTGGCTTACGGTGTTACTCGCAGTGTGACT
AAGCGTTCAGTCATGACGCTGGCTTACGGGTCCAAAGAGTTCGGC
TTCCGTCAACAAGTGCTGGAAGATACCATTCAGCCAGCTATTGAT
TCCGGCAAGGGTCTGATGTTCACTCAGCCGAATCAGGCTGCTGGA
TACATGGCTAAGCTGATTTGGGAATCTGTGAGCGTGACGGTGGTA
GCTGCGGTTGAAGCAATGAACTGGCTTAAGTCTGCTGCTAAGCTG
CTGGCTGCTGAGGTCAAAGATAAGAAGACTGGAGAGATTCTTCGC
AAGCGTTGCGCTGTGCATTGGGTAACTCCTGATGGTTTCCCTGTG
TGGCAGGAATACAAGAAGCCTATTCAGACGCGCTTGAACCTGATG
TTCCTCGGTCAGTTCCGCTTACAGCCTACCATTAACACCAACAAA
GATAGCGAGATTGATGCACACAAACAGGAGTCTGGTATCGCTCCT
AACTTTGTACACAGCCAAGACGGTAGCCACCTTCGTAAGACTGTA
GTGTGGGCACACGAGAAGTACGGAATCGAATCTTTTGCACTGATT
CACGACTCCTTCGGTACCATTCCGGCTGACGCTGCGAACCTGTTC
AAAGCAGTGCGCGAAACTATGGTTGACACATATGAGTCTTGTGAT
GTACTGGCTGATTTCTACGACCAGTTCGCTGACCAGTTGCACGAG
TCTCAATTGGACAAAATGCCAGCACTTCCGGCTAAAGGTAACTTG
AACCTCCGTGACATCTTAGAGTCGGACTTCGCGTTCGCGTAA
Recoded SEQ ID NO: 107:
ATGAACACGATTAACATCGCTAAGAACGACTTCTCTGACATCGAA
CTGGCTGCTATCCCGTTCAACACTCTGGCTGACCATTACGGTGAG
CGTTTgGCTCGCGAACAGTTGGCCCTTGAGCATGAGTCTTACGAG
ATGGGTGAAGCACGCTTCCGCAAGATGTTTGAGCGTCAACTTAAA
GCTGGTGAGGTTGCGGATAACGCTGCCGCCAAGCCTCTCATCACT
ACCCTACTCCCTAAGATGATTGCACGCATCAACGACTGGTTTGAG
GAAGTGAAAGCTAAGCGCGGCAAGCGCCCGACAGCCTTCCAGTTC
CTGCAAGAAATCAAGCCGGAAGCCGTAGCGTACATCACCATTAAG
ACCACTCTGGCTTGCCTAACCAGTGCTGACAATACAACCGTTCAG
GCTGTAGCAAGCGCAATCGGTCGGGCCATTGAGGACGAGGCTCGC
TTCGGTCGTATCCGTGACCTTGAAGCTAAGCACTTCAAGAAAAAC
GTTGAGGAACAACTCAACAAGCGCGTAGGGCACGTCTACAAGAAA
GCATTTATGCAAGTTGTCGAGGCTGACATGCTCTCTAAGGGTCTA
CTCGGTGGCGAGGCGTGGTCTTCGTGGCATAAGGAAGACTCTATT
CATGTAGGAGTACGCTGCATCGAGATGCTCATTGAGTCAACCGGA
ATGGTTAGCTTgCACCGCCAAAATGCTGGCGTAGTAGGTCAAGAC
TCTGAGACTATCGAACTCGCACCTGAATACGCTGAGGCTATCGCA
ACCCGTGCAGGTGCGCTGGCTGGCATCTCTCCGATGTTCCAACCT
TGCGTAGTTCCTCCTAAGCCGTGGACTGGCATTACTGGTGGTGGC
TATTGGGCTAACGGTCGTCGTCCTCTGGCGCTGGTGCGTACTCAC
AGTAAGAAAGCACTGATGCGCTACGAAGACGTTTACATGCCTGAG
GTGTACAAAGCGATTAACATTGCGCAAAACACCGCATGGAAAATC
AACAAGAAAGTCCTAGCGGTCGCCAACGTAATCACCAAGTGGAAG
CATTGTCCGGTCGAGGACATCCCTGCGATTGAGCGTGAAGAACTC
CCGATGAAACCGGAAGACATCGACATGAATCCTGAGGCTCTCACC
GCGTGGAAACGTGCTGCCGCTGCTGTGTACCGCAAGGACAAGGCT
CGCAAGTCTCGCCGTATCAGCCTTGAGTTCATGCTTGAGCAAGCC
AATAAGTTTGCTAACCATAAGGCCATCTGGTTCCCTTACAACATG
GACTGGCGCGGTCGTGTTTACGCTGTGTCAATGTTCAACCCGCAA
GGTAACGATATGACCAAAGGACTGCTTACGCTGGCGAAAGGTAAA
CCAATCGGTAAGGAAGGTTACTACTGGCTGAAAATCCACGGTGCA
AACTGTGCGGGTGTCGATAAGGTTCCGTTCCCTGAGCGCATCAAG
TTCATTGAGGAAAACCACGAGAACATCATGGCTTGCGCTAAGTCT
CCACTGGAGAACACTTGGTGGGCTGAGCAAGATTCTCCGTTCTGC
TTCCTTGCGTTCTGCTTTGAGTACGCTGGGGTACAGCACCACGGC
CTGAGCTATAACTGCTCCCTTCCGCTGGCGTTTGACGGGTCTTGC
TCTGGCATCCAGCACTTCTCCGCGATGCTCCGAGATGAGGTAGGT
GGTCGCGCGGTTAACTTGCTTCCTAGTGAAACCGTTCAGGACATC
TACGGGATTGTTGCTAAGAAAGTCAACGAGATTCTACAAGCAGAC
GCAATCAATGGGACCGATAACGAAGTAGTTACCGTGACCGATGAG
AACACTGGTGAAATCTCTGAGAAAGTCAAGCTGGGCACTAAGGCA
CTGGCTGGTCAATGGCTGGCTTACGGTGTTACTCGCAGTGTGACT
AAGCGTTCAGTCATGACGCTGGCTTACGGGTCCAAAGAGTTCGGC
TTCCGTCAACAAGTGCTGGAAGATACCATTCAGCCAGCTATTGAT
TCCGGCAAGGGTCTGATGTTCACTCAGCCGAATCAGGCTGCTGGA
TACATGGCTAAGCTGATTTGGGAATCTGTGAGCGTGACGGTGGTA
GCTGCGGTTGAAGCAATGAACTGGCTTAAGTCTGCTGCTAAGCTG
CTGGCTGCTGAGGTCAAAGATAAGAAGACTGGAGAGATTCTTCGC
AAGCGTTGCGCTGTGCATTGGGTAACTCCTGATGGTTTCCCTGTG
TGGCAGGAATACAAGAAGCCTATTCAGACGCGCTTGAACCTGATG
TTCCTCGGTCAGTTCCGCTTgCAGCCTACCATTAACACCAACAAA
GATAGCGAGATTGATGCACACAAACAGGAGTCTGGTATCGCTCCT
AACTTTGTACACAGCCAAGACGGTAGCCACCTTCGTAAGACTGTA
GTGTGGGCACACGAGAAGTACGGAATCGAATCTTTTGCACTGATT
CACGACTCCTTCGGTACCATTCCGGCTGACGCTGCGAACCTGTTC
AAAGCAGTGCGCGAAACTATGGTTGACACATATGAGTCTTGTGAT
GTACTGGCTGATTTCTACGACCAGTTCGCTGACCAGTTGCACGAG
TCTCAATTGGACAAAATGCCAGCACTTCCGGCTAAAGGTAACTTG
AACCTCCGTGACATCTTgGAGTCGGACTTCGCGTTCGCGTAA

### pT7 Variant Sequences:

Wild Type SEQ ID NO: 108:
TAATACGACTCACTATAGGGAGA
Mutant H9 SEQ ID NO: 109:
TAATACGACTCACTAATACTGAA

### Final Complete Circuits:

T0 SEQ ID NO: 110:
TCAGAATTGGTTAATTGGTTGTAACACTGGTTGGCAGCACAATggTAAGGAGGCA
ACAAGATGAACACGATTAACATCGCTAAGAACGACTTCTCTGACATCGAACTGGC
TGCTATCCCGTTCAACACTCTGGCTGACCATTACGGTGAGCGTTTAGCTCGCGAA
CAGTTGGCCCTTGAGCATGAGTCTTACGAGATGGGTGAAGCACGCTTCCGCAAGA
TGTTTGAGCGTCAACTTAAAGCTGGTGAGGTTGCGGATAACGCTGCCGCCAAGCC
TCTCATCACTACCCTACTCCCTAAGATGATTGCACGCATCAACGACTGGTTTGAG
GAAGTGAAAGCTAAGCGCGGCAAGCGCCCGACAGCCTTCCAGTTCCTGCAAGAAA
TCAAGCCGGAAGCCGTAGCGTACATCACCATTAAGACCACTCTGGCTTGCCTAAC
CAGTGCTGACAATACAACCGTTCAGGCTGTAGCAAGCGCAATCGGTCGGGCCATT
GAGGACGAGGCTCGCTTCGGTCGTATCCGTGACCTTGAAGCTAAGCACTTCAAGA
AAAACGTTGAGGAACAACTCAACAAGCGCGTAGGGCACGTCTACAAGAAAGCATT
TATGCAAGTTGTCGAGGCTGACATGCTCTCTAAGGGTCTACTCGGTGGCGAGGCG
TGGTCTTCGTGGCATAAGGAAGACTCTATTCATGTAGGAGTACGCTGCATCGAGA
TGCTCATTGAGTCAACCGGAATGGTTAGCTTACACCGCCAAAATGCTGGCGTAGT
AGGTCAAGACTCTGAGACTATCGAACTCGCACCTGAATACGCTGAGGCTATCGCA
ACCCGTGCAGGTGCGCTGGCTGGCATCTCTCCGATGTTCCAACCTTGCGTAGTTC
CTCCTAAGCCGTGGACTGGCATTACTGGTGGTGGCTATTGGGCTAACGGTCGTCG
TCCTCTGGCGCTGGTGCGTACTCACAGTAAGAAAGCACTGATGCGCTACGAAGAC
GTTTACATGCCTGAGGTGTACAAAGCGATTAACATTGCGCAAAACACCGCATGGA
AAATCAACAAGAAAGTCCTAGCGGTCGCCAACGTAATCACCAAGTGGAAGCATTG
TCCGGTCGAGGACATCCCTGCGATTGAGCGTGAAGAACTCCCGATGAAACCGGAA
GACATCGACATGAATCCTGAGGCTCTCACCGCGTGGAAACGTGCTGCCGCTGCTG
TGTACCGCAAGGACAAGGCTCGCAAGTCTCGCCGTATCAGCCTTGAGTTCATGCT
TGAGCAAGCCAATAAGTTTGCTAACCATAAGGCCATCTGGTTCCCTTACAACATG
GACTGGCGCGGTCGTGTTTACGCTGTGTCAATGTTCAACCCGCAAGGTAACGATA
TGACCAAAGGACTGCTTACGCTGGCGAAAGGTAAACCAATCGGTAAGGAAGGTTA
CTACTGGCTGAAAATCCACGGTGCAAACTGTGCGGGTGTCGATAAGGTTCCGTTC
CCTGAGCGCATCAAGTTCATTGAGGAAAACCACGAGAACATCATGGCTTGCGCTA
AGTCTCCACTGGAGAACACTTGGTGGGCTGAGCAAGATTCTCCGTTCTGCTTCCT
TGCGTTCTGCTTTGAGTACGCTGGGGTACAGCACCACGGCCTGAGCTATAACTGC
TCCCTTCCGCTGGCGTTTGACGGGTCTTGCTCTGGCATCCAGCACTTCTCCGCGA
TGCTCCGAGATGAGGTAGGTGGTCGCGCGGTTAACTTGCTTCCTAGTGAAACCGT
TCAGGACATCTACGGGATTGTTGCTAAGAAAGTCAACGAGATTCTACAAGCAGAC
GCAATCAATGGGACCGATAACGAAGTAGTTACCGTGACCGATGAGAACACTGGTG
AAATCTCTGAGAAAGTCAAGCTGGGCACTAAGGCACTGGCTGGTCAATGGCTGGC
TTACGGTGTTACTCGCAGTGTGACTAAGCGTTCAGTCATGACGCTGGCTTACGGG
TCCAAAGAGTTCGGCTTCCGTCAACAAGTGCTGGAAGATACCATTCAGCCAGCTA
TTGATTCCGGCAAGGGTCTGATGTTCACTCAGCCGAATCAGGCTGCTGGATACAT
GGCTAAGCTGATTTGGGAATCTGTGAGCGTGACGGTGGTAGCTGCGGTTGAAGCA
ATGAACTGGCTTAAGTCTGCTGCTAAGCTGCTGGCTGCTGAGGTCAAAGATAAGA
AGACTGGAGAGATTCTTCGCAAGCGTTGCGCTGTGCATTGGGTAACTCCTGATGG
TTTCCCTGTGTGGCAGGAATACAAGAAGCCTATTCAGACGCGCTTGAACCTGATG
TTCCTCGGTCAGTTCCGCTTACAGCCTACCATTAACACCAACAAAGATAGCGAGA
TTGATGCACACAAACAGGAGTCTGGTATCGCTCCTAACTTTGTACACAGCCAAGA
CGGTAGCCACCTTCGTAAGACTGTAGTGTGGGCACACGAGAAGTACGGAATCGAA
TCTTTTGCACTGATTCACGACTCCTTCGGTACCATTCCGGCTGACGCTGCGAACC
TGTTCAAAGCAGTGCGCGAAACTATGGTTGACACATATGAGTCTTGTGATGTACT
GGCTGATTTCTACGACCAGTTCGCTGACCAGTTGCACGAGTCTCAATTGGACAAA
ATGCCAGCACTTCCGGCTAAAGGTAACTTGAACCTCCGTGACATCTTAGAGTCGG
ACTTCGCGTTCGCGTAA
T1 SEQ ID NO: 111:
TCAGAATTGGTTAATTGGTTGTAACACTGGTACCGGTGATACCAGCATCGTCTTG
ATGCCCTTGGCAGCACCCTGCTAAGGAGGCAACAAGATGAACACGATTAACATCG
CTAAGAACGACTTCTCTGACATCGAACTGGCTGCTATCCCGTTCAACACTCTGGC
TGACCATTACGGTGAGCGTTTAGCTCGCGAACAGTTGGCCCTTGAGCATGAGTCT
TACGAGATGGGTGAAGCACGCTTCCGCAAGATGTTTGAGCGTCAACTTAAAGCTG
GTGAGGTTGCGGATAACGCTGCCGCCAAGCCTCTCATCACTACCCTACTCCCTAA
GATGATTGCACGCATCAACGACTGGTTTGAGGAAGTGAAAGCTAAGCGCGGCAAG
CGCCCGACAGCCTTCCAGTTCCTGCAAGAAATCAAGCCGGAAGCCGTAGCGTACA
TCACCATTAAGACCACTCTGGCTTGCCTAACCAGTGCTGACAATACAACCGTTCA
GGCTGTAGCAAGCGCAATCGGTCGGGCCATTGAGGACGAGGCTCGCTTCGGTCGT
ATCCGTGACCTTGAAGCTAAGCACTTCAAGAAAAACGTTGAGGAACAACTCAACA
AGCGCGTAGGGCACGTCTACAAGAAAGCATTTATGCAAGTIGTCGAGGCTGACAT
GCTCTCTAAGGGTCTACTCGGTGGCGAGGCGTGGTCTTCGTGGCATAAGGAAGAC
TCTATTCATGTAGGAGTACGCTGCATCGAGATGCTCATTGAGTCAACCGGAATGG
TTAGCTTACACCGCCAAAATGCTGGCGTAGTAGGTCAAGACTCTGAGACTATCGA
ACTCGCACCTGAATACGCTGAGGCTATCGCAACCCGTGCAGGTGCGCTGGCTGGC
ATCTCTCCGATGTTCCAACCTTGCGTAGTTCCTCCTAAGCCGTGGACTGGCATTA
CTGGTGGTGGCTATTGGGCTAACGGTCGTCGTCCTCTGGCGCTGGTGCGTACTCA
CAGTAAGAAAGCACTGATGCGCTACGAAGACGTTTACATGCCTGAGGTGTACAAA
GCGATTAACATTGCGCAAAACACCGCATGGAAAATCAACAAGAAAGTCCTAGCGG
TCGCCAACGTAATCACCAAGTGGAAGCATTGTCCGGTCGAGGACATCCCTGCGAT
TGAGCGTGAAGAACTCCCGATGAAACCGGAAGACATCGACATGAATCCTGAGGCT
CTCACCGCGTGGAAACGTGCTGCCGCTGCTGTGTACCGCAAGGACAAGGCTCGCA
AGTCTCGCCGTATCAGCCTTGAGTTCATGCTTGAGCAAGCCAATAAGTTTGCTAA
CCATAAGGCCATCTGGTTCCCTTACAACATGGACTGGCGCGGTCGTGTTTACGCT
GTGTCAATGTTCAACCCGCAAGGTAACGATATGACCAAAGGACTGCTTACGCTGG
CGAAAGGTAAACCAATCGGTAAGGAAGGTTACTACTGGCTGAAAATCCACGGTGC
AAACTGTGCGGGTGTCGATAAGGTTCCGTTCCCTGAGCGCATCAAGTTCATTGAG
GAAAACCACGAGAACATCATGGCTTGCGCTAAGTCTCCACTGGAGAACACTTGGT
GGGCTGAGCAAGATTCTCCGTTCTGCTTCCTTGCGTTCTGCTTTGAGTACGCTGG
GGTACAGCACCACGGCCTGAGCTATAACTGCTCCCTTCCGCTGGCGTTTGACGGG
TCTTGCTCTGGCATCCAGCACTTCTCCGCGATGCTCCGAGATGAGGTAGGTGGTC
GCGCGGTTAACTTGCTTCCTAGTGAAACCGTTCAGGACATCTACGGGATTGTTGC
TAAGAAAGTCAACGAGATTCTACAAGCAGACGCAATCAATGGGACCGATAACGAA
GTAGTTACCGTGACCGATGAGAACACTGGTGAAATCTCTGAGAAAGTCAAGCTGG
GCACTAAGGCACTGGCTGGTCAATGGCTGGCTTACGGTGTTACTCGCAGTGTGAC
TAAGCGTTCAGTCATGACGCTGGCTTACGGGTCCAAAGAGTTCGGCTTCCGTCAA
CAAGTGCTGGAAGATACCATTCAGCCAGCTATTGATTCCGGCAAGGGTCTGATGT
TCACTCAGCCGAATCAGGCTGCTGGATACATGGCTAAGCTGATTTGGGAATCTGT
GAGCGTGACGGTGGTAGCTGCGGTTGAAGCAATGAACTGGCTTAAGTCTGCTGCT
AAGCTGCTGGCTGCTGAGGTCAAAGATAAGAAGACTGGAGAGATTCTTCGCAAGC
GTTGCGCTGTGCATTGGGTAACTCCTGATGGTTTCCCTGTGTGGCAGGAATACAA
GAAGCCTATTCAGACGCGCTTGAACCTGATGTTCCTCGGTCAGTTCCGCTTACAG
CCTACCATTAACACCAACAAAGATAGCGAGATTGATGCACACAAACAGGAGTCTG
GTATCGCTCCTAACTTTGTACACAGCCAAGACGGTAGCCACCTTCGTAAGACTGT
AGTGTGGGCACACGAGAAGTACGGAATCGAATCTTTTGCACTGATTCACGACTCC
TTCGGTACCATTCCGGCTGACGCTGCGAACCTGTTCAAAGCAGTGCGCGAAACTA
TGGTTGACACATATGAGTCTTGTGATGTACTGGCTGATTTCTACGACCAGTTCGC
TGACCAGTTGCACGAGTCTCAATTGGACAAAATGCCAGCACTTCCGGCTAAAGGT
AACTTGAACCTCCGTGACATCTTAGAGTCGGACTTCGCGTTCGCGTAA
T2 SEQ ID NO: 112:
TCAGAATTGGTTAATTGGTTGTAACACTGGaagtgataccagcatcgtcttgatg
cccttggcagcacttcatttacatactcggtaaactgaagtgctgccattttttt
tGGTACCGGTGATACCAGCATCGTCTTGATGCCCTTGGCAGCACCCTGCTAAGGA
GGCAACAAGATGAACACGATTAACATCGCTAAGAACGACTTCTCTGACATCGAAC
TGGCTGCTATCCCGTTCAACACTCTGGCTGACCATTACGGTGAGCGTTTAGCTCG
CGAACAGTTGGCCCTTGAGCATGAGTCTTACGAGATGGGTGAAGCACGCTTCCGC
AAGATGTTTGAGCGTCAACTTAAAGCTGGTGAGGTTGCGGATAACGCTGCCGCCA
AGCCTCTCATCACTACCCTACTCCCTAAGATGATTGCACGCATCAACGACTGGTT
TGAGGAAGTGAAAGCTAAGCGCGGCAAGCGCCCGACAGCCTTCCAGTTCCTGCAA
GAAATCAAGCCGGAAGCCGTAGCGTACATCACCATTAAGACCACTCTGGCTTGCC
TAACCAGTGCTGACAATACAACCGTTCAGGCTGTAGCAAGCGCAATCGGTCGGGC
CATTGAGGACGAGGCTCGCTTCGGTCGTATCCGTGACCTTGAAGCTAAGCACTTC
AAGAAAAACGTTGAGGAACAACTCAACAAGCGCGTAGGGCACGTCTACAAGAAAG
CATTTATGCAAGTTGTCGAGGCTGACATGCTCTCTAAGGGTCTACTCGGTGGCGA
GGCGTGGTCTTCGTGGCATAAGGAAGACTCTATTCATGTAGGAGTACGCTGCATC
GAGATGCTCATTGAGTCAACCGGAATGGTTAGCTTACACCGCCAAAATGCTGGCG
TAGTAGGTCAAGACTCTGAGACTATCGAACTCGCACCTGAATACGCTGAGGCTAT
CGCAACCCGTGCAGGTGCGCTGGCTGGCATCTCTCCGATGTTCCAACCTTGCGTA
GTTCCTCCTAAGCCGTGGACTGGCATTACTGGTGGTGGCTATTGGGCTAACGGTC
GTCGTCCTCTGGCGCTGGTGCGTACTCACAGTAAGAAAGCACTGATGCGCTACGA
AGACGTTTACATGCCTGAGGTGTACAAAGCGATTAACATTGCGCAAAACACCGCA
TGGAAAATCAACAAGAAAGTCCTAGCGGTCGCCAACGTAATCACCAAGTGGAAGC
ATTGTCCGGTCGAGGACATCCCTGCGATTGAGCGTGAAGAACTCCCGATGAAACC
GGAAGACATCGACATGAATCCTGAGGCTCTCACCGCGTGGAAACGTGCTGCCGCT
GCTGTGTACCGCAAGGACAAGGCTCGCAAGTCTCGCCGTATCAGCCTTGAGTTCA
TGCTTGAGCAAGCCAATAAGTTTGCTAACCATAAGGCCATCTGGTTCCCTTACAA
CATGGACTGGCGCGGTCGTGTTTACGCTGTGTCAATGTTCAACCCGCAAGGTAAC
GATATGACCAAAGGACTGCTTACGCTGGCGAAAGGTAAACCAATCGGTAAGGAAG
GTTACTACTGGCTGAAAATCCACGGTGCAAACTGTGCGGGTGTCGATAAGGTTCC
GTTCCCTGAGCGCATCAAGTTCATTGAGGAAAACCACGAGAACATCATGGCTTGC
GCTAAGTCTCCACTGGAGAACACTTGGTGGGCTGAGCAAGATTCTCCGTTCTGCT
TCCTTGCGTTCTGCTTTGAGTACGCTGGGGTACAGCACCACGGCCTGAGCTATAA
CTGCTCCCTTCCGCTGGCGTTTGACGGGTCTTGCTCTGGCATCCAGCACTTCTCC
GCGATGCTCCGAGATGAGGTAGGTGGTCGCGCGGTTAACTTGCTTCCTAGTGAAA
CCGTTCAGGACATCTACGGGATTGTTGCTAAGAAAGTCAACGAGATTCTACAAGC
AGACGCAATCAATGGGACCGATAACGAAGTAGTTACCGTGACCGATGAGAACACT
GGTGAAATCTCTGAGAAAGTCAAGCTGGGCACTAAGGCACTGGCTGGTCAATGGC
TGGCTTACGGTGTTACTCGCAGTGTGACTAAGCGTTCAGTCATGACGCTGGCTTA
CGGGTCCAAAGAGTTCGGCTTCCGTCAACAAGTGCTGGAAGATACCATTCAGCCA
GCTATTGATTCCGGCAAGGGTCTGATGTTCACTCAGCCGAATCAGGCTGCTGGAT
ACATGGCTAAGCTGATTTGGGAATCTGTGAGCGTGACGGTGGTAGCTGCGGTTGA
AGCAATGAACTGGCTTAAGTCTGCTGCTAAGCTGCTGGCTGCTGAGGTCAAAGAT
AAGAAGACTGGAGAGATTCTTCGCAAGCGTTGCGCTGTGCATTGGGTAACTCCTG
ATGGTTTCCCTGTGTGGCAGGAATACAAGAAGCCTATTCAGACGCGCTTGAACCT
GATGTTCCTCGGTCAGTTCCGCTTACAGCCTACCATTAACACCAACAAAGATAGC
GAGATTGATGCACACAAACAGGAGTCTGGTATCGCTCCTAACTTTGTACACAGCC
AAGACGGTAGCCACCTTCGTAAGACTGTAGTGTGGGCACACGAGAAGTACGGAAT
CGAATCTTTTGCACTGATTCACGACTCCTTCGGTACCATTCCGGCTGACGCTGCG
AACCTGTTCAAAGCAGTGCGCGAAACTATGGTTGACACATATGAGTCTTGTGATG
TACTGGCTGATTTCTACGACCAGTTCGCTGACCAGTTGCACGAGTCTCAATTGGA
CAAAATGCCAGCACTTCCGGCTAAAGGTAACTTGAACCTCCGTGACATCTTAGAG
TCGGACTTCGCGTTCGCGTAA
T3 SEQ ID NO: 113:
TCAGAATTGGTTAATTGGTTGTAACACTGGTAATACGACTCACTAATACTGAAaa
gtgataccagcatcgtcttgatgcccttggcagcacttcatttacatactcggta
aactgaagtgctgccattttttttGGTACCGGTGATACCAGCATCGTCTTGATGC
CCTTGGCAGCACCCTGCTAAGGAGGCAACAAGATGAACACGATTAACATCGCTAA
GAACGACTTCTCTGACATCGAACTGGCTGCTATCCCGTTCAACACTCTGGCTGAC
CATTACGGTGAGCGTTTAGCTCGCGAACAGTTGGCCCTTGAGCATGAGTCTTACG
AGATGGGTGAAGCACGCTTCCGCAAGATGTTTGAGCGTCAACTTAAAGCTGGTGA
GGTTGCGGATAACGCTGCCGCCAAGCCTCTCATCACTACCCTACTCCCTAAGATG
ATTGCACGCATCAACGACTGGTTTGAGGAAGTGAAAGCTAAGCGCGGCAAGCGCC
CGACAGCCTTCCAGTTCCTGCAAGAAATCAAGCCGGAAGCCGTAGCGTACATCAC
CATTAAGACCACTCTGGCTTGCCTAACCAGTGCTGACAATACAACCGTTCAGGCT
GTAGCAAGCGCAATCGGTCGGGCCATTGAGGACGAGGCTCGCTTCGGTCGTATCC
GTGACCTTGAAGCTAAGCACTTCAAGAAAAACGTTGAGGAACAACTCAACAAGCG
CGTAGGGCACGTCTACAAGAAAGCATTTATGCAAGTTGTCGAGGCTGACATGCTC
TCTAAGGGTCTACTCGGTGGCGAGGCGTGGTCTTCGTGGCATAAGGAAGACTCTA
TTCATGTAGGAGTACGCTGCATCGAGATGCTCATTGAGTCAACCGGAATGGTTAG
CTTACACCGCCAAAATGCTGGCGTAGTAGGTCAAGACTCTGAGACTATCGAACTC
GCACCTGAATACGCTGAGGCTATCGCAACCCGTGCAGGTGCGCTGGCTGGCATCT
CTCCGATGTTCCAACCTTGCGTAGTTCCTCCTAAGCCGTGGACTGGCATTACTGG
TGGTGGCTATTGGGCTAACGGTCGTCGTCCTCTGGCGCTGGTGCGTACTCACAGT
AAGAAAGCACTGATGCGCTACGAAGACGTTTACATGCCTGAGGTGTACAAAGCGA
TTAACATTGCGCAAAACACCGCATGGAAAATCAACAAGAAAGTCCTAGCGGTCGC
CAACGTAATCACCAAGTGGAAGCATTGTCCGGTCGAGGACATCCCTGCGATTGAG
CGTGAAGAACTCCCGATGAAACCGGAAGACATCGACATGAATCCTGAGGCTCTCA
CCGCGTGGAAACGTGCTGCCGCTGCTGTGTACCGCAAGGACAAGGCTCGCAAGTC
TCGCCGTATCAGCCTTGAGTTCATGCTTGAGCAAGCCAATAAGTTTGCTAACCAT
AAGGCCATCTGGTTCCCTTACAACATGGACTGGCGCGGTCGTGTTTACGCTGTGT
CAATGTTCAACCCGCAAGGTAACGATATGACCAAAGGACTGCTTACGCTGGCGAA
AGGTAAACCAATCGGTAAGGAAGGTTACTACTGGCTGAAAATCCACGGTGCAAAC
TGTGCGGGTGTCGATAAGGTTCCGTTCCCTGAGCGCATCAAGTTCATTGAGGAAA
ACCACGAGAACATCATGGCTTGCGCTAAGTCTCCACTGGAGAACACTTGGTGGGC
TGAGCAAGATTCTCCGTTCTGCTTCCTTGCGTTCTGCTTTGAGTACGCTGGGGTA
CAGCACCACGGCCTGAGCTATAACTGCTCCCTTCCGCTGGCGTTTGACGGGTCTT
GCTCTGGCATCCAGCACTTCTCCGCGATGCTCCGAGATGAGGTAGGTGGTCGCGC
GGTTAACTTGCTTCCTAGTGAAACCGTTCAGGACATCTACGGGATTGTTGCTAAG
AAAGTCAACGAGATTCTACAAGCAGACGCAATCAATGGGACCGATAACGAAGTAG
TTACCGTGACCGATGAGAACACTGGTGAAATCTCTGAGAAAGTCAAGCTGGGCAC
TAAGGCACTGGCTGGTCAATGGCTGGCTTACGGTGTTACTCGCAGTGTGACTAAG
CGTTCAGTCATGACGCTGGCTTACGGGTCCAAAGAGTTCGGCTTCCGTCAACAAG
TGCTGGAAGATACCATTCAGCCAGCTATTGATTCCGGCAAGGGTCTGATGTTCAC
TCAGCCGAATCAGGCTGCTGGATACATGGCTAAGCTGATTTGGGAATCTGTGAGC
GTGACGGTGGTAGCTGCGGTTGAAGCAATGAACTGGCTTAAGTCTGCTGCTAAGC
TGCTGGCTGCTGAGGTCAAAGATAAGAAGACTGGAGAGATTCTTCGCAAGCGTTG
CGCTGTGCATTGGGTAACTCCTGATGGTTTCCCTGTGTGGCAGGAATACAAGAAG
CCTATTCAGACGCGCTTGAACCTGATGTTCCTCGGTCAGTTCCGCTTACAGCCTA
CCATTAACACCAACAAAGATAGCGAGATTGATGCACACAAACAGGAGTCTGGTAT
CGCTCCTAACTTTGTACACAGCCAAGACGGTAGCCACCTTCGTAAGACTGTAGTG
TGGGCACACGAGAAGTACGGAATCGAATCTTTTGCACTGATTCACGACTCCTTCG
GTACCATTCCGGCTGACGCTGCGAACCTGTTCAAAGCAGTGCGCGAAACTATGGT
TGACACATATGAGTCTTGTGATGTACTGGCTGATTTCTACGACCAGTTCGCTGAC
CAGTTGCACGAGTCTCAATTGGACAAAATGCCAGCACTTCCGGCTAAAGGTAACT
TGAACCTCCGTGACATCTTAGAGTCGGACTTCGCGTTCGCGTAA
T4 SEQ ID NO: 114:
TCAGAATTGGTTAATTGGTTGTAACACTGGTCTATCATTGATAGGTATAAATTAA
TACGACTCACTAATACTGAACCTATCAGTGATAGATCCAAACCCAAAAACACAGG
AGTTTTTAGAATGTCTAGATTAGATAAAAGTAAAGTGATTAACAGCGCATTAGAG
CTGCTTAATGAGGTCGGAATCGAAGGTTTAACAACCCGTAAACTCGCCCAGAAGC
TAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTA
GAAGGGGAAAGCTGGCAAGATTTTTTACGTAATAACGCTAAAAGTTTTAGATGTG
CTTTACTAAGTCATCGCGATGGAGCAAAAGTACATTTAGGTACACGGCCTACAGA
AAAACAGTATGAAACTCTCGAAAATCAATTAGCCTTTTTATGCCAACAAGGTTTT
TCACTAGAGAATGCATTATATGCACTCAGCGCTGTGGGGCATTTTACTTTAGGTT
GCGTATTGGAAGATCAAGAGCATCAAGTCGCTAAAGAAGAAAGGGAAACACCTAC
TACTGATAGTATGCCGCCATTATTACGACAAGCTATCGAATTATTTGATCACCAA
GGTGCAGAGCCAGCCTTCTTATTCGGCCTTGAATTGATCATATGCGGATTAGAAA
AACAACTTAAATGTGAAAGTGGGTCTTAAaagtgataccagcatcgtcttgatgc
ccliggcagcacticatitacatactcggtaaactgaagtgctgccatttttttt
GGTACCGGTGATACCAGCATCGTCTTGATGCCCTTGGCAGCACCCTGCTAAGGAG
GCAACAAGATGAACACGATTAACATCGCTAAGAACGACTTCTCTGACATCGAACT
GGCTGCTATCCCGTTCAACACTCTGGCTGACCATTACGGTGAGCGTTTAGCTCGC
GAACAGTTGGCCCTTGAGCATGAGTCTTACGAGATGGGTGAAGCACGCTTCCGCA
AGATGTTTGAGCGTCAACTTAAAGCTGGTGAGGTTGCGGATAACGCTGCCGCCAA
GCCTCTCATCACTACCCTACTCCCTAAGATGATTGCACGCATCAACGACTGGTTT
GAGGAAGTGAAAGCTAAGCGCGGCAAGCGCCCGACAGCCTTCCAGTTCCTGCAAG
AAATCAAGCCGGAAGCCGTAGCGTACATCACCATTAAGACCACTCTGGCTTGCCT
AACCAGTGCTGACAATACAACCGTTCAGGCTGTAGCAAGCGCAATCGGTCGGGCC
ATTGAGGACGAGGCTCGCTTCGGTCGTATCCGTGACCTTGAAGCTAAGCACTTCA
AGAAAAACGTTGAGGAACAACTCAACAAGCGCGTAGGGCACGTCTACAAGAAAGC
ATTTATGCAAGTTGTCGAGGCTGACATGCTCTCTAAGGGTCTACTCGGTGGCGAG
GCGTGGTCTTCGTGGCATAAGGAAGACTCTATTCATGTAGGAGTACGCTGCATCG
AGATGCTCATTGAGTCAACCGGAATGGTTAGCTTACACCGCCAAAATGCTGGCGT
AGTAGGTCAAGACTCTGAGACTATCGAACTCGCACCTGAATACGCTGAGGCTATC
GCAACCCGTGCAGGTGCGCTGGCTGGCATCTCTCCGATGTTCCAACCTTGCGTAG
TTCCTCCTAAGCCGTGGACTGGCATTACTGGTGGTGGCTATTGGGCTAACGGTCG
TCGTCCTCTGGCGCTGGTGCGTACTCACAGTAAGAAAGCACTGATGCGCTACGAA
GACGTTTACATGCCTGAGGTGTACAAAGCGATTAACATTGCGCAAAACACCGCAT
GGAAAATCAACAAGAAAGTCCTAGCGGTCGCCAACGTAATCACCAAGTGGAAGCA
TTGTCCGGTCGAGGACATCCCTGCGATTGAGCGTGAAGAACTCCCGATGAAACCG
GAAGACATCGACATGAATCCTGAGGCTCTCACCGCGTGGAAACGTGCTGCCGCTG
CTGTGTACCGCAAGGACAAGGCTCGCAAGTCTCGCCGTATCAGCCTTGAGTTCAT
GCTTGAGCAAGCCAATAAGTTTGCTAACCATAAGGCCATCTGGTTCCCTTACAAC
ATGGACTGGCGCGGTCGTGTTTACGCTGTGTCAATGTTCAACCCGCAAGGTAACG
ATATGACCAAAGGACTGCTTACGCTGGCGAAAGGTAAACCAATCGGTAAGGAAGG
TTACTACTGGCTGAAAATCCACGGTGCAAACTGTGCGGGTGTCGATAAGGTTCCG
TTCCCTGAGCGCATCAAGTTCATTGAGGAAAACCACGAGAACATCATGGCTTGCG
CTAAGTCTCCACTGGAGAACACTTGGTGGGCTGAGCAAGATTCTCCGTTCTGCTT
CCTTGCGTTCTGCTTTGAGTACGCTGGGGTACAGCACCACGGCCTGAGCTATAAC
TGCTCCCTTCCGCTGGCGTTTGACGGGTCTTGCTCTGGCATCCAGCACTTCTCCG
CGATGCTCCGAGATGAGGTAGGTGGTCGCGCGGTTAACTTGCTTCCTAGTGAAAC
CGTTCAGGACATCTACGGGATTGTTGCTAAGAAAGTCAACGAGATTCTACAAGCA
GACGCAATCAATGGGACCGATAACGAAGTAGTTACCGTGACCGATGAGAACACTG
GTGAAATCTCTGAGAAAGTCAAGCTGGGCACTAAGGCACTGGCTGGTCAATGGCT
GGCTTACGGTGTTACTCGCAGTGTGACTAAGCGTTCAGTCATGACGCTGGCTTAC
GGGTCCAAAGAGTTCGGCTTCCGTCAACAAGTGCTGGAAGATACCATTCAGCCAG
CTATTGATTCCGGCAAGGGTCTGATGTTCACTCAGCCGAATCAGGCTGCTGGATA
CATGGCTAAGCTGATTTGGGAATCTGTGAGCGTGACGGTGGTAGCTGCGGTTGAA
GCAATGAACTGGCTTAAGTCTGCTGCTAAGCTGCTGGCTGCTGAGGTCAAAGATA
AGAAGACTGGAGAGATTCTTCGCAAGCGTTGCGCTGTGCATTGGGTAACTCCTGA
TGGTTTCCCTGTGTGGCAGGAATACAAGAAGCCTATTCAGACGCGCTTGAACCTG
ATGTTCCTCGGTCAGTTCCGCTTACAGCCTACCATTAACACCAACAAAGATAGCG
AGATTGATGCACACAAACAGGAGTCTGGTATCGCTCCTAACTTTGTACACAGCCA
AGACGGTAGCCACCTTCGTAAGACTGTAGTGTGGGCACACGAGAAGTACGGAATC
GAATCTTTTGCACTGATTCACGACTCCTTCGGTACCATTCCGGCTGACGCTGCGA
ACCTGTTCAAAGCAGTGCGCGAAACTATGGTTGACACATATGAGTCTTGTGATGT
ACTGGCTGATTTCTACGACCAGTTCGCTGACCAGTTGCACGAGTCTCAATTGGAC
AAAATGCCAGCACTTCCGGCTAAAGGTAACTTGAACCTCCGTGACATCTTAGAGT
CGGACTTCGCGTTCGCGTAAAAGCTTGATGGGGGATCCCATGGTACGCGTGCTAG
T5 SEQ ID NO: 115:
TCAGAATTGGTTAATTGGTTGTAACACTGGTCTATCATTGATAGGTATAAATTAA
TACGACTCACTAATACTGAACCTATCAGTGATAGATCCAAACCCAAAAACACAGG
AGTTTTTAGAATGTCTAGATTAGATAAAAGTAAAGTGATTAACAGCGCATTAGAG
CTGCTTAATGAGGTCGGAATCGAAGGTTTAACAACCCGTAAACTCGCCCAGAAGC
TAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTA
GAAGGGGAAAGCTGGCAAGATTTTTTACGTAATAACGCTAAAAGTTTTAGATGTG
CTTTACTAAGTCATCGCGATGGAGCAAAAGTACATTTAGGTACACGGCCTACAGA
AAAACAGTATGAAACTCTCGAAAATCAATTAGCCTTTTTATGCCAACAAGGTTTT
TCACTAGAGAATGCATTATATGCACTCAGCGCTGTGGGGCATTTTACTTTAGGTT
GCGTATTGGAAGATCAAGAGCATCAAGTCGCTAAAGAAGAAAGGGAAACACCTAC
TACTGATAGTATGCCGCCATTATTACGACAAGCTATCGAATTATTTGATCACCAA
GGTGCAGAGCCAGCCTTCTTATTCGGCCTTGAATTGATCATATGCGGATTAGAAA
AACAACTTAAATGTGAAAGTGGGTCTTAAGCGGATCTTTACAGATTCTATACCGG
TGATACCAGCATCGTCTTGATGCCCTTGGCAGCACCCTGCTAAGGAGGCAACAAG
ATGAACACGATTAACATCGCTAAGAACGACTTCTCTGACATCGAACTGGCTGCTA
TCCCGTTCAACACTCTGGCTGACCATTACGGTGAGCGTTTAGCTCGCGAACAGTT
GGCCCTTGAGCATGAGTCTTACGAGATGGGTGAAGCACGCTTCCGCAAGATGTTT
GAGCGTCAACTTAAAGCTGGTGAGGTTGCGGATAACGCTGCCGCCAAGCCTCTCA
TCACTACCCTACTCCCTAAGATGATTGCACGCATCAACGACTGGTTTGAGGAAGT
GAAAGCTAAGCGCGGCAAGCGCCCGACAGCCTTCCAGTTCCTGCAAGAAATCAAG
CCGGAAGCCGTAGCGTACATCACCATTAAGACCACTCTGGCTTGCCTAACCAGTG
CTGACAATACAACCGTTCAGGCTGTAGCAAGCGCAATCGGTCGGGCCATTGAGGA
CGAGGCTCGCTTCGGTCGTATCCGTGACCTTGAAGCTAAGCACTTCAAGAAAAAC
GTTGAGGAACAACTCAACAAGCGCGTAGGGCACGTCTACAAGAAAGCATTTATGC
AAGTTGTCGAGGCTGACATGCTCTCTAAGGGTCTACTCGGTGGCGAGGCGTGGTC
TTCGTGGCATAAGGAAGACTCTATTCATGTAGGAGTACGCTGCATCGAGATGCTC
ATTGAGTCAACCGGAATGGTTAGCTTACACCGCCAAAATGCTGGCGTAGTAGGTC
AAGACTCTGAGACTATCGAACTCGCACCTGAATACGCTGAGGCTATCGCAACCCG
TGCAGGTGCGCTGGCTGGCATCTCTCCGATGTTCCAACCTTGCGTAGTTCCTCCT
AAGCCGTGGACTGGCATTACTGGTGGTGGCTATTGGGCTAACGGTCGTCGTCCTC
TGGCGCTGGTGCGTACTCACAGTAAGAAAGCACTGATGCGCTACGAAGACGTTTA
CATGCCTGAGGTGTACAAAGCGATTAACATTGCGCAAAACACCGCATGGAAAATC
AACAAGAAAGTCCTAGCGGTCGCCAACGTAATCACCAAGTGGAAGCATTGTCCGG
TCGAGGACATCCCTGCGATTGAGCGTGAAGAACTCCCGATGAAACCGGAAGACAT
CGACATGAATCCTGAGGCTCTCACCGCGTGGAAACGTGCTGCCGCTGCTGTGTAC
CGCAAGGACAAGGCTCGCAAGTCTCGCCGTATCAGCCTTGAGTTCATGCTTGAGC
AAGCCAATAAGTTTGCTAACCATAAGGCCATCTGGTTCCCTTACAACATGGACTG
GCGCGGTCGTGTTTACGCTGTGTCAATGTTCAACCCGCAAGGTAACGATATGACC
AAAGGACTGCTTACGCTGGCGAAAGGTAAACCAATCGGTAAGGAAGGTTACTACT
GGCTGAAAATCCACGGTGCAAACTGTGCGGGTGTCGATAAGGTTCCGTTCCCTGA
GCGCATCAAGTTCATTGAGGAAAACCACGAGAACATCATGGCTTGCGCTAAGTCT
CCACTGGAGAACACTTGGTGGGCTGAGCAAGATTCTCCGTTCTGCTTCCTTGCGT
TCTGCTTTGAGTACGCTGGGGTACAGCACCACGGCCTGAGCTATAACTGCTCCCT
TCCGCTGGCGTTTGACGGGTCTTGCTCTGGCATCCAGCACTTCTCCGCGATGCTC
CGAGATGAGGTAGGTGGTCGCGCGGTTAACTTGCTTCCTAGTGAAACCGTTCAGG
ACATCTACGGGATTGTTGCTAAGAAAGTCAACGAGATTCTACAAGCAGACGCAAT
CAATGGGACCGATAACGAAGTAGTTACCGTGACCGATGAGAACACTGGTGAAATC
TCTGAGAAAGTCAAGCTGGGCACTAAGGCACTGGCTGGTCAATGGCTGGCTTACG
GTGTTACTCGCAGTGTGACTAAGCGTTCAGTCATGACGCTGGCTTACGGGTCCAA
AGAGTTCGGCTTCCGTCAACAAGTGCTGGAAGATACCATTCAGCCAGCTATTGAT
TCCGGCAAGGGTCTGATGTTCACTCAGCCGAATCAGGCTGCTGGATACATGGCTA
AGCTGATTTGGGAATCTGTGAGCGTGACGGTGGTAGCTGCGGTTGAAGCAATGAA
CTGGCTTAAGTCTGCTGCTAAGCTGCTGGCTGCTGAGGTCAAAGATAAGAAGACT
GGAGAGATTCTTCGCAAGCGTTGCGCTGTGCATTGGGTAACTCCTGATGGTTTCC
CTGTGTGGCAGGAATACAAGAAGCCTATTCAGACGCGCTTGAACCTGATGTTCCT
CGGTCAGTTCCGCTTACAGCCTACCATTAACACCAACAAAGATAGCGAGATTGAT
GCACACAAACAGGAGTCTGGTATCGCTCCTAACTTTGTACACAGCCAAGACGGTA
GCCACCTTCGTAAGACTGTAGTGTGGGCACACGAGAAGTACGGAATCGAATCTTT
TGCACTGATTCACGACTCCTTCGGTACCATTCCGGCTGACGCTGCGAACCTGTTC
AAAGCAGTGCGCGAAACTATGGTTGACACATATGAGTCTTGTGATGTACTGGCTG
ATTTCTACGACCAGTTCGCTGACCAGTTGCACGAGTCTCAATTGGACAAAATGCC
AGCACTTCCGGCTAAAGGTAACTTGAACCTCCGTGACATCTTAGAGTCGGACTTC
GCGTTCGCGTAAAAGCTTGATGGGGGATCCCATGGTACGCGTGCTAG
T6 SEQ ID NO: 116:
TCAGAATTGGTTAATTGGTTGTAACACTGGTCTATCATTGATAGGTATAAATTAA
TACGACTCACTATAGGGAGACCTATCAGTGATAGATCCAAACCCAAAAACACAGG
AGTTTTTAGAATGTCTAGATTAGATAAAAGTAAAGTGATTAACAGCGCATTAGAG
CTGCTTAATGAGGTCGGAATCGAAGGTTTAACAACCCGTAAACTCGCCCAGAAGC
TAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTA
GAAGGGGAAAGCTGGCAAGATTTTTTACGTAATAACGCTAAAAGTTTTAGATGTG
CTTTACTAAGTCATCGCGATGGAGCAAAAGTACATTTAGGTACACGGCCTACAGA
AAAACAGTATGAAACTCTCGAAAATCAATTAGCCTTTTTATGCCAACAAGGTTTT
TCACTAGAGAATGCATTATATGCACTCAGCGCTGTGGGGCATTTTACTTTAGGTT
GCGTATTGGAAGATCAAGAGCATCAAGTCGCTAAAGAAGAAAGGGAAACACCTAC
TACTGATAGTATGCCGCCATTATTACGACAAGCTATCGAATTATTTGATCACCAA
GGTGCAGAGCCAGCCTTCTTATTCGGCCTTGAATTGATCATATGCGGATTAGAAA
AACAACTTAAATGTGAAAGTGGGTCTTAAGCGGATCTTTACAGATTCTATACCGG
TGATACCAGCATCGTCTTGATGCCCTTGGCAGCACCCTGCTAAGGAGGCAACAAG
ATGAACACGATTAACATCGCTAAGAACGACTTCTCTGACATCGAACTGGCTGCTA
TCCCGTTCAACACTCTGGCTGACCATTACGGTGAGCGTTTAGCTCGCGAACAGTT
GGCCCTTGAGCATGAGTCTTACGAGATGGGTGAAGCACGCTTCCGCAAGATGTTT
GAGCGTCAACTTAAAGCTGGTGAGGTTGCGGATAACGCTGCCGCCAAGCCTCTCA
TCACTACCCTACTCCCTAAGATGATTGCACGCATCAACGACTGGTTTGAGGAAGT
GAAAGCTAAGCGCGGCAAGCGCCCGACAGCCTTCCAGTTCCTGCAAGAAATCAAG
CCGGAAGCCGTAGCGTACATCACCATTAAGACCACTCTGGCTTGCCTAACCAGTG
CTGACAATACAACCGTTCAGGCTGTAGCAAGCGCAATCGGTCGGGCCATTGAGGA
CGAGGCTCGCTTCGGTCGTATCCGTGACCTTGAAGCTAAGCACTTCAAGAAAAAC
GTTGAGGAACAACTCAACAAGCGCGTAGGGCACGTCTACAAGAAAGCATTTATGC
AAGTTGTCGAGGCTGACATGCTCTCTAAGGGTCTACTCGGTGGCGAGGCGTGGTC
TTCGTGGCATAAGGAAGACTCTATTCATGTAGGAGTACGCTGCATCGAGATGCTC
ATTGAGTCAACCGGAATGGTTAGCTTACACCGCCAAAATGCTGGCGTAGTAGGTC
AAGACTCTGAGACTATCGAACTCGCACCTGAATACGCTGAGGCTATCGCAACCCG
TGCAGGTGCGCTGGCTGGCATCTCTCCGATGTTCCAACCTTGCGTAGTTCCTCCT
AAGCCGTGGACTGGCATTACTGGTGGTGGCTATTGGGCTAACGGTCGTCGTCCTC
TGGCGCTGGTGCGTACTCACAGTAAGAAAGCACTGATGCGCTACGAAGACGTTTA
CATGCCTGAGGTGTACAAAGCGATTAACATTGCGCAAAACACCGCATGGAAAATC
AACAAGAAAGTCCTAGCGGTCGCCAACGTAATCACCAAGTGGAAGCATTGTCCGG
TCGAGGACATCCCTGCGATTGAGCGTGAAGAACTCCCGATGAAACCGGAAGACAT
CGACATGAATCCTGAGGCTCTCACCGCGTGGAAACGTGCTGCCGCTGCTGTGTAC
CGCAAGGACAAGGCTCGCAAGTCTCGCCGTATCAGCCTTGAGTTCATGCTTGAGC
AAGCCAATAAGTTTGCTAACCATAAGGCCATCTGGTTCCCTTACAACATGGACTG
GCGCGGTCGTGTTTACGCTGTGTCAATGTTCAACCCGCAAGGTAACGATATGACC
AAAGGACTGCTTACGCTGGCGAAAGGTAAACCAATCGGTAAGGAAGGTTACTACT
GGCTGAAAATCCACGGTGCAAACTGTGCGGGTGTCGATAAGGTTCCGTTCCCTGA
GCGCATCAAGTTCATTGAGGAAAACCACGAGAACATCATGGCTTGCGCTAAGTCT
CCACTGGAGAACACTTGGTGGGCTGAGCAAGATTCTCCGTTCTGCTTCCTTGCGT
TCTGCTTTGAGTACGCTGGGGTACAGCACCACGGCCTGAGCTATAACTGCTCCCT
TCCGCTGGCGTTTGACGGGTCTTGCTCTGGCATCCAGCACTTCTCCGCGATGCTC
CGAGATGAGGTAGGTGGTCGCGCGGTTAACTTGCTTCCTAGTGAAACCGTTCAGG
ACATCTACGGGATTGTTGCTAAGAAAGTCAACGAGATTCTACAAGCAGACGCAAT
CAATGGGACCGATAACGAAGTAGTTACCGTGACCGATGAGAACACTGGTGAAATC
TCTGAGAAAGTCAAGCTGGGCACTAAGGCACTGGCTGGTCAATGGCTGGCTTACG
GTGTTACTCGCAGTGTGACTAAGCGTTCAGTCATGACGCTGGCTTACGGGTCCAA
AGAGTTCGGCTTCCGTCAACAAGTGCTGGAAGATACCATTCAGCCAGCTATTGAT
TCCGGCAAGGGTCTGATGTTCACTCAGCCGAATCAGGCTGCTGGATACATGGCTA
AGCTGATTTGGGAATCTGTGAGCGTGACGGTGGTAGCTGCGGTTGAAGCAATGAA
CTGGCTTAAGTCTGCTGCTAAGCTGCTGGCTGCTGAGGTCAAAGATAAGAAGACT
GGAGAGATTCTTCGCAAGCGTTGCGCTGTGCATTGGGTAACTCCTGATGGTTTCC
CTGTGTGGCAGGAATACAAGAAGCCTATTCAGACGCGCTTGAACCTGATGTTCCT
CGGTCAGTTCCGCTTACAGCCTACCATTAACACCAACAAAGATAGCGAGATTGAT
GCACACAAACAGGAGTCTGGTATCGCTCCTAACTTTGTACACAGCCAAGACGGTA
GCCACCTTCGTAAGACTGTAGTGTGGGCACACGAGAAGTACGGAATCGAATCTTT
TGCACTGATTCACGACTCCTTCGGTACCATTCCGGCTGACGCTGCGAACCTGTTC
AAAGCAGTGCGCGAAACTATGGTTGACACATATGAGTCTTGTGATGTACTGGCTG
ATTTCTACGACCAGTTCGCTGACCAGTTGCACGAGTCTCAATTGGACAAAATGCC
AGCACTTCCGGCTAAAGGTAACTTGAACCTCCGTGACATCTTAGAGTCGGACTTC
GCGTTCGCGTAAAAGCTTGATGGGGGATCCCATGGTACGCGTGCTAG
T7 SEQ ID NO: 117:
TCAGAATTGGTTAATTGGTTGTAACACTGGTCTATCATTGATAGGTATAAATTAA
TACGACTCACTATAGGGAGACCTATCAGTGATAGATCCAAACCCAAAAACACAGG
AGTTTTTAGAATGTCTAGATTAGATAAAAGTAAAGTGATTAACAGCGCATTAGAG
CTGCTTAATGAGGTCGGAATCGAAGGTTTAACAACCCGTAAACTCGCCCAGAAGC
TAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTA
GAAGGGGAAAGCTGGCAAGATTTTTTACGTAATAACGCTAAAAGTTTTAGATGTG
CTTTACTAAGTCATCGCGATGGAGCAAAAGTACATTTAGGTACACGGCCTACAGA
AAAACAGTATGAAACTCTCGAAAATCAATTAGCCTTTTTATGCCAACAAGGTTTT
TCACTAGAGAATGCATTATATGCACTCAGCGCTGTGGGGCATTTTACTTTAGGTT
GCGTATTGGAAGATCAAGAGCATCAAGTCGCTAAAGAAGAAAGGGAAACACCTAC
TACTGATAGTATGCCGCCATTATTACGACAAGCTATCGAATTATTTGATCACCAA
GGTGCAGAGCCAGCCTTCTTATTCGGCCTTGAATTGATCATATGCGGATTAGAAA
AACAACTTAAATGTGAAAGTGGGTCTTAAaagtgataccagcatcgtcttgatgc
ccttggcagcacttcatttacatactcggtaaactgaagtgctgccatttttttt
GGTACCGGTGATACCAGCATCGTCTTGATGCCCTTGGCAGCACCCTGCTAAGGAG
GCAACAAGATGAACACGATTAACATCGCTAAGAACGACTTCTCTGACATCGAACT
GGCTGCTATCCCGTTCAACACTCTGGCTGACCATTACGGTGAGCGTTTAGCTCGC
GAACAGTTGGCCCTTGAGCATGAGTCTTACGAGATGGGTGAAGCACGCTTCCGCA
AGATGTTTGAGCGTCAACTTAAAGCTGGTGAGGTTGCGGATAACGCTGCCGCCAA
GCCTCTCATCACTACCCTACTCCCTAAGATGATTGCACGCATCAACGACTGGTTT
GAGGAAGTGAAAGCTAAGCGCGGCAAGCGCCCGACAGCCTTCCAGTTCCTGCAAG
AAATCAAGCCGGAAGCCGTAGCGTACATCACCATTAAGACCACTCTGGCTTGCCT
AACCAGTGCTGACAATACAACCGTTCAGGCTGTAGCAAGCGCAATCGGTCGGGCC
ATTGAGGACGAGGCTCGCTTCGGTCGTATCCGTGACCTTGAAGCTAAGCACTTCA
AGAAAAACGTTGAGGAACAACTCAACAAGCGCGTAGGGCACGTCTACAAGAAAGC
ATTTATGCAAGTTGTCGAGGCTGACATGCTCTCTAAGGGTCTACTCGGTGGCGAG
GCGTGGTCTTCGTGGCATAAGGAAGACTCTATTCATGTAGGAGTACGCTGCATCG
AGATGCTCATTGAGTCAACCGGAATGGTTAGCTTACACCGCCAAAATGCTGGCGT
AGTAGGTCAAGACTCTGAGACTATCGAACTCGCACCTGAATACGCTGAGGCTATC
GCAACCCGTGCAGGTGCGCTGGCTGGCATCTCTCCGATGTTCCAACCTTGCGTAG
TTCCTCCTAAGCCGTGGACTGGCATTACTGGTGGTGGCTATTGGGCTAACGGTCG
TCGTCCTCTGGCGCTGGTGCGTACTCACAGTAAGAAAGCACTGATGCGCTACGAA
GACGTTTACATGCCTGAGGTGTACAAAGCGATTAACATTGCGCAAAACACCGCAT
GGAAAATCAACAAGAAAGTCCTAGCGGTCGCCAACGTAATCACCAAGTGGAAGCA
TTGTCCGGTCGAGGACATCCCTGCGATTGAGCGTGAAGAACTCCCGATGAAACCG
GAAGACATCGACATGAATCCTGAGGCTCTCACCGCGTGGAAACGTGCTGCCGCTG
CTGTGTACCGCAAGGACAAGGCTCGCAAGTCTCGCCGTATCAGCCTTGAGTTCAT
GCTTGAGCAAGCCAATAAGTTTGCTAACCATAAGGCCATCTGGTTCCCTTACAAC
ATGGACTGGCGCGGTCGTGTTTACGCTGTGTCAATGTTCAACCCGCAAGGTAACG
ATATGACCAAAGGACTGCTTACGCTGGCGAAAGGTAAACCAATCGGTAAGGAAGG
TTACTACTGGCTGAAAATCCACGGTGCAAACTGTGCGGGTGTCGATAAGGTTCCG
TTCCCTGAGCGCATCAAGTTCATTGAGGAAAACCACGAGAACATCATGGCTTGCG
CTAAGTCTCCACTGGAGAACACTTGGTGGGCTGAGCAAGATTCTCCGTTCTGCTT
CCTTGCGTTCTGCTTTGAGTACGCTGGGGTACAGCACCACGGCCTGAGCTATAAC
TGCTCCCTTCCGCTGGCGTTTGACGGGTCTTGCTCTGGCATCCAGCACTTCTCCG
CGATGCTCCGAGATGAGGTAGGTGGTCGCGCGGTTAACTTGCTTCCTAGTGAAAC
CGTTCAGGACATCTACGGGATTGTTGCTAAGAAAGTCAACGAGATTCTACAAGCA
GACGCAATCAATGGGACCGATAACGAAGTAGTTACCGTGACCGATGAGAACACTG
GTGAAATCTCTGAGAAAGTCAAGCTGGGCACTAAGGCACTGGCTGGTCAATGGCT
GGCTTACGGTGTTACTCGCAGTGTGACTAAGCGTTCAGTCATGACGCTGGCTTAC
GGGTCCAAAGAGTTCGGCTTCCGTCAACAAGTGCTGGAAGATACCATTCAGCCAG
CTATTGATTCCGGCAAGGGTCTGATGTTCACTCAGCCGAATCAGGCTGCTGGATA
CATGGCTAAGCTGATTTGGGAATCTGTGAGCGTGACGGTGGTAGCTGCGGTTGAA
GCAATGAACTGGCTTAAGTCTGCTGCTAAGCTGCTGGCTGCTGAGGTCAAAGATA
AGAAGACTGGAGAGATTCTTCGCAAGCGTTGCGCTGTGCATTGGGTAACTCCTGA
TGGTTTCCCTGTGTGGCAGGAATACAAGAAGCCTATTCAGACGCGCTTGAACCTG
ATGTTCCTCGGTCAGTTCCGCTTACAGCCTACCATTAACACCAACAAAGATAGCG
AGATTGATGCACACAAACAGGAGTCTGGTATCGCTCCTAACTTTGTACACAGCCA
AGACGGTAGCCACCTTCGTAAGACTGTAGTGTGGGCACACGAGAAGTACGGAATC
GAATCTTTTGCACTGATTCACGACTCCTTCGGTACCATTCCGGCTGACGCTGCGA
ACCTGTTCAAAGCAGTGCGCGAAACTATGGTTGACACATATGAGTCTTGTGATGT
ACTGGCTGATTTCTACGACCAGTTCGCTGACCAGTTGCACGAGTCTCAATTGGAC
AAAATGCCAGCACTTCCGGCTAAAGGTAACTTGAACCTCCGTGACATCTTAGAGT
CGGACTTCGCGTTCGCGTAAAAGCTTGATGGGGGATCCCATGGTACGCGTGCTAG
T8 SEQ ID NO: 118:
TCAGAATTGGTTAATTGGTTGTAACACTGGTAATACGACTCACTAATACTGAATA
CCGGTGATACCAGCATCGTCTTGATGCCCTTGGCAGCACCCTGCTAAGGAGGCAA
CAAGATGAACACGATTAACATCGCTAAGAACGACTTCTCTGACATCGAACTGGCT
GCTATCCCGTTCAACACTCTGGCTGACCATTACGGTGAGCGTTTAGCTCGCGAAC
AGTTGGCCCTTGAGCATGAGTCTTACGAGATGGGTGAAGCACGCTTCCGCAAGAT
GTTTGAGCGTCAACTTAAAGCTGGTGAGGTTGCGGATAACGCTGCCGCCAAGCCT
CTCATCACTACCCTACTCCCTAAGATGATTGCACGCATCAACGACTGGTTTGAGG
AAGTGAAAGCTAAGCGCGGCAAGCGCCCGACAGCCTTCCAGTTCCTGCAAGAAAT
CAAGCCGGAAGCCGTAGCGTACATCACCATTAAGACCACTCTGGCTTGCCTAACC
AGTGCTGACAATACAACCGTTCAGGCTGTAGCAAGCGCAATCGGTCGGGCCATTG
AGGACGAGGCTCGCTTCGGTCGTATCCGTGACCTTGAAGCTAAGCACTTCAAGAA
AAACGTTGAGGAACAACTCAACAAGCGCGTAGGGCACGTCTACAAGAAAGCATTT
ATGCAAGTTGTCGAGGCTGACATGCTCTCTAAGGGTCTACTCGGTGGCGAGGCGT
GGTCTTCGTGGCATAAGGAAGACTCTATTCATGTAGGAGTACGCTGCATCGAGAT
GCTCATTGAGTCAACCGGAATGGTTAGCTTACACCGCCAAAATGCTGGCGTAGTA
GGTCAAGACTCTGAGACTATCGAACTCGCACCTGAATACGCTGAGGCTATCGCAA
CCCGTGCAGGTGCGCTGGCTGGCATCTCTCCGATGTTCCAACCTTGCGTAGTTCC
TCCTAAGCCGTGGACTGGCATTACTGGTGGTGGCTATTGGGCTAACGGTCGTCGT
CCTCTGGCGCTGGTGCGTACTCACAGTAAGAAAGCACTGATGCGCTACGAAGACG
TTTACATGCCTGAGGTGTACAAAGCGATTAACATTGCGCAAAACACCGCATGGAA
AATCAACAAGAAAGTCCTAGCGGTCGCCAACGTAATCACCAAGTGGAAGCATTGT
CCGGTCGAGGACATCCCTGCGATTGAGCGTGAAGAACTCCCGATGAAACCGGAAG
ACATCGACATGAATCCTGAGGCTCTCACCGCGTGGAAACGTGCTGCCGCTGCTGT
GTACCGCAAGGACAAGGCTCGCAAGTCTCGCCGTATCAGCCTTGAGTTCATGCTT
GAGCAAGCCAATAAGTTTGCTAACCATAAGGCCATCTGGTTCCCTTACAACATGG
ACTGGCGCGGTCGTGTTTACGCTGTGTCAATGTTCAACCCGCAAGGTAACGATAT
GACCAAAGGACTGCTTACGCTGGCGAAAGGTAAACCAATCGGTAAGGAAGGTTAC
TACTGGCTGAAAATCCACGGTGCAAACTGTGCGGGTGTCGATAAGGTTCCGTTCC
CTGAGCGCATCAAGTTCATTGAGGAAAACCACGAGAACATCATGGCTTGCGCTAA
GTCTCCACTGGAGAACACTTGGTGGGCTGAGCAAGATTCTCCGTTCTGCTTCCTT
GCGTTCTGCTTTGAGTACGCTGGGGTACAGCACCACGGCCTGAGCTATAACTGCT
CCCTTCCGCTGGCGTTTGACGGGTCTTGCTCTGGCATCCAGCACTTCTCCGCGAT
GCTCCGAGATGAGGTAGGTGGTCGCGCGGTTAACTTGCTTCCTAGTGAAACCGTT
CAGGACATCTACGGGATTGTTGCTAAGAAAGTCAACGAGATTCTACAAGCAGACG
CAATCAATGGGACCGATAACGAAGTAGTTACCGTGACCGATGAGAACACTGGTGA
AATCTCTGAGAAAGTCAAGCTGGGCACTAAGGCACTGGCTGGTCAATGGCTGGCT
TACGGTGTTACTCGCAGTGTGACTAAGCGTTCAGTCATGACGCTGGCTTACGGGT
CCAAAGAGTTCGGCTTCCGTCAACAAGTGCTGGAAGATACCATTCAGCCAGCTAT
TGATTCCGGCAAGGGTCTGATGTTCACTCAGCCGAATCAGGCTGCTGGATACATG
GCTAAGCTGATTTGGGAATCTGTGAGCGTGACGGTGGTAGCTGCGGTTGAAGCAA
TGAACTGGCTTAAGTCTGCTGCTAAGCTGCTGGCTGCTGAGGTCAAAGATAAGAA
GACTGGAGAGATTCTTCGCAAGCGTTGCGCTGTGCATTGGGTAACTCCTGATGGT
TTCCCTGTGTGGCAGGAATACAAGAAGCCTATTCAGACGCGCTTGAACCTGATGT
TCCTCGGTCAGTTCCGCTTACAGCCTACCATTAACACCAACAAAGATAGCGAGAT
TGATGCACACAAACAGGAGTCTGGTATCGCTCCTAACTTTGTACACAGCCAAGAC
GGTAGCCACCTTCGTAAGACTGTAGTGTGGGCACACGAGAAGTACGGAATCGAAT
CTTTTGCACTGATTCACGACTCCTTCGGTACCATTCCGGCTGACGCTGCGAACCT
GTTCAAAGCAGTGCGCGAAACTATGGTTGACACATATGAGTCTTGTGATGTACTG
GCTGATTTCTACGACCAGTTCGCTGACCAGTTGCACGAGTCTCAATTGGACAAAA
TGCCAGCACTTCCGGCTAAAGGTAACTTGAACCTCCGTGACATCTTAGAGTCGGA
CTTCGCGTTCGCGTAA
T9 SEQ ID NO: 119:
TCAGAATTGGTTAATTGGTTGTAACACTGGTAATACGACTCACTATAGGGAGATA
CCGGTGATACCAGCATCGTCTTGATGCCCTTGGCAGCACCCTGCTAAGGAGGCAA
CAAGATGAACACGATTAACATCGCTAAGAACGACTTCTCTGACATCGAACTGGCT
GCTATCCCGTTCAACACTCTGGCTGACCATTACGGTGAGCGTTTAGCTCGCGAAC
AGTTGGCCCTTGAGCATGAGTCTTACGAGATGGGTGAAGCACGCTTCCGCAAGAT
GTTTGAGCGTCAACTTAAAGCTGGTGAGGTTGCGGATAACGCTGCCGCCAAGCCT
CTCATCACTACCCTACTCCCTAAGATGATTGCACGCATCAACGACTGGTTTGAGG
AAGTGAAAGCTAAGCGCGGCAAGCGCCCGACAGCCTTCCAGTTCCTGCAAGAAAT
CAAGCCGGAAGCCGTAGCGTACATCACCATTAAGACCACTCTGGCTTGCCTAACC
AGTGCTGACAATACAACCGTTCAGGCTGTAGCAAGCGCAATCGGTCGGGCCATTG
AGGACGAGGCTCGCTTCGGTCGTATCCGTGACCTTGAAGCTAAGCACTTCAAGAA
AAACGTTGAGGAACAACTCAACAAGCGCGTAGGGCACGTCTACAAGAAAGCATTT
ATGCAAGTTGTCGAGGCTGACATGCTCTCTAAGGGTCTACTCGGTGGCGAGGCGT
GGTCTTCGTGGCATAAGGAAGACTCTATTCATGTAGGAGTACGCTGCATCGAGAT
GCTCATTGAGTCAACCGGAATGGTTAGCTTACACCGCCAAAATGCTGGCGTAGTA
GGTCAAGACTCTGAGACTATCGAACTCGCACCTGAATACGCTGAGGCTATCGCAA
CCCGTGCAGGTGCGCTGGCTGGCATCTCTCCGATGTTCCAACCTTGCGTAGTTCC
TCCTAAGCCGTGGACTGGCATTACTGGTGGTGGCTATTGGGCTAACGGTCGTCGT
CCTCTGGCGCTGGTGCGTACTCACAGTAAGAAAGCACTGATGCGCTACGAAGACG
TTTACATGCCTGAGGTGTACAAAGCGATTAACATTGCGCAAAACACCGCATGGAA
AATCAACAAGAAAGTCCTAGCGGTCGCCAACGTAATCACCAAGTGGAAGCATTGT
CCGGTCGAGGACATCCCTGCGATTGAGCGTGAAGAACTCCCGATGAAACCGGAAG
ACATCGACATGAATCCTGAGGCTCTCACCGCGTGGAAACGTGCTGCCGCTGCTGT
GTACCGCAAGGACAAGGCTCGCAAGTCTCGCCGTATCAGCCTTGAGTTCATGCTT
GAGCAAGCCAATAAGTTTGCTAACCATAAGGCCATCTGGTTCCCTTACAACATGG
ACTGGCGCGGTCGTGTTTACGCTGTGTCAATGTTCAACCCGCAAGGTAACGATAT
GACCAAAGGACTGCTTACGCTGGCGAAAGGTAAACCAATCGGTAAGGAAGGTTAC
TACTGGCTGAAAATCCACGGTGCAAACTGTGCGGGTGTCGATAAGGTTCCGTTCC
CTGAGCGCATCAAGTTCATTGAGGAAAACCACGAGAACATCATGGCTTGCGCTAA
GTCTCCACTGGAGAACACTTGGTGGGCTGAGCAAGATTCTCCGTTCTGCTTCCTT
GCGTTCTGCTTTGAGTACGCTGGGGTACAGCACCACGGCCTGAGCTATAACTGCT
CCCTTCCGCTGGCGTTTGACGGGTCTTGCTCTGGCATCCAGCACTTCTCCGCGAT
GCTCCGAGATGAGGTAGGTGGTCGCGCGGTTAACTTGCTTCCTAGTGAAACCGTT
CAGGACATCTACGGGATTGTTGCTAAGAAAGTCAACGAGATTCTACAAGCAGACG
CAATCAATGGGACCGATAACGAAGTAGTTACCGTGACCGATGAGAACACTGGTGA
AATCTCTGAGAAAGTCAAGCTGGGCACTAAGGCACTGGCTGGTCAATGGCTGGCT
TACGGTGTTACTCGCAGTGTGACTAAGCGTTCAGTCATGACGCTGGCTTACGGGT
CCAAAGAGTTCGGCTTCCGTCAACAAGTGCTGGAAGATACCATTCAGCCAGCTAT
TGATTCCGGCAAGGGTCTGATGTTCACTCAGCCGAATCAGGCTGCTGGATACATG
GCTAAGCTGATTTGGGAATCTGTGAGCGTGACGGTGGTAGCTGCGGTTGAAGCAA
TGAACTGGCTTAAGTCTGCTGCTAAGCTGCTGGCTGCTGAGGTCAAAGATAAGAA
GACTGGAGAGATTCTTCGCAAGCGTTGCGCTGTGCATTGGGTAACTCCTGATGGT
TTCCCTGTGTGGCAGGAATACAAGAAGCCTATTCAGACGCGCTTGAACCTGATGT
TCCTCGGTCAGTTCCGCTTACAGCCTACCATTAACACCAACAAAGATAGCGAGAT
TGATGCACACAAACAGGAGTCTGGTATCGCTCCTAACTTTGTACACAGCCAAGAC
GGTAGCCACCTTCGTAAGACTGTAGTGTGGGCACACGAGAAGTACGGAATCGAAT
CTTTTGCACTGATTCACGACTCCTTCGGTACCATTCCGGCTGACGCTGCGAACCT
GTTCAAAGCAGTGCGCGAAACTATGGTTGACACATATGAGTCTTGTGATGTACTG
GCTGATTTCTACGACCAGTTCGCTGACCAGTTGCACGAGTCTCAATTGGACAAAA
TGCCAGCACTTCCGGCTAAAGGTAACTTGAACCTCCGTGACATCTTAGAGTCGGA
CTTCGCGTTCGCGTAA
T10 SEQ ID NO: 136:
TCAGAATTGGTTAATTGGTTGTAACACTGGTCTATCATTGATAGGTATAAATTAA
TACGACTCACTAATACTGAACCTATCAGTGATAGATCCAAACCCAAAAACACAGG
AGTTTTTAGAATGTCTAGATTAGATAAAAGTAAAGTGATTAACAGCGCATTAGAG
CTGCTTAATGAGGTCGGAATCGAAGGTTTAACAACCCGTAAACTCGCCCAGAAGC
TAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTA
GAAGGGGAAAGCTGGCAAGATTTTTTACGTAATAACGCTAAAAGTTTTAGATGTG
CTTTACTAAGTCATCGCGATGGAGCAAAAGTACATTTAGGTACACGGCCTACAGA
AAAACAGTATGAAACTCTCGAAAATCAATTAGCCTTTTTATGCCAACAAGGTTTT
TCACTAGAGAATGCATTATATGCACTCAGCGCTGTGGGGCATTTTACTTTAGGTT
GCGTATTGGAAGATCAAGAGCATCAAGTCGCTAAAGAAGAAAGGGAAACACCTAC
TACTGATAGTATGCCGCCATTATTACGACAAGCTATCGAATTATTTGATCACCAA
GGTGCAGAGCCAGCCTTCTTATTCGGCCTTGAATTGATCATATGCGGATTAGAAA
AACAACTTAAATGTGAAAGTGGGTCTTAAGCGGATCTTTACAGATTCTAGACGGG
ACTCTCACCAGGTACCGGAGATACCAGCATCGTCTTGATGCCCTTGGCAGCTCCA
GCTGCTAAGGAGGTATCAAGATGAACACGATTAACATCGCTAAGAACGACTTCTC
TGACATCGAACTGGCTGCTATCCCGTTCAACACTCTGGCTGACCATTACGGTGAG
CGTTTAGCTCGCGAACAGTTGGCCCTTGAGCATGAGTCTTACGAGATGGGTGAAG
CACGCTTCCGCAAGATGTTTGAGCGTCAACTTAAAGCTGGTGAGGTTGCGGATAA
CGCTGCCGCCAAGCCTCTCATCACTACCCTACTCCCTAAGATGATTGCACGCATC
AACGACTGGTTTGAGGAAGTGAAAGCTAAGCGCGGCAAGCGCCCGACAGCCTTCC
AGTTCCTGCAAGAAATCAAGCCGGAAGCCGTAGCGTACATCACCATTAAGACCAC
TCTGGCTTGCCTAACCAGTGCTGACAATACAACCGTTCAGGCTGTAGCAAGCGCA
ATCGGTCGGGCCATTGAGGACGAGGCTCGCTTCGGTCGTATCCGTGACCTTGAAG
CTAAGCACTTCAAGAAAAACGTTGAGGAACAACTCAACAAGCGCGTAGGGCACGT
CTACAAGAAAGCATTTATGCAAGTTGTCGAGGCTGACATGCTCTCTAAGGGTCTA
CTCGGTGGCGAGGCGTGGTCTTCGTGGCATAAGGAAGACTCTATTCATGTAGGAG
TACGCTGCATCGAGATGCTCATTGAGTCAACCGGAATGGTTAGCTTACACCGCCA
AAATGCTGGCGTAGTAGGTCAAGACTCTGAGACTATCGAACTCGCACCTGAATAC
GCTGAGGCTATCGCAACCCGTGCAGGTGCGCTGGCTGGCATCTCTCCGATGTTCC
AACCTTGCGTAGTTCCTCCTAAGCCGTGGACTGGCATTACTGGTGGTGGCTATTG
GGCTAACGGTCGTCGTCCTCTGGCGCTGGTGCGTACTCACAGTAAGAAAGCACTG
ATGCGCTACGAAGACGTTTACATGCCTGAGGTGTACAAAGCGATTAACATTGCGC
AAAACACCGCATGGAAAATCAACAAGAAAGTCCTAGCGGTCGCCAACGTAATCAC
CAAGTGGAAGCATTGTCCGGTCGAGGACATCCCTGCGATTGAGCGTGAAGAACTC
CCGATGAAACCGGAAGACATCGACATGAATCCTGAGGCTCTCACCGCGTGGAAAC
GTGCTGCCGCTGCTGTGTACCGCAAGGACAAGGCTCGCAAGTCTCGCCGTATCAG
CCTTGAGTTCATGCTTGAGCAAGCCAATAAGTTTGCTAACCATAAGGCCATCTGG
TTCCCTTACAACATGGACTGGCGCGGTCGTGTTTACGCTGTGTCAATGTTCAACC
CGCAAGGTAACGATATGACCAAAGGACTGCTTACGCTGGCGAAAGGTAAACCAAT
CGGTAAGGAAGGTTACTACTGGCTGAAAATCCACGGTGCAAACTGTGCGGGTGTC
GATAAGGTTCCGTTCCCTGAGCGCATCAAGTTCATTGAGGAAAACCACGAGAACA
TCATGGCTTGCGCTAAGTCTCCACTGGAGAACACTTGGTGGGCTGAGCAAGATTC
TCCGTTCTGCTTCCTTGCGTTCTGCTTTGAGTACGCTGGGGTACAGCACCACGGC
CTGAGCTATAACTGCTCCCTTCCGCTGGCGTTTGACGGGTCTTGCTCTGGCATCC
AGCACTTCTCCGCGATGCTCCGAGATGAGGTAGGTGGTCGCGCGGTTAACTTGCT
TCCTAGTGAAACCGTTCAGGACATCTACGGGATTGTTGCTAAGAAAGTCAACGAG
ATTCTACAAGCAGACGCAATCAATGGGACCGATAACGAAGTAGTTACCGTGACCG
ATGAGAACACTGGTGAAATCTCTGAGAAAGTCAAGCTGGGCACTAAGGCACTGGC
TGGTCAATGGCTGGCTTACGGTGTTACTCGCAGTGTGACTAAGCGTTCAGTCATG
ACGCTGGCTTACGGGTCCAAAGAGTTCGGCTTCCGTCAACAAGTGCTGGAAGATA
CCATTCAGCCAGCTATTGATTCCGGCAAGGGTCTGATGTTCACTCAGCCGAATCA
GGCTGCTGGATACATGGCTAAGCTGATTTGGGAATCTGTGAGCGTGACGGTGGTA
GCTGCGGTTGAAGCAATGAACTGGCTTAAGTCTGCTGCTAAGCTGCTGGCTGCTG
AGGTCAAAGATAAGAAGACTGGAGAGATTCTTCGCAAGCGTTGCGCTGTGCATTG
GGTAACTCCTGATGGTTTCCCTGTGTGGCAGGAATACAAGAAGCCTATTCAGACG
CGCTTGAACCTGATGTTCCTCGGTCAGTTCCGCTTACAGCCTACCATTAACACCA
ACAAAGATAGCGAGATTGATGCACACAAACAGGAGTCTGGTATCGCTCCTAACTT
TGTACACAGCCAAGACGGTAGCCACCTTCGTAAGACTGTAGTGTGGGCACACGAG
AAGTACGGAATCGAATCTTTTGCACTGATTCACGACTCCTTCGGTACCATTCCGG
CTGACGCTGCGAACCTGTTCAAAGCAGTGCGCGAAACTATGGTTGACACATATGA
GTCTTGTGATGTACTGGCTGATTTCTACGACCAGTTCGCTGACCAGTTGCACGAG
TCTCAATTGGACAAAATGCCAGCACTTCCGGCTAAAGGTAACTTGAACCTCCGTG
ACATCTTAGAGTCGGACTTCGCGTTCGCGTAAAAGCTTGATGGGGGATCCCATGG
TACGCGTGCTAG
T11 SEQ ID NO: 120:
TCAGAATTGGTTAATTGGTTGTAACACTGGTCTATCATTGATAGGTATAAATTAA
TACGACTCACTAATACTGAACCTATCAGTGATAGATCCAAACCCAAAAACACAGG
AGTTTTTAGAATGTCTAGATTAGATAAAAGTAAAGTGATTAACAGCGCATTAGAG
CTGCTTAATGAGGTCGGAATCGAAGGTTTAACAACCCGTAAACTCGCCCAGAAGC
TAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTA
GAAGGGGAAAGCTGGCAAGATTTTTTACGTAATAACGCTAAAAGTTTTAGATGTG
CTTTACTAAGTCATCGCGATGGAGCAAAAGTACATTTAGGTACACGGCCTACAGA
AAAACAGTATGAAACTCTCGAAAATCAATTAGCCTTTTTATGCCAACAAGGTTTT
TCACTAGAGAATGCATTATATGCACTCAGCGCTGTGGGGCATTTTACTTTAGGTT
GCGTATTGGAAGATCAAGAGCATCAAGTCGCTAAAGAAGAAAGGGAAACACCTAC
TACTGATAGTATGCCGCCATTATTACGACAAGCTATCGAATTATTTGATCACCAA
GGTGCAGAGCCAGCCTTCTTATTCGGCCTTGAATTGATCATATGCGGATTAGAAA
AACAACTTAAATGTGAAAGTGGGTCTTAAGCGGATCTTTACAGATTCTAGACGGG
ACTCTCACCAGGTACCGGAGATACCAGCATCGTCTTGATGCCCTTGGCAGCTCCA
GCTGCTAAGGAGGTATCAAGATGGAAGACGCCAAAAACATAAAGAAAGGCCCGGC
GAACACGATTAACATCGCTAAGAACGACTTCTCTGACATCGAACTGGCTGCTATC
CCGTTCAACACTCTGGCTGACCATTACGGTGAGCGTTTAGCTCGCGAACAGTTGG
CCCTTGAGCATGAGTCTTACGAGATGGGTGAAGCACGCTTCCGCAAGATGTTTGA
GCGTCAACTTAAAGCTGGTGAGGTTGCGGATAACGCTGCCGCCAAGCCTCTCATC
ACTACCCTACTCCCTAAGATGATTGCACGCATCAACGACTGGTTTGAGGAAGTGA
AAGCTAAGCGCGGCAAGCGCCCGACAGCCTTCCAGTTCCTGCAAGAAATCAAGCC
GGAAGCCGTAGCGTACATCACCATTAAGACCACTCTGGCTTGCCTAACCAGTGCT
GACAATACAACCGTTCAGGCTGTAGCAAGCGCAATCGGTCGGGCCATTGAGGACG
AGGCTCGCTTCGGTCGTATCCGTGACCTTGAAGCTAAGCACTTCAAGAAAAACGT
TGAGGAACAACTCAACAAGCGCGTAGGGCACGTCTACAAGAAAGCATTTATGCAA
GTTGTCGAGGCTGACATGCTCTCTAAGGGTCTACTCGGTGGCGAGGCGTGGTCTT
CGTGGCATAAGGAAGACTCTATTCATGTAGGAGTACGCTGCATCGAGATGCTCAT
TGAGTCAACCGGAATGGTTAGCTTACACCGCCAAAATGCTGGCGTAGTAGGTCAA
GACTCTGAGACTATCGAACTCGCACCTGAATACGCTGAGGCTATCGCAACCCGTG
CAGGTGCGCTGGCTGGCATCTCTCCGATGTTCCAACCTTGCGTAGTTCCTCCTAA
GCCGTGGACTGGCATTACTGGTGGTGGCTATTGGGCTAACGGTCGTCGTCCTCTG
GCGCTGGTGCGTACTCACAGTAAGAAAGCACTGATGCGCTACGAAGACGTTTACA
TGCCTGAGGTGTACAAAGCGATTAACATTGCGCAAAACACCGCATGGAAAATCAA
CAAGAAAGTCCTAGCGGTCGCCAACGTAATCACCAAGTGGAAGCATTGTCCGGTC
GAGGACATCCCTGCGATTGAGCGTGAAGAACTCCCGATGAAACCGGAAGACATCG
ACATGAATCCTGAGGCTCTCACCGCGTGGAAACGTGCTGCCGCTGCTGTGTACCG
CAAGGACAAGGCTCGCAAGTCTCGCCGTATCAGCCTTGAGTTCATGCTTGAGCAA
GCCAATAAGTTTGCTAACCATAAGGCCATCTGGTTCCCTTACAACATGGACTGGC
GCGGTCGTGTTTACGCTGTGTCAATGTTCAACCCGCAAGGTAACGATATGACCAA
AGGACTGCTTACGCTGGCGAAAGGTAAACCAATCGGTAAGGAAGGTTACTACTGG
CTGAAAATCCACGGTGCAAACTGTGCGGGTGTCGATAAGGTTCCGTTCCCTGAGC
GCATCAAGTTCATTGAGGAAAACCACGAGAACATCATGGCTTGCGCTAAGTCTCC
ACTGGAGAACACTTGGTGGGCTGAGCAAGATTCTCCGTTCTGCTTCCTTGCGTTC
TGCTTTGAGTACGCTGGGGTACAGCACCACGGCCTGAGCTATAACTGCTCCCTTC
CGCTGGCGTTTGACGGGTCTTGCTCTGGCATCCAGCACTTCTCCGCGATGCTCCG
AGATGAGGTAGGTGGTCGCGCGGTTAACTTGCTTCCTAGTGAAACCGTTCAGGAC
ATCTACGGGATTGTTGCTAAGAAAGTCAACGAGATTCTACAAGCAGACGCAATCA
ATGGGACCGATAACGAAGTAGTTACCGTGACCGATGAGAACACTGGTGAAATCTC
TGAGAAAGTCAAGCTGGGCACTAAGGCACTGGCTGGTCAATGGCTGGCTTACGGT
GTTACTCGCAGTGTGACTAAGCGTTCAGTCATGACGCTGGCTTACGGGTCCAAAG
AGTTCGGCTTCCGTCAACAAGTGCTGGAAGATACCATTCAGCCAGCTATTGATTC
CGGCAAGGGTCTGATGTTCACTCAGCCGAATCAGGCTGCTGGATACATGGCTAAG
CTGATTTGGGAATCTGTGAGCGTGACGGTGGTAGCTGCGGTTGAAGCAATGAACT
GGCTTAAGTCTGCTGCTAAGCTGCTGGCTGCTGAGGTCAAAGATAAGAAGACTGG
AGAGATTCTTCGCAAGCGTTGCGCTGTGCATTGGGTAACTCCTGATGGTTTCCCT
GTGTGGCAGGAATACAAGAAGCCTATTCAGACGCGCTTGAACCTGATGTTCCTCG
GTCAGTTCCGCTTACAGCCTACCATTAACACCAACAAAGATAGCGAGATTGATGC
ACACAAACAGGAGTCTGGTATCGCTCCTAACTTTGTACACAGCCAAGACGGTAGC
CACCTTCGTAAGACTGTAGTGTGGGCACACGAGAAGTACGGAATCGAATCTTTTG
CACTGATTCACGACTCCTTCGGTACCATTCCGGCTGACGCTGCGAACCTGTTCAA
AGCAGTGCGCGAAACTATGGTTGACACATATGAGTCTTGTGATGTACTGGCTGAT
TTCTACGACCAGTTCGCTGACCAGTTGCACGAGTCTCAATTGGACAAAATGCCAG
CACTTCCGGCTAAAGGTAACTTGAACCTCCGTGACATCTTAGAGTCGGACTTCGC
GTTCGCGTAAAAGCTTGATGGGGGATCCCATGGTACGCGTGCTAG
T12 SEQ ID NO: 121:
TCAGAATTGGTTAATTGGTTGTAACACTGGTCTATCATTGATAGGTATAAATTAA
TACGACTCACTAATACTGAACCTATCAGTGATAGATCCAAACCCTCGTTAGGGGA
GCGTCTAATTTTAGGAGATCCAAAATGTCAAGGCTGGATAAATCAAAAGTAATCA
ATAGCGCGCTGGAACTGCTGAACGAGGTCGGCATCGAAGGTCTGACCACCCGCAA
GCTGGCGCAAAAACTGGGCGTCGAACAACCGACGCTGTACTGGCACGTAAAAAAT
AAGCGTGCGCTGCTGGACGCACTGGCAATTGAAATGCTGGATCGTCACCACACCC
ACTTCTGTCCGCTGGAGGGTGAATCATGGCAAGATTTCCTTCGCAACAACGCGAA
GTCATTTCGCTGCGCGCTGCTGAGCCACCGCGATGGAGCAAAAGTTCATCTGGGC
ACCCGCCCAACGGAGAAACAATATGAAACGCTGGAAAACCAGCTTGCCTTCCTGT
GCCAGCAGGGTTTCAGCCTTGAGAACGCGCTGTACGCGCTGAGCGCCGTAGGTCA
CTTCACCCTGGGCTGTGTTCTGGAAGACCAAGAACATCAAGTAGCAAAAGAAGAG
CGAGAAACCCCTACGACCGATTCGATGCCGCCGCTGCTGCGTCAGGCGATTGAAC
TGTTCGATCACCAGGGCGCGGAACCGGCATTCCTGTTTGGTCTGGAACTTATTAT
ATGCGGCCTAGAAAAACAACTGAAGTGCGAAAGCGGTAGCTAAGCGGATCTTTAC
AGATTCTATACCGGTGATACCAGCATCGTCTTGATGCCCTTGGCAGCACCCTGCT
AAGGAGGCAACAAGATGAACACGATTAACATCGCTAAGAACGACTTCTCTGACAT
CGAACTGGCTGCTATCCCGTTCAACACTCTGGCTGACCATTACGGTGAGCGTTTg
GCTCGCGAACAGTTGGCCCTTGAGCATGAGTCTTACGAGATGGGTGAAGCACGCT
TCCGCAAGATGTTTGAGCGTCAACTTAAAGCTGGTGAGGTTGCGGATAACGCTGC
CGCCAAGCCTCTCATCACTACCCTACTCCCTAAGATGATTGCACGCATCAACGAC
TGGTTTGAGGAAGTGAAAGCTAAGCGCGGCAAGCGCCCGACAGCCTTCCAGTTCC
TGCAAGAAATCAAGCCGGAAGCCGTAGCGTACATCACCATTAAGACCACTCTGGC
TTGCCTAACCAGTGCTGACAATACAACCGTTCAGGCTGTAGCAAGCGCAATCGGT
CGGGCCATTGAGGACGAGGCTCGCTTCGGTCGTATCCGTGACCTTGAAGCTAAGC
ACTTCAAGAAAAACGTTGAGGAACAACTCAACAAGCGCGTAGGGCACGTCTACAA
GAAAGCATTTATGCAAGTTGTCGAGGCTGACATGCTCTCTAAGGGTCTACTCGGT
GGCGAGGCGTGGTCTTCGTGGCATAAGGAAGACTCTATTCATGTAGGAGTACGCT
GCATCGAGATGCTCATTGAGTCAACCGGAATGGTTAGCTTgCACCGCCAAAATGC
TGGCGTAGTAGGTCAAGACTCTGAGACTATCGAACTCGCACCTGAATACGCTGAG
GCTATCGCAACCCGTGCAGGTGCGCTGGCTGGCATCTCTCCGATGTTCCAACCTT
GCGTAGTTCCTCCTAAGCCGTGGACTGGCATTACTGGTGGTGGCTATTGGGCTAA
CGGTCGTCGTCCTCTGGCGCTGGTGCGTACTCACAGTAAGAAAGCACTGATGCGC
TACGAAGACGTTTACATGCCTGAGGTGTACAAAGCGATTAACATTGCGCAAAACA
CCGCATGGAAAATCAACAAGAAAGTCCTAGCGGTCGCCAACGTAATCACCAAGTG
GAAGCATTGTCCGGTCGAGGACATCCCTGCGATTGAGCGTGAAGAACTCCCGATG
AAACCGGAAGACATCGACATGAATCCTGAGGCTCTCACCGCGTGGAAACGTGCTG
CCGCTGCTGTGTACCGCAAGGACAAGGCTCGCAAGTCTCGCCGTATCAGCCTTGA
GTTCATGCTTGAGCAAGCCAATAAGTTTGCTAACCATAAGGCCATCTGGTTCCCT
TACAACATGGACTGGCGCGGTCGTGTTTACGCTGTGTCAATGTTCAACCCGCAAG
GTAACGATATGACCAAAGGACTGCTTACGCTGGCGAAAGGTAAACCAATCGGTAA
GGAAGGTTACTACTGGCTGAAAATCCACGGTGCAAACTGTGCGGGTGTCGATAAG
GTTCCGTTCCCTGAGCGCATCAAGTTCATTGAGGAAAACCACGAGAACATCATGG
CTTGCGCTAAGTCTCCACTGGAGAACACTTGGTGGGCTGAGCAAGATTCTCCGTT
CTGCTTCCTTGCGTTCTGCTTTGAGTACGCTGGGGTACAGCACCACGGCCTGAGC
TATAACTGCTCCCTTCCGCTGGCGTTTGACGGGTCTTGCTCTGGCATCCAGCACT
TCTCCGCGATGCTCCGAGATGAGGTAGGTGGTCGCGCGGTTAACTTGCTTCCTAG
TGAAACCGTTCAGGACATCTACGGGATTGTTGCTAAGAAAGTCAACGAGATTCTA
CAAGCAGACGCAATCAATGGGACCGATAACGAAGTAGTTACCGTGACCGATGAGA
ACACTGGTGAAATCTCTGAGAAAGTCAAGCTGGGCACTAAGGCACTGGCTGGTCA
ATGGCTGGCTTACGGTGTTACTCGCAGTGTGACTAAGCGTTCAGTCATGACGCTG
GCTTACGGGTCCAAAGAGTTCGGCTTCCGTCAACAAGTGCTGGAAGATACCATTC
AGCCAGCTATTGATTCCGGCAAGGGTCTGATGTTCACTCAGCCGAATCAGGCTGC
TGGATACATGGCTAAGCTGATTTGGGAATCTGTGAGCGTGACGGTGGTAGCTGCG
GTTGAAGCAATGAACTGGCTTAAGTCTGCTGCTAAGCTGCTGGCTGCTGAGGTCA
AAGATAAGAAGACTGGAGAGATTCTTCGCAAGCGTTGCGCTGTGCATTGGGTAAC
TCCTGATGGTTTCCCTGTGTGGCAGGAATACAAGAAGCCTATTCAGACGCGCTTG
AACCTGATGTTCCTCGGTCAGTTCCGCTTgCAGCCTACCATTAACACCAACAAAG
ATAGCGAGATTGATGCACACAAACAGGAGTCTGGTATCGCTCCTAACTTTGTACA
CAGCCAAGACGGTAGCCACCTTCGTAAGACTGTAGTGTGGGCACACGAGAAGTAC
GGAATCGAATCTTTTGCACTGATTCACGACTCCTTCGGTACCATTCCGGCTGACG
CTGCGAACCTGTTCAAAGCAGTGCGCGAAACTATGGTTGACACATATGAGTCTTG
TGATGTACTGGCTGATTTCTACGACCAGTTCGCTGACCAGTTGCACGAGTCTCAA
TTGGACAAAATGCCAGCACTTCCGGCTAAAGGTAACTTGAACCTCCGTGACATCT
TgGAGTCGGACTTCGCGTTCGCGTAAAAGCTTGATATCGAATTCCTGCAGCCCCG
GGGATCCCATGGTACGCGTGCTAG
T13 SEQ ID NO: 122:
TCAGAATTGGTTAATTGGTTGTAACACTGGTCTATCATTGATAGGTATAAATTAA
TACGACTCACTATAGGGAGACCTATCAGTGATAGATCCAAACCCTCGTTAGGGGA
GCGTCTAATTTTAGGAGATCCAAAATGTCAAGGCTGGATAAATCAAAAGTAATCA
ATAGCGCGCTGGAACTGCTGAACGAGGTCGGCATCGAAGGTCTGACCACCCGCAA
GCTGGCGCAAAAACTGGGCGTCGAACAACCGACGCTGTACTGGCACGTAAAAAAT
AAGCGTGCGCTGCTGGACGCACTGGCAATTGAAATGCTGGATCGTCACCACACCC
ACTTCTGTCCGCTGGAGGGTGAATCATGGCAAGATTTCCTTCGCAACAACGCGAA
GTCATTTCGCTGCGCGCTGCTGAGCCACCGCGATGGAGCAAAAGTTCATCTGGGC
ACCCGCCCAACGGAGAAACAATATGAAACGCTGGAAAACCAGCTTGCCTTCCTGT
GCCAGCAGGGTTTCAGCCTTGAGAACGCGCTGTACGCGCTGAGCGCCGTAGGTCA
CTTCACCCTGGGCTGTGTTCTGGAAGACCAAGAACATCAAGTAGCAAAAGAAGAG
CGAGAAACCCCTACGACCGATTCGATGCCGCCGCTGCTGCGTCAGGCGATTGAAC
TGTTCGATCACCAGGGCGCGGAACCGGCATTCCTGTTTGGTCTGGAACTTATTAT
ATGCGGCCTAGAAAAACAACTGAAGTGCGAAAGCGGTAGCTAAGCGGATCTTTAC
AGATTCTATACCGGTGATACCAGCATCGTCTTGATGCCCTTGGCAGCACCCTGCT
AAGGAGGCAACAAGATGAACACGATTAACATCGCTAAGAACGACTTCTCTGACAT
CGAACTGGCTGCTATCCCGTTCAACACTCTGGCTGACCATTACGGTGAGCGTTTg
GCTCGCGAACAGTTGGCCCTTGAGCATGAGTCTTACGAGATGGGTGAAGCACGCT
TCCGCAAGATGTTTGAGCGTCAACTTAAAGCTGGTGAGGTTGCGGATAACGCTGC
CGCCAAGCCTCTCATCACTACCCTACTCCCTAAGATGATTGCACGCATCAACGAC
TGGTTTGAGGAAGTGAAAGCTAAGCGCGGCAAGCGCCCGACAGCCTTCCAGTTCC
TGCAAGAAATCAAGCCGGAAGCCGTAGCGTACATCACCATTAAGACCACTCTGGC
TTGCCTAACCAGTGCTGACAATACAACCGTTCAGGCTGTAGCAAGCGCAATCGGT
CGGGCCATTGAGGACGAGGCTCGCTTCGGTCGTATCCGTGACCTTGAAGCTAAGC
ACTTCAAGAAAAACGTTGAGGAACAACTCAACAAGCGCGTAGGGCACGTCTACAA
GAAAGCATTTATGCAAGTTGTCGAGGCTGACATGCTCTCTAAGGGTCTACTCGGT
GGCGAGGCGTGGTCTTCGTGGCATAAGGAAGACTCTATTCATGTAGGAGTACGCT
GCATCGAGATGCTCATTGAGTCAACCGGAATGGTTAGCTTgCACCGCCAAAATGC
TGGCGTAGTAGGTCAAGACTCTGAGACTATCGAACTCGCACCTGAATACGCTGAG
GCTATCGCAACCCGTGCAGGTGCGCTGGCTGGCATCTCTCCGATGTTCCAACCTT
GCGTAGTTCCTCCTAAGCCGTGGACTGGCATTACTGGTGGTGGCTATTGGGCTAA
CGGTCGTCGTCCTCTGGCGCTGGTGCGTACTCACAGTAAGAAAGCACTGATGCGC
TACGAAGACGTTTACATGCCTGAGGTGTACAAAGCGATTAACATTGCGCAAAACA
CCGCATGGAAAATCAACAAGAAAGTCCTAGCGGTCGCCAACGTAATCACCAAGTG
GAAGCATTGTCCGGTCGAGGACATCCCTGCGATTGAGCGTGAAGAACTCCCGATG
AAACCGGAAGACATCGACATGAATCCTGAGGCTCTCACCGCGTGGAAACGTGCTG
CCGCTGCTGTGTACCGCAAGGACAAGGCTCGCAAGTCTCGCCGTATCAGCCTTGA
GTTCATGCTTGAGCAAGCCAATAAGTTTGCTAACCATAAGGCCATCTGGTTCCCT
TACAACATGGACTGGCGCGGTCGTGTTTACGCTGTGTCAATGTTCAACCCGCAAG
GTAACGATATGACCAAAGGACTGCTTACGCTGGCGAAAGGTAAACCAATCGGTAA
GGAAGGTTACTACTGGCTGAAAATCCACGGTGCAAACTGTGCGGGTGTCGATAAG
GTTCCGTTCCCTGAGCGCATCAAGTTCATTGAGGAAAACCACGAGAACATCATGG
CTTGCGCTAAGTCTCCACTGGAGAACACTTGGTGGGCTGAGCAAGATTCTCCGTT
CTGCTTCCTTGCGTTCTGCTTTGAGTACGCTGGGGTACAGCACCACGGCCTGAGC
TATAACTGCTCCCTTCCGCTGGCGTTTGACGGGTCTTGCTCTGGCATCCAGCACT
TCTCCGCGATGCTCCGAGATGAGGTAGGTGGTCGCGCGGTTAACTTGCTTCCTAG
TGAAACCGTTCAGGACATCTACGGGATTGTTGCTAAGAAAGTCAACGAGATTCTA
CAAGCAGACGCAATCAATGGGACCGATAACGAAGTAGTTACCGTGACCGATGAGA
ACACTGGTGAAATCTCTGAGAAAGTCAAGCTGGGCACTAAGGCACTGGCTGGTCA
ATGGCTGGCTTACGGTGTTACTCGCAGTGTGACTAAGCGTTCAGTCATGACGCTG
GCTTACGGGTCCAAAGAGTTCGGCTTCCGTCAACAAGTGCTGGAAGATACCATTC
AGCCAGCTATTGATTCCGGCAAGGGTCTGATGTTCACTCAGCCGAATCAGGCTGC
TGGATACATGGCTAAGCTGATTTGGGAATCTGTGAGCGTGACGGTGGTAGCTGCG
GTTGAAGCAATGAACTGGCTTAAGTCTGCTGCTAAGCTGCTGGCTGCTGAGGTCA
AAGATAAGAAGACTGGAGAGATTCTTCGCAAGCGTTGCGCTGTGCATTGGGTAAC
TCCTGATGGTTTCCCTGTGTGGCAGGAATACAAGAAGCCTATTCAGACGCGCTTG
AACCTGATGTTCCTCGGTCAGTTCCGCTTgCAGCCTACCATTAACACCAACAAAG
ATAGCGAGATTGATGCACACAAACAGGAGTCTGGTATCGCTCCTAACTTTGTACA
CAGCCAAGACGGTAGCCACCTTCGTAAGACTGTAGTGTGGGCACACGAGAAGTAC
GGAATCGAATCTTTTGCACTGATTCACGACTCCTTCGGTACCATTCCGGCTGACG
CTGCGAACCTGTTCAAAGCAGTGCGCGAAACTATGGTTGACACATATGAGTCTTG
TGATGTACTGGCTGATTTCTACGACCAGTTCGCTGACCAGTTGCACGAGTCTCAA
TTGGACAAAATGCCAGCACTTCCGGCTAAAGGTAACTTGAACCTCCGTGACATCT
TgGAGTCGGACTTCGCGTTCGCGTAAAAGCTTGATGGGGGATCCCATGGTACGCG
TGCTAG
T14 SEQ ID NO: 123:
TCAGAATTGGTTAATTGGTTGTAACACTGGTCTATCATTGATAGGTATAAATTAA
TACGACTCACTAATACTGAACCTATCAGTGATAGATCCAAACCCAAAAACACAGG
AGTTTTTAGAATGTCTAGATTAGATAAAAGTAAAGTGATTAACAGCGCATTAGAG
CTGCTTAATGAGGTCGGAATCGAAGGTTTAACAACCCGTAAACTCGCCCAGAAGC
TAGGTGTAGAGCAGCCTACATTGTATTGGCATGTAAAAAATAAGCGGGCTTTGCT
CGACGCCTTAGCCATTGAGATGTTAGATAGGCACCATACTCACTTTTGCCCTTTA
GAAGGGGAAAGCTGGCAAGATTTTTTACGTAATAACGCTAAAAGTTTTAGATGTG
CTTTACTAAGTCATCGCGATGGAGCAAAAGTACATTTAGGTACACGGCCTACAGA
AAAACAGTATGAAACTCTCGAAAATCAATTAGCCTTTTTATGCCAACAAGGTTTT
TCACTAGAGAATGCATTATATGCACTCAGCGCTGTGGGGCATTTTACTTTAGGTT
GCGTATTGGAAGATCAAGAGCATCAAGTCGCTAAAGAAGAAAGGGAAACACCTAC
TACTGATAGTATGCCGCCATTATTACGACAAGCTATCGAATTATTTGATCACCAA
GGTGCAGAGCCAGCCTTCTTATTCGGCCTTGAATTGATCATATGCGGATTAGAAA
AACAACTTAAATGTGAAAGTGGGTCTTAATTGGCAGCACAATggTAAGGAGGCAA
CAAGATGAACACGATTAACATCGCTAAGAACGACTTCTCTGACATCGAACTGGCT
GCTATCCCGTTCAACACTCTGGCTGACCATTACGGTGAGCGTTTAGCTCGCGAAC
AGTTGGCCCTTGAGCATGAGTCTTACGAGATGGGTGAAGCACGCTTCCGCAAGAT
GTTTGAGCGTCAACTTAAAGCTGGTGAGGTTGCGGATAACGCTGCCGCCAAGCCT
CTCATCACTACCCTACTCCCTAAGATGATTGCACGCATCAACGACTGGTTTGAGG
AAGTGAAAGCTAAGCGCGGCAAGCGCCCGACAGCCTTCCAGTTCCTGCAAGAAAT
CAAGCCGGAAGCCGTAGCGTACATCACCATTAAGACCACTCTGGCTTGCCTAACC
AGTGCTGACAATACAACCGTTCAGGCTGTAGCAAGCGCAATCGGTCGGGCCATTG
AGGACGAGGCTCGCTTCGGTCGTATCCGTGACCTTGAAGCTAAGCACTTCAAGAA
AAACGTTGAGGAACAACTCAACAAGCGCGTAGGGCACGTCTACAAGAAAGCATTT
ATGCAAGTIGTCGAGGCTGACATGCTCTCTAAGGGTCTACTCGGTGGCGAGGCGT
GGTCTTCGTGGCATAAGGAAGACTCTATTCATGTAGGAGTACGCTGCATCGAGAT
GCTCATTGAGTCAACCGGAATGGTTAGCTTACACCGCCAAAATGCTGGCGTAGTA
GGTCAAGACTCTGAGACTATCGAACTCGCACCTGAATACGCTGAGGCTATCGCAA
CCCGTGCAGGTGCGCTGGCTGGCATCTCTCCGATGTTCCAACCTTGCGTAGTTCC
TCCTAAGCCGTGGACTGGCATTACTGGTGGTGGCTATTGGGCTAACGGTCGTCGT
CCTCTGGCGCTGGTGCGTACTCACAGTAAGAAAGCACTGATGCGCTACGAAGACG
TTTACATGCCTGAGGTGTACAAAGCGATTAACATTGCGCAAAACACCGCATGGAA
AATCAACAAGAAAGTCCTAGCGGTCGCCAACGTAATCACCAAGTGGAAGCATTGT
CCGGTCGAGGACATCCCTGCGATTGAGCGTGAAGAACTCCCGATGAAACCGGAAG
ACATCGACATGAATCCTGAGGCTCTCACCGCGTGGAAACGTGCTGCCGCTGCTGT
GTACCGCAAGGACAAGGCTCGCAAGTCTCGCCGTATCAGCCTTGAGTTCATGCTT
GAGCAAGCCAATAAGTTTGCTAACCATAAGGCCATCTGGTTCCCTTACAACATGG
ACTGGCGCGGTCGTGTTTACGCTGTGTCAATGTTCAACCCGCAAGGTAACGATAT
GACCAAAGGACTGCTTACGCTGGCGAAAGGTAAACCAATCGGTAAGGAAGGTTAC
TACTGGCTGAAAATCCACGGTGCAAACTGTGCGGGTGTCGATAAGGTTCCGTTCC
CTGAGCGCATCAAGTTCATTGAGGAAAACCACGAGAACATCATGGCTTGCGCTAA
GTCTCCACTGGAGAACACTTGGTGGGCTGAGCAAGATTCTCCGTTCTGCTTCCTT
GCGTTCTGCTTTGAGTACGCTGGGGTACAGCACCACGGCCTGAGCTATAACTGCT
CCCTTCCGCTGGCGTTTGACGGGTCTTGCTCTGGCATCCAGCACTTCTCCGCGAT
GCTCCGAGATGAGGTAGGTGGTCGCGCGGTTAACTTGCTTCCTAGTGAAACCGTT
CAGGACATCTACGGGATTGTTGCTAAGAAAGTCAACGAGATTCTACAAGCAGACG
CAATCAATGGGACCGATAACGAAGTAGTTACCGTGACCGATGAGAACACTGGTGA
AATCTCTGAGAAAGTCAAGCTGGGCACTAAGGCACTGGCTGGTCAATGGCTGGCT
TACGGTGTTACTCGCAGTGTGACTAAGCGTTCAGTCATGACGCTGGCTTACGGGT
CCAAAGAGTTCGGCTTCCGTCAACAAGTGCTGGAAGATACCATTCAGCCAGCTAT
TGATTCCGGCAAGGGTCTGATGTTCACTCAGCCGAATCAGGCTGCTGGATACATG
GCTAAGCTGATTTGGGAATCTGTGAGCGTGACGGTGGTAGCTGCGGTTGAAGCAA
TGAACTGGCTTAAGTCTGCTGCTAAGCTGCTGGCTGCTGAGGTCAAAGATAAGAA
GACTGGAGAGATTCTTCGCAAGCGTTGCGCTGTGCATTGGGTAACTCCTGATGGT
TTCCCTGTGTGGCAGGAATACAAGAAGCCTATTCAGACGCGCTTGAACCTGATGT
TCCTCGGTCAGTTCCGCTTACAGCCTACCATTAACACCAACAAAGATAGCGAGAT
TGATGCACACAAACAGGAGTCTGGTATCGCTCCTAACTTTGTACACAGCCAAGAC
GGTAGCCACCTTCGTAAGACTGTAGTGTGGGCACACGAGAAGTACGGAATCGAAT
CTTTTGCACTGATTCACGACTCCTTCGGTACCATTCCGGCTGACGCTGCGAACCT
GTTCAAAGCAGTGCGCGAAACTATGGTTGACACATATGAGTCTTGTGATGTACTG
GCTGATTTCTACGACCAGTTCGCTGACCAGTTGCACGAGTCTCAATTGGACAAAA
TGCCAGCACTTCCGGCTAAAGGTAACTTGAACCTCCGTGACATCTTAGAGTCGGA
CTTCGCGTTCGCGTAAAAGCTTGATGGGGGATCCCATGGTACGCGTGCTAG
T15 SEQ ID NO: 124:
TCAGAATTGGTTAATTGGTTGTAACACTGGTCTATCATTGATAGGTATAAATTAA
TACGACTCACTAATACTGAACCTATCAGTGATAGATACCGGTGATACCAGCATCG
TCTTGATGCCCTTGGCAGCACCCTGCTAAGGAGGCAACAAGATGAACACGATTAA
CATCGCTAAGAACGACTTCTCTGACATCGAACTGGCTGCTATCCCGTTCAACACT
CTGGCTGACCATTACGGTGAGCGTTTgGCTCGCGAACAGTTGGCCCTTGAGCATG
AGTCTTACGAGATGGGTGAAGCACGCTTCCGCAAGATGTTTGAGCGTCAACTTAA
AGCTGGTGAGGTTGCGGATAACGCTGCCGCCAAGCCTCTCATCACTACCCTACTC
CCTAAGATGATTGCACGCATCAACGACTGGTTTGAGGAAGTGAAAGCTAAGCGCG
GCAAGCGCCCGACAGCCTTCCAGTTCCTGCAAGAAATCAAGCCGGAAGCCGTAGC
GTACATCACCATTAAGACCACTCTGGCTTGCCTAACCAGTGCTGACAATACAACC
GTTCAGGCTGTAGCAAGCGCAATCGGTCGGGCCATTGAGGACGAGGCTCGCTTCG
GTCGTATCCGTGACCTTGAAGCTAAGCACTTCAAGAAAAACGTTGAGGAACAACT
CAACAAGCGCGTAGGGCACGTCTACAAGAAAGCATTTATGCAAGTTGTCGAGGCT
GACATGCTCTCTAAGGGTCTACTCGGTGGCGAGGCGTGGTCTTCGTGGCATAAGG
AAGACTCTATTCATGTAGGAGTACGCTGCATCGAGATGCTCATTGAGTCAACCGG
AATGGTTAGCTTgCACCGCCAAAATGCTGGCGTAGTAGGTCAAGACTCTGAGACT
ATCGAACTCGCACCTGAATACGCTGAGGCTATCGCAACCCGTGCAGGTGCGCTGG
CTGGCATCTCTCCGATGTTCCAACCTTGCGTAGTTCCTCCTAAGCCGTGGACTGG
CATTACTGGTGGTGGCTATTGGGCTAACGGTCGTCGTCCTCTGGCGCTGGTGCGT
ACTCACAGTAAGAAAGCACTGATGCGCTACGAAGACGTTTACATGCCTGAGGTGT
ACAAAGCGATTAACATTGCGCAAAACACCGCATGGAAAATCAACAAGAAAGTCCT
AGCGGTCGCCAACGTAATCACCAAGTGGAAGCATTGTCCGGTCGAGGACATCCCT
GCGATTGAGCGTGAAGAACTCCCGATGAAACCGGAAGACATCGACATGAATCCTG
AGGCTCTCACCGCGTGGAAACGTGCTGCCGCTGCTGTGTACCGCAAGGACAAGGC
TCGCAAGTCTCGCCGTATCAGCCTTGAGTTCATGCTTGAGCAAGCCAATAAGTTT
GCTAACCATAAGGCCATCTGGTTCCCTTACAACATGGACTGGCGCGGTCGTGTTT
ACGCTGTGTCAATGTTCAACCCGCAAGGTAACGATATGACCAAAGGACTGCTTAC
GCTGGCGAAAGGTAAACCAATCGGTAAGGAAGGTTACTACTGGCTGAAAATCCAC
GGTGCAAACTGTGCGGGTGTCGATAAGGTTCCGTTCCCTGAGCGCATCAAGTTCA
TTGAGGAAAACCACGAGAACATCATGGCTTGCGCTAAGTCTCCACTGGAGAACAC
TTGGTGGGCTGAGCAAGATTCTCCGTTCTGCTTCCTTGCGTTCTGCTTTGAGTAC
GCTGGGGTACAGCACCACGGCCTGAGCTATAACTGCTCCCTTCCGCTGGCGTTTG
ACGGGTCTTGCTCTGGCATCCAGCACTTCTCCGCGATGCTCCGAGATGAGGTAGG
TGGTCGCGCGGTTAACTTGCTTCCTAGTGAAACCGTTCAGGACATCTACGGGATT
GTTGCTAAGAAAGTCAACGAGATTCTACAAGCAGACGCAATCAATGGGACCGATA
ACGAAGTAGTTACCGTGACCGATGAGAACACTGGTGAAATCTCTGAGAAAGTCAA
GCTGGGCACTAAGGCACTGGCTGGTCAATGGCTGGCTTACGGTGTTACTCGCAGT
GTGACTAAGCGTTCAGTCATGACGCTGGCTTACGGGTCCAAAGAGTTCGGCTTCC
GTCAACAAGTGCTGGAAGATACCATTCAGCCAGCTATTGATTCCGGCAAGGGTCT
GATGTTCACTCAGCCGAATCAGGCTGCTGGATACATGGCTAAGCTGATTTGGGAA
TCTGTGAGCGTGACGGTGGTAGCTGCGGTTGAAGCAATGAACTGGCTTAAGTCTG
CTGCTAAGCTGCTGGCTGCTGAGGTCAAAGATAAGAAGACTGGAGAGATTCTTCG
CAAGCGTTGCGCTGTGCATTGGGTAACTCCTGATGGTTTCCCTGTGTGGCAGGAA
TACAAGAAGCCTATTCAGACGCGCTTGAACCTGATGTTCCTCGGTCAGTTCCGCT
TgCAGCCTACCATTAACACCAACAAAGATAGCGAGATTGATGCACACAAACAGGA
GTCTGGTATCGCTCCTAACTTTGTACACAGCCAAGACGGTAGCCACCTTCGTAAG
ACTGTAGTGTGGGCACACGAGAAGTACGGAATCGAATCTTTTGCACTGATTCACG
ACTCCTTCGGTACCATTCCGGCTGACGCTGCGAACCTGTTCAAAGCAGTGCGCGA
AACTATGGTTGACACATATGAGTCTTGTGATGTACTGGCTGATTTCTACGACCAG
TTCGCTGACCAGTTGCACGAGTCTCAATTGGACAAAATGCCAGCACTTCCGGCTA
AAGGTAACTTGAACCTCCGTGACATCTTgGAGTCGGACTTCGCGTTCGCGTAAAA
GCTTGATATCGAATTCCTGCAGCCCCGGGGATCCCATGGTACGCGTGCTAGTAAT
ACGACTCACTAATACTGAATCCAAACCCTCGTTAGGGGAGCGTCTAATTTTAGGA
GATCCAAAATGTCAAGGCTGGATAAATCAAAAGTAATCAATAGCGCGCTGGAACT
GCTGAACGAGGTCGGCATCGAAGGTCTGACCACCCGCAAGCTGGCGCAAAAACTG
GGCGTCGAACAACCGACGCTGTACTGGCACGTAAAAAATAAGCGTGCGCTGCTGG
ACGCACTGGCAATTGAAATGCTGGATCGTCACCACACCCACTTCTGTCCGCTGGA
GGGTGAATCATGGCAAGATTTCCTTCGCAACAACGCGAAGTCATTTCGCTGCGCG
CTGCTGAGCCACCGCGATGGAGCAAAAGTTCATCTGGGCACCCGCCCAACGGAGA
AACAATATGAAACGCTGGAAAACCAGCTTGCCTTCCTGTGCCAGCAGGGTTTCAG
CCTTGAGAACGCGCTGTACGCGCTGAGCGCCGTAGGTCACTTCACCCTGGGCTGT
GTTCTGGAAGACCAAGAACATCAAGTAGCAAAAGAAGAGCGAGAAACCCCTACGA
CCGATTCGATGCCGCCGCTGCTGCGTCAGGCGATTGAACTGTTCGATCACCAGGG
CGCGGAACCGGCATTCCTGTTTGGTCTGGAACTTATTATATGCGGCCTAGAAAAA
CAACTGAAGTGCGAAAGCGGTAGCTAA

TABLE 3
GFP/OD600 nm

| | | anhydrotetracycline (ng/mL) | | | | | |
|---|---|---|---|---|---|---|---|
| | | 0 | 0.16 | 0.8 | 4 | 20 | 100 |
| Theophylline | 10 | 8060 | 8280 | 10408 | 13832 | 14945 | 15746 |
| (mM) | 3.33 | 6236 | 5839 | 6234 | 7842 | 8497 | 8826 |
| | 1 | 5285 | 5054 | 5337 | 6637 | 7298 | 7757 |
| | 0.33 | 4881 | 4872 | 5081 | 6333 | 6988 | 7250 |
| | 0.16 | 3096 | 3567 | 4620 | 6052 | 6250 | 6735 |
| | 0 | 563 | 610 | 860 | 1170 | 1146 | 1233 |

TABLE 4
Max OD600 nm (percent of uninduced sample)

| | | anhydrotetracycline (ng/mL) | | | | | |
|---|---|---|---|---|---|---|---|
| | | 0 | 0.16 | 0.8 | 4 | 20 | 100 |
| Theophylline | 10 | 43 | 42 | 40 | 40 | 40 | 42 |
| (mM) | 3.33 | 79 | 76 | 77 | 74 | 71 | 75 |
| | 1 | 89 | 81 | 77 | 72 | 70 | 74 |
| | 0.33 | 95 | 89 | 87 | 84 | 79 | 86 |
| | 0.16 | 94 | 91 | 83 | 83 | 80 | 84 |
| | 0 | 100 | 95 | 94 | 92 | 92 | 98 |

To assess if this T7 RNAP gene circuit can function in both Gram-negative and positive strains, variant T15 and the eGFP reporter were cloned onto an ultra-broad host-range shuttle vector, consisting of the RSF1010 (mobAY25F) (Bishe et al., 2019) and pAMβ1 (Bruand et al., 1993) origins of replication, pBroad (FIG. 7A). Plate reader analysis demonstrated titratable T7RNAP function in both E. coli and B. subtilis (FIG. 7B).

### Development of Chromosomal-Integrated Landing Pads for SGE Mobilization

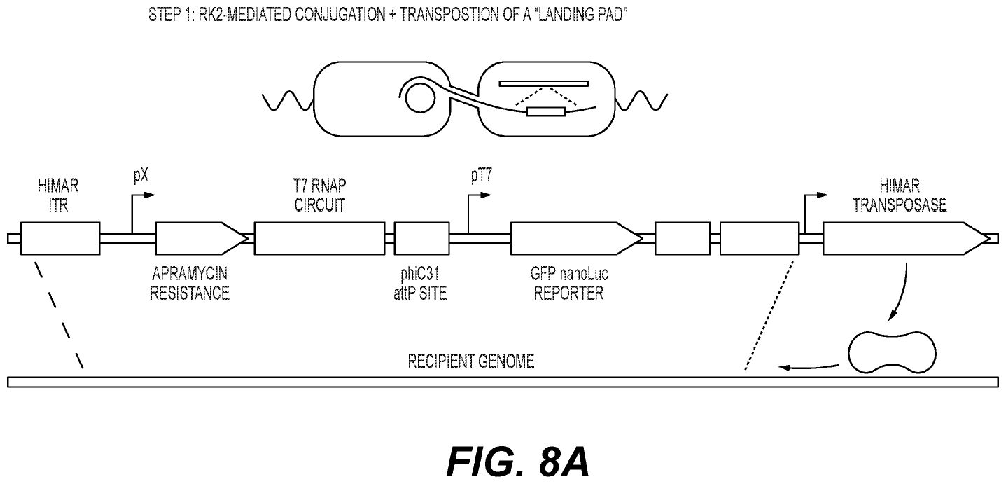

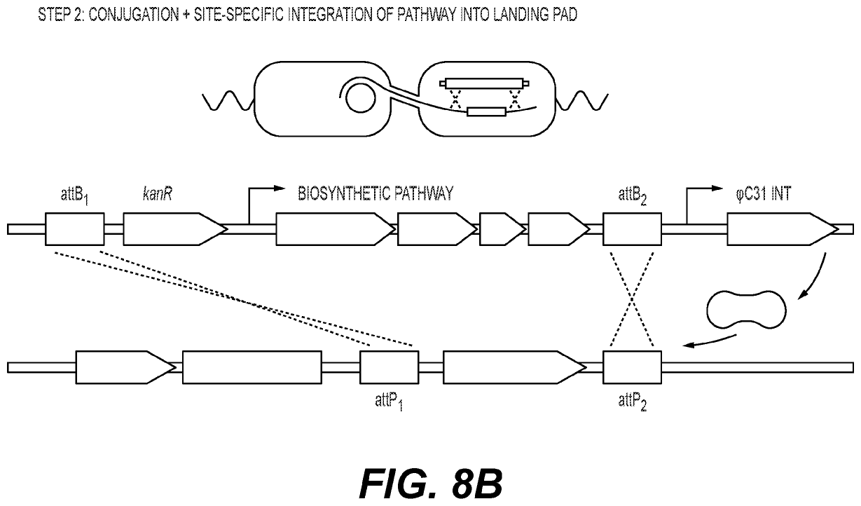

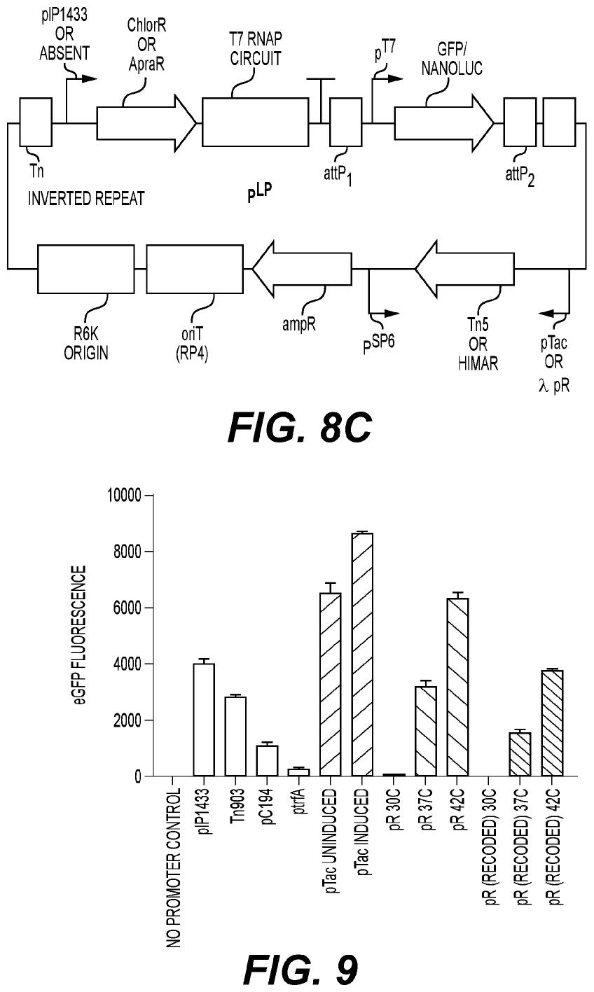

A chromosomal integration strategy for stable transfer of SGEs across diverse hosts was developed to complement the plasmid-based mobilization approach, given that integration can increase genetic stability and biosynthetic pathway productivity (Tyo et al., 2009). A two-staged approach to integrate large SGEs into the genome was developed. First, conjugative transposition was used to empirically identify safe landing sites that can stably express the T7 RNAP circuit (FIG. 8A). Second, site-specific integration was used to introduce SGEs into those safe landing sites (FIG. 8B.

To identify “safe” landing sites throughout the genome, a cassette was constructed containing the titratable variant T15 of the T7RNAP circuit, a pT7-GFP-nanoluc luciferase fusion reporter, an antibiotic selectable marker, and asymmetric phiC31 attP sites for pathway integration (Colloms et al., 2014) (FIG. 8A). This cassette was flanked by transposase terminal repeats, followed by the transposase gene, which itself does not mobilize into the recipient genome. This transposase was independent of host-specific factors and shows little bias in random integration, requiring only a TA dinucleotide target (Lampe et al., 1999). An R6K-based suicide plasmid was used for mobilization into diverse recipient bacteria via incP-mediated conjugation (Thomas and Smith, 1987), pLP (FIG. 8C).

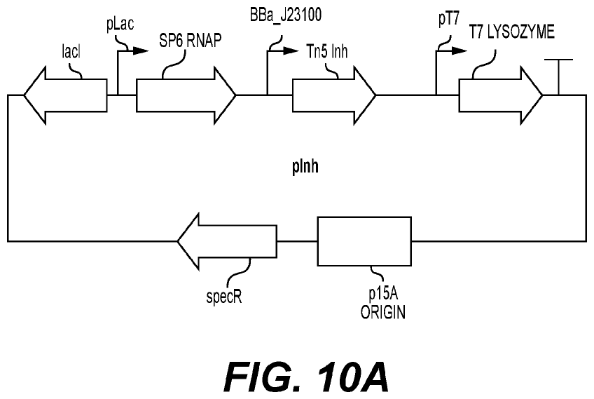

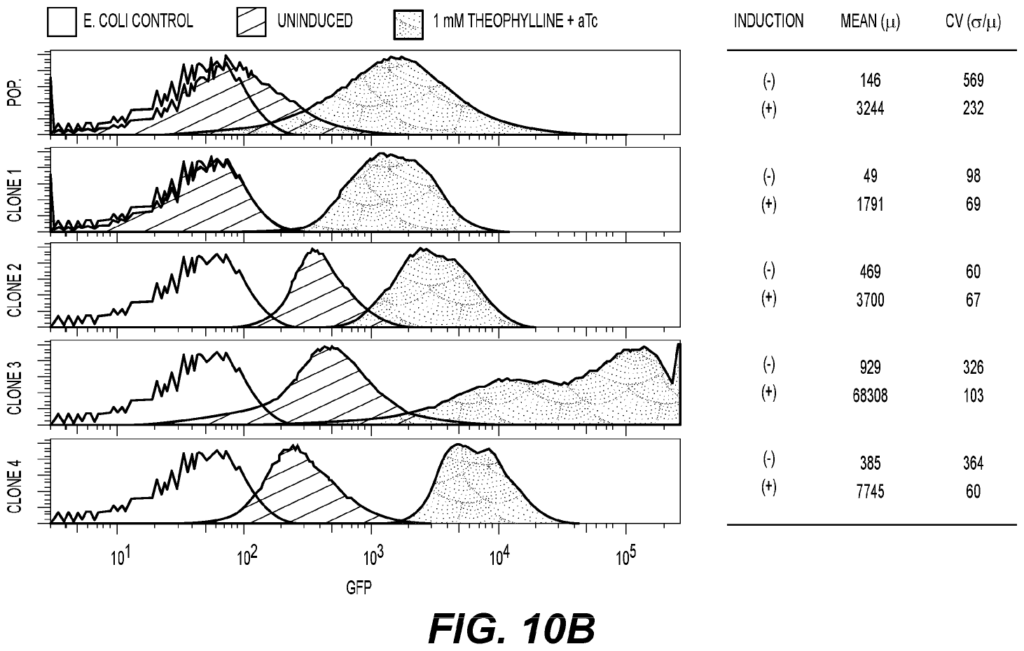

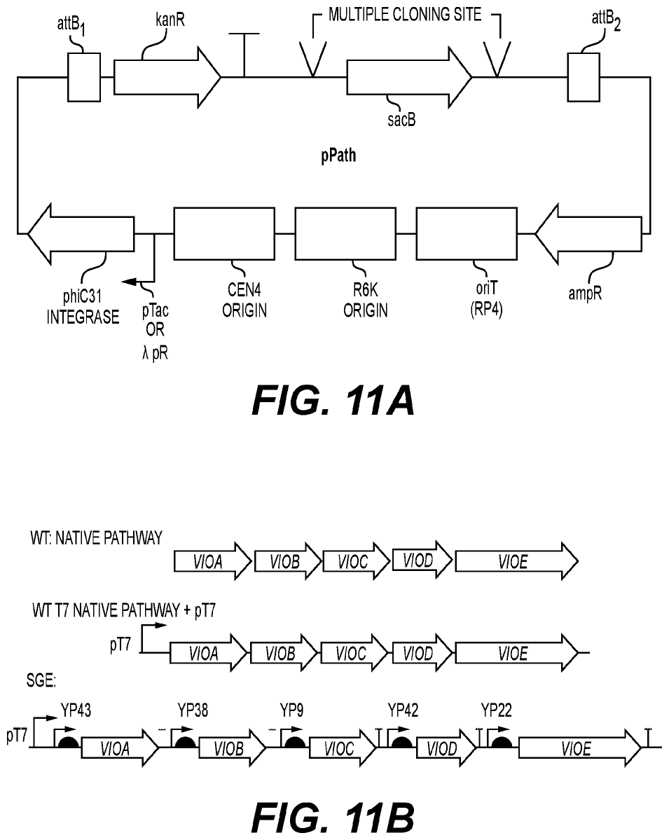

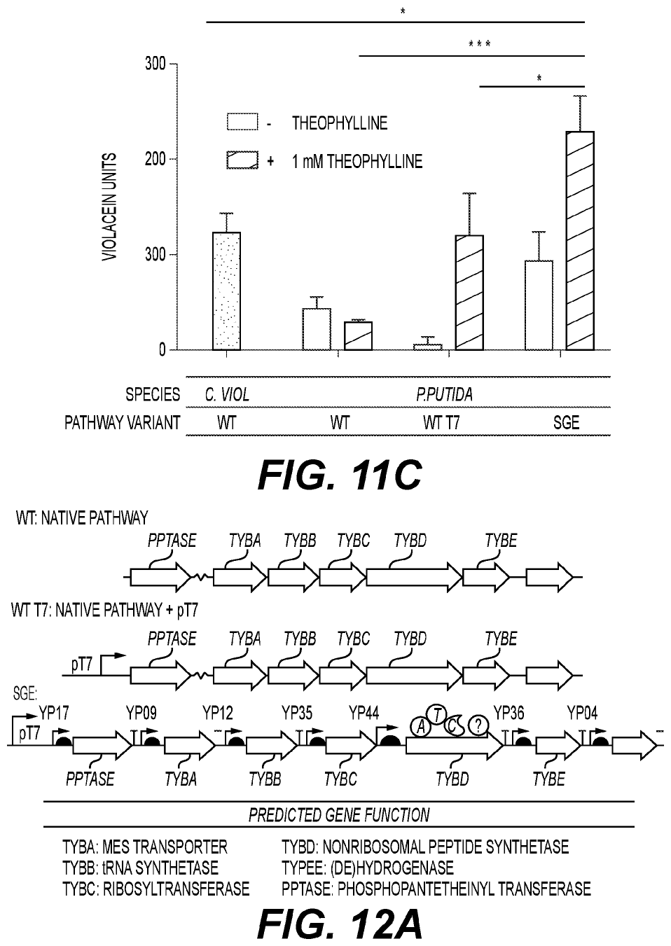

To overcome toxicity associated with high transposase activity, hyperactive variants of both the Himar (Lampe et al., 1999) and Tn5 transposases (Martinez-Garcia et al., 2011) were tested. Initially, these transposases were driven by a pTac promoter, which is highly active due to its consensus −10 and −35 promoter elements (de Boer et al., 1983). It was predicted that strong activity could counterbalance the exponentially decreasing efficiency associated with transposing large genetic constructs (e.g., ˜6 kb landing pad, FIG. 8A) (Lampe et al., 1998). Thus, it was hypothesized that with pTac, transposase expression would be repressed in a LacR+E. coli conjugation donor strain, while derepressed in recipient strains. However, attempts to clone this construct consistently resulted in mutations due to elevated basal expression from the pTac promoter (FIG. 9). Natively, transposases are negatively regulated, and synthetic overexpression is toxic (Weinreich et al., 1994). This issue is often resolved by using recipient-specific promoters that are transiently active (Dempwolff et al., 2019). However, to maintain broad host range, two solutions were developed. First, a trans-inhibiting plasmid, pInh, was constructed (FIG. 10A), expressing a dominant-negative Tn5 inhibitor gene (de la Cruz et al., 1993), as well as a SP6 RNA Polymerase that produced an anti-sense silencing transcript of the transposase gene. This inhibitor plasmid was designed only to replicate in the conjugal donor strain. Presence of this plasmid in the conjugal donor strain allowed successful cloning of landing pad constructs without mutation. In a second strategy, pTac was replaced with the bacteriophage λ pR promoter, repressed by a temperature sensitive CI857 gene (Valdez-Cruz et al., 2010). This promoter exhibited better repression in E. coli. Recoding the CI857 gene and appending a strong synthetic RBS with the disclosed CAD algorithm permitted stable construction and further reduced background by 25-fold (p<0.001) (FIG. 9). Taken together, these strategies successfully inhibited transposase activity in the conjugal donor, while allowing uninhibited transient expression in recipient microbes.

An apramycin selectable landing pad was tested, where seed transcription for the T7 RNAP circuit was provided either by the active, broad host-range promoter P1 from pIP1433 (Trieu-Cuot et al., 1985) (FIG. 9) or by relying on background transcription at the host integration locus. Upon mobilizing this landing pad into E. coli MG1655 (transconjugation frequency=1.5×10-5 per recipient), flow cytometry was used to evaluate the transposed population with and without T7 RNAP circuit induction (n˜2000 clones). It was observed that the resulting population had broad fluorescence distributions evidenced by elevated coefficient of variation (CV) (FIG. 10B) indicating that there was substantial clonal heterogeneity in expression, attributable to the context-dependent effects of individual genomic locus integration sites. Four individual clones were evaluated by flow cytometry, and analysis of the results indicated heterogeneity at several levels, including lower uninduced background fluorescence (Clone 1: σ=49 AU vs σ=146 AU for the population), tighter distributions: (Clone 2: CV=67 AU vs CV=232 AU for the population), higher induction strength (Clone 3: σ=68308 AU vs σ=3244 AU for the population), and overall shape of the fluorescence distribution (FIG. 10B). This approach permitted leveraging genetic context as a variable for tuning heterologous expression systems by selecting clones possessing the desired expression profile (FIG. 10B). The ability to survey multiple genetic loci allowed identification of a “privileged” clone (Clone 3), which upon theophylline induction, showed 20-fold stronger GFP expression than the population average (FIG. 10B). To further confirm this variability, the landing pad was introduced into various bacterial strains, and GFP induction was quantified for 7 randomly selected transconjugant clones. In E. coli, a version of the landing pad was used with and without a pIP1433 promoter acting to seed the T7 RNAP circuit. Both versions served to create clonal variability in fluorescence. Fluorescence was collected on a plate reader 12 hours after induction. aTc was kept constant at 100 ng/mL for all induced conditions. The variability in expression profiles emerged in landing pads that both contained and lacked the pIP1433 seeding promoter, indicating that the presence of a strong promoter at the 5′ edge of the landing pad did not preclude heterogeneity caused by the integration locus. (Table 5)

TABLE 5

| | | GFP Fluorescence | | | |
|---|---|---|---|---|---|
| | | | 0.1 mM | 1 mM | 10 mM |
| Strain | | Uninduced | Theophylline | Theophylline | Theophylline |
| Salmonella | Clone 7 | 550.8929 | 958.9748333 | 4311.245 | 11.90066667 |
| enterica | Clone 6 | 618.0985667 | 1034.874 | 3157.827667 | 74.83755556 |
| | Clone 5 | 1495.502 | 3383.883667 | 10618.99667 | 409.2835556 |
| | Clone 4 | 506.416 | 1021.5268 | 3843.365667 | 137.2382222 |
| | Clone 3 | 628.1293333 | 1276.701333 | 4927.18 | 190.1844444 |
| | Clone 2 | 649.1901333 | 1113.571667 | 4209.912667 | 104.5397778 |
| | Clone 1 | 741.1437333 | 4177.706 | 41297.93667 | 1249.181556 |
| Salmonella | Clone 7 | 5026.103 | 17149.38333 | 41049.13667 | 16652.645 |
| enterica | Clone 6 | 1554.845 | 4255.388333 | 9621.679667 | 3884.591667 |
| | Clone 5 | 6308.657667 | 14169.15 | 21389.51667 | 18618.591 |
| | Clone 4 | 743.1821667 | 1133.489333 | 3724.189667 | 4437.747333 |
| | Clone 3 | 6447.694667 | 13465.69667 | 42432.87667 | 8787.521667 |
| | Clone 2 | 755.1979 | 2122.732667 | 3266.568 | 4416.765333 |
| | Clone 1 | 1582.662333 | 2780.384 | 9381.291667 | 2976.09 |
| Pseudomonas | Clone 7 | 534.0645333 | 827.5156333 | 932.8945333 | 1071.232233 |
| putida | Clone 6 | 1535.413333 | 4670.779667 | 16869.19 | 3822.157333 |
| | Clone 5 | 497.4325333 | 942.2438333 | 1489.172333 | 983.2516667 |
| | Clone 4 | 691.4611667 | 2000.247333 | 7895.343667 | 1932.406 |
| | Clone 3 | 589.4761667 | 1138.410333 | 2194.684333 | 1150.183333 |
| | Clone 2 | 585.7280333 | 819.9375333 | 1209.503667 | 1138.214 |
| | Clone 1 | 3481.999 | 6091.941667 | 8871.198 | 3357.382667 |
| Pseudomonas | Clone 7 | 1512.676 | 2351.177 | 5717.102667 | 3824.183667 |
| veronii | Clone 6 | 838.6788667 | 906.3426667 | 1102.03 | 1077.436 |
| | Clone 5 | 727.0143333 | 1153.566667 | 1668.095333 | 1684.570333 |
| | Clone 4 | 1256.036 | 2235.054667 | 3124.381333 | 2224.855667 |
| | Clone 3 | 1057.5976 | 949.5893 | 1331.286667 | 2196.862333 |
| | Clone 2 | 734.9514 | 933.5809333 | 1916.017 | 1868.858667 |
| | Clone 1 | 891.3468667 | 1108.554333 | 1130.139267 | 1064.158133 |
| Escherichia | Clone 7 | 373.0561667 | 964.6233333 | 1979.388 | 5199.345667 |
| coli | Clone 6 | 2358.704333 | 2811.421333 | 2720.062 | 3936.797333 |
| pX = none | Clone 5 | 111.5697733 | 576.9899667 | 1025.992233 | 2556.777333 |
| | Clone 4 | 3213.067 | 3845.299667 | 3284.533667 | 5993.486667 |
| | Clone 3 | 872.7158 | 1659.394333 | 4009.909 | 9279.276667 |
| | Clone 2 | 623.2191667 | 882.6008333 | 1438.412333 | 3572.082667 |
| | Clone 1 | 4084.066667 | 4646.208 | 4925.66 | 10802.29167 |
| Escherichia | Clone 7 | 1062.691667 | 21089.71 | 23087.19333 | 20288.26 |
| coli | Clone 6 | 172.4307 | 2599.763667 | 2195.747333 | 4162.932333 |
| pX = 1433 | Clone 5 | 1258.311 | 2977.02 | 5495.354667 | 15721.70667 |
| | Clone 4 | 860.0710333 | 2587.806 | 5087.94 | 7284.247667 |
| | Clone 3 | 2429.164 | 39819.07333 | 45285.39 | 38273.75667 |
| | Clone 2 | 1150.501 | 2143.023667 | 3236.631333 | 7036.524333 |
| | Clone 1 | 153.10852 | 494.2229333 | 804.0765 | 1790.556333 |

To determine if this strategy works in diverse microbes, the disclosed conjugation-transposition system was tested on a select number of Gammaproteobacterial clades—Klebsiella aerogenes, Salmonella enterica, Pseudomonas putida, and Pseudomonas veronii, which exhibited transconjugation frequencies of 1.6×10⁻⁵, 9.2×10-8, 4.4×10⁻⁷, and 2.1×10⁻⁷ per recipient, respectively. Upon transposition, seven random clones were selected to assay for inducible GFP production. It was consistently found that within each strain, individual clones differ in the levels of GFP expression in response to theophylline induction (Table 5). These data demonstrate loci-specific variability of gene expression and the ability to use screened loci as a tunable property to control expression levels across strains and isolation of hosts with functional landing pads for introduction of genetic elements.

Once a selected strain has been domesticated with the landing pad, diverse SGEs can be readily introduced. SGEs are cloned into an R6K-based suicide vector, pPath (FIG. 12A), containing the phiC31 integrase and aminoglycoside resistance element functional in both prokaryotes (kanamycin) and S. cerevisiae (G418). Pathways were flanked with asymmetrical attB sites, such that when conjugated into recipient hosts, the site-specific integrase stably integrates the SGE cargo into the landing pad, displacing the GFP-luciferase reporter (FIG. 5B).

The functionality of this landing pad was demonstrated by introducing an SGE consisting of a redesigned pathway for the biosynthesis of the antimicrobial and immunomodulatory pigment violacein, produced natively by human pathogenic isolates of Chromobacterium violaceum (Kumar, 2012) (FIG. 5E). First, the consequences of redesigning this pathway into an SGE was quantified using the disclosed CAD-SGE algorithm. The native wildtype pathway sequence was compared to one where transcription is driven by pT7, and to a fully redesigned SGE. Prior metabolic engineering literature, which suggested that vioA and vioC should be expressed more strongly that the other three genes (Jones et al., 2015) was used to guide the selection of yeast promoter strength in the SGE. In the heterologous host P. putida, the wildtype sequence poorly produced pigment compared to pigment production by native C. violaceum (FIG. 5F). It was hypothesized that a bottleneck in the wildtype sequence is at the level of transcription since violacein production in its native host is controlled through a strain-specific quorum sensing mechanism (McClean et al., 1997). This was confirmed, as the wildtype pathway under the control of an orthogonal pT7 rescued metabolite production; this rescue was theophylline-dependent, indicating the landing pad activated the pathway transcriptionally (FIG. 5F). Full redesign into an SGE also rescued production. Pigment production with the SGE, induced with 1 mM theophylline+100 ng/mL aTc, was 8-fold over the wildtype pathway in P. putida (p<0.001) and 2-fold over that of C. violaceum (p<0.05). Production of the pigment could be titrated through the addition of theophylline (FIG. 5F). This SGE was transferred to other landing pad-domesticated microbes and production of violacein at OD, violacein units and strain fitness via final OD₆₆₀ were quantified. Strong pigment production was observed in a theophylline-inducible manner (FIG. 5G). In P. putida, levels of uninduced pigment were high, but could be boosted through the addition of up to 10 mM theophylline. This contrasted with K. aerogenes and S. enterica, where peak pigment production was at sub-maximal 1 mM levels of induction. The final OD₆₆₀ of induced strains was measured and the results indicated that these stains are less fit at high induction concentrations. Collectively, this highlights the importance of titratable induction as a method to mitigate toxicity and strain-to-strain variations in optimal expression levels uniquely achieved by the CAD-based re-design of the synthetic violacein pathway (Table 6).

TABLE 6

| | Violacein Units | | |
|---|---|---|---|
| | | 1 mM Theophylline + | 10 mM Theophylline + |
| Strain | Uninduced | aTc Induction | aTc Induction |
| Chromobacterium | 123.2348372 | | |
| violaceum | | | |
| Pseudomonas | 93.35522434 | 229.4066591 | 271.7803015 |
| putida | | | |
| Klebsiella | 144.9036812 | 215.8099063 | 56.29629251 |
| aerogenes | | | |
| Salmonella | 20.59086008 | 119.9689798 | 50.22623669 |
| enterica | | | |

### Characterization of a Human Commensal Biosynthetic Pathway

To evaluate its functional capability, the disclosed system was tested for natural product discovery using an uncharacterized BGC that had previously been computationally predicted (Donia et al., 2014) from the genome of Lactobacillus iners LEAF 2052A-d (cataloged in this publication as ‘BGC08’). This strain was isolated from the vagina of a bacterial vaginosis patient. The predicted gene cluster, referred to herein as the tyrocitabine (tyb) pathway, was initially annotated to contain a non-ribosomal peptide synthetase (tybD), a regulatory gene, a major facilitator family drug transporter (tybA), and several genes of unknown function. BLAST and InterPro searches also allowed the prediction of tRNA synthetase (tybB), ribosyltransferase (tybC), and (de)hydrogenase (tybE) functions for the unknown genes. Domain analysis of the NRPS indicated a single adenylation (A) domain, a peptidyl- or acyl-carrier protein thiolation domain (T), an incomplete condensation (C) domain, and a fourth domain of unknown function (?). Analysis of the tRNA synthetase suggested homology to Class Ic TyrRS. This gene contained a Rossmann ATP binding fold which carries out the amino acid activation and acylation reactions. However, it lacked the C-terminal RNA binding domain of canonical tRNA synthetases (Pang et al., 2014). Characterizing this pathway was prioritized due to human disease pathology of the source strain, implicating that the product is secreted, and the unusual pairing of a non-canonical tRNA synthetase and NRPS machinery in the operon. A closely situated downstream gene of unknown function was included in the cloning, as well as the native phosphopantetheinyl transferase (PPTase) gene located elsewhere in the genome, which would facilitate NRPS posttranslational activation. The upstream XRE family transcriptional regulator gene was emitted for SGE design (FIG. 12A). As done for the violacein pathway, the native wildtype pathway sequence was compared to one with orthogonal pT7 transcription, to a fully redesigned SGE (FIG. 12A). Motivated by and to initially compare experiments conducted with the violacein pathway, the orphan constructs were initially mobilized into the landing pad-domesticated P. putida host for metabolite analysis.

Two of the most highly abundant pathway-dependent metabolite ions (m/z 314.1195 and m/z 627.1771) were mobilized into processed landing pad-domesticated Pseudomonas putida. The wild-type, wild-type+pT7, and SGE variants of the pathway are compared using high-resolution liquid chromatography quadrupole-time of-flight mass spectrometry (LC-QTOF-MS) and analyzed through pathway-targeted metabolite analysis of the SGE. With the wildtype pathway, only trace amounts of the m/z 314 metabolite were detected, and quantifiable amounts of the m/z 627 metabolite were not detectable (Table 7) It was hypothesized that because the wildtype pathway was regulated by an immediate upstream transcriptional regulator, transcription could be one major bottleneck. However, complementation with heterologous pT7 overexpression in the Gram-negative Pseudomonas host of the phylum Proteobacteria was unable to rescue metabolite production. This highlights the relevance of multi-layer regulation that governs BGC functionality. For this native BGC from Gram-positive Lactobacillus of the phylum Firmicutes, the wildtype sequence contains a very low GC content of 27.7%, indicating possible maladapted codon usage in this case. Importantly, the fully redesigned SGE, which accounts for these multiple layers of regulation, successfully rescued metabolite production in P. putida.

TABLE 7

| | LC/MS Counts | | |
|---|---|---|---|
| | | 1 mM | 10 mM |
| | | Theophylline + aTc | Theophylline + aTc |
| Strain | Uninduced | Induction | Induction |
| Pseudomonas putida [Native Pathway] | 0 | 0 | 0 |
| Pseudomonas putida [Native Pathway | 0 | 0 | 0 |
| with T7 Promoter] | | | |
| Pseudomonas putida [Refactored | 18527 | 996440 | 0 |
| Pathway] | | | |

To further interrogate the biosynthesis of this pathway, E. coli BL21(DE3) was used to perform detailed reverse genetic analysis and scale-up production of intermediates and products for isolation and characterization. Here, expression was driven by the DE3 lysogen for T7 RNAP expression. Eleven new pathway-dependent entities [i.e., m/z 394.0858 tyrolose-phosphate (1), 314.1195 tyrolose (2), 627.1771 tyrocitabine (3), 669.1877 (M+H) acyl-tyrocitabine-696 (4ab), 697.2190 (M+H) acyl-tyrocitabine-696 (5ab), 725.2503 (M+H) acyl-tyrocitabine-724 (6ab), and 753.2816 (M+H) acyl-tyrocitabine-752 (7ab)] were characterized using a combination of mass-directed isolation from a 20 L culture, ultraviolet/visible (UV/Vis) spectroscopy, tandem MS (MS/MS), multidimensional NMR techniques (1H, 13C, and 31P), NMR computational analysis, and/or synthetic validation. Briefly, UV and multidimensional NMR analyses revealed the structure of m/z 314, which was termed tyrolose (2), to be a ribosylated tyrosine that had undergone an Amadori rearrangement. The configuration of the tyrosine motif was established as S via Marfey's analysis (Bhushan and Brtickner, 2004). The stereochemical assignment of the carbohydrate moiety was accomplished utilizing rotating frame Overhauser effect spectroscopy (ROESY) NMR analysis, and the absolute structure of 2 was confirmed using a synthetic standard (via a Zn²⁺-catalyzed reaction (Chanda and Harohally, 2018)). A phosphorylated variant of 2 termed tyrolose-phosphate (1) was also confirmed using a synthetic standard. MS/MS fragmentation analysis and molecular formula assignment of m/z 627, which was termed tyrocitabine (3), suggested that this compound could be generated via an adenylation-rearrangement sequence of the tyrolose substrate(s) (FIG. 12C). 1D and 2D NMR experiments on tyrocitabine established the presence of AMP and an orthoester-phosphate-type motif as two key structural building blocks. The connectivity of these two moieties was established via a 1D 31P NMR decoupling experiment. The 3D structure of the ring system in 3 constructed by the orthoester-phosphate moiety was confirmed by the comparison of experimental interproton distances calibrated from ROESY with those from computational simulation of plausible diastereomers. The remaining pathway-dependent entities (m/z 669, 697, 725, and 753) were structurally related to 3 with varying acyl modifications [i.e., m/z 669, acetylation, acyl-tyrocitabine-668 (4ab); m/z 697, butyrylation, acyl-tyrocitabine-696 (5ab); m/z 725, hexanoylation, acyl-tyrocitabine-724 (6ab); and m/z 753, octanoylation, acyl-tyrocitabine-752 (7ab)]. ROESY and heteronuclear multiple bond correlation (HMBC) NMR analyses confirmed that these acyl chains were substituted at the 3′-position (major, a series) of the AMP ribosyl moiety with some observed spontaneous intramolecular transesterification to the 2′-position (minor, b series).

Single gene deletions of the multigene pathway in E. coli (FIG. 12C) supported an order of operations in tyrocitabine assembly (FIG. 12D-12E). Indeed, genetic studies support a stepwise biosynthesis in which the anthranilate phosphoribosyltransferase-family ribosyltransferase (tybC) is required for the ribosylation of tyrosine and the subsequent Amadori rearrangement leading to tyrolose-phosphate (1) and tyrolose (2), revealing a transformation for this class of enzymes. It was observed that the (de)hydrogenase (tybE) and tRNA synthetase (tybB) are required for the “abortive” tRNA synthetase reaction, leading to tyrocitabine, the free adenylated product 3 featuring an orthoester linkage at the phosphate moiety. Orthoesters appear in a variety of natural products, but their biosyntheses remain largely unknown (Li et al., 2018; Matsuda et al., 2018). Abortive tRNA synthetase reactions, side reactions of canonical tRNA synthetases, have only recently been described, which lead to stress-enhanced signaling molecules that modulate quorum sensing responses (Kim et al., 2020). The non-canonical tRNA synthetase TvbB that lacks the C-terminal RNA binding motif is required for a dedicated abortive reaction to access tyrocitabine (3), indicating an evolutionary selection for loss of traditional tRNA synthetase functionality in favor of specialized metabolite biosynthesis. Acylation of 3 to access major 4a-7a required the tybD NRPS gene, indicating that the NRPS plays an acyl-ligase role.

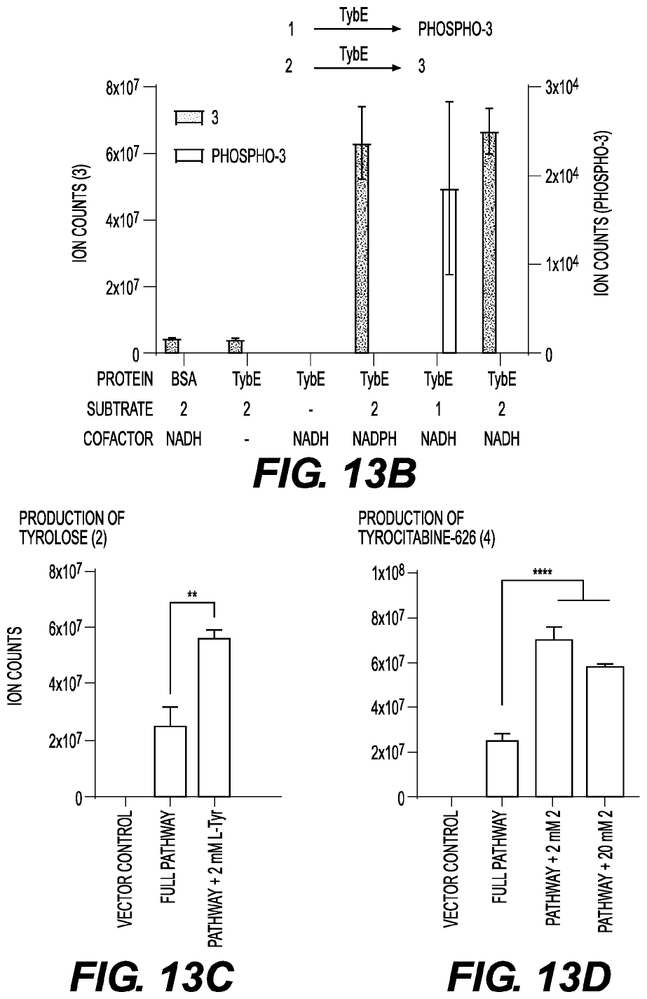

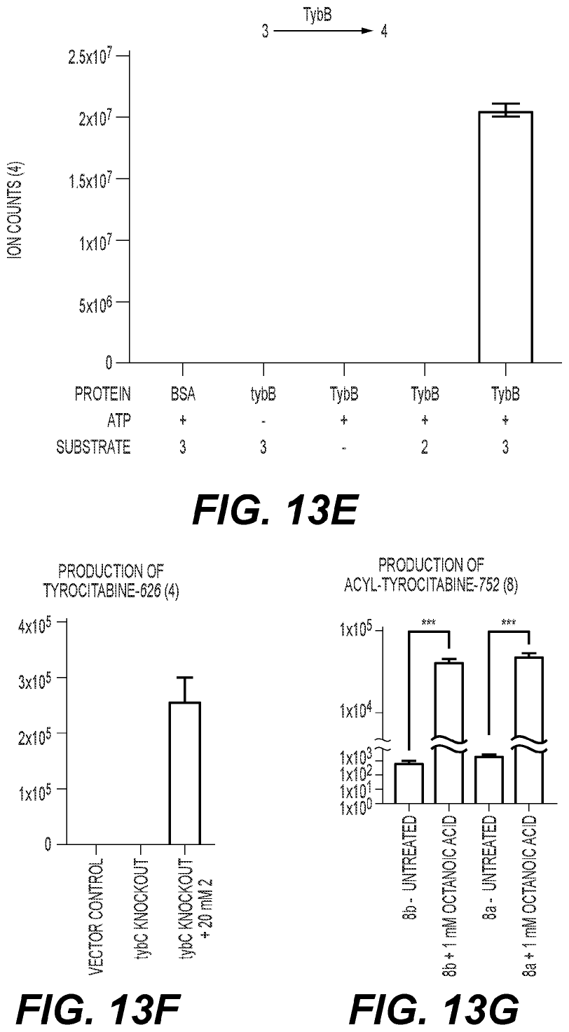

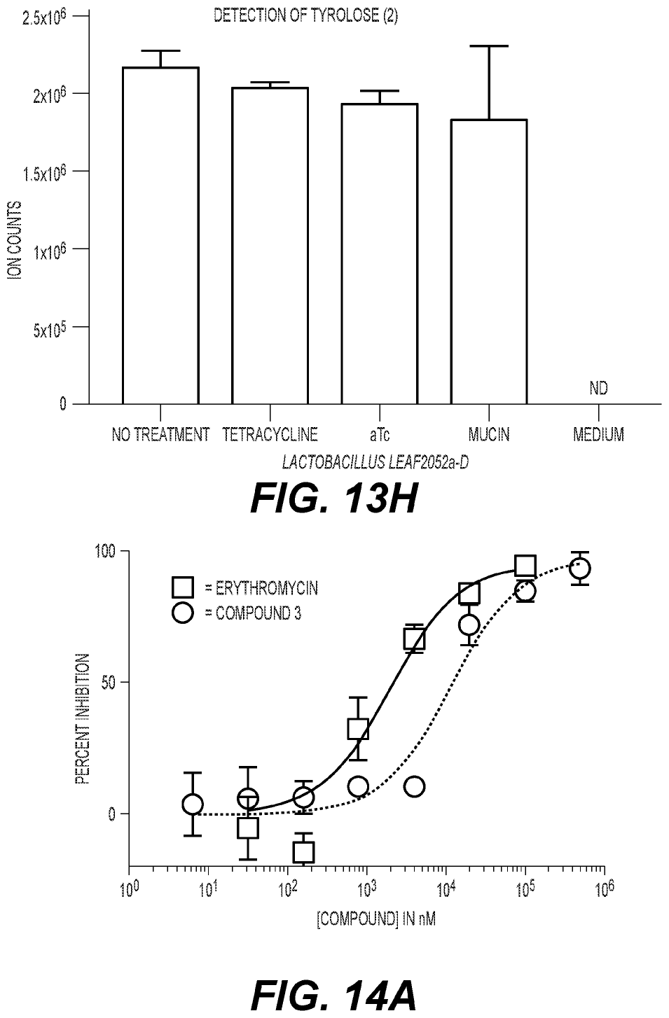

To confirm the biosynthetic route, in vitro protein biochemical studies were conducted using individually purified enzymes and substrate feeding studies in E. coli expressing the tyb pathway (tyb+). It was first established that isolated TybC uses L-Tyr and PRPP as a ribosyl donor to produce the Amadori rearrangement products 1 and 2 (FIG. 13A). Next, it was demonstrated that TybE catalyzes a stereoselective hydrogenation of both 1 and 2 into phospho-3 and 3, respectively (FIG. 13B). However, phospho-3 was not detected in cell extracts from tyb+E. coli, indicating early phosphate hydrolysis in cells. L-Tyr supplementation in tyb+E. coli enhanced production of 2 (FIG. 13C) consistent with TybC's proposed Amadori synthase role. Moreover, supplementation with synthetic 2 enhanced production of 4 in tyb+E. coli and was capable of “chemically complementing” a knockout of the ribosyltransferase tybC. These studies strongly support the intermediacy of 2 (FIGS. 13D and 13E). It was further demonstrated that isolated TybB could directly transform the polyol-amino acid 3 into the orthoester-phosphate 4 in an ATP-dependent manner (FIG. 13E), confirming the “abortive” tRNA synthetase role of this tRNA synthetase family. Finally, feeding free fatty acids, such as octanoic acid, to tyb+E. coli significantly enhanced production of the ultimate NRPS-dependent acyl-tyrocitabines (FIG. 13G).

To establish a biological activity for the tryocitabine family, the similarity ensemble approach was used according to previous studies (SEA) (Keiser et al., 2007) to computationally predict candidate targets, and various components of protein translation were among the hits. PURExpress (NEB) protein synthesis technologies was used to probe a molecular mechanism and it was established that metabolite 3 inhibited translation of a GFP reporter with a half-maximal inhibition (IC₅₀) of 13 μM, which was comparable to an erythromycin control (IC₅₀ 2 μM)(FIG. 14A). This inhibition occurred with either DNA or RNA substrates, indicating that inhibition in the in vitro system was largely occurring at the post-transcriptional level (FIG. 14B, 14C). The activity was abrogated when tyrocitabine was acylated, indicating a possible prodrug mechanism where the acylated molecules would require hydrolytic activation by esterases in the recipient organism(s) (FIG. 14D).

The SGE was mobilized into various bacteria (E. coli MG1655, K. aerogenes, P. putida, B. subtilis, and S. enterica) as well as S. cerevisiae to test broad-host mobilization and expression. It was observed that although the disclosed SGE can successfully produce the bioactive tyrocitabine (3) in all strains, variation in the relative abundances of the various tyrocitabines and their intermediates were also observed, indicating strain-specific differences in metabolic flux through the pathway (FIG. 15A).

TABLE 8
LC/MS Counts

| | | | | 1 mM | | 10 mM | |
|---|---|---|---|---|---|---|---|
| | | | | Theophylline + | | Theophylline + | |
| | Uninduced | | | aTc Induction | | aTc Induction | |
| Escherichia coli | 0 | 0 | 0 | 168001 | 138542 | 88723 | 71706 |
| Klebsiella | 0 | 0 | 0 | 261014 | 29185 | 0 | 0 |
| aerogenes | | | | | | | |
| Pseudomonas | 0 | 0 | 0 | 309611 | 277159 | 0 | 0 |
| putida | | | | | | | |
| Salmonella | 0 | 0 | 0 | 39850 | 38772 | 0 | 21475 |
| enterica | | | | | | | |
| Bacillus subtilis | 0 | 0 | 0 | 0 | 0 | 0 | 0 |
| Saccharomyces | 0 | 0 | 0 | | | | |
| cerevisiae | | | | | | | |

The P. putida host was found to be particularly gifted in producing the largest molecule acyl-tyrocitabine-752 (7), as assessed by relative LC-QTOF-MS analysis. In contrast, tyrocitabine and its precursors, but not the acyl-tyrocitabines, were detected in B. subtilis or S. cerevisiae. This diversity of outcomes highlights the utility of the disclosed approach in enabling rapid dissemination of genetic material across numerous strains belonging to broad taxonomic groups. Attempts to detect and induce production of the tyrocitabines in the native Lactobacillus iners LEAF 2052a-D failed to detect pathway-dependent metabolites beyond tyrolose (2) under the conditions of the current studies (FIGS. 13G and 13H), highlighting the importance of employing a robust strategy to elucidate this pathway in heterologous hosts.

To analyze the broader phylogenetic distribution of this new class of molecules, amino acid BLAST homology searches of microbial genome sequences hosted on JGI-IMG were performed, using the abortive tRNA synthetase TybB as a base. Approximately 100 close hits were found, with a 1×10⁻⁵ E-value cutoff, largely distributed across other Firmicutes as well as Actinobacteria (FIG. 15B, Table 2). 92 of these hits clustered with at least one other biosynthetic enzyme. To identify homologs at a gene cluster level and provide secondary support, a search using cBlaster was performed, binning on clusters that contained at least two genes with at least 20% amino acid similarity with proteins encoded in the Lactobacillus iners tyb pathway (Gilchrist et al., 2021). Though the resulting hits largely overlapped, cBlaster identified additional hits among archaeal species, indicating cross-domain transfer of this pathway. These hits, like TybB, resembled Class 1c tRNA synthetases, but lacked an RNA binding domain, as predicted through InterPro (FIG. 15C). Hits annotated as both TrpRS and TyrRS were found, indicating potential differences in amino acid substrate specificity. Interestingly, these annotated “tRNA synthetases” were encoded in highly diversified operon contexts. Among the various operons (24 illustrated in FIG. 15B), the common feature was presence of the TybC-like ribosyltransferase, but co-localizing accessory genes ranged from predicted NRPSs to hydrolases, glycosyltransferases, (de)hydrogenases, methyltransferases, and radical SAM enzymes. Notably, several operons contained multiple Class Ic synthetase-like enzymes in tandem, both lacking RNA binding domains. Based on the proposed biosynthetic route of the tyrocitabines and observation of various accessory enzymes in the related uncharacterized pathways, these data indicate that the tyrocitabines represent the founding members of a much broader class of specialized nucleotide metabolites and motivate further investigation in future studies.

### Example 2: A Range of RNA Polymerase are Effective in the System

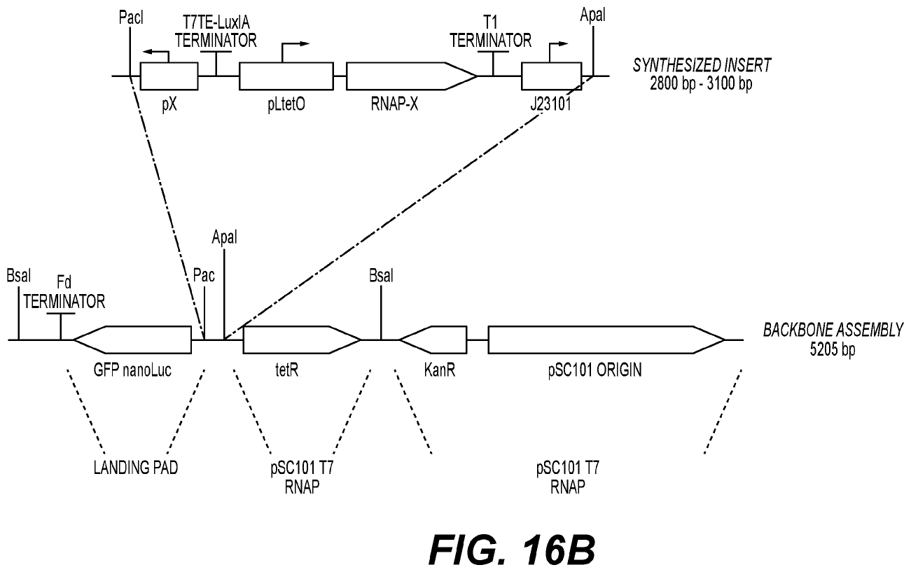

The disclosed orthogonal RNA polymerase system is an innovative synthetic biology tool that facilitates the precise control of gene transcription, independent of the host cell's native RNA polymerase machinery. This allows for the expression of genes that may be toxic or incompatible with the host cell's biological processes. To expand the orthogonality of the current T7 polymerase system, additional phage RNA polymerases such as T3, SP6, KP34, and K11 polymerases were introduced into the system. This involves designing different codon-optimized RNA polymerases, which can recognize specific promoter sequences placed upstream of genes of interest (FIG. 16A). Inserts for each of the selected polymerases were synthesized and their transcription regulated using pLtetO so that transcription can be controlled by aTc. The synthesized inserts were then cloned into an entry vector to drive the expression of GFP-nanLuc under the control of specific promoters for each polymerase (FIG. 16B).

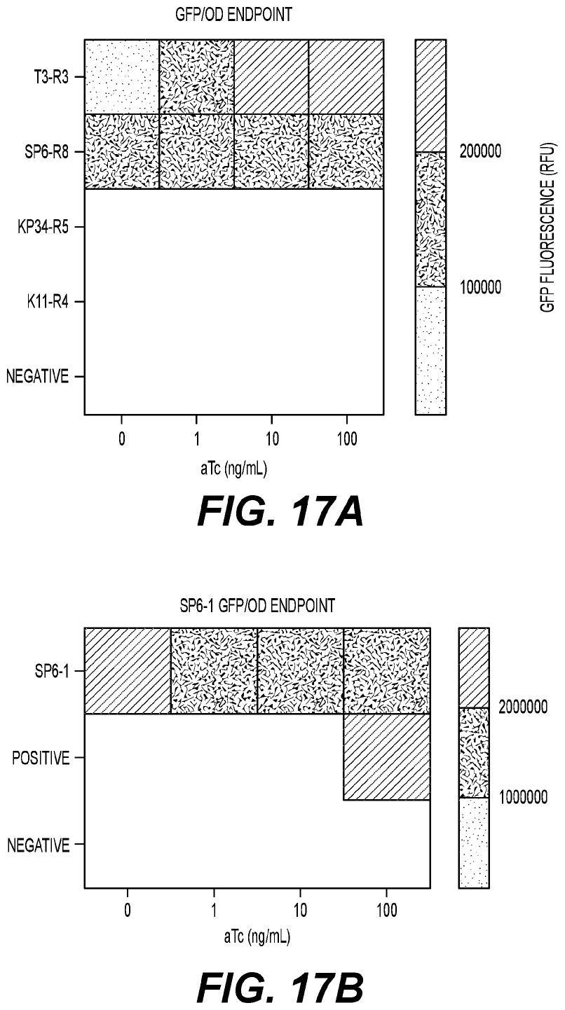

The activity and tunability of the four RNA polymerases, T3, SP6, KP34, and K11 were tested, and the results showed that T3-R3 displayed better tunability under aTc induction, with increasing GFP fluorescence in response to increasing inducer concentration. SP6-R8 showed constitutive GFP expression, indicating that the SP6 polymerase may be highly active, given that only baseline expression was enough to drive GFP production. In contrast, KP34 and K11 displayed much lower GFP readouts (FIG. 17A).

Sequencing of the T3-R3 clone revealed a deletion that resulted in a premature stop codon for the T3 polymerase. Despite this, partial expression of T3 polymerase was still functional. Additionally, another clone, SP6-1, was identified, with confirmed sequence, which exhibited high GFP expression without aTc induction that decreased with aTc addition (FIG. 17B).

This approach and these results further illustrate the versatility of the disclosed compositions and methods, and their ability to improve the precision and versatility of gene expression control in synthetic biology applications.

### Example 3: Alternative Regulatory Circuits are Effective in the System

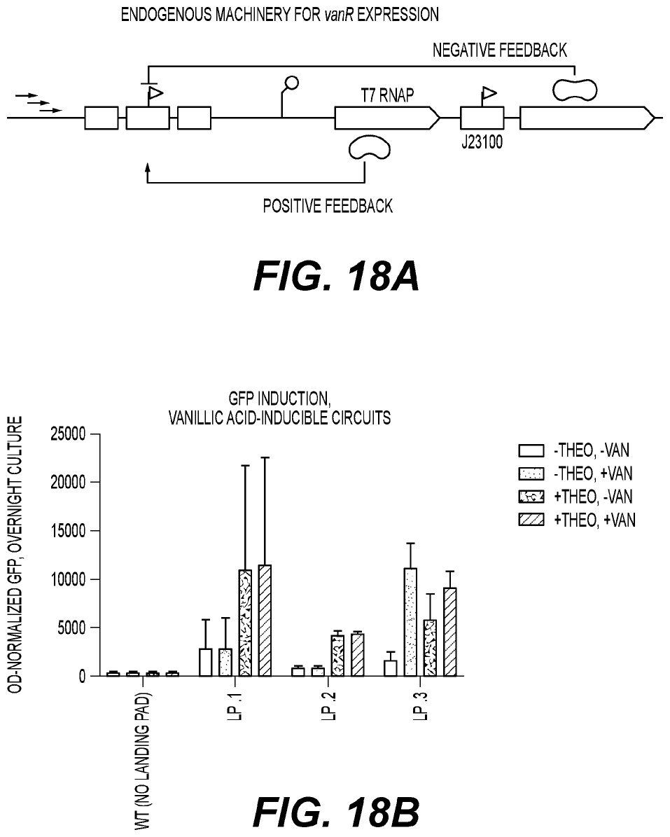

To further illustrate the versatility of the system, a Vanillic acid-regulated circuit was tested in place of aTc. See, e.g., FIGS. 18A-18B. This circuit is essentially regulated exclusively by theophylline. LP.1-3 are genetic system architecture variants and show some associated differences in expression data. For example, results show LP.2 has a similar dynamic range to pT7-vanR architecture (LP.1) and lower “off” and “on” states than LP.1. Higher levels of repressor were obtained from the constitutive promoter (FIG. 18B). These data further illustrate the SGE systems work with different inducer-promoter-protein variants (i.e., beyond the aTc-TetR tested in Example 1) and thus further establishes the modularity and scalability of the system.

### Example 4: SGE Functions in Cyanobacteria and Cupriavidus necator

Genomic integration and heterologous expression of a genetic element occur through a two-step process:
1. Random transposition of landing pad into genome
   Inducible (host-orthogonal T7RNAP)
   Reporter to assess inducibility at integration site
2. phiC31 sites allow site-specific integration of a gene or pathway of interest
   Inducible expression of versatile genetic elements across a wide range of hosts

See, e.g., all of Example 1, particularly FIGS. 8A and 8B and its description, Patel, et al., Cell. 2022; 185(9):1487-1505.e14, which is specifically incorporated by reference herein in its entirety.

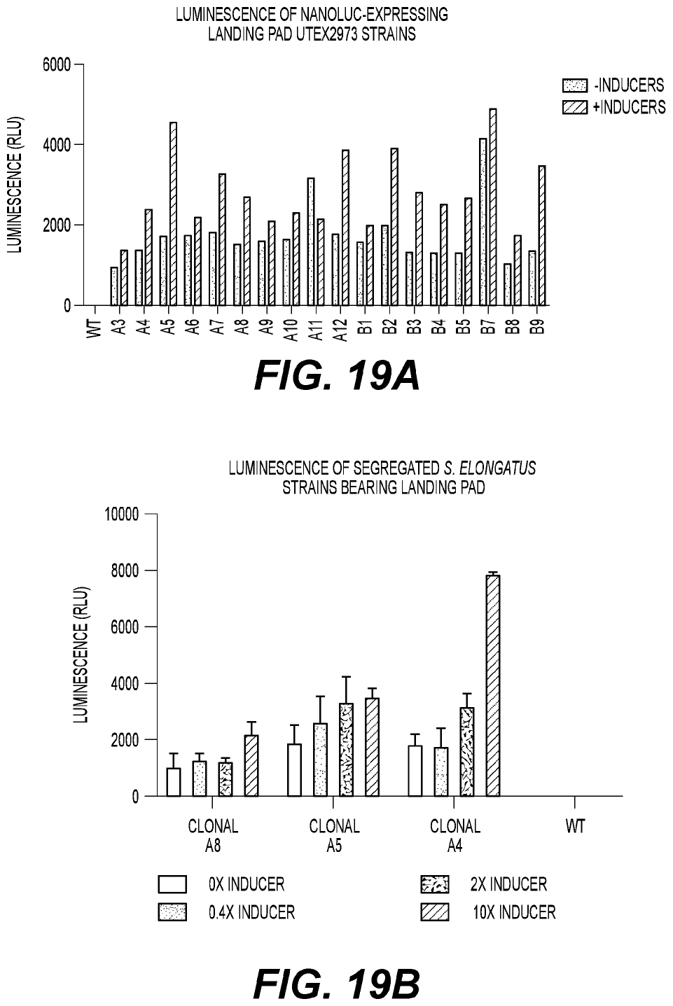

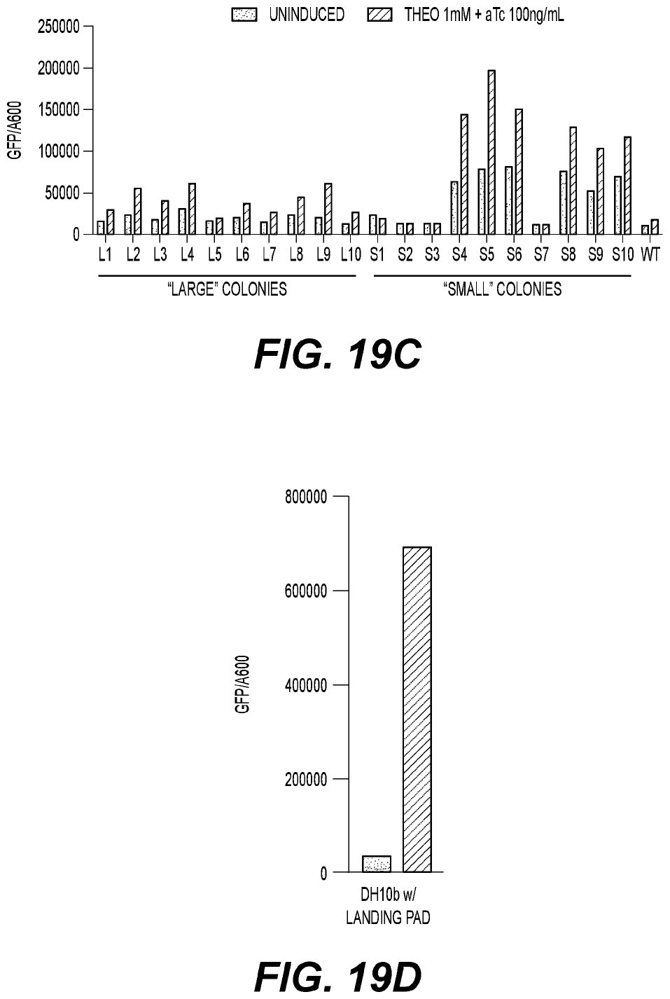

To further illustrate the versatility of the system, it was also tested in UTEX 2973 and Synechococcus elongatus cyanobacterias and Cupriavidus necator bacteria using GFP as an expression indicator. Results are illustrated in FIGS. 19A-19D, and indicate this system is also effective in these hosts.

SGE function in cyanobacteria in these experiments was characterized by low dynamic range (greatest induction ˜2.5 for UTEX 2973 and ˜4× for Synechococcus elongatus) and high background expression, but nonetheless further illustrates the system's activity across diverse organisms.

TABLE 9
Key Resources
Key Resources
Table

| | | |
|---|---|---|
| REAGENT or | | |
| RESOURCE | SOURCE | IDENTIFIER |

Chemicals

| | | |
|---|---|---|
| Chloramphenicol | Sigma | C0378 |
| Spectinomycin | DOT Scientific | DSS23000-5 |
| Kanamycin | American Bio | AB01100 |
| Apramycin | Fisher | AAJ66616 |
| Carbenicillin | Sigma | C1389 |
| G418 | Fisher | 10131035 |
| Hygromycin B | Fisher | 10687010 |
| Luria Broth | American Bio | AB01198 |
| M9 Minimal Media | Fisher | DF0485-17 |
| Erythromycin | Acros | 227330050 |
| Amberlite XAD-7 | | |
| Resin | Acros | 202245000 |
| Celite | Acros | 349675000 |

Commercial Assays

| | | |
|---|---|---|
| Luna Universal qRT- | NEB | E3005 |
| PCR kit | | |
| Purexpress translation | NEB | E6800S |
| kit | | |
| HiScribe T7 RNA Kit | NEB | E2040S |

Experimental Models: Organisms and Strains

| See Extended Data S4 | Indicated when externally acquired |
|---|---|
| for detailed list | |

Recombinant DNA

| | | |
|---|---|---|
| Source of PhiC31 | (Groth et al., 2000) | Addgene 18941 |
| Integrase | | |
| Source of Tn5 | (Martínez-García et | Addgene 61564 |
| Transposase | al., 2011) | |
| Source of miniR6K | (Puri et al., 2015) | Addgene 61263 |
| origin | | |
| Source of pAMβ1 | (O’Sullivan and | Addgene 71312 |
| origin | Klaenhammer, 1993) | |

(See Extended Data S4 and FIG. S6, for detailed description
of all additional contructs designed for this study)
Software and Algorithms

| | | |
|---|---|---|
| ChemDraw 20 | PerkinElmer | perkinelmerinformatics.com/products/research/chemdraw/ |
| Mnova | Mestrelab Research | mestrelab.com/download/mnova/ |
| Prism 7 | Graphpad | graphpad.com/ |
| Adobe Illustrator CC | Adobe | Adobe.com |
| Python 3 | | python.org/ |
| Transtermhp 2.08 | (Kingsford et al., 2007) | transterm.cbcb.umd.edu |
| Vienna RNA Suite | (Lorenz et al., 2011) | tbi.univie.ac.at/RNA/ |
| 2.4.14 | | |
| NuPoP 3 | (Xi et al., 2010) | bioconductor.org/packages/release/bioc/html/NuPoP.html |
| R 4 | | r-project.org/ |
| phyloT v2 | | phylot.biobyte.de/ |
| iTOL v6 | (Letunic and Bork, 2021) | itol.embl.de/ |
| DNAplotlib | (Der et al., 2017) | github.com/VoigtLab/dnaplotlib |

```
Appendix I: Additional Sequences: pLP (ptac-himar
transposase, apramycinR)
(SEQ ID NO: 125)
taacaggttggatgataagtccccggtctagattgccttgaatataTTGACAatactgataagataataTATAATatatctttA
ctaccaagacgataaatgcgtcggaaaagtttaatactTTTGttagatatatttttttgtgTAatTTTGtaatcgttatgcggcagt
aaaaggatctattataaggaggcactcaccATGCAATACGAATGGCGAAAAGCCGAGCTCATCGG
TCAGCTTCTCAACCTTGGGGTTACCCCCGGCGGTGTGCTGCTGGTCCACAGCTCC
TTCCGTAGCGTCCGGCCCCTCGAAGATGGGCCACTTGGACTGATCGAGGCCCTG
CGTGCTGCGCTGGGTCCGGGAGGGACGCTCGTCATGCCCTCGTGGTCAGGTCTG
GACGACGAGCCGTTCGATCCTGCCACGTCGCCCGTTACACCGGACCTTGGAGTT
GTCTCTGACACATTCTGGCGCCTGCCAAATGTAAAGCGCAGCGCCCATCCATTT
GCCTTTGCGGCAGCGGGGCCACAGGCAGAGCAGATCATCTCTGATCCATTGCCC
CTGCCACCTCACTCGCCTGCAAGCCCGGTCGCCCGTGTCCATGAACTCGATGGG
CAGGTACTTCTCCTCGGCGTGGGACACGATGCCAACACGACGCTGCATCTTGCC
GAGTTGATGGCAAAGGTTCCCTATGGGGTGCCGAGACACTGCACCATTCTTCAG
GATGGCAAGTTGGTACGCGTCGATTATCTCGAGAATGACCACTGCTGTGAGCGC
TTTGCCTTGGCGGACAGGTGGCTCAAGGAGAAGAGCCTTCAGAAGGAAGGTCC
AGTCGGTCATGCCTTTGCTCGGTTGATCCGCTCCCGCGACATTGTGGCGACAGCC
CTGGGTCAACTGGGCCGAGATCCGTTGATCTTCCTGCATCCGCCAGAGGCGGGA
TGCGAAGAATGCGATGCCGCTCGCCAGTCGATTGGCTAATAGGGATAATCAGAA
TTGGTTAATTGGTTGTAACACTGGTCTATCATTGATAGGTATAAATTAATACGAC
TCACTAATACTGAACCTATCAGTGATAGATACCGGTGATACCAGCATCGTCTTG
ATGCCCTTGGCAGCACCCTGCTAAGGAGGCAACAAGATGAACACGATTAACATC
GCTAAGAACGACTTCTCTGACATCGAACTGGCTGCTATCCCGTTCAACACTCTGG
CTGACCATTACGGTGAGCGTTTgGCTCGCGAACAGTTGGCCCTTGAGCATGAGTC
TTACGAGATGGGTGAAGCACGCTTCCGCAAGATGTTTGAGCGTCAACTTAAAGC
TGGTGAGGTTGCGGATAACGCTGCCGCCAAGCCTCTCATCACTACCCTACTCCCT
AAGATGATTGCACGCATCAACGACTGGTTTGAGGAAGTGAAAGCTAAGCGCGG
CAAGCGCCCGACAGCCTTCCAGTTCCTGCAAGAAATCAAGCCGGAAGCCGTAGC
GTACATCACCATTAAGACCACTCTGGCTTGCCTAACCAGTGCTGACAATACAAC
CGTTCAGGCTGTAGCAAGCGCAATCGGTCGGGCCATTGAGGACGAGGCTCGCTT
CGGTCGTATCCGTGACCTTGAAGCTAAGCACTTCAAGAAAAACGTTGAGGAACA
ACTCAACAAGCGCGTAGGGCACGTCTACAAGAAAGCATTTATGCAAGTTGTCGA
GGCTGACATGCTCTCTAAGGGTCTACTCGGTGGCGAGGCGTGGTCTTCGTGGCA
TAAGGAAGACTCTATTCATGTAGGAGTACGCTGCATCGAGATGCTCATTGAGTC
AACCGGAATGGTTAGCTTgCACCGCCAAAATGCTGGCGTAGTAGGTCAAGACTC
TGAGACTATCGAACTCGCACCTGAATACGCTGAGGCTATCGCAACCCGTGCAGG
TGCGCTGGCTGGCATCTCTCCGATGTTCCAACCTTGCGTAGTTCCTCCTAAGCCG
TGGACTGGCATTACTGGTGGTGGCTATTGGGCTAACGGTCGTCGTCCTCTGGCGC
TGGTGCGTACTCACAGTAAGAAAGCACTGATGCGCTACGAAGACGTTTACATGC
CTGAGGTGTACAAAGCGATTAACATTGCGCAAAACACCGCATGGAAAATCAAC
AAGAAAGTCCTAGCGGTCGCCAACGTAATCACCAAGTGGAAGCATTGTCCGGTC
GAGGACATCCCTGCGATTGAGCGTGAAGAACTCCCGATGAAACCGGAAGACAT
CGACATGAATCCTGAGGCTCTCACCGCGTGGAAACGTGCTGCCGCTGCTGTGTA
CCGCAAGGACAAGGCTCGCAAGTCTCGCCGTATCAGCCTTGAGTTCATGCTTGA
GCAAGCCAATAAGTTTGCTAACCATAAGGCCATCTGGTTCCCTTACAACATGGA
CTGGCGCGGTCGTGTTTACGCTGTGTCAATGTTCAACCCGCAAGGTAACGATAT
GACCAAAGGACTGCTTACGCTGGCGAAAGGTAAACCAATCGGTAAGGAAGGTT
ACTACTGGCTGAAAATCCACGGTGCAAACTGTGCGGGTGTCGATAAGGTTCCGT
TCCCTGAGCGCATCAAGTTCATTGAGGAAAACCACGAGAACATCATGGCTTGCG
CTAAGTCTCCACTGGAGAACACTTGGTGGGCTGAGCAAGATTCTCCGTTCTGCTT
CCTTGCGTTCTGCTTTGAGTACGCTGGGGTACAGCACCACGGCCTGAGCTATAA
CTGCTCCCTTCCGCTGGCGTTTGACGGGTCTTGCTCTGGCATCCAGCACTTCTCC
GCGATGCTCCGAGATGAGGTAGGTGGTCGCGCGGTTAACTTGCTTCCTAGTGAA
ACCGTTCAGGACATCTACGGGATTGTTGCTAAGAAAGTCAACGAGATTCTACAA
GCAGACGCAATCAATGGGACCGATAACGAAGTAGTTACCGTGACCGATGAGAA
CACTGGTGAAATCTCTGAGAAAGTCAAGCTGGGCACTAAGGCACTGGCTGGTCA
ATGGCTGGCTTACGGTGTTACTCGCAGTGTGACTAAGCGTTCAGTCATGACGCT
GGCTTACGGGTCCAAAGAGTTCGGCTTCCGTCAACAAGTGCTGGAAGATACCAT
TCAGCCAGCTATTGATTCCGGCAAGGGTCTGATGTTCACTCAGCCGAATCAGGC
TGCTGGATACATGGCTAAGCTGATTTGGGAATCTGTGAGCGTGACGGTGGTAGC
TGCGGTTGAAGCAATGAACTGGCTTAAGTCTGCTGCTAAGCTGCTGGCTGCTGA
GGTCAAAGATAAGAAGACTGGAGAGATTCTTCGCAAGCGTTGCGCTGTGCATTG
GGTAACTCCTGATGGTTTCCCTGTGTGGCAGGAATACAAGAAGCCTATTCAGAC
GCGCTTGAACCTGATGTTCCTCGGTCAGTTCCGCTTgCAGCCTACCATTAACACC
AACAAAGATAGCGAGATTGATGCACACAAACAGGAGTCTGGTATCGCTCCTAAC
TTTGTACACAGCCAAGACGGTAGCCACCTTCGTAAGACTGTAGTGTGGGCACAC
GAGAAGTACGGAATCGAATCTTTTGCACTGATTCACGACTCCTTCGGTACCATTC
CGGCTGACGCTGCGAACCTGTTCAAAGCAGTGCGCGAAACTATGGTTGACACAT
ATGAGTCTTGTGATGTACTGGCTGATTTCTACGACCAGTTCGCTGACCAGTTGCA
CGAGTCTCAATTGGACAAAATGCCAGCACTTCCGGCTAAAGGTAACTTGAACCT
CCGTGACATCTTgGAGTCGGACTTCGCGTTCGCGTAAAAGCTTGATATCGAATTC
CTGCAGCCCCGGGGATCCCATGGTACGCGTGCTAGTAATACGACTCACTAATAC
TGAATCCAAACCCTCGTTAGGGGAGCGTCTAATTTTAGGAGATCCAAAATGTCA
AGGCTGGATAAATCAAAAGTAATCAATAGCGCGCTGGAACTGCTGAACGAGGT
CGGCATCGAAGGTCTGACCACCCGCAAGCTGGCGCAAAAACTGGGCGTCGAAC
AACCGACGCTGTACTGGCACGTAAAAAATAAGCGTGCGCTGCTGGACGCACTGG
CAATTGAAATGCTGGATCGTCACCACACCCACTTCTGTCCGCTGGAGGGTGAAT
CATGGCAAGATTTCCTTCGCAACAACGCGAAGTCATTTCGCTGCGCGCTGCTGA
GCCACCGCGATGGAGCAAAAGTTCATCTGGGCACCCGCCCAACGGAGAAACAA
TATGAAACGCTGGAAAACCAGCTTGCCTTCCTGTGCCAGCAGGGTTTCAGCCTT
GAGAACGCGCTGTACGCGCTGAGCGCCGTAGGTCACTTCACCCTGGGCTGTGTT
CTGGAAGACCAAGAACATCAAGTAGCAAAAGAAGAGCGAGAAACCCCTACGAC
CGATTCGATGCCGCCGCTGCTGCGTCAGGCGATTGAACTGTTCGATCACCAGGG
CGCGGAACCGGCATTCCTGTTTGGTCTGGAACTTATTATATGCGGCCTAGAAAA
ACAACTGAAGTGCGAAAGCGGTAGCTAAcgccgaaaaccccgcttcggcggggttttgccgcATAA
CAGGGTAATccccaactggggtaacctGTgagttctctcagttggggAAAAAAAAACCCCGCCCCTG
ACAGGGCGGGGTTTTTTTTTTTCGCCGCGTTGGCTAgTAATACGACTCACTATAG
GGAGACTTAAGTATAAGGAGGAAAAAATATGAGCAAGGGCGAAGAACTGTTTA
CGGGCGTGGTGCCGATTCTGGTGGAACTGGATGGTGATGTCAATGGTCACAAAT
TCAGCGTGCGCGGCGAAGGTGAAGGCGATGCAACCAATGGTAAACTGACGCTG
AAGTTTATTTGCACCACGGGTAAACTGCCGGTTCCGTGGCCGACCCTGGTCACC
ACGCTGACGTATGGTGTTCAGTGTTTCAGTCGTTACCCGGATCACATGAAACGC
CACGACTTTTTCAAGTCCGCGATGCCGGAAGGTTATGTCCAAGAACGTACCATC
TCATTTAAAGATGACGGCACCTACAAAACGCGCGCCGAAGTGAAATTCGAAGGT
GATACGCTGGTTAACCGTATTGAACTGAAAGGCATCGATTTTAAGGAAGACGGT
AATATTCTGGGCCATAAACTGGAATATAACTTCAATTCGCACAACGTGTACATC
ACCGCAGATAAGCAGAAGAACGGTATCAAGGCTAACTTCAAGATCCGCCATAA
TGTGGAAGATGGCAGCGTTCAACTGGCCGACCACTATCAGCAAAACACCCCGAT
TGGTGATGGCCCGGTCCTGCTGCCGGACAATCATTACCTGAGCACGCAGTCTGT
GCTGAGTAAAGATCCGAACGAAAAGCGTGACCACATGGTCCTGCTGGAATTCGT
GACCGCGGCCGGCATCACGCACGGTATGGACGAACTGTATAAAGGCTCAgatatatc
gggcggtATGgtttttactctggaagattttgttggcgattggcgtcagaccgcgggttataatttggatcaagtcctggaacagggt
ggcgtaagctctctgttccagaacctgggtgtgagcgtgacgccgattcagcgcatcgttctgtccggcgagaacggtctgaaaattg
atattcatgtgatcatcccgtacgaaggcctgagcggtgaccaaatgggtcaaatcgagaaaatctttaaagtcgtctacccagttgac
gatcaccacttcaaggttatcttgcattacggtacgctggtgattgatggtgtgaccccgaatatgattgactatttcggccgtccgtatg
aaggcattgccgtttttgacggtaaaaagatcaccgtcaccggtaccctgtggaatggcaataagattattgacgagcgtctgattaac
ccggacggcagcctgctgttccgcgtgaccatcaacggtgtcacgggttggcgtctgtgcgagcgcatcctggcataaccccaact
ggggtaacctCAgagttctctcagttggggagaccggggacttatcatccaacctgttaCAAAATTTTAGCCGCTAg
agctgttgacaattaatcatcggctcgtataatgtgtggaattgtgagcggataacaattcaaatttgcgcgccacattattattcatacctt
tgtggaccgtattacaaagTGACAATATCTTAATTTAAAAAGGAGGCTTAAATAATGGAAA
AAAAAGAATTTAGGGTACTTATAAAATACTGCTTTCTGAAGGGTAAGAACACCG
TCGAAGCAAAGACGTGGCTGGATAACGAATTTCCGGACAGCGCCCCGGGTAAA
TCAACCATTATCGACTGGTACGCCAAGTTTAAGCGTGGTGAAATGTCGACCGAA
GATGGTGAGCGTTCCGGTCGTCCGAAGGAGGTTGTCACCGACGAAAATATAAAA
AAAATTCACAAAATGATTCTGAACGACCGCAAAATGAAGCTGATCGAAATTGCG
GAAGCTCTGAAAATTAGCAAAGAGCGCGTTGGTCACATCATCCACCAATATCTT
GACATGCGTAAACTGTGTGCGAAATGGGTTCCGCGCGAACTGACCTTTGATCAG
AAACAGCGTCGTGTCGACGATTCTAAGCGTTGCCTGCAGCTGCTGACCCGCAAC
ACGCCGGAGTTCTTCCGCCGCTACGTAACCATGGATGAGACGTGGCTGCACCAC
TATACCCCGGAGTCTAACCGTCAGTCAGCAGAATGGACGGCAACTGGCGAACCG
AGCCCGAAACGCGGCAAAACCCAAAAGAGCGCGGGCAAAGTCATGGCGAGCGT
ATTTTGGGATGCACATGGTATTATCTTCATTGACTACCTGGAAAAGGGTAAGAC
CATAAATTCCGATTACTATATGGCGCTGCTGGAACGCCTGAAAGTTGAAATCGC
GGCAAAACGCCCGCACATGAAAAAGAAGAAGGTACTGTTTCACCAGGACAACG
CCCCCTGTCATAAGTCCCTGCGTACCATGGCGAAGATTCATGAGCTGGGTTTCG
AACTGCTGCCGCACCCGCCGTACAGCCCGGATCTGGCTCCGTCGGATTTTTTTCT
GTTTAGCGACCTGAAGCGTATGCTGGCGGGTAAAAAATTTGGTTGCAACGAAGA
AGTTATCGCTGAAACCGAAGCGTACTTTGAAGCGAAGCCGAAAGAATATTACCA
GAACGGCATTAAGAAACTGGAAGGTCGTTACAATCGCTGTATCGCGCTGGAGGG
CAATTACGTAGAGTAAtctatagtgtcacctaaatGGACCAAAACGAAAAAAGGCCCCCCTT
TCGGGAGGCCTCTTTTCTGGAATTTGGTACCGAGtaatcgatttaaattagtagcccgcctaatgagcgggctt
ttttttaattcccctatttgtttatttttctaaatacattcaaatatgtatccgctcatgagacaataaccctgataaatgcttcaataatat
tgaaaaaggaagagtatgagcattcagcattttcgtgtggcgctgattccgttttttgcggcgttttgcctgccggtgtttgcgcatccgg
aaaccctggtgaaagtgaaagatgcggaagatcaactgggtgcgcgcgtgggctatattgaactggatctgaacagcggcaaaatt
ctggaatcttttcgtccggaagaacgttttccgatgatgagcacctttaaagtgctgctgtgcggtgcggttctgagccgtgtggatgcg
ggccaggaacaactgggccgtcgtattcattatagccagaacgatctggtggaatatagcccggtgaccgaaaaacatctgaccgat
ggcatgaccgtgcgtgaactgtgcagcgcggcgattaccatgagcgataacaccgcggcgaacctgctgctgacgaccattggcg
gtccgaaagaactgaccgcgtttctgcataacatgggcgatcatgtgacccgtctggatcgttgggaaccggaactgaacgaagcg
attccgaacgatgaacgtgataccaccatgccggcagcaatggcgaccaccctgcgtaaactgctgacgggtgagctgctgaccct
ggcaagccgccagcaactgattgattggatggaagcggataaagtggcgggtccgctgctgcgtagcgcgctgccggctggctgg
tttattgcggataaaagcggtgcgggcgaacgtggcagccgtggcattattgcggcgctgggcccggatggtaaaccgagccgtat
tgtggtgatttataccaccggcagccaggcgacgatggatgaacgtaaccgtcagattgcggaaattggcgcgagcctgattaaaca
ttggtaaaccgatacaattaaaggctccttttggagcctttttttttggacgacccttgtccttttccgctgcataaccctgcttcggggtcat
tatagcgattttttcggtatatccatcctttttcgcacgatatacaggattttgccaaagggttcgtgtagactttccttggtgtatccaacgg
cgtcagccgggcaggataggtgaagtaggcccacccgcgagcgggtgttccttcttcactgtcccttattcgcacctggcggtgctc
aacgggaatcctgctctgcgaggctggccgtaggccggccggcgcgccgatctgaagatcagcagttcaacctgttgatagtacgt
actaagctctcatgtttcacgtactaagctctcatgtttaacgtactaagctctcatgtttaacgaactaaaccctcatggctaacgtactaa
gctctcatggctaacgtactaagctctcatgtttcacgtactaagctctcatgtttgaacaataaaattaatataaatcagcaacttaaatag
cctctaaggttttaagttttataagaaaaaaaagaatatataaggcttttaaagcctttaaggtttaacggttgtggacaacaagccaggg
atgtaacgcactgagaagcccttagagcctctcaaagcaattttgagtgacacaggaacacttaacggctgacatggggcgcgccca
g
>Plp (pλ-himar transposase; apramycinR)
(SEQ ID NO: 127)
taacaggttggatgataagtccccggtctgagcacccattagttcaacaaacgaaaattggataaagtgggatatttttaaaatatatattt
atgttacagtaatattgacttttaaaaaaggattgattctaatgaagaaagcagacaagtaagcctatttaaatttgtgtctcaaaatctctg
atgttacattgcacaagataaaaatatatcatcatgaacaataaaactgtctgcttacataaacagtaattactTTTGttagatatatttttt
tgtgTAatTTTGtaatcgttatgcggcagtaaaaggatctattataaggaggcactcaccatgcaatacgaatggcgaaaagccg
agctcatcggtcagcttctcaaccttggggttacccccggcggtgtgctgctggtccacagctccttccgtagcgtccggcccctcga
agatgggccacttggactgatcgaggccctgcgtgctgcgctgggtccgggagggacgctcgtcatgccctcgtggtcaggtctgg
acgacgagccgttcgatcctgccacgtcgcccgttacaccggaccttggagttgtctctgacacattctggcgcctgccaaatgtaaa
gcgcagcgcccatccatttgcctttgcggcagcggggccacaggcagagcagatcatctctgatccattgcccctgccacctcactc
gcctgcaagcccggtcgcccgtgtccatgaactcgatgggcaggtacttctcctcggcgtgggacacgatgccaacacgacgctgc
atcttgccgagttgatggcaaaggttccctatggggtgccgagacactgcaccattcttcaggatggcaagttggtacgcgtcgattat
ctcgagaatgaccactgctgtgagcgctttgccttggcggacaggtggctcaaggagaagagccttcagaaggaaggtccagtcgg
tcatgcctttgctcggttgatccgctcccgcgacattgtggcgacagccctgggtcaactgggccgagatccgttgatcttcctgcatc
cgccagaggcgggatgcgaagaatgcgatgccgctcgccagtcgattggctgagctcataaTAGGGATAATCAGAA
TTGGTTAATTGGTTGTAACACTGGTCTATCATTGATAGGTATAAATTAATACGAC
TCACTAATACTGAACCTATCAGTGATAGATACCGGTGATACCAGCATCGTCTTG
ATGCCCTTGGCAGCACCCTGCTAAGGAGGCAACAAGATGAACACGATTAACATC
GCTAAGAACGACTTCTCTGACATCGAACTGGCTGCTATCCCGTTCAACACTCTGG
CTGACCATTACGGTGAGCGTTTgGCTCGCGAACAGTTGGCCCTTGAGCATGAGTC
TTACGAGATGGGTGAAGCACGCTTCCGCAAGATGTTTGAGCGTCAACTTAAAGC
TGGTGAGGTTGCGGATAACGCTGCCGCCAAGCCTCTCATCACTACCCTACTCCCT
AAGATGATTGCACGCATCAACGACTGGTTTGAGGAAGTGAAAGCTAAGCGCGG
CAAGCGCCCGACAGCCTTCCAGTTCCTGCAAGAAATCAAGCCGGAAGCCGTAGC
GTACATCACCATTAAGACCACTCTGGCTTGCCTAACCAGTGCTGACAATACAAC
CGTTCAGGCTGTAGCAAGCGCAATCGGTCGGGCCATTGAGGACGAGGCTCGCTT
CGGTCGTATCCGTGACCTTGAAGCTAAGCACTTCAAGAAAAACGTTGAGGAACA
ACTCAACAAGCGCGTAGGGCACGTCTACAAGAAAGCATTTATGCAAGTIGTCGA
GGCTGACATGCTCTCTAAGGGTCTACTCGGTGGCGAGGCGTGGTCTTCGTGGCA
TAAGGAAGACTCTATTCATGTAGGAGTACGCTGCATCGAGATGCTCATTGAGTC
AACCGGAATGGTTAGCTTgCACCGCCAAAATGCTGGCGTAGTAGGTCAAGACTC
TGAGACTATCGAACTCGCACCTGAATACGCTGAGGCTATCGCAACCCGTGCAGG
TGCGCTGGCTGGCATCTCTCCGATGTTCCAACCTTGCGTAGTTCCTCCTAAGCCG
TGGACTGGCATTACTGGTGGTGGCTATTGGGCTAACGGTCGTCGTCCTCTGGCGC
TGGTGCGTACTCACAGTAAGAAAGCACTGATGCGCTACGAAGACGTTTACATGC
CTGAGGTGTACAAAGCGATTAACATTGCGCAAAACACCGCATGGAAAATCAAC
AAGAAAGTCCTAGCGGTCGCCAACGTAATCACCAAGTGGAAGCATTGTCCGGTC
GAGGACATCCCTGCGATTGAGCGTGAAGAACTCCCGATGAAACCGGAAGACAT
CGACATGAATCCTGAGGCTCTCACCGCGTGGAAACGTGCTGCCGCTGCTGTGTA
CCGCAAGGACAAGGCTCGCAAGTCTCGCCGTATCAGCCTTGAGTTCATGCTTGA
GCAAGCCAATAAGTTTGCTAACCATAAGGCCATCTGGTTCCCTTACAACATGGA
CTGGCGCGGTCGTGTTTACGCTGTGTCAATGTTCAACCCGCAAGGTAACGATAT
GACCAAAGGACTGCTTACGCTGGCGAAAGGTAAACCAATCGGTAAGGAAGGTT
ACTACTGGCTGAAAATCCACGGTGCAAACTGTGCGGGTGTCGATAAGGTTCCGT
TCCCTGAGCGCATCAAGTTCATTGAGGAAAACCACGAGAACATCATGGCTTGCG
CTAAGTCTCCACTGGAGAACACTTGGTGGGCTGAGCAAGATTCTCCGTTCTGCTT
CCTTGCGTTCTGCTTTGAGTACGCTGGGGTACAGCACCACGGCCTGAGCTATAA
CTGCTCCCTTCCGCTGGCGTTTGACGGGTCTTGCTCTGGCATCCAGCACTTCTCC
GCGATGCTCCGAGATGAGGTAGGTGGTCGCGCGGTTAACTTGCTTCCTAGTGAA
ACCGTTCAGGACATCTACGGGATTGTTGCTAAGAAAGTCAACGAGATTCTACAA
GCAGACGCAATCAATGGGACCGATAACGAAGTAGTTACCGTGACCGATGAGAA
CACTGGTGAAATCTCTGAGAAAGTCAAGCTGGGCACTAAGGCACTGGCTGGTCA
ATGGCTGGCTTACGGTGTTACTCGCAGTGTGACTAAGCGTTCAGTCATGACGCT
GGCTTACGGGTCCAAAGAGTTCGGCTTCCGTCAACAAGTGCTGGAAGATACCAT
TCAGCCAGCTATTGATTCCGGCAAGGGTCTGATGTTCACTCAGCCGAATCAGGC
TGCTGGATACATGGCTAAGCTGATTTGGGAATCTGTGAGCGTGACGGTGGTAGC
TGCGGTTGAAGCAATGAACTGGCTTAAGTCTGCTGCTAAGCTGCTGGCTGCTGA
GGTCAAAGATAAGAAGACTGGAGAGATTCTTCGCAAGCGTTGCGCTGTGCATTG
GGTAACTCCTGATGGTTTCCCTGTGTGGCAGGAATACAAGAAGCCTATTCAGAC
GCGCTTGAACCTGATGTTCCTCGGTCAGTTCCGCTTgCAGCCTACCATTAACACC
AACAAAGATAGCGAGATTGATGCACACAAACAGGAGTCTGGTATCGCTCCTAAC
TTTGTACACAGCCAAGACGGTAGCCACCTTCGTAAGACTGTAGTGTGGGCACAC
GAGAAGTACGGAATCGAATCTTTTGCACTGATTCACGACTCCTTCGGTACCATTC
CGGCTGACGCTGCGAACCTGTTCAAAGCAGTGCGCGAAACTATGGTTGACACAT
ATGAGTCTTGTGATGTACTGGCTGATTTCTACGACCAGTTCGCTGACCAGTTGCA
CGAGTCTCAATTGGACAAAATGCCAGCACTTCCGGCTAAAGGTAACTTGAACCT
CCGTGACATCTTgGAGTCGGACTTCGCGTTCGCGTAAAAGCTTGATATCGAATTC
CTGCAGCCCCGGGGATCCCATGGTACGCGTGCTAGTAATACGACTCACTAATAC
TGAATCCAAACCCTCGTTAGGGGAGCGTCTAATTTTAGGAGATCCAAAATGTCA
AGGCTGGATAAATCAAAAGTAATCAATAGCGCGCTGGAACTGCTGAACGAGGT
CGGCATCGAAGGTCTGACCACCCGCAAGCTGGCGCAAAAACTGGGCGTCGAAC
AACCGACGCTGTACTGGCACGTAAAAAATAAGCGTGCGCTGCTGGACGCACTGG
CAATTGAAATGCTGGATCGTCACCACACCCACTTCTGTCCGCTGGAGGGTGAAT
CATGGCAAGATTTCCTTCGCAACAACGCGAAGTCATTTCGCTGCGCGCTGCTGA
GCCACCGCGATGGAGCAAAAGTTCATCTGGGCACCCGCCCAACGGAGAAACAA
TATGAAACGCTGGAAAACCAGCTTGCCTTCCTGTGCCAGCAGGGTTTCAGCCTT
GAGAACGCGCTGTACGCGCTGAGCGCCGTAGGTCACTTCACCCTGGGCTGTGTT
CTGGAAGACCAAGAACATCAAGTAGCAAAAGAAGAGCGAGAAACCCCTACGAC
CGATTCGATGCCGCCGCTGCTGCGTCAGGCGATTGAACTGTTCGATCACCAGGG
CGCGGAACCGGCATTCCTGTTTGGTCTGGAACTTATTATATGCGGCCTAGAAAA
ACAACTGAAGTGCGAAAGCGGTAGCTAAcgccgaaaaccccgcttcggcggggttttgccgcATAA
CAGGGTAATccccaactggggtaacctGTgagttctctcagttggggAAAAAAAAACCCCGCCCCTG
ACAGGGCGGGGTTTTTTTTTTTCGCCGCGTTGGCTAgTAATACGACTCACTATAG
GGAGAtctcTTAAGTATAAGGAGGAAAAAATATGAGCAAGGGCGAAGAACTGTTT
ACGGGCGTGGTGCCGATTCTGGTGGAACTGGATGGTGATGTCAATGGTCACAAA
TTCAGCGTGCGCGGCGAAGGTGAAGGCGATGCAACCAATGGTAAACTGACGCT
GAAGTTTATTTGCACCACGGGTAAACTGCCGGTTCCGTGGCCGACCCTGGTCAC
CACGCTGACGTATGGTGTTCAGTGTTTCAGTCGTTACCCGGATCACATGAAACG
CCACGACTTTTTCAAGTCCGCGATGCCGGAAGGTTATGTCCAAGAACGTACCAT
CTCATTTAAAGATGACGGCACCTACAAAACGCGCGCCGAAGTGAAATTCGAAG
GTGATACGCTGGTTAACCGTATTGAACTGAAAGGCATCGATTTTAAGGAAGACG
GTAATATTCTGGGCCATAAACTGGAATATAACTTCAATTCGCACAACGTGTACA
TCACCGCAGATAAGCAGAAGAACGGTATCAAGGCTAACTTCAAGATCCGCCATA
ATGTGGAAGATGGCAGCGTTCAACTGGCCGACCACTATCAGCAAAACACCCCGA
TTGGTGATGGCCCGGTCCTGCTGCCGGACAATCATTACCTGAGCACGCAGTCTG
TGCTGAGTAAAGATCCGAACGAAAAGCGTGACCACATGGTCCTGCTGGAATTCG
TGACCGCGGCCGGCATCACGCACGGTATGGACGAACTGTATAAAGGCTCAgatatat
cgggcggtATGgtttttactctggaagattttgttggcgattggcgtcagaccgcgggttataatttggatcaagtcctggaacaggg
tggcgtaagctctctgttccagaacctgggtgtgagcgtgacgccgattcagcgcatcgttctgtccggcgagaacggtctgaaaatt
gatattcatgtgatcatcccgtacgaaggcctgagcggtgaccaaatgggtcaaatcgagaaaatctttaaagtcgtctacccagttga
cgatcaccacttcaaggttatcttgcattacggtacgctggtgattgatggtgtgaccccgaatatgattgactatttcggccgtccgtat
gaaggcattgccgtttttgacggtaaaaagatcaccgtcaccggtaccctgtggaatggcaataagattattgacgagcgtctgattaa
cccggacggcagcctgctgttccgcgtgaccatcaacggtgtcacgggttggcgtctgtgcgagcgcatcctggcataatctagacc
ccaactggggtaacctCAgagttctctcagttggggtagaccggggacttatcatccaacctgttactgtctatagtgtcacctaaattaat
cgatttaaattagtagcccgcctaatgagcgggcttttttttaattcccctatttgtttatttttctaaatacattcaaatatgtatccgctca
tgagacaataaccctgataaatgcttcaataatattgaaaaaggaagagtatgagcattcagcattttcgtgtggcgctgattccgtttttt
gcggcgttttgcctgccggtgtttgcgcatccggaaaccctggtgaaagtgaaagatgcggaagatcaactgggtgcgcgcgtggg
ctatattgaactggatctgaacagcggcaaaattctggaatcttttcgtccggaagaacgttttccgatgatgagcacctttaaagtgctg
ctgtgcggtgcggttctgagccgtgtggatgcgggccaggaacaactgggccgtcgtattcattatagccagaacgatctggtggaa
tatagcccggtgaccgaaaaacatctgaccgatggcatgaccgtgcgtgaactgtgcagcgcggcgattaccatgagcgataacac
cgcggcgaacctgctgctgacgaccattggcggtccgaaagaactgaccgcgtttctgcataacatgggcgatcatgtgacccgtct
ggatcgttgggaaccggaactgaacgaagcgattccgaacgatgaacgtgataccaccatgccggcagcaatggcgaccaccctg
cgtaaactgctgacgggtgagctgctgaccctggcaagccgccagcaactgattgattggatggaagcggataaagtggcgggtcc
gctgctgcgtagcgcgctgccggctggctggtttattgcggataaaagcggtgcgggcgaacgtggcagccgtggcattattgcgg
cgctgggcccggatggtaaaccgagccgtattgtggtgatttataccaccggcagccaggcgacgatggatgaacgtaaccgtcag
attgcggaaattggcgcgagcctgattaaacattggtaaaccgatacaattaaaggctccttttggagcctttttttttggacgacccttgt
ccttttccgctgcataaccctgcttcggggtcattatagcgattttttcggtatatccatcctttttcgcacgatatacaggattttgccaaag
ggttcgtgtagactttccttggtgtatccaacggcgtcagccgggcaggataggtgaagtaggcccacccgcgagcgggtgttcctt
cttcactgtcccttattcgcacctggcggtgctcaacgggaatcctgctctgcgaggctggccgtaggccggTTACTCTACG
TAATTGCCCTCCAGCGCGATACAGCGATTGTAACGACCTTCCAGTTTCTTAATGC
CGTTCTGGTAATATTCTTTCGGCTTCGCTTCAAAGTACGCTTCGGTTTCAGCGAT
AACTTCTTCGTTGCAACCAAATTTTTTACCCGCCAGCATACGCTTCAGGTCGCTA
AACAGAAAAAAATCCGACGGAGCCAGATCCGGGCTGTACGGCGGGTGCGGCAG
CAGTTCGAAACCCAGCTCATGAATCTTCGCCATGGTACGCAGGGACTTATGACA
GGGGGCGTTGTCCTGGTGAAACAGTACCTTCTTCTTTTTCATGTGCGGGCGTTTT
GCCGCGATTTCAACTTTCAGGCGTTCCAGCAGCGCCATATAGTAATCGGAATTT
ATGGTCTTACCCTTTTCCAGGTAGTCAATGAAGATAATACCATGTGCATCCCAAA
ATACGCTCGCCATGACTTTGCCCGCGCTCTTTTGGGTTTTGCCGCGTTTCGGGCT
CGGTTCGCCAGTTGCCGTCCATTCTGCTGACTGACGGTTAGACTCCGGGGTATAG
TGGTGCAGCCACGTCTCATCCATGGTTACGTAGCGGCGGAAGAACTCCGGCGTG
TTGCGGGTCAGCAGCTGCAGGCAACGCTTAGAATCGTCGACACGACGCTGTTTC
TGATCAAAGGTCAGTTCGCGCGGAACCCATTTCGCACACAGTTTACGCATGTCA
AGATATTGGTGGATGATGTGACCAACGCGCTCTTTGCTAATTTTCAGAGCTTCCG
CAATTTCGATCAGCTTCATTTTGCGGTCGTTCAGAATCATTTTGTGAATTTTTTTT
ATATTTTCGTCGGTGACAACCTCCTTCGGACGACCGGAACGCTCACCATCTTCGG
TCGACATTTCACCACGCTTAAACTTGGCGTACCAGTCGATAATGGTTGATTTACC
CGGGGCGCTGTCCGGAAATTCGTTATCCAGCCACGTCTTTGCTTCGACGGTGTTC
TTACCCTTCAGAAAGCAGTATTTTATAAGTACCCTAAATTCTTTtTTTTCCATTAT
TTAAGCCTCCTTTTTAAATTAAGATATTGTCActttgtaatacggtccacaaaggtatgaataataatgtg
gcgcgcaaatttgATGCAACCATTATCACCGCCAGAGGTAAAATAGTCAACACGCACGG
TGTTAGATATTTATCCCTTGCGGTGATAGATTTAACGTTCCGATTTAGTACCTCC
ATATAAAGGAGGATCAAAATGTCAACGAAGAAAAAGCCGCTTACACAAGAGCA
GCTAGAGGACGCACGTCGTCTGAAAGCAATCTATGAGAAGAAAAAGAATGAGC
TGGGTCTGTCTCAGGAAAGCGTAGCCGACAAGATGGGCATGGGTCAGAGCGGC
GTTGGCGCTCTGTTTAACGGTATTAATGCGCTGAACGCGTACAACGCCGCACTG
CTGACCAAGATTCTGAAAGTTTCCGTCGAGGAGTTCTCTCCTTCTATAGCTCGTG
AAATCTATGAAATGTATGAAGCGGTTAGCATGCAACCGTCTCTGCGCTCTGAAT
ACGAATACCCGGTCTTCAGCCACGTTCAAGCAGGTATGTTTAGCCCGgAACTGC
GTACCTTCACCAAAGGTGACGCTGAGCGTTGGGTATCGACTACCAAAAAAGCGA
GCGATAGCGCGTTTTGGCTGGAAGTAGAAGGCAACAGCATGACGGCCCCGACG
GGCAGCAAGCCGTCATTTCCGGATGGTATGCTGATCCTGGTTGATCCTGAGCAG
GCGGTTGAGCCGGGAGACTTTTGCATTGCGCGCCTGGGTGGTGATGAATTCACC
TTTAAGAAGCTGATCCGCGACTCTGGCCAAGTTTTCCTGCAGCCGCTGAATCCGC
AATACCCAATGATCCCGTGCAACGAATCCTGTAGCGTTGTTGGTAAGGTCATTG
CATCCCAGTGGCCGGAAGAAACCTTCGGTTAATTTGTCAGTTACGGCAAGATccg
gccggcgcgccgatctgaagatcagcagttcaacctgttgatagtacgtactaagctctcatgtttcacgtactaagctctcatgtttaac
gtactaagctctcatgtttaacgaactaaaccctcatggctaacgtactaagctctcatggctaacgtactaagctctcatgtttcacgtac
taagctctcatgtttgaacaataaaattaatataaatcagcaacttaaatagcctctaaggttttaagttttataagaaaaaaaagaatatat
aaggcttttaaagcctttaaggtttaacggttgtggacaacaagccagggatgtaacgcactgagaagcccttagagcctctcaaagc
aattttgagtgacacaggaacacttaacggctgacatggggcgcgcccag
Bacterial Promoter sequences
pKan Pl from pIP1433
  (SEQ ID NO: 128)
agattgccttgaatataTTGACAatactgataagataataTATAATatatctttActaccaagacgataaatgcgtcggaaaa
gtttaa
pKan from Tn903
  (SEQ ID NO: 129)
atttaaatttgtgtctcaaaatctctgatgttacattgcacaagataaaaatatatcatcatgaacaataaaactgtctgcttacataaacagt
aat
pChlor from pC194
  (SEQ ID NO: 130)
agcacccattagttcaacaaacgaaaattggataaagtgggatatttttaaaatatatatttatgttacagtaatattgacttttaaaaaagg
attgattctaatgaagaaagcagacaagtaagcctCTTAAGTATAAGGAGGAAAAAAT
ptrfA from RK2
  (SEQ ID NO: 131)
GTTCTTGACAGCGGAACCAATGTTTAGCTAAACTAGAGTCTCCT
pTac
  (SEQ ID NO: 132)
gagctgttgacaattaatcatcggctcgtataatgtgtggaattgtgagcggataacaattCTTAAGTATAAGGAGGA
AAAAAT
pR from Bacteriophage λ
  (SEQ ID NO: 133)
ACGTTAAATCTATCACCGCAAGGGATAAATATCTAACACCGTGCGTGTTGACTA
TTTTACCTCTGGCGGTGATAATGGTTGCAT
CI857 repressor for pR (wildtype)
  (SEQ ID NO: 134)
ATGAGCACAAAAAAGAAACCATTAACACAAGAGCAGCTTGAGGACGCACGTCG
CCTTAAAGCAATTTATGAAAAAAAGAAAAATGAACTTGGCTTATCCCAGGAATC
TGTCGCAGACAAGATGGGGATGGGGCAGTCAGGCGTTGGTGCTTTATTTAATGG
CATCAATGCATTAAATGCTTATAACGCCGCATTGCTTACAAAAATTCTCAAAGTT
AGCGTTGAAGAATTTAGCCCTTCAATCGCCAGAGAAATCTACGAGATGTATGAA
GCGGTTAGTATGCAGCCGTCACTTAGAAGTGAGTATGAGTACCCTGTTTTTTCTC
ATGTTCAGGCAGGGATGTTCTCACCTAAGCTTAGAACCTTTACCAAAGGTGATG
CGGAGAGATGGGTAAGCACAACCAAAAAAGCCAGTGATTCTGCATTCTGGCTTG
AGGTTGAAGGTAATTCCATGACCGCACCAACAGGCTCCAAGCCAAGCTTTCCTG
ACGGAATGTTAATTCTCGTTGACCCTGAGCAGGCTGTTGAGCCAGGTGATTTCTG
CATAGCCAGACTTGGGGGTGATGAGTTTACCTTCAAGAAACTGATCAGGGATAG
CGGTCAGGTGTTTTTACAACCACTAAACCCACAGTACCCAATGATCCCATGCAA
TGAGAGTTGTTCCGTTGTGGGGAAAGTTATCGCTAGTCAGTGGCCTGAAGAGAC
GTTTGGCTGA
CI857 repressor for pR (recoded with synthetic 35bp 5′ UTR)
  (SEQ ID NO: 135)
TCCGATTTAGTACCTCCATATAAAGGAGGATCAAAatgTCAACGAAGAAAAAGCC
GCTTACACAAGAGCAGCTAGAGGACGCACGTCGTCTGAAAGCAATCTATGAGA
AGAAAAAGAATGAGCTGGGTCTGTCTCAGGAAAGCGTAGCCGACAAGATGGGC
ATGGGTCAGAGCGGCGTTGGCGCTCTGTTTAACGGTATTAATGCGCTGAACGCG
TACAACGCCGCACTGCTGACCAAGATTCTGAAAGTTTCCGTCGAGGAGTTCTCT
CCTTCTATAGCTCGTGAAATCTATGAAATGTATGAAGCGGTTAGCATGCAACCG
TCTCTGCGCTCTGAATACGAATACCCGGTCTTCAGCCACGTTCAAGCAGGTATGT
TTAGCCCGgAACTGCGTACCTTCACCAAAGGTGACGCTGAGCGTTGGGTATCGA
CTACCAAAAAAGCGAGCGATAGCGCGTTTTGGCTGGAAGTAGAAGGCAACAGC
ATGACGGCCCCGACGGGCAGCAAGCCGTCATTTCCGGATGGTATGCTGATCCTG
GTTGATCCTGAGCAGGCGGTTGAGCCGGGAGACTTTTGCATTGCGCGCCTGGGT
GGTGATGAATTCACCTTTAAGAAGCTGATCCGCGACTCTGGCCAAGTTTTCCTGC
AGCCGCTGAATCCGCAATACCCAATGATCCCGTGCAACGAATCCTGTAGCGTTG
TTGGTAAGGTCATTGCATCCCAGTGGCCGGAAGAAACCTTCGGTTAA
```

### REFERENCES

Agrawal, A. A. (2001). Phenotypic Plasticity in the Interactions and Evolution of Species. Science 294, 321.
Ajikumar, P. K., Xiao, W.-H., Tyo, K. E. J., Wang, Y., Simeon, F., Leonard, E., Mucha, O., Phon, T. H., Pfeifer, B., and Stephanopoulos, G. (2010). Isoprenoid Pathway Optimization for Taxol Precursor Overproduction in <em>Escherichia coli</em>. Science 330, 70-74.
Angov, E. (2011). Codon usage: nature's roadmap to expression and folding of proteins. Biotechnol J 6, 650-659.
Anzalone, A. V., Koblan, L. W., and Liu, D. R. (2020). Genome editing with CRISPR-Cas nucleases, base editors, transposases and prime editors. Nat Biotechnol 38, 824-844.
Austin, M. J., and Rosales, A. M. (2019). Tunable biomaterials from synthetic, sequence-controlled polymers. Biomaterials Science 7, 490-505.
Bhushan, R., and Bruckner, H. (2004). Marfey's reagent for chiral amino acid analysis: a review. Amino Acids 27, 231-247.
Birchler, J. A. (2015). Promises and pitfalls of synthetic chromosomes in plants. Trends in Biotechnology 33, 189-194.
Bishé, B., Taton, A., and Golden, J. W. (2019). Modification of RSF1010-Based Broad-Host-Range Plasmids for Improved Conjugation and Cyanobacterial Bioprospecting. iScience 20, 216-228.
Blin, K., Shaw, S., Steinke, K., Villebro, R., Ziemert, N., Lee, S. Y., Medema, M. H., and Weber, T. (2019). antiSMASH 5.0: updates to the secondary metabolite genome mining pipeline. Nucleic Acids Research 47, W81-W87.
Blosser, R. S., and Gray, K. M. (2000). Extraction of violacein from Chromobacterium violaceum provides a new quantitative bioassay for N-acyl homoserine lactone autoinducers. Journal of Microbiological Methods 40, 47-55.
Bodor, A., Bounedjoum, N., Vincze, G. E., Erdeiné Kis, Á., Laczi, K., Bende, G., Szilágyi, Á., Kovács, T., Perei, K., and Rákhely, G. (2020). Challenges of unculturable bacteria: environmental perspectives. Reviews in Environmental Science and Bio/Technology 19, 1-22.
Brophy, J. A. N., Triassi, A. J., Adams, B. L., Renberg, R. L., Stratis-Cullum, D. N., Grossman, A. D., and Voigt, C. A. (2018). Engineered integrative and conjugative elements for efficient and inducible DNA transfer to undomesticated bacteria. Nature Microbiology 3, 1043-1053.
Bruand, C., Le Chatelier, E., Ehrlich, S. D., and Janniére, L. (1993). A fourth class of theta-replicating plasmids: the pAM beta 1 family from gram-positive bacteria. Proc Natl Acad Sci USA 90, 11668-11672.
Casini, A., Chang, F.-Y., Eluere, R., King, A. M., Young, E. M., Dudley, Q. M., Karim, A., Pratt, K., Bristol, C., Forget, A., et al. (2018). A Pressure Test to Make 10 Molecules in 90 Days: External Evaluation of Methods to Engineer Biology. Journal of the American Chemical Society 140, 4302-4316.
Cetnar, D. P., and Salis, H. M. (2021). Systematic Quantification of Sequence and Structural Determinants Controlling mRNA stability in Bacterial Operons. ACS Synthetic Biology 10, 318-332.
Chan, L. Y., Kosuri, S., and Endy, D. (2005). Refactoring bacteriophage T7. Mol Syst Biol 1, 2005.0018-2005.0018.
Chanda, D., and Harohally, N. V. (2018). Revisiting Amadori and Heyns synthesis: Critical percentage of acyclic form play the trick in addition to catalyst. Tetrahedron Letters 59, 2983-2988.
Chen, S. P., and Wang, H. H. (2019). An Engineered Cas-Transposon System for Programmable and Site-Directed DNA Transpositions. CRISPR J 2, 376-394.
Choe, J. H., Williams, J. Z., and Lim, W. A. (2020). Engineering T Cells to Treat Cancer: The Convergence of Immuno-Oncology and Synthetic Biology. Annual Review of Cancer Biology 4, 121-139.
Cimermancic, P., Medema, Mamix H., Claesen, J., Kurita, K., Wieland Brown, Laura C., Mavrommatis, K., Pati, A., Godfrey, Paul A., Koehrsen, M., Clardy, J., et al. (2014). Insights into Secondary Metabolism from a Global Analysis of Prokaryotic Biosynthetic Gene Clusters. Cell 158, 412-421.
Clevenger, K. D., Bok, J. W., Ye, R., Miley, G. P., Verdan, M. H., Velk, T., Chen, C., Yang, K., Robey, M. T., Gao, P., et al. (2017). A scalable platform to identify fungal secondary metabolites and their gene clusters. Nat Chem Biol 13, 895-901.
Colloms, S. D., Merrick, C. A., Olorunniji, F. J., Stark, W. M., Smith, M. C. M., Osbourn, A., Keasling, J. D., and Rosser, S. J. (2014). Rapid metabolic pathway assembly and modification using serine integrase site-specific recombination. Nucleic Acids Research 42, e23-e23.
Covington, B. C., Xu, F., and Seyedsayamdost, M. R. (2021). A Natural Product Chemist's Guide to Unlocking Silent Biosynthetic Gene Clusters. Annual Review of Biochemistry 90, 763-788.
Craig, J. W., Chang, F. Y., Kim, J. H., Obiajulu, S. C., and Brady, S. F. (2010). Expanding small-molecule functional metagenomics through parallel screening of broad-host-range cosmid environmental DNA libraries in diverse proteobacteria. Appl Environ Microbiol 76, 1633-1641.
Cuperus, J. T., Groves, B., Kuchina, A., Rosenberg, A. B., Jojic, N., Fields, S., and Seelig, G. (2017). Deep learning of the regulatory grammar of yeast 5′ untranslated regions from 500,000 random sequences. Genome Research.
Curran, K. A., Morse, N.J., Markham, K. A., Wagman, A. M., Gupta, A., and Alper, H. S. (2015). Short Synthetic Terminators for Improved Heterologous Gene Expression in Yeast. ACS Synthetic Biology 4, 824-832.
Davison, E. K., and Brimble, M. A. (2019). Natural product derived privileged scaffolds in drug discovery. Current Opinion in Chemical Biology 52, 1-8.
de Boer, H. A., Comstock, L. J., and Vasser, M. (1983). The tac promoter: a functional hybrid derived from the trp and lac promoters. Proceedings of the National Academy of Sciences of the United States of America 80, 21-25.
de la Cruz, N. B., Weinreich, M. D., Wiegand, T. W., Krebs, M. P. and Reznikoff, W. S. (1993). Characterization of the Tn5 transposase and inhibitor proteins: a model for the inhibition of transposition. J Bacteriol 175, 6932-6938.
Dempwolff, F., Sanchez, S., and Keams, D. B. (2019). TnFLX: a third-generation mariner-based transposon system for &lt;em&gt;Bacillus subtilis&lt;/em&gt. bioRxiv, 825950.
Der, B. S., Glassey, E., Bartley, B. A., Enghuus, C., Goodman, D. B., Gordon, D. B., Voigt, C. A., and Gorochowski, T. E. (2017). DNAplotlib: Programmable Visualization of Genetic Designs and Associated Data. ACS Synthetic Biology 6, 1115-1119.
DeVito, J. A. (2008). Recombineering with tolC as a selectable/counter-selectable marker: remodeling the rRNA operons of Escherichia coli. Nucleic acids research 36, e4-e4.
Donia, M. S., Cimermancic, P., Schulze, C. J., Wieland Brown, L. C., Martin, J., Mitreva, M., Clardy, J., Linington, R. G., and Fischbach, M. A. (2014). A systematic analysis of biosynthetic gene clusters in the human microbiome reveals a common family of antibiotics. Cell 158, 1402-1414.
Donia, M. S., and Fischbach, M. A. (2015). HUMAN MICROBIOTA. Small molecules from the human microbiota. Science (New York, NY) 349, 1254766-1254766.
Du, D., Wang, L., Tian, Y., Liu, H., Tan, H., and Niu, G. (2015). Genome engineering and direct cloning of antibiotic gene clusters via phage ϕBT1 integrase-mediated site-specific recombination in Streptomyces. Scientific Reports 5, 8740.
Elowitz, M. B., and Leibler, S. (2000). A synthetic oscillatory network of transcriptional regulators. Nature 403, 335-338.
Espah Borujeni, A., Mishler, D. M., Wang, J., Huso, W., and Salis, H. M. (2016). Automated physics-based design of synthetic riboswitches from diverse RNA aptamers. Nucleic Acids Res 44, 1-13.
Farkona, S., Diamandis, E. P., and Blasutig, I. M. (2016). Cancer immunotherapy: the beginning of the end of cancer?BMC Medicine 14, 73.
Fredens, J., Wang, K., de la Torre, D., Funke, L. F. H., Robertson, W. E., Christova, Y., Chia, T., Schmied, W. H., Dunkelmann, D. L., Berinek, V., et al. (2019). Total synthesis of Escherichia coli with a recoded genome. Nature 569, 514-518.
Galanie, S., Thodey, K., Trenchard, I. J., Filsinger Interrante, M., and Smolke, C. D. (2015). Complete biosynthesis of opioids in yeast. Science (New York, NY) 349, 1095-1100.
Garcie, C., Tronnet, S., Garénaux, A., McCarthy, A. J., Brachmann, A. O., Pénary, M., Houle, S., Nougayrėde, J.-P., Piel, J., Taylor, P. W., et al. (2016). The Bacterial Stress-Responsive Hsp90 Chaperone (HtpG) Is Required for the Production of the Genotoxin Colibactin and the Siderophore Yersiniabactin in Escherichia coli. The Journal of Infectious Diseases 214, 916-924.
Ghoneim, D. H., Zhang, X., Brule, C. E., Mathews, D. H., and Grayhack, E. J. (2019). Conservation of location of several specific inhibitory codon pairs in the Saccharomyces sensu stricto yeasts reveals translational selection. Nucleic Acids Research 47, 1164-1177.
Glasner, M. E., Truong, D. P., and Morse, B. C. (2020). How enzyme promiscuity and horizontal gene transfer contribute to metabolic innovation. The FEBS Journal 287, 1323-1342.
Goodman, D. B., Church, G. M., and Kosuri, S. (2013). Causes and Effects of N-Terminal Codon Bias in Bacterial Genes. Science 342, 475-479.
Groth, A. C., Olivares, E. C., Thyagarajan, B., and Calos, M. P. (2000). A phage integrase directs efficient site-specific integration in human cells. Proc Natl Acad Sci USA 97, 5995-6000.
Hamilton, R., Watanabe, C. K., and de Boer, H. A. (1987). Compilation and comparison of the sequence context around the AUG startcodons in Saccharomyces cerevisiae mRNAs. Nucleic Acids Research 15, 3581-3593.
Hover, B. M., Kim, S.-H., Katz, M., Charlop-Powers, Z., Owen, J. G., Ternei, M. A., Maniko, J., Estrela, A. B., Molina, H., Park, S., et al. (2018). Culture-independent discovery of the malacidins as calcium-dependent antibiotics with activity against multidrug-resistant Gram-positive pathogens. Nature Microbiology 3, 415-422.
Ichikawa, Y., Morohashi, N., Tomita, N., Mitchell, A. P., Kurumizaka, H., and Shimizu, M. (2016). Sequence-directed nucleosome-depletion is sufficient to activate transcription from a yeast core promoter in vivo. Biochem Biophys Res Commun 476, 57-62.
Inda, M. E., Broset, E., Lu, T. K., and de la Fuente-Nunez, C. (2019). Emerging Frontiers in Microbiome Engineering. Trends in Immunology 40, 952-973.
Iqbal, H. A., Low-Beinart, L., Obiajulu, J. U., and Brady, S. F. (2016). Natural Product Discovery through Improved Functional Metagenomics in Streptomyces. Journal of the American Chemical Society 138, 9341-9344.
Isabella, V. M., Ha, B. N., Castillo, M. J., Lubkowicz, D. J., Rowe, S. E., Millet, Y. A., Anderson, C. L., Li, N., Fisher, A. B., West, K. A., et al.
(2018). Development of a synthetic live bacterial therapeutic for the human metabolic disease phenylketonuria. Nature Biotechnology 36, 857-864.
Jones, J. A., Vernacchio, V. R., Lachance, D. M., Lebovich, M., Fu, L., Shirke, AN., Schultz, V. L., Cress, B., Linhardt, R. J., and Koffas, M. A. G.
(2015). ePathOptimize: A Combinatorial Approach for Transcriptional Balancing of Metabolic Pathways. Scientific Reports 5, 11301.
Kaishima, M., Ishii, J., Matsuno, T., Fukuda, N., and Kondo, A. (2016). Expression of varied GFPs in Saccharomyces cerevisiae: codon optimization yields stronger than expected expression and fluorescence intensity. Scientific Reports 6, 35932.
Keiser, M. J., Roth, B. L., Armbruster, B. N., Ernsberger, P., Irwin, J. J., and Shoichet, B. K. (2007). Relating protein pharmacology by ligand chemistry. Nature Biotechnology 25, 197-206.
Khalil, A. S., and Collins, J. J. (2010). Synthetic biology: applications come of age. Nature Reviews Genetics 11, 367-379.
Kim, C. S., Gatsios, A., Cuesta, S., Lam, Y. C., Wei, Z., Chen, H., Russell, R. M., Shine, E. E., Wang, R., Wyche, T. P., et al. (2020). Characterization of Autoinducer-3 Structure and Biosynthesis in E. coli. ACS Central Science 6, 197-206.
Kingsford, C. L., Ayanbule, K., and Salzberg, S. L. (2007). Rapid, accurate, computational discovery of Rho-independent transcription terminators illuminates their relationship to DNA uptake. Genome Biology 8, R22.
Kudla, G., Murray, A. W., Tollervey, D., and Plotkin, J. B. (2009). Coding-Sequence Determinants of Gene Expression in <em>Escherichia coli</em>. Science 324, 255-258.
Kumar, M. R. (2012). Chromobacterium violaceum: A rare bacterium isolated from a wound over the scalp. Int J Appl Basic Med Res 2, 70-72.
Kushwaha, M., and Salis, H. M. (2015). A portable expression resource for engineering cross-species genetic circuits and pathways. Nature Communications 6, 7832.
Lajoie, M. J., Rovner, A. J., Goodman, D. B., Aeri, H.-R., Haimovich, A. D., Kuznetsov, G., Mercer, J. A., Wang, H. H., Carr, P. A., Mosberg, J. A., et al. (2013). Genomically Recoded Organisms Expand Biological Functions. Science 342, 357.
Lampe, D. J., Akerley, B. J., Rubin, E. J., Mekalanos, J. J., and Robertson, H. M. (1999). Hyperactive transposase mutants of the Himarl mariner transposon. Proceedings of the National Academy of Sciences of the United States of America 96, 11428-11433.
Lampe, D. J., Grant, T. E., and Robertson, H. M. (1998). Factors Affecting Transposition of the &lt;em&gt;Himarl mariner&lt;/em&gt; Transposon &lt;em&gt;in Vitro&lt;/em&gt. Genetics 149, 179.
Lee, S. Y., and Kim, H. U. (2015). Systems strategies for developing industrial microbial strains. Nature Biotechnology 33, 1061-1072. Leskiw, B. K., Lawlor, E. J., Femandez-Abalos, J. M., and Chater, K. F. (1991). TTA codons in some genes prevent their expression in a class of developmental, antibiotic-negative, Streptomyces mutants. Proceedings of the National Academy of Sciences 88, 2461.
Letunic, I., and Bork, P. (2021). Interactive Tree Of Life (iTOL) v5: an online tool for phylogenetic tree display and annotation. Nucleic Acids Research 49, W293-W296.
Leventhal, D. S., Sokolovska, A., Li, N., Plescia, C., Kolodziej, S. A., Gallant, C. W., Christmas, R., Gao, J.-R., James, M. J., Abin-Fuentes, A., et al. (2020). Immunotherapy with engineered bacteria by targeting the STING pathway for anti-tumor immunity. Nature Communications 11, 2739.
Li, G.-W., Oh, E., and Weissman, J. S. (2012). The anti-Shine-Dalgarno sequence drives translational pausing and codon choice in bacteria. Nature 484, 538-541.
Li, S., Zhang, J., Liu, Y., Sun, G., Deng, Z., and Sun, Y. (2018). Direct Genetic and Enzymatic Evidence for Oxidative Cyclization in Hygromycin B Biosynthesis. ACS Chemical Biology 13, 2203-2210.
Li, Y., Li, Z., Yamanaka, K., Xu, Y., Zhang, W., Vlamakis, H., Kolter, R., Moore, B. S., and Qian, P.-Y. (2015). Directed natural product biosynthesis gene cluster capture and expression in the model bacterium Bacillus subtilis. Scientific Reports 5, 9383.
Lithwick, G., and Margalit, H. (2003). Hierarchy of sequence-dependent features associated with prokaryotic translation. Genome Res 13, 2665-2673.
Livak, K. J., and Schmittgen, T. D. (2001). Analysis of Relative Gene Expression Data Using Real-Time Quantitative PCR and the 2-AACT Method. Methods 25, 402-408.
Lopatkin, A. J., and Collins, J. J. (2020). Predictive biology: modelling, understanding and harnessing microbial complexity. Nature Reviews Microbiology 18, 507-520.
Lorenz, R., Bernhart, S. H., Höner zu Siederdissen, C., Tafer, H., Flamm, C., Stadler, P. F., and Hofacker, I. L. (2011). ViennaRNA Package 2.0. Algorithms for Molecular Biology 6, 26.
MacPherson, M., and Saka, Y. (2017). Short Synthetic Terminators for Assembly of Transcription Units in Vitro and Stable Chromosomal Integration in Yeast S. cerevisiae. ACS Synthetic Biology 6, 130-138.
Martinez-Garcia, E., Calles, B., Arévalo-Rodríguez, M., and de Lorenzo, V. (2011). pBAMI: an all-synthetic genetic tool for analysis and construction of complex bacterial phenotypes. BMC Microbiology 11, 38.
Matsuda, Y., Bai, T., Phippen, C. B. W., Nodvig, C. S., Kjaerbølling, I., Vesth, T. C., Andersen, M. R., Mortensen, U. H., Gotfredsen, C. H., Abe, I., et al. (2018). Novofumigatonin biosynthesis involves a non-heme iron-dependent endoperoxide isomerase for orthoester formation. Nature communications 9, 2587-2587.
Mauro, V. P., and Chappell, S. A. (2014). A critical analysis of codon optimization in human therapeutics. Trends Mol Med 20, 604-613.
McClean, K. H., Winson, M. K., Fish, L., Taylor, A., Chhabra, S. R., Camara, M., Daykin, M., Lamb, J. H., Swift, S., Bycroft, B. W., et al. (1997). Quorum sensing and Chromobacterium violaceum: exploitation of violacein production and inhibition for the detection of N-acylhomoserine lactones. Microbiology (Reading) 143 (Pt 12), 3703-3711.
Monteiro, P. T., Oliveira, J., Pais, P., Antunes, M., Palma, M., Cavalheiro, M., Galocha, M., Godinho, C. P., Martins, L. C., Bourbon, N., et al. (2020). YEASTRACT+: a portal for cross-species comparative genomics of transcription regulation in yeasts. Nucleic Acids Research 48, D642-D649.
Morse, N.J., Gopal, M. R., Wagner, J. M., and Alper, H. S. (2017). Yeast Terminator Function Can Be Modulated and Designed on the Basis of Predictions of Nucleosome Occupancy. ACS Synthetic Biology 6, 2086-2095.
Navarro-Muñoz, J. C., Selem-Mojica, N., Mullowney, M. W., Kautsar, S. A., Tryon, J. H., Parkinson, E. I., De Los Santos, E. L. C., Yeong, M., Cruz-Morales, P., Abubucker, S., et al. (2020). A computational framework to explore large-scale biosynthetic diversity. Nature Chemical Biology 16, 60-68.
Newman, D. J., and Cragg, G. M. (2020). Natural Products as Sources of New Drugs over the Nearly Four Decades from 01/1981 to 09/2019. Journal of Natural Products 83, 770-803.
Nielsen, A. A. K., Der, B. S., Shin, J., Vaidyanathan, P., Paralanov, V., Strychalski, E. A., Ross, D., Densmore, D., and Voigt, C. A. (2016). Genetic circuit design automation. Science 352, aac7341.
Nougayrède, J. P., Homburg, S., Taieb, F., Boury, M., Brzuszkiewicz, E., Gottschalk, G., Buchrieser, C., Hacker, J., Dobrindt, U., and Oswald, E. (2006). Escherichia coli induces DNA double-strand breaks in eukaryotic cells. Science 313, 848-851.
Nyerges, Á., Csörgő, B., Nagy, I., Bálint, B., Bihari, P., Lázár, V.,
Apjok, G., Umenhoffer, K., Bogos, B., Pósfai, G., et al. (2016). A highly precise and portable genome engineering method allows comparison of mutational effects across bacterial species. Proceedings of the National Academy of Sciences 113, 2502.
O'Sullivan, D. J., and Klaenhammer, T. R. (1993). High- and low-copy-number Lactococcus shuttle cloning vectors with features for clone screening. Gene 137, 227-231.
Orth, J. D., Thiele, I., and Palsson, B. O. (2010). What is flux balance analysis?Nature biotechnology 28, 245-248.
Ostrov, N., Landon, M., Guell, M., Kuznetsov, G., Teramoto, J., Cervantes, N., Zhou, M., Singh, K., Napolitano, M. G., Moosburner, M., et al. (2016). Design, synthesis, and testing toward a 57-codon genome. Science 353, 819.
Paddon, C. J., Westfall, P. J., Pitera, D. J., Benjamin, K., Fisher, K., McPhee, D., Leavell, M. D., Tai, A., Main, A., Eng, D., et al. (2013). High-level semi-synthetic production of the potent antimalarial artemisinin. Nature 496, 528-532.
Palaniappan, K., Chen, I. M. A., Chu, K., Ratner, A., Seshadri, R., Kyrpides, N.C., Ivanova, N. N., and Mouncey, N.J. (2019). IMG-ABC v.5.0: an update to the IMG/Atlas of Biosynthetic Gene Clusters Knowledgebase. Nucleic Acids Research 48, D422-D430.
Pang, Y. L. J., Poruri, K., and Martinis, S. A. (2014). tRNA synthetase: tRNA aminoacylation and beyond. Wiley Interdiscip Rev RNA 5, 461-480.
Puigbò, P., Romeu, A., and Garcia-Vallvé, S. (2008). HEG-DB: a database of predicted highly expressed genes in prokaryotic complete genomes under translational selection. Nucleic Acids Res 36, D524-527.
Puri, A. W., Owen, S., Chu, F., Chavkin, T., Beck, D. A., Kalyuzhnaya, M. G., and Lidstrom, M. E. (2015). Genetic tools for the industrially promising methanotroph Methylomicrobium buryatense. Appl Environ Microbiol 81, 1775-1781.
Rainey, P. B., and Travisano, M. (1998). Adaptive radiation in a heterogeneous environment. Nature 394, 69-72.
Redden, H., and Alper, H. S. (2015). The development and characterization of synthetic minimal yeast promoters. Nature Communications 6, 7810.
Ren, H., Wang, B., and Zhao, H. (2017). Breaking the silence: new strategies for discovering novel natural products. Current Opinion in Biotechnology 48, 21-27.
Riglar, D. T., Giessen, T. W., Baym, M., Kerns, S. J., Niederhuber, M. J., Bronson, R. T., Kotula, J. W., Gerber, C. K., Way, J. C., and Silver, P. A. (2017). Engineered bacteria can function in the mammalian gut long-term as live diagnostics of inflammation. Nature biotechnology 35, 653-658.
Ronda, C., Chen, S. P., Cabral, V., Yaung, S. J., and Wang, H. H. (2019). Metagenomic engineering of the mammalian gut microbiome in situ. Nature Methods 16, 167-170.
Ross, A. C., Gulland, L. E. S., Dorrestein, P. C., and Moore, B. S. (2015). Targeted Capture and Heterologous Expression of the Pseudoalteromonas Alterochromide Gene Cluster in Escherichia coli Represents a Promising Natural Product Exploratory Platform. ACS Synthetic Biology 4, 414-420.
Saito, K., Green, R., and Buskirk, A. R. (2020). Translational initiation in E. coli occurs at the correct sites genome-wide in the absence of mRNA-rRNA base-pairing. eLife 9, e55002.
Salis, H. M., Mirsky, E. A., and Voigt, C. A. (2009). Automated design of synthetic ribosome binding sites to control protein expression. Nature Biotechnology 27, 946.
Santos, C. N. S., Regitsky, D. D., and Yoshikuni, Y. (2013). Implementation of stable and complex biological systems through recombinase-assisted genome engineering. Nature Communications 4, 2503.
Scherlach, K., and Hertweck, C. (2021). Mining and unearthing hidden biosynthetic potential. Nature Communications 12, 3864.
Scott, M., Gunderson, C. W., Mateescu, E. M., Zhang, Z., and Hwa, T. (2010). Interdependence of Cell Growth and Gene Expression: Origins and Consequences. Science 330, 1099.
Segall-Shapiro, T. H., Meyer, A. J., Ellington, A. D., Sontag, E. D., and Voigt, C. A. (2014). A ‘resource allocator’ for transcription based on a highly fragmented T7 RNA polymerase. Mol Syst Biol 10, 742-742.
Seyedsayamdost, M. R. (2014). High-throughput platform for the discovery of elicitors of silent bacterial gene clusters. Proceedings of the National Academy of Sciences 111, 7266.
Shen, B. (2015). A New Golden Age of Natural Products Drug Discovery. Cell 163, 1297-1300.
Shine, E. E., and Crawford, J. M. (2021). Molecules from the Microbiome. Annual Review of Biochemistry 90, 789-815.
Sidda, J. D., Song, L., Poon, V., Al-Bassam, M., Lazos, O., Buttner, M. J., Challis, G. L., and Corre, C. (2014). Discovery of a family of γ-aminobutyrate ureas via rational derepression of a silent bacterial gene cluster. Chemical Science 5, 86-89.
Skinnider, M. A., Merwin, N.J., Johnston, C. W., and Magarvey, N. A. (2017). PRISM 3: expanded prediction of natural product chemical structures from microbial genomes. Nucleic Acids Res 45, W49-W54.
Smanski, M. J., Bhatia, S., Zhao, D., Park, Y., B A Woodruff, L., Giannoukos, G., Ciulla, D., Busby, M., Calderon, J., Nicol, R., et al. (2014). Functional optimization of gene clusters by combinatorial design and assembly. Nature Biotechnology 32, 1241-1249.
Sugimoto, Y., Camacho, F. R., Wang, S., Chankhamjon, P., Odabas, A., Biswas, A., Jeffrey, P. D., and Donia, M. S. (2019). A metagenomic strategy for harnessing the chemical repertoire of the human microbiome. Science 366, eaax9176.
Tabor, S. (2001). Expression using the T7 RNA polymerase/promoter system. Curr Protoc Mol Biol Chapter 16, Unit16.12.
Temme, K., Zhao, D., and Voigt, C. A. (2012). Refactoring the nitrogen fixation gene cluster from &lt;em&gt;Klebsiella oxytoca&lt;/em&gt. Proceedings of the National Academy of Sciences 109, 7085.
Thomas, C. M., and Smith, C. A. (1987). Incompatibility Group P Plasmids: Genetics, Evolution, and Use in Genetic Manipulation. Annual Review of Microbiology 41, 77-101.
Tian, J., Yan, Y., Yue, Q., Liu, X., Chu, X., Wu, N., and Fan, Y. (2017). Predicting synonymous codon usage and optimizing the heterologous gene for expression in E. coli. Scientific Reports 7, 9926.
Tobias, N.J., and Bode, H. B. (2019). Heterogeneity in Bacterial Specialized Metabolism. Journal of Molecular Biology 431, 4589-4598.
Topp, S., Reynoso, C. M. K., Seeliger, J. C., Goldlust, I. S., Desai, S. K., Murat, D., Shen, A., Puri, A. W., Komeili, A., Bertozzi, C. R., et al. (2010). Synthetic riboswitches that induce gene expression in diverse bacterial species. Applied and environmental microbiology 76, 7881-7884.
Trieu-Cuot, P., Gerbaud, G., Lambert, T., and Courvalin, P. (1985). In vivo transfer of genetic information between gram-positive and gram-negative bacteria. EMBO J 4, 3583-3587.
Tuckey, C., Asahara, H., Zhou, Y., and Chong, S. (2014). Protein synthesis using a reconstituted cell-free system. Current protocols in molecular biology 108, 16.31.11-16.31.22.
Tyo, K. E. J., Ajikumar, P. K., and Stephanopoulos, G. (2009). Stabilized gene duplication enables long-term selection-free heterologous pathway expression. Nature Biotechnology 27, 760-765.
Valdez-Cruz, N. A., Caspeta, L., Perez, N. O., Ramirez, O. T., and Trujillo-Roldán, M. A. (2010). Production of recombinant proteins in E. coli by the heat inducible expression system based on the phage lambda pL and/or pR promoters. Microbial Cell Factories 9, 18.
Vellanoweth, R. L., and Rabinowitz, J. C. (1992). The influence of ribosome-binding-site elements on translational efficiency in Bacillus subtilis and Escherichia coli in vivo. Molecular Microbiology 6, 1105-1114.
Vizcaino, M. I., Guo, X., and Crawford, J. M. (2014). Merging chemical ecology with bacterial genome mining for secondary metabolite discovery. J Ind Microbiol Biotechnol 41, 285-299.
Wachsmuth, M., Findeiβ, S., Weissheimer, N., Stadler, P. F., and Mörl, M. (2013). De novo design of a synthetic riboswitch that regulates transcription termination. Nucleic Acids Res 41, 2541-2551.
Wang, G., Zhao, Z., Ke, J., Engel, Y., Shi, Y.-M., Robinson, D., Bingol, K., Zhang, Z., Bowen, B., Louie, K., et al. (2019a). CRAGE enables rapid activation of biosynthetic gene clusters in undomesticated bacteria. Nature Microbiology 4, 2498-2510.
Wang, H. H., Isaacs, F. J., Carr, P. A., Sun, Z. Z., Xu, G., Forest, C. R., and Church, G. M. (2009). Programming cells by multiplex genome engineering and accelerated evolution. Nature 460, 894-898.
Wang, Z., Wei, L., Sheng, Y., and Zhang, G. (2019b). Yeast Synthetic Terminators: Fine Regulation of Strength through Linker Sequences. ChemBioChem 20, 2383-2389.
Wannier, T. M., Ciaccia, P. N., Ellington, A. D., Filsinger, G. T., Isaacs, F. J., Javanmardi, K., Jones, M. A., Kunjapur, A. M., Nyerges, A., Pal, C., et al. (2021). Recombineering and MAGE. Nature Reviews Methods Primers 1, 7.
Weinreich, M. D., Yigit, H., and Reznikoff, W. S. (1994). Overexpression of the Tn5 transposase in Escherichia coli results in filamentation, aberrant nucleoid segregation, and cell death: analysis of E. coli and transposase suppressor mutations. J Bacteriol 176, 5494-5504.
Wu, S., Ma, X., Zhou, A., Valenzuela, A., Zhou, K., and Li, Y. (2021). Establishment of Strigolactone-Producing Bacterium-Yeast Consortium. bioRxiv, 2021.2006.2029.450423.
Xi, L., Fondufe-Mittendorf, Y., Xia, L., Flatow, J., Widom, J., and Wang, J.-P. (2010). Predicting nucleosome positioning using a duration Hidden Markov Model. BMC Bioinformatics 11, 346.
Xiong, L., Zeng, Y., Tang, R.-Q., Alper, H. S., Bai, F.-W., and Zhao, X.-Q. (2018). Condition-specific promoter activities in Saccharomyces cerevisiae. Microbial Cell Factories 17, 58.
Xu, J., Dong, Q., Yu, Y., Niu, B., Ji, D., Li, M., Huang, Y., Chen, X., and Tan, A. (2018). Mass spider silk production through targeted gene replacement in &lt;em&gt;Bombyx mori&lt;/em&gt. Proceedings of the National Academy of Sciences 115, 8757.
Xue, M., Kim, C. S., Healy, A. R., Wernke, K. M., Wang, Z., Frischling, M. C., Shine, E. E., Wang, W., Herzon, S. B., and Crawford, J. M. (2019). Structure elucidation of colibactin and its DNA cross-links. Science 365, eaax2685.
Yamanaka, K., Reynolds, K. A., Kersten, R. D., Ryan, K. S., Gonzalez, D. J., Nizet, V., Dorrestein, P. C., and Moore, B. S. (2014). Direct cloning and refactoring of a silent lipopeptide biosynthetic gene cluster yields the antibiotic taromycin A. Proceedings of the National Academy of Sciences 111, 1957.
Zhang, M. M., Wong, F. T., Wang, Y., Luo, S., Lim, Y. H., Heng, E., Yeo, W. L., Cobb, R. E., Enghiad, B., Ang, E. L., et al. (2017). CRISPR-Cas9 strategy for activation of silent Streptomyces biosynthetic gene clusters. Nature Chemical Biology 13, 607-609.
Zhang, Z., and Dietrich, F. S. (2005). Mapping of transcription start sites in Saccharomyces cerevisiae using 5′ SAGE. Nucleic Acids Research 33, 2838-2851.
Zhou, K., Qiao, K., Edgar, S., and Stephanopoulos, G. (2015). Distributing a metabolic pathway among a microbial consortium enhances production of natural products. Nature biotechnology 33, 377-383.
Zhou, Z., Chen, X., Sheng, H., Shen, X., Sun, X., Yan, Y., Wang, J., and Yuan, Q. (2020). Engineering probiotics as living diagnostics and therapeutics for improving human health. Microbial cell factories 19, 56-56.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of recoding a nucleic acid coding sequence comprising two, three, four, five, or all six of steps:
(1) selecting the codons of the coding sequence,
(2) implementing N-terminal codon bias;
(3) creating a synthetic or hybrid 5′ regulatory element;
(4) screening for internal ribosome binding sites (RBSs);
(5) randomizing one or more codons upstream of internal RBSs, and
(6) screening for internal terminators,
optionally, wherein the recoding improves expression of the nucleic acid coding sequence in one or more heterologous organisms of interest.

2. The method of claim 1, wherein the nucleic acid coding sequence is a naturally occurring sequence.

3. The method of claims 1 or 2 comprising step (1), wherein codon selection is based partially or completely on the preferred codon distribution in the heterologous organism(s).

4. The method of claim 3, wherein codon usage is selected based on that of highly expressed genes in the heterologous organism(s).

5. The method of any one of claims 1-4 comprising step (1), wherein codon selection is based on codon usage information derived from the genome sequence of a strain(s) of the heterologous organism or downloaded directly from a database(s).

6. The method of any one of claims 3-5 comprising step (1), wherein step (1) comprises depletion of canonically-inhibiting codons, optionally wherein the inhibiting codons are selected from TTA, AGG, CTA, CGA, CGG, CGA, TTG and/or GTG, or a combination thereof.

7. The method of any one of claims 1-6 comprising step (2), wherein step (2) comprises recoding the nucleic acid sequence encoding the N-terminus of a polypeptide encoded by the nucleic acid coding sequence to reduce secondary and/or tertiary structure.

8. The method of claim 7, wherein reducing secondary structure comprises recoding a 5′ terminal stretch of 15-75 base pairs, or any subrange or specific integer therebetween, of the nucleic acid coding sequence.

9. The method of claims 7 or 8 comprising step (2), wherein step (2) comprises using a hybrid codon distribution that biases toward privileged or preferred codons encoding the N-terminus that correlate with high expression levels in the heterologous organism(s).

10. The method of any one of claim 7-9, wherein the recoding of the nucleic acid sequence encoding the N-terminus of a polypeptide comprises the codon adaptation index (CAI) approach and/or the tRNA adaptation index (TAI).

11. The method of any one of claims 1-10 comprising step (3) wherein the synthetic or hybrid regulatory element is designed for versatile regulation across diverse prokaryotes and eukaryotes.

12. The method of any one of claims 1-11 comprising step (3), wherein step (3) comprises creation of a hybrid of eukaryotic and prokaryotic element(s) that can impact gene expression in one, two, three, or more microbial taxa, optionally wherein one or more of the taxa include the heterologous organism(s).

13. The method of any one of claims 1-11 comprising step (3), wherein step (3) comprises utilizing a thermodynamic translation initiation model optionally wherein the thermodynamic translation initiation model defines sequence and/or structural determinants of ribosomal entry, optionally bacterial ribosome entry, and allows predictions of translation initiation rates using a ribosomal binding site (RBS) calculator.

14. The method of any one of claims 1-13 comprising step (3), wherein step (3) comprises consideration of parameters that increase the range of host cells in which the nucleic acid coding sequence can be expressed, optionally highly expressed, optionally wherein the such parameters comprise incorporation of Shine-Dalgamo sequence requirements and/or start codon spacing preferences for the heterologous organism(s).

15. The method of any one of claims 1-14 comprising step (3), wherein step (3) comprises maintaining or recoding the nucleic acid sequence to enrich for poly AT sequence and/or a “AAA” sequence motif immediately upstream of the start codon.

16. The method of any one of claims 1-15 comprising step (3), wherein step (3) comprises maintaining, recoding, or adding to the nucleic acid sequence a synthetic 5′ untranslated region comprising N₁₇(A/U)₆AGGAGN₄AAA (SEQ ID NO:1), and optionally iteratively mutating/varying ‘N’ positions until a desired translation initiation strength is reached, optionally wherein the translation initiation strength is reached by prediction or empirically determined.

17. The method of any one of claims 1-16 comprising step (4), wherein step (4) comprises recoding one or more alternative NTG start codon (s), one or more internal RBS (s), one or more terminator(s), or a combination thereof.

18. The method of claim 17, wherein internal RBSs are NTG sites throughout the CDS in all three coding frames.

19. The method of any one of claims 1-18 comprising step (4), wherein step (4) comprises recoding the sequence upstream of one or more RBS(s) to structurally reduce internal ribosomal entry.

20. The method of any one of claims 1-19 comprising step (4), wherein step (4) comprises predicting ribosome bind strength, calculating thermodynamic parameters, or a combination thereof.

21. The method of any one of claims 1-20 comprising step (5).

22. The method of any one of claims 1-21 comprising step (6), optionally wherein step (6) comprises identifying and optionally recoding rho-independent transcriptional terminators.

23. The method of any one of claims 1-22 comprising iteratively repeating steps (4) and (5) in two or more cycles.

24. The method of claim 23, wherein translation initiation strength is predicted or determined empirically after each cycle, and wherein the cycles are terminated when a desired translation initiation strength is reached.

25. The method of any one of claims 1-24 comprising steps (1), (2), and (3).

26. The method of claim 25 comprising step (4).

27. The method of claims 25 or 26 comprising step (5).

28. The method of any one of claims 25-27 comprising step (6).

29. The method of any one of claims 1-28, wherein one or more steps are computer implemented.

30. A recoded nucleic acid sequence prepared according to the method of any one of claims 1-29.

31. An inducible polymerase promoter expression circuit comprising seed elements or a seed promoter operably linked to an RNA polymerase promoter operable linked to the polymerase coding sequence, wherein the seed element drive initial transcription of the RNA polymerase, and subsequent transcription is auto-regulated through a positive and/or negative regulation of the RNA polymerase promoter.

32. The expression circuit of claim 31, comprising one or more of repressor/operator pair, CRISPRi and/or CRISPRa.

33. The expression circuit of claims 31 or 32, wherein the promoter is pT7 and the RNA polymerase is T7/RNAP, the promoter is pT3 and the RNA polymerase T3/RNAP, or the promoter is pSP6 and the RNA polymerase SP6 RNA polymerase.

34. The expression circuit of any one of claims 31-33, comprising tetO tet-on tetracycline-controlled transcriptional activator sequence, an anhydrotetracyline (aTc) responsive TetR repressor, Tet-off tetracycline-controlled transcriptional repressor, riboswitch (e.g., a theophylline-responsive translational riboswitch), or a combination thereof;
or vanO van-on Vanillin acid-controlled transcriptional activator sequence, an vanillin acid responsive VanR repressor, Van-off tetracycline-controlled transcriptional repressor, riboswitch (e.g., a theophylline-responsive translational riboswitch), or a combination thereof.

35. The expression circuit according to any one of claims 31-34 comprising the architecture of FIG. 4A or any of a, b, c, d, or e of FIG. 4B.

36. The expression circuit of claim 35 comprising a tetO tet-on tetracycline-controlled transcriptional activator sequence, a pT7 promoter driving expression of T7 RNAP through an intervening theophylline-responsive riboswitch, and a pT7 promoter driving expression of a tetR tetracycline repressor.

37. A synthetic genetic element comprising a coding sequence (CDS) operably linked to a hybrid regulatory element suitable for expressing the coding sequence in organisms from two or more different kingdoms.

38. The synthetic genetic element of claim 37, wherein one of the kingdoms is Monera.

39. The synthetic genetic element of claims 37 and 38, wherein one of the kingdoms is Animalia, Plantae, Fungi, or Protista.

40. The synthetic genetic element of any one of claims 37-39, wherein the hybrid regulatory element is suitable for expressing the CDS in prokaryotes and eukaryotes.

41. The synthetic genetic element of any one of claims 37-40, wherein the hybrid regulatory element comprises one or more of a promoter, a 5′ UTR, and 3′ terminator.

42. The synthetic genetic element of any one of claims 37-41, comprising one or more upstream activity sequences (UASs), a core sequence, a TATA box, one or more spacer sequence, or a combination thereof.

43. The synthetic genetic element of claim 42 wherein, the hybrid regulatory element comprises 1-10 UASs operably linked to the promoter.

44. The synthetic genetic element of any one of claims 37-43, wherein the hybrid regulatory element(s) comprises one or more spacer sequence, optionally comprising poly-A or poly-T in an effective amount to deplete the probability of nucleosome occupancy at a TATA box (e.g., TATAAAG) and/or a transcriptional start site (TSS).

45. The synthetic genetic element of any one of claims 37-44, comprising a TATA box.

46. The synthetic genetic element of any one of claims 41-44 wherein the promoter is a natural or synthetic eukaryotic promoter, optionally a natural or synthetic yeast promoter, or a variant thereof.

47. The synthetic genetic element of any one of claims 37-46, wherein the hybrid regulatory element comprises a transcription start site (TSS), optionally comprising the consensus motif [A(A_{rich})₅ NPy A (A/T)NN(A_{rich})₆].

48. The synthetic genetic element of any one of claims 37-47, wherein the hybrid regulatory element comprises any one of SEQ ID NOS:50-98, or variant thereof with at least 70% sequence identity thereto.

49. The synthetic genetic element of any one of claims 37-48, optionally further comprising one or more intervening terminators, optionally flanking the promotor sequence.

50. The synthetic genetic element of any one of claims 37-49, comprising two or more CDS, wherein each CDS is operatively linked its own hybrid regulatory element, wherein the hybrid regulatory element of each CDS are the same, different, or a combination thereof.

51. The synthetic genetic element of claim 50, wherein the two or more CDS together form part or all of a biosynthetic pathway.

52. The synthetic genetic element of claim 51, wherein the biosynthetic pathway is present as a gene cluster in an organism's genome.

53. The synthetic genetic element of any one of claims 39-52, wherein
(i) no pair of UASs is used more than 5, 4, 3, 2, or, 1 time, optionally no more than 3 times, and optionally no triplet of UASs is used more than once;
(ii) promoters range from 100 bp to 250 bp inclusive, or any subrange thereof, or specific integer therefore, optionally 161 bp to 181 bp, in length; and/or
(iii) no spacer or TSS sequence is used more than once.

54. The synthetic genetic element of any one of claims 37-53, wherein
(iv) no ‘NTG’ sequence is used in any spacer to avoid internal start codons; and/or
(v) predicted terminators and RBSs in promoters are removed by randomly inserting or substituting mutating spacer sequences.

55. The synthetic genetic element of any one of claims 37-54, wherein one of more of CDS and optionally the hybrid regulatory sequence operably linked thereto are prepared according to the method of any one of claims 1-30.

56. The synthetic genetic element of any one of claims 37-55 comprising the recoded CDS of claim 30.

57. The synthetic genetic element of any one of claims 37-56 comprising a prokaryotic RBS, a bacterial promoter, a eukaryotic promoter for each CDS, and a eukaryotic terminator.

58. The synthetic genetic element of any one of claims 37-57 further comprising an inducible polymerase promoter expression circuit.

59. The synthetic genetic element of any one of claims 37-58 further comprising an inducible polymerase promoter expression circuit of any one of claims 31-36.

60. The synthetic genetic element of any one of claims 37-59 comprising the architecture of one or more of FIG. 3A, 3B, or 3C.

61. A landing pad for a synthetic genetic element comprising a nucleic acid cassette comprising a nucleic acid sequence encoding an inducible expression control circuit, a promoter operably linked to a reporter gene, a selectable marker, and integration sites flanking the reporter gene.

62. The landing pad of claim 61, further comprising transposase terminal repeats flanking the cassette, followed by a sequence encoding the transposase, preferably which itself does not mobilize into the recipient genome.

63. The landing pad of claim 62, wherein the transposase is independent of host-specific factors and shows little bias in random integration, optionally wherein the transposase is Himar or Tn5.

64. The landing pad of claims 61 and 62, wherein sequence encoding the selectable marker is operably linked to a seed promoter.

65. The landing pad of any one of claims 61-64, wherein the selectable marker is antibiotic selectable.

66. The landing pad of any one of claims 61-65 wherein the inducible expression control circuit is of any one of claims 31-36.

67. The landing pad of any one of claims 61-66 comprising the architecture of FIG. 5A.

68. A method of introducing a landing pad into a host organism comprising introducing into the host cell with the landing pad of any one of claims 61-67.

69. The method of claim 68, wherein introduction comprises transformation or transfection of a vector encoding the landing pad into a first host organism.

70. The method of claims 68 and 69 comprising expressing the transposase.

71. The method of any one of claims 68-70, further comprising introduction of the landing pad into a second host organism by conjugation with the first host organism.

72. The method of any one of claims 68-71 comprising step 1 of FIG. 5A.

73. A host cell comprising the landing pad of any one of claims 61-67 integrated into its genome.

74. The host cell of claim 73 prepared according to the method of any one of claims 67-72.

75. The synthetic genetic element of any one of claims 37-56 flanked by integration sequences.

76. The synthetic genetic element of claim 75 wherein the integration sequences are asymmetrical attB sites.

77. The synthetic genetic element of claims 75 or 76 comprising the architecture of cassette of FIG. 5B.

78. A vector, optionally a suicide vector, comprising encoding or comprising the synthetic genetic element of any one of claims 75-77.

79. The vector of claim 78 further comprising a sequence encoding an integrase optionally phiC31 integrase.

80. The vector of claims 78 and 79 comprising a sequence encoding a selectable marker.

81. A host cell comprising the vector of any one of claims 78-80.

82. A method of introducing a synthetic genetic element into a host cell comprising conjugation of host cell of claim 81 with the host cell of claims 73 or 74.

83. The method of claim 82, wherein the integrase is expressed is facilitates integration of the synthetic genetic element into the landing pad.

84. The method of claim 83, wherein the synthetic genetic element replaces the landing pad's selectable marker.

85. A host cell prepared according to the method of any one of claims 82-84.

86. A host cell comprising the synthetic genetic element of any one of claims 37-60.

87. Any one of sequences disclosed herein including, but not limited to, SEQ ID NOS:1-136, or a variant thereof with at least 70% sequence identity thereto.

88. A hybrid yeast promoter comprising the sequence of any one of SEQ ID NOS:50-98, or a variant thereof with at least 70% sequence identity thereto.

89. A transcriptional start site comprising the sequence of any one of SEQ ID NOS:2-49.
